(19) United States
(12) Patent Application Publication
(10) Pub. No.: US 2026/0315653 A1
(43) Pub. Date: Oct. 15, 2026

(54) ANTICOAGULANT COMPOSITIONS COMPRISING ANTI-PLATELET AND ANTI-INFLAMMATORY DRUGS AND METHODS AND DEVICES FOR THEIR USE

(71) Applicant: Elixir Medical Corporation, Milpitas, CA (US)

(72) Inventors: Motasim SIRHAN, Los Altos, CA (US); Xiaoxia ZHENG, Fremont, CA (US); Vinayak Bhat, Cupertino, CA (US)

(73) Assignee: Elixir Medical Corporation, Milpitas, CA (US)

(21) Appl. No.: 19/383,392

(22) Filed: Nov. 7, 2025

Related U.S. Application Data
(63) Continuation of application No. PCT/US24/29533, filed on May 15, 2024.
(60) Provisional application No. 63/502,527, filed on May 16, 2023.
(60) Provisional application No. 63/592,857, filed on Oct. 24, 2023.

(51) Int. Cl. A61L 27/28; A61L 27/54

(52) U.S. Cl. CPC A61L 27/28; A61L 27/54; A61L 2300/204; A61L 2300/41; A61L 2300/42; A61L 2300/45; A61L 2300/606

(57) ABSTRACT

Devices, systems, methods, and compositions are provided herein including a composition for coupling to a surface of an implantable article, said composition comprising: an anti-coagulant comprising a direct factor Xa inhibitor and at least one of (a) an anti-platelet drug, (b) anti-coagulant comprising a direct factor IIa inhibitor, and (c) an anti-inflammatory drug comprising colchicine.

### CROSS-REFERENCE TO RELATED APPLICATIONS

This application is a continuation of PCT Application No. PCT/US24/29533, filed May 15, 2024, which claims the benefit of U.S. Provisional Application 63/502,527 (Attorney Docket No. 32016-736.101), filed on May 16, 2023, and of U.S. Provisional Application 63/592,857 (Attorney Docket No. 32016-736.102), filed on Oct. 24, 2023, the full disclosures of which are incorporated herein by reference.

### BACKGROUND

Field of the Disclosure: The present disclosure relates generally to medical devices and methods and more particularly to the coating of implantable and other devices with anticoagulant compositions.

Blood coagulation, commonly referred to as “clotting,” is a process designed to stop bleeding from damaged blood vessels. While mammals could not long survive without coagulation to staunch bleeding after injury, clotting in blood vessels after stenting and other intravascular interventions can cause hyperplasia, restenosis, and vessel calcification which are serious complications that can lead to death.

The blood coagulation process requires coagulation factors, calcium, and phospholipids. It is initiated by extrinsic tenase, which forms when factor VIIIa binds to tissue factor. Extrinsic tenase activates factors IX and X. In the presence of calcium, factor IXa binds to negatively charged phospholipid surfaces where it interacts with factor VIIIa to form intrinsic tenase, a complex that efficiently activates factor X. Factor Xa binds to factor Va on negatively charged phospholipid surfaces to form prothrombinase, the complex that activates prothrombin (factor II) to thrombin (factor IIa). Thrombin then converts fibrinogen to fibrin. Activated platelets or monocytes provide negatively charged phospholipid surfaces on which these clotting reactions occur. The intrinsic pathway is initiated by negatively charged surfaces—mediated activation of factor XII (FXII). Such contact activation further propagates thrombin generation by sequential activation of FXI, FIX, FX, and prothrombin. Importantly, thrombin can further activate FXI in a feedback mechanism. Thrombin also activates platelets, which can subsequently support FXI activation. Activation of FXI leads to enhanced thrombin formation, thus forming a positive feedback loop for thrombin formation and consolidation of coagulation. Disorders of coagulation can lead to obstructive clotting (thrombosis) or occlusion of the blood vessel.

Damage to a blood vessel can be caused by, e.g., injurious contact of a device employed in a surgery or intervention with the blood vessel (e.g., a surgical knife cutting a tissue containing the blood vessel, or a deployed stent embedding into the wall of the blood vessel). Damage to the blood vessel can lead to abnormal or undesired recruitment, activation, and/or proliferation of proteins (e.g., fibrin) and cells (e.g., platelets) involved in the coagulation process and other processes at the site of injury, which can result in obstructive clotting or occlusion of the blood vessel.

After vascular injury von Willebrand factor acts as a bridge between endothelial collagen and platelet surface receptors GpIb and promotes platelet adhesion. After adhesion, degranulation from both types of granules takes place with the release of various factors. Release of calcium occurs here simultaneously. Calcium binds to the phospholipids that appear secondary to the platelet activation and provides a surface for assembly of various coagulation factors.

Calcium ions play an important role in the tight regulation of coagulation cascade that is paramount in the maintenance of hemostasis. Calcium ions are essential in coagulation cascade as it is a cofactor for membrane-bound complexes, including intrinsic tenase (FIXa-FVIIIa), extrinsic tenase (FVIIa-TF) and prothrombinase (FXa-FVa) complex.

Under normal physiological conditions, normal vascular endothelium minimizes contact between tissue factor (TF) and plasma procoagulants, but vascular insult expose tissue factor which binds with factor VIIa and calcium to promote the conversion of factor X to Xa.

Other than platelet activation, calcium ions are responsible for complete activation of several coagulation factors, including coagulation Factor XIII (FXIII). FXIII is responsible for covalently cross-linking preformed fibrin clots preventing their premature fibrinolysis, by maintaining the clot architecture and strength. The time lag in generation of first FXIIIa molecule is about 10 minutes to 20 minutes. To prevent the crosslinking of premature fibrin clot, this 10 minutes to 20 minutes is highly critical.

Anticoagulants can be used to prevent the formation of blood clots. Some are used for the prevention or treatment of disorders characterized by abnormal blood clots and emboli. By reducing blood clotting, anticoagulants can prevent deep vein thrombosis, pulmonary embolism, myocardial infarction, and ischemic stroke.

The blood coagulation process requires coagulation factors, calcium, and phospholipids. There are three strategies to prevent coagulation: (1) inhibit the clotting factors, (2) removal of free calcium in the blood, and (3) change of negatively charged phospholipid surfaces as neutral or positive charged such that the clotting reactions cannot occur because these clotting factors can only bind to a negatively charged phospholipid surface which is provided by activated platelets or monocytes. Some anticoagulant drugs act by inactivating thrombin and several other clotting factors that are required for a clot to form. There are other anticoagulant drugs act by removal free calcium ions or inhibit phospholipid such as platelet activating factor. Removal of free calcium ions can be accomplished by the chelating agent EDTA, citate or oxalate. EDTA inhibits the clotting factors intrinsic XII, XI, IX, X and extrinsic VIIa/TF activation and inhibit initial the clotting cycles by depleting/chelating free calcium ions. This is critical for extrinsic pathway, which is the only step (VIIa/TF activated by Ca2+) involved in the cascade of coagulation. The mechanism of EDTA as an agent to prevent clotting induced by a medicated stent is that its inhibition of adenosine, epinephrine, and thrombin-induced platelet aggregation might be more effective than mechanisms that inhibit platelet aggregation more narrowly, as is the case of clopidogrel, which inhibits only adenosine-induced aggregation.

Direct Factor XIa inhibitor is an active-site inhibitor, and it achieves antithrombotic activity without increasing bleeding risk. It also delays the time to clot formation, decreases fibrin incorporation into the clot, and reduces the resistance of clots to fibrinolysis.

Systemic administration of an anticoagulant may be ineffective in preventing or treating disorders associated with coagulation. For example, the concentration of the anticoagulant at or adjacent the site of injury may be insufficient at the appropriate time to prevent or treat disorders associated with coagulation. Furthermore, deficiencies of systemic administration of an anticoagulant can be exacerbated where the patient has a condition (e.g., cardiovascular disease, hypercholesterolemia, or diabetes) that renders the patient more susceptible to a vaso-occlusive event.

Previous attempts to provide local administration of an anticoagulant have had limited to no success in preventing coagulation disorders and/or preventing thrombus (clot) formation particularly after local tissue injury. Furthermore, local injury to a tissue is commonly associated with additional injury to the tissue adjacent (e.g., proximal, distal, etc.) the site of the first injury.

To reduce the partial or total occlusion of the artery by plaque or the collapse of the arterial lining and to reduce the chance of restenosis, a stent or drug coated balloon may be used in the artery to keep the artery open. The agents coated on stent or balloon unfortunately may delay the healing period of the injured tissue, increase tissue factor which may generate or amplify thrombin, fibrin, and/or clot formation, especially within the first 3 hours to 72 hours or more; however, the time lag to generate the first molecule FXIIIa, which is responsible for covalently cross-linking pre-matured fibrin clots and activated by calcium ions is about 10 minutes to 20 minutes. To prevent the crosslinking of premature fibrin clot, this 10 minutes to 20 minutes is highly critical.

Anticoagulants can be used to prevent the formation of blood clots. Some are used for the prevention or treatment of disorders characterized by abnormal blood clots and emboli. By reducing blood clotting, anticoagulants can prevent deep vein thrombosis, pulmonary embolism, myocardial infarction, and ischemic stroke.

The purpose of adding implanted medication to a stent is to prevent thrombin accumulation and restenosis. However, due to an increase in thrombosis at the site of the stent, the risk of death and the risk of myocardial infarction (MI) increased. Although long term treatment with clopidogrel bisulfate plus aspirin for at least 12 months has been suggested as a preventive treatment, there is no evidence that this treatment is effective for more than six months. Clopidogrel also increases the risk of major bleeding episodes.

There is still a need to develop specialized therapeutic compositions for medical devices that can rapidly deliver therapeutic agents, drugs, or bioactive materials directly into a localized tissue area during or following a medical procedure, so as to treat or prevent vascular and nonvascular diseases or conditions such as restenosis or thrombosis. The device should release the therapeutic agent in an effective and efficient manner at the desired target location, where the therapeutic agent should rapidly permeate the target tissue at a local therapeutic level to inhibit one or more of thrombin, fibrin, and/or clot formation prior to amplification of the clotting factors.

It would therefore be desirable to provide devices that locally deliver thrombin/clot formation-inhibiting agents, and optionally other kinds of biologically active agents (e.g., anti-proliferative agents, anti-inflammatory agents, etc.), to the site of injury of a body part or to an area adjacent thereto before, during, and/or after injury.

One approach for inhibiting hyperplasia and related adverse events after stenting has been to coat the stents with immunosuppressive and antiproliferative drugs, such as paclitaxel (taxol), sirolimus (rapamycin) and everolimus. Each of these drugs has found widespread clinical use.

Colchicine is a small molecule anti-inflammatory (anti-mitotic) drug which blocks mitotic cells in metaphase. Colchicine is administered orally for the systemic treatment of inflammatory disease, such as gout and pericarditis, and its systemic use has been proposed for treating restenosis (O'Keefe (1992) JACC 19:1597-1600). Systemic colchicine, however, has side effects including toxicity which require treatment at lower doses and/or for short durations, reducing its effectiveness and preventing widespread adoption for the treatment of restenosis. Other anti-inflammatory agents, such NSAIDs (nonsteroidal) and steroids, are used as anti-inflammatories for treating restenosis, but colchicine has advantages such as inhibiting endothelial cell dysfunction and inflammation, inhibiting vascular inflammation, inhibiting vascular inflammation after injury such as PCI, inflammation from vascular plaque or disease condition, inhibiting inflammation from implantable material, inhibiting or reducing cardiovascular chest pain (angina), reducing hsCRP (an inflammation marker), inhibiting one or more of NLRP3 inflammasome, 1β, IL-18, and/or IL-6, so it would be useful to provide improved formulations for using colchicine to treat restenosis in the vasculature after stenting and other vascular interventions.

Coating stents with colchicine in a polymer carrier for treating restenosis has been proposed as an alternative to oral administration, see e.g., U.S. Pat. No. 5,776,184, but colchicine has not been clinically adopted for such a use.

It would therefore be desirable to provide improved drug coatings for stents and other implantable devices that employ colchicine as a primary active agent. In particular, it would be desirable to identify other drug(s) which could be combined with colchicine or other immunosuppressive drugs such as sirolimus, or other antiplatelet drugs such as tirofiban, ticagrelor, aspirin in drug coatings for stents and other implantable devices where the combination achieves a synergistic inhibitory effect greater than achieved by coatings employing either drug alone. At least some of these objectives will be met by the disclosures described and claimed herein.

The disclosure also provides methods of using such devices and other forms of therapy in treating clotting, and in improving or promoting wound healing, at the injury site or at an area adjacent thereto.

Listing of Background Art. Relevant background art includes U.S. Pat. Nos. 9,770,349; 9,901,663; 6,500,855; 8,409,272; 8,946,219; US2003/0158120; US2005004663; US2005/0064006; US2009/0075949; US2010/0003542; US2010/0130543; US2010/0184729; US2010/049328; US2013/189329; US2015/157771; US2018/0000490; EP1849434; CA2464290; WO2013:007840; WO2013:056060; WO2020/210613; WO2020/210629; U.S. Pat. Nos. 8,617,104; 8,715,771; 9,066,990; 9,439,792; 9,492,294; 9,656,003; U.S. Ser. No. 10/278,839; US20070043418; and US202000384246; O'Keefe (1992) JACC 19:1597-1600 and U.S. Pat. No. 5,776,184 have been described above.

The subject matter of this application is also related to that of the following commonly owned applications: International Patent Application PCT/US2022/50099 filed Nov. 16, 2022, entitled “Anticoagulant compounds comprising chelating agents and cationic anti-coagulation enhancers and methods and devices for their use” (Attorney Docket No. 32016-729.601); International Patent Application PCT/US2021/034108 filed May 25, 2021, entitled “Anticoagulant compositions and methods and devices for their use” (Attorney Docket No. 32016-720.601); International Patent Application PCT/US2021/044414 filed Aug. 3, 2021, entitled “Anticoagulant compositions and methods and devices for their pulmonary use” (Attorney Docket No. 32016-730.601); International Patent Application PCT/US2021/049964 filed Sep. 10, 2021, entitled “Anticoagulant compositions and methods and devices for their ophthalmic use” (Attorney Docket No. 32016-720.601); International Patent Application No. PCT/US2007/078317, filed Sep. 12, 2007, entitled “Macrocyclic lactone compounds and methods for their use” (Attorney Docket No. 32016-704.601); International Patent Application No. PCT/US2008/056501, filed Mar. 11, 2008, entitled “Macrocyclic lactone compounds and methods for their use” (Attorney Docket No. 32016-704.602); International Patent Application No. PCT/US2009/059396, filed Oct. 2, 2009, entitled “Macrocyclic lactone compounds and methods for their use” (Attorney Docket No. 32016-709.601); International Patent Application No. PCT/US2011/054637, filed Oct. 3, 2011, entitled “Macrocyclic lactone compounds and methods for their use” (Attorney Docket No. 32016-711.601); each of which are incorporated herein by reference for all purposes in their entireties.

### SUMMARY OF THE DISCLOSURE

The disclosed technology provides a composition for coupling to a surface of an implantable article, typically a vascular implant, such as stent, a stent-graft, a prosthetic valve, an occlusion device, or the like. Use of the compositions, however, in not limited to vascular implants and extends to a variety of other prosthetic, diagnostic, and other implants as described further hereinbelow. Such implants will usually comprise base structure, such as an expandable scaffold in the case of stents and stent-grafts or housings in the case of other implants. The implants will typically have an outer surface and/or surface or internal cavities, suitable to coating or other deposition of the compositions described and claimed herein.

The terms “coupling” and “couple” as used to herein and in the claims broadly denote that the disclosed compositions and/or any components thereof are coated, attached, sequestered, loaded, linked, or otherwise immobilized on a surface of a base structure of a surgical article, including permanently implantable articles such as scaffolds, stents, stent-grafts, prosthetic valves, orthopedic implants, electrical stimulators, such as pacemakers, nerve stimulators, and the like. In other cases, the surfaces may be on a catheters and other surgical tools that are temporarily introduced into a patient's vasculature or vascularized space, such as vascular catheters, guidewires, guide catheters, sheaths, and the like. In some instances, the disclosed compositions and/or any components may be sequestered otherwise loaded into receptacles or depots formed in or on a surface of the base structure of the surgical article.

The coupling surface can be any surface on the surgical article, particularly those which are exposed directly or indirectly to patient blood, often being an external surface and at other times being an internal surface. For example, on stents and other scaffolds, the compositions and/or any components thereof may be coupled to outer surfaces, inner (luminal) surfaces, and/or edges. On catheters, the compositions and/or any components thereof may be coupled to an outer balloon surface, an outer shaft surface, or the like. On more complexly structured surgical articles, such as prosthetic heart valves, the surfaces may be on an outer base or anchor feature as well as on internal valve and other mechanical components.

The disclosed compositions and/or components thereof may be coated or otherwise coupled to the surgical article surface directly and/or indirectly and in one, two, three, or more layers, where individual layers may be homogeneous or non-homogenous. Successive layers of the composition may be formed directly or indirectly over one another, and intermediate “non-active” layers (not containing drugs of the disclosed technology), such as barrier layers, rate-controlling layers, permeable and semi-permeable membranes, and the like, may be disposed between successive “active” layers (containing drugs of the disclosed technology).

In a first aspect, the compositions comprise an anti-coagulant comprising a direct factor Xa inhibitor and at least one of (a) an anti-platelet drug, (b) anti-coagulant comprising a direct factor IIa inhibitor, and (c) an anti-inflammatory drug comprising colchicine, often including at least at least two of (a) the anti-platelet drug, (b) the direct factor IIa inhibitor, and (c) the anti-inflammatory drug comprising colchicine, and frequently, including all three of (a) an anti-platelet drug, (b) a direct factor IIa inhibitor, and (c) anti-inflammatory drug comprising colchicine.

In some instances, the direct factor Xa inhibitor comprises at least one drug selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052). For example, the direct factor Xa inhibitor may comprises rivaroxaban, apixaban, or a salt, isomer, solvate, derivative, metabolite, or prodrug thereof.

In some instances, the compositions comprise at least one an anti-platelet drug selected from the group consisting of a Cox 1 inhibitor, a Cox 2 inhibitor, a IIb/IIIa inhibitor, and a P2Y12 inhibitor, or a salt or analog thereof. For example, the anti-platelet drug may comprise tirofiban, a salt, analog, or metabolite thereof. In further examples, the anti-platelet drug may comprise ticagrclor, a salt, isomer, solvate, derivative, metabolite, or prodrug thereof. In still further examples, the anti-platelet drug may comprise acetylsalicylic acid (aspirin, a Cox 1/2 inhibitor). In additional examples, the antiplatelet drug may comprise clopidogrel or an active metabolite of clopidogrel, salt, isomer, solvate, prodrug, metabolites, or derivative thereof.

In some instances, the at least one direct factor IIa inhibitor may be selected from the group consisting of argatroban, dabigatran, ximelagatran, melagatran, efegatran, inogatran, atecegatran metoxil (AZD-0837), hirudin, hirudin analogs, bivalirudin, desirudin, and lepirudin. For example, the direct factor IIa inhibitor drug may comprise argatroban, a salt, solvate, isomer, derivative, or metabolite, or prodrug thereof.

In a second aspect, the compositions may comprise an anti-coagulant comprising a direct factor IIa inhibitor and at least one of (a) an anti-platelet drug, (b) an anti-coagulant comprising a direct factor IIa inhibitor, and (c) an anti-inflammatory drug comprising colchicine, often including at least two of (a) the anti-platelet drug, (b) the anti-coagulant comprising the direct factor IIa inhibitor, and (c) the anti-inflammatory drug comprising colchicine, and frequently including all three of (a) an anti-platelet drug, (b) the anti-coagulant comprising the direct factor IIa inhibitor, and (c) the anti-inflammatory drug comprising colchicine.

In some instances, the direct factor IIa inhibitor is selected from the group consisting of argatroban, dabigatran, ximelagatran, melagatran, efegatran, inogatran, atecegatran metoxil (AZD-0837), hirudin, hirudin analogs, bivalirudin, desirudin, and lepirudin. For example, the direct factor IIa inhibitor drug may comprise rivaroxaban, argatroban, or a salt, analog, or metabolite thereof.

In some instances, the compositions may comprise an anti-platelet drug selected from the group consisting of a Cox 1 inhibitor, a Cox 2 inhibitor, a IIb/IIIa inhibitor, and a P2Y12 inhibitor, or a salt or analog thereof. For example, the anti-platelet drug may comprise tirofiban, ticagrelor, or a salt, analog, or metabolite thereof; acetylsalicylic acid (a Cox1/2 inhibitor); or clopidogrel or the active metabolite of clopidogrel, or a salt, solvate, isomer, derivative, prodrug, or metabolite thereof.

In some instances, the direct factor XIIa inhibitor may comprise at least one drug selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1 h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052). For example, the direct factor Xa inhibitor may comprise apixaban, a salt, isomer, solvate, isomer, derivative metabolite, or prodrug thereof and/or rivaroxaban, a salt, solvate, isomer, derivative, or metabolite thereof.

In a third aspect, the composition for coupling to a surface of an implantable article may comprise a direct factor Xa inhibitor and at least two and often three of (a) a direct factor IIa inhibitor, (b) an anti-platelet drug selected from the group consisting of a Cox 1 inhibitor, a Cox 2 inhibitor, a IIb/IIIa inhibit, and a P2y12 inhibitor, and (c) an anti-inflammatory drug.

In some instances, the direct factor Xa inhibitor may comprise at least one drug selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052), or a salt, analog or metabolite thereof. For example, the direct factor Xa inhibitor may comprise rivaroxaban, or apixaban, a salt, isomer, solvate, derivative, metabolite, or prodrug thereof or an analog thereof and/or rivaroxaban, or a salt, isomer, derivative solvate, metabolite, or prodrug thereof or may comprise argatroban, or a salt, isomer, derivative, solvate, prodrug, or metabolite thereof.

In some instances, the anti-platelet drug may comprise at least one drug selected from the group consisting of tirofiban, ticagrelor, a Cox 1 inhibitor, a Cox 2 inhibitor, a IIb/IIIa inhibitor, and a P2Y12 inhibitor, or a salt, isomer, solvate, derivative, prodrug, or metabolite thereof.

In some instances, the anti-inflammatory drug may comprise colchicine or n isomer, derivative metabolite, or prodrug thereof.

In a fourth aspect, the composition for coupling to a surface of an implantable article may comprise apixaban, rivaroxaban or a salt, isomer, solvate, derivative prodrug, or metabolite thereof, argatroban, or a salt, isomer, derivative, solvate, prodrug, or metabolite thereof,
tirofiban, or a salt, analog or metabolite thereof, and colchicine.

In some instances, the compositions may further comprise an additional an anti-platelet drug,

In some instances, the compositions may further comprise an additional anti-inflammatory drug.

In a fifth aspect, the disclosed technology provides a surgical article comprising a base structure, where the disclosed composition and/or one or more components thereof are coupled to a surface or interior of the base structure.

In some instances, the composition, and/or individual components thereof, is/are coupled to the surface in a single layer, where the single layer may be homogeneous or non-non-homogeneous. By “homogeneous,” it is meant that the drug components of the composition are mixed and distributed relatively uniformly in the individual layer. By “non-homogeneous,” it is meant that the drug components of the composition are distributed non-uniformly in the individual layer.

In some instances, the composition, and/or individual components thereof, is/are coupled to the surface in two layers of the composition. In such instances, each layer may comprise some or all drug components of the composition, and the drug components of each layer may be present in homogenous or non-homogeneous mixtures.

In addition to “active” layers comprising some or all drug components of the disclosed compositions, the surfaces may be directly or indirectly cover, in whole or in part, by intermediate, typically “inactive,” layers which do not carry active amounts of the compositions and/or drug components thereof. Exemplary intermediate layers comprise barrier layers, rate-controlling layers, permeable and semi-permeable membranes, and the like, and may be disposed between successive active layers. Intermediate layer may be disposed over and/or between other adjacent layer and may comprise a drug in addition to the drugs in the composition.

The drugs in the composition coupled to the surface of the surgical article typically have a release rate in a range from 1 day to one year, often having a release rate in a range from 1 day to 3 days, from 1 day to 30 days, or 1 day to 90 days.

The present disclosure provides an implantable scaffold comprising a scaffold structure having a surface configured to be expanded in a patient's body. A therapeutic composition, comprising at least one chelating agent is present on a surface of the scaffold, where the therapeutic composition is formulated for a rapid release of the chelating agent into an environment surrounding the scaffold upon implantation of the scaffold structure in said environment.

The present disclosure also provides the therapeutic composition comprises colchicine and/or tirofiban and at least one antiproliferative drug. The colchicine and/or tirofiban is present in a carrier configured to release the colchicine and/or tirofiban over a period of at least one week in an amount sufficient to inhibit inflammation resulting from the implantation of the scaffold. The at least one antiproliferative drug is present in the same or a different carrier and is configured to release the antiproliferative drug over a period of at least one week in an amount sufficient to inhibit smooth cell muscle (SMC) proliferation.

The at least one antiproliferative drug typically comprises an mTOR inhibitor, usually being selected from a group consisting of sirolimus, biolimus, everolimus, myolimus, novolimus, ridaforolimus, temsirolimus, zotarolimus, and salts, isomers, solvates, analogs, derivatives, metabolites, or prodrugs thereof more usually comprising sirolimus, or a salts, isomer, solvate, analog, derivative, metabolite, or prodrug thereof.

The present disclosure also provides the therapeutic composition comprises colchicine and/or tirofiban and at least one antiproliferative drug. The colchicine and/or tirofiban is present in a carrier configured to release the colchicine and/or tirofiban over a period of at least one week in an amount sufficient to inhibit inflammation resulting from the implantation of the scaffold. The at least one antiproliferative drug is present in the same or a different carrier and is configured to release the antiproliferative drug over a period of at least one week in an amount sufficient to inhibit smooth cell muscle (SMC) proliferation.

The at least one antiproliferative drug typically comprises an mTOR inhibitor, usually being selected from a group consisting of sirolimus, biolimus, everolimus, myolimus, novolimus, ridaforolimus, temsirolimus, zotarolimus, and salts, isomers, solvates, analogs, derivatives, metabolites, or prodrugs thereof more usually comprising sirolimus, or a salts, isomer, solvate, analog, derivative, metabolite, or prodrug thereof.

The present disclosure provides devices, compositions, and methods for their use. The devices comprise implantable scaffolds and other structures having one or more surfaces configured for internal use within a patient's body most commonly implantable stents and grafts, but also vascular, coronary, orthopedic, and other articles intended for long-term and short-term implantation. The coatings are configured for fully or partially covering the surfaces of the devices, including outer and inner device surfaces, typically being deposited on, over, or in one or more of the surfaces, including within depots formed on or by the surfaces.

The coatings include compositions having one or more drugs or other active substances such, as anti-coagulant drugs, antiplatelet drugs, antiproliferative drugs, colchicine, and chelating agents. The compositions are formulated to release one or more of the drugs into an implanted environment, such as a blood vessel lumen, a coronary valve, or solid tissue, to inhibit clot formation, promote clot dissolution, inhibit inflammation, inhibit vessel injury, increase time before clotting, and/or inhibit cell proliferation. The compositions will typically be formulated to provide delayed or controlled release of the drugs from the structure into the implanted environment.

The compositions may provide all drugs, excipients, binders, and other components as a single formulation intended for simultaneous coating onto a surface of an article but will more often be proved as a plurality of separate components to allow sequential application or deposition onto the article so that the composition is formed in situ on the article.

In some examples, the scaffold structure may be configured to be expanded in a vascular lumen or any other target site in the patient's body. The therapeutic composition may be present at least partly on the surface of scaffold structure. Alternatively, the therapeutic composition is present at least partly within a cavity or reservoir within the scaffold structure.

In further aspects, the present disclosure provides compositions suitable for coating on or in surfaces of the various implantable articles described herein. The compositions will each include a plurality of drugs or other active and inactive components, where the active components are classified as anti-coagulant drugs, antiplatelet drugs, antiproliferative drugs, colchicine, and chelating agents. The compositions will typically be formulated to provide delayed or controlled release of the drugs from the structure into an implanted environment and, upon release and over time, to inhibit clot formation, promote clot dissolution, inhibit inflammation, inhibit vessel injury, increase time before clotting, and/or inhibit cell proliferation. The compositions may provide all drugs, excipients, binders, and other components as a single formulation intended for simultaneous coating onto a surface of an article but will more often be proved as a plurality of separate components to allow sequential application or deposition onto the article so that the composition is formed in situ on the article.

A first composition in accordance with the principles of the present disclosure comprises (a) at least one of a direct factor IIa inhibitor and a direct factor Xa inhibitor, (b) colchicine, and (c) a first antiplatelet drug comprising tirofiban. The (a) at least one of a direct factor IIa inhibitor and a direct factor Xa inhibitor, (b) colchicine, and (c) first antiplatelet drug are formulated to release from the surface of an implantable article in amounts sufficient to inhibit inflammation and/or thrombosis resulting from an injury.

In some instances, the (a) at least one of a direct factor IIa inhibitor and a direct factor Xa inhibitor, (b) colchicine, and (c) first antiplatelet drug of the first composition are formulated to treat an injury that resulted from implantation of the article.

In some instances, the (a) at least one of a direct factor IIa inhibitor and a direct factor Xa inhibitor, (b) colchicine, and (c) first antiplatelet drug of the first composition are formulated to treat the injury without causing any one or more of bleeding, a risk of bleeding risk, and bleeding associated with an administration of a systemic antithrombotic drug.

In some instances, the first composition comprises at least one factor IIa inhibitor selected from a group consisting of argatroban, dabigatran, ximelagatran, melagatran, efegatran, inogatran, atecegatran metoxil (AZD-0837), hirudin, hirudin analogs, bivalirudin, desirudin, and lepirudin, where the at least one direct factor IIa inhibitor may comprise argatroban, or a salt, isomer, solvate, analog, derivative, metabolite, or prodrugs thereof.

In some instances, the first composition comprises at least one factor Xa inhibitor selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052). For example, the direct factor Xa inhibitor comprises apixaban, or a salt, isomer, solvate, analog, derivative, metabolite, or prodrugs thereof. For example, the direct factor Xa inhibitor comprises rivaroxaban, or a salt, isomer, solvate, analog, derivative, metabolite, or prodrugs thereof.

In some instances, the first composition comprises at least a second antiplatelet drug different from the first antiplatelet drug, where the second antiplatelet drug may comprise one or more of a glycoprotein IIb/IIIa inhibitor, a COX inhibitor, an ADP receptor/P2Y12 inhibitor, a prostaglandin analogue, a thromboxane inhibitor, and a phosphodiesterase inhibitors.

In some instances, the first composition comprises at least a third antiplatelet drug different than the first and second antiplatelet drugs, wherein the third antiplatelet drug may comprise one or more of a glycoprotein IIb/IIIa inhibitor, a COX inhibitor, an ADP receptor/P2Y12 inhibitor, a prostaglandin analogue, a thromboxane inhibitor, and a phosphodiesterase inhibitors.

In some instances, the first composition comprises a chelating agent, where the chelating agent may be selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentascetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), deferoxamine (DFO), a surfactant-EDTA complex, quaternary ammonium EDTA salt, benzalkonium EDTA salt, EDTA complex, salts, analogue, solvate, hydrate and derivatives thereof.

In some instances, the first composition comprises at least one antiproliferative drug, where the antiproliferative drug may comprise an mTOR inhibitor.

A second composition for coating a surface of an implantable article in accordance with the principles of the present disclosure comprises (a) a direct factor IIa inhibitor, (b) a direct factor Xa inhibitor, and (c) a first antiplatelet drug comprising a glycoprotein IIb/IIIa inhibitor, where (a) the direct factor IIa inhibitor, (b) the direct factor Xa inhibitor, and (c) the first antiplatelet drug are formulated to release from the surface of the article in amounts sufficient to inhibit inflammation and/or thrombosis resulting from an injury.

In some instances, the (a) factor IIa inhibitor, (b) factor Xa inhibitor, and (c) first antiplatelet drug of the second composition are formulated to treat an injury that resulted from implantation of the article.

In some instances, the (a) factor IIa inhibitor, (b) factor Xa inhibitor, and (c) first antiplatelet drug of the second composition are formulated to treat the injury without causing any one or more of bleeding, a risk of bleeding risk, and bleeding associated with an administration of a systemic antithrombotic drug.

In some instances, the second composition further comprises (d) colchicine which in combination with (a) the direct factor IIa inhibitor, (b) the direct factor Xa inhibitor, and (c) first antiplatelet drug is formulated to release from the surface of the article in an amount sufficient to enhance inhibition of inflammation and/or thrombosis resulting from the injury.

In some instances, the direct factor IIa inhibitor of the second composition is selected from a group consisting of argatroban, dabigatran, ximelagatran, melagatran, efegatran, inogatran, atecegatran metoxil (AZD-0837), hirudin, hirudin analogs, bivalirudin, desirudin, and lepirudin, where the direct factor IIa inhibitor typically comprises argatroban, or a salt, isomer, solvate, analog, derivative, metabolite, or prodrugs thereof.

In some instances, the direct factor Xa inhibitor the second composition is selected from a group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1 h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or cribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052). For example, the direct factor Xa inhibitor comprises apixaban, or a salt, isomer, solvate, analog, derivative, metabolite, or prodrugs thereof. For example, the direct factor Xa inhibitor comprises rivaroxaban, or a salt, isomer, solvate, analog, derivative, metabolite, or prodrugs thereof.

In some instances, the glycoprotein IIb/IIIa inhibitor of the second composition is selected from a group consisting of abciximab, eptifibatide, orbofiban, roxifiban, sibrafiban, and tirofiban.

In some instances, the second composition further comprises at least a second antiplatelet drug different than the first antiplatelet drug, wherein the second antiplatelet drug may comprise one or more of a glycoprotein IIb/IIIa inhibitor, a COX inhibitor, an ADP receptor/P2Y12 inhibitor, a prostaglandin analogue, a thromboxane inhibitor, and a phosphodiesterase inhibitors.

In some instances, the second composition further comprises at least a third antiplatelet drug different than the first and second antiplatelet drugs, where the third antiplatelet drug may comprise one or more of a glycoprotein IIb/IIIa inhibitor, a COX inhibitor, an ADP receptor/P2Y12 inhibitor, a prostaglandin analogue, a thromiboxane inhibitor, and a phosphodiesterase inhibitors.

In some instances, the second composition further comprises a chelating agent, where the chelating agent may be selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TrFD), dimercaptosuccinic acid (DMSA), diethylenetriaminepcntaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), deferoxamine (DFO), a surfactant-EDTA complex, quaternary ammonium EDTA salt, benzalkonium EDTA salt, EDTA complex, salts, analogue, solvate, hydrate and derivatives thereof.

In some instances, the second composition further comprises at least one antiproliferative drug, wherein the antiproliferative drug may comprise an mTOR inhibitor.

A third composition for coating a surface of an implantable article in accordance with the principles of the present disclosure comprising (a) a direct factor IIa inhibitor, (b) a direct factor Xa inhibitor, (c) a first antiplatelet drug comprising a glycoprotein IIb/IIIa inhibitor, and (d) a second antiplatelet drug comprising an NSAID, wherein (a) the direct factor IIa inhibitor, (b) the direct factor Xa inhibitor, (c) the first antiplatelet drug and (d) the second antiplatelet drug are formulated to release from the surface of the article in amounts sufficient to inhibit inflammation resulting from the implantation of the article when implanted in a patient.

In some instances, the (a) a direct factor IIa inhibitor, (b) a direct factor Xa inhibitor, (c) first antiplatelet drug, and (d) second antiplatelet drug of the third composition are formulated to treat an injury that resulted from implantation of the article.

In some instances, the (a) a direct factor IIa inhibitor, (b) a direct factor Xa inhibitor, (c) first antiplatelet drug, and (d) second antiplatelet drug of the third composition are formulated to treat the injury without causing any one or more of bleeding, a risk of bleeding risk, and bleeding associated with an administration of a systemic antithrombotic drug.

In some instances, the third composition further comprises (e) colchicine which in combination with (a) the direct factor IIa inhibitor, (b) the direct factor Xa inhibitor, (c) the first antiplatelet drug, and (d) the second antiplatelet drug is formulated to release from the surface of the article in amounts sufficient to enhance inhibition of inflammation and/or thrombosis resulting from the injury.

In some instances, the direct factor IIa inhibitor of the third compositions of the present disclosure is selected from a group consisting of argatroban, dabigatran, ximelagatran, melagatran, efegatran, inogatran, atecegatran metoxil (AZD-0837), hirudin, hirudin analogs, bivalirudin, desirudin, and lepirudin, typically comprising argatroban, or a salt, isomer, solvate, analog, derivative, metabolite, or prodrugs thereof.

In some instances, the direct factor Xa inhibitor of the third compositions of the present disclosure is selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or cribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052), typically comprising (a) apixaban, or a salt, isomer, solvate, analog, derivative, metabolite, or prodrugs thereof, and/or (b) rivaroxaban, or a salt, isomer, solvate, analog, derivative, metabolite, or prodrugs thereof.

In some instances, the glycoprotein IIb/IIIa inhibitor of the third compositions of the present disclosure is selected from a group consisting of abciximab, eptifibatide, orbofiban, roxifiban, sibrafiban, and tirofiban.

In some instances, the NSAID of the third compositions comprises a salicylate.

In some instances, the third compositions further comprise at least a third antiplatelet drug different than the first and second antiplatelet drugs, where the third antiplatelet drug may comprise one or more of a glycoprotein IIb/IIIa inhibitor, a COX inhibitor, an ADP receptor/P2Y12 inhibitor, a prostaglandin analogue, a thromboxane inhibitor, and a phosphodiesterase inhibitors.

In some instances, the third compositions further comprises a chelating agent, wherein the chelating agent may be selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), deferoxamine (DFO), a surfactant-EDTA complex, quaternary ammonium EDTA salt, benzalkonium EDTA salt, EDTA complex, salts, analogue, solvate, hydrate and derivatives thereof.

In some instances, the third compositions of the present disclosure further comprise at least one antiproliferative drug, where the antiproliferative drug may comprise an mTOR inhibitor.

A fourth composition according to the present disclosure for coating a surface of an implantable article comprises (a) a direct factor IIa inhibitor, (b) a direct factor Xa inhibitor, (c) a first antiplatelet drug comprising a glycoprotein IIb/IIIa inhibitor, and (d) a second antiplatelet drug comprising an ADP receptor/P2Y12 inhibitors, where the (a) a direct factor IIa inhibitor, (b) factor Xa inhibitor, (c) first antiplatelet drug and (d) second antiplatelet drug are formulated to release from the surface of the article in amounts sufficient to inhibit inflammation resulting from the implantation of the article when implanted in a patient.

In some instances, the (a) a direct factor IIa inhibitor, (b) factor Xa inhibitor, (c) first antiplatelet drug, and (d) second antiplatelet drug of the fourth compositions are formulated to treat an injury that resulted from implantation of the article.

In some instances, the (a) a direct factor IIa inhibitor, (b) factor Xa inhibitor, (c) first antiplatelet drug, and (d) second antiplatelet drug of the fourth compositions are formulated to treat the injury without causing any one or more of bleeding, a risk of bleeding risk, and bleeding associated with an administration of a systemic antithrombotic drug.

In some instances, the fourth compositions of the present disclosure further comprise ((e) colchicine which in combination with (a) the direct factor IIa inhibitor, (b) the direct factor Xa inhibitor, (c) the first antiplatelet drug, and (d) the second antiplatelet drug is formulated to release from the surface of the article in amounts sufficient to enhance inhibition of inflammation and/or thrombosis resulting from the injury.

In some instances, the fourth compositions of the present disclosure further comprise (f) a third antiplatelet drug comprising aspirin and/or another NSAID which in combination with (a) the direct factor IIa inhibitor, (b) the direct factor Xa inhibitor, (c) the first antiplatelet drug, and (d) the second antiplatelet drug is formulated to release from the surface of the article in amounts sufficient to enhance inhibition of inflammation and/or thrombosis resulting from the injury, wherein the direct factor IIa inhibitor is selected from a group consisting of argatroban, dabigatran, ximelagatran, melagatran, efegatran, inogatran, atecegatran metoxil (AZD-0837), hirudin, hirudin analogs, bivalirudin, desirudin, and lepirudin, typically comprising argatroban, or a salt, isomer, solvate, analog, derivative, metabolite, or prodrugs thereof.

In some instances, the direct factor Xa inhibitor of the fourth compositions of the present disclosure may be selected from a group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052). For example, the direct factor Xa inhibitor may comprise apixaban, or a salt, isomer, solvate, analog, derivative, metabolite, or prodrugs thereof. For example, the direct factor Xa inhibitor may comprise rivaroxaban, or a salt, isomer, solvate, analog, derivative, metabolite, or prodrugs thereof.

In some instances, the glycoprotein IIb/IIIa inhibitor of the fourth compositions may be selected from a group consisting of abciximab, eptifibatide, orbofiban, roxifiban, sibrafiban, and tirofiban.

In some instances, the ADP receptor/P2Y12 inhibitor of the fourth compositions may be selected from a group consisting of clopidogrel, ticlopidine, prasugrel, cangrelor, elinogrel, and ticagrelor.

In some instances, the fourth compositions of the present disclosure further comprise at least a third antiplatelet drug different than the first and second antiplatelet drugs, where the third antiplatelet drug may comprise one or more of a glycoprotein 11b/41a inhibitor, a COX inhibitor, an ADP receptor/P2Y12 inhibitor, a prostaglandin analogue, a thromboxane inhibitor, and a phosphodiesterase inhibitor. For example, the third antiplatelet drug may comprises a salicylate such as aspirin or another NSAID.

In some instances, the fourth compositions of the present disclosure further comprise a chelating agent, where the chelating agent may be selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenctriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), deferoxamine (DFO), a surfactant-EDTA complex, quaternary ammonium EDTA salt, benzalkonium EDTA salt, EDTA complex, salts, analogue, solvate, hydrate and derivatives thereof.

In some instances, the fourth compositions of the present disclosure further comprise at least one antiproliferative drug, such as an mTOR inhibitor.

A fifth composition for coating a surface of an implantable article in accordance with the principles of the present disclosure comprises a direct factor Xa inhibitor and one or more of (a) colchicine, (b) a first antiplatelet drug comprising a glycoprotein IIb/IIIa inhibitor, (c) a second antiplatelet drug comprising an NSAID, and (d) a third antiplatelet drug comprising an ADP inhibitor, where the direct factor Xa inhibitor and the one or more of (a) the colchicine, (b) the first antiplatelet drug, (c) the second antiplatelet drug and (d) the third antiplatelet drug are formulated to release from the surface of the article in amounts sufficient to inhibit inflammation resulting from the implantation of the article when implanted in a patient.

In some instances, the direct factor Xa inhibitor and the one or more of the (a) colchicine, (b) first antiplatelet drug comprising a glycoprotein IIb/IIa inhibitor, (c) second antiplatelet drug comprising an NSAID, and (d) third antiplatelet drug comprising an ADP inhibitor of the fifth compositions of the present disclosure are formulated to treat an injury that resulted from implantation of the article.

In some instances, the direct factor Xa inhibitor and the one or more of the (a) colchicine, (b) first antiplatelet drug comprising a glycoprotein IIb/IIIa inhibitor, (c) second antiplatelet drug comprising an NSAID, and (d) third antiplatelet drug comprising an ADP inhibitor of the fifth compositions of the present disclosure are formulated to treat the injury without causing any one or more of bleeding, a risk of bleeding risk, and bleeding associated with an administration of a systemic antithrombotic drug.

In some instances, the fifth compositions of the present disclosure further comprise a direct factor IIa inhibitor is selected from a group consisting of argatroban, dabigatran, ximelagatran, melagatran, efegatran, inogatran, atecegatran metoxil (AZD-0837), hirudin, hirudin analogs, bivalirudin, desirudin, and lepirudin, where the direct factor IIa inhibitor may comprise argatroban, or a salt, isomer, solvate, analog, derivative, metabolite, or prodrugs thereof.

In some instances, factor Xa inhibitor of the fifth compositions may be selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052). For example, the direct factor Xa inhibitor may comprise apixaban, or a salt, isomer, solvate, analog, derivative, metabolite, or prodrugs thereof. For example, the direct factor Xa inhibitor may comprise rivaroxaban, or a salt, isomer, solvate, analog, derivative, metabolite, or prodrugs thereof.

In some instances, the glycoprotein IIb/IIIa inhibitor of the present disclosure may be selected from a group consisting of abciximab, eptifibatide, orbofiban, roxifiban, sibrafiban, and tirofiban.

In some instances, the ADP receptor/P2Y12 inhibitors of the present disclosure may be selected from a group consisting of clopidogrel, ticlopidine, prasugrel, cangrelor, elinogrel, and ticagrelor.

In some instances, the fifth compositions of the present disclosure further comprise at least a fourth antiplatelet drug different than the first and second antiplatelet drugs, where the fourth antiplatelet drug may comprises one or more of a prostaglandin analogue, a thromboxane inhibitor, and a phosphodiesterase inhibitors.

In some instances, the fifth compositions of the present disclosure further comprise a chelating agent, wherein the chelating agent may be selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), deferoxamine (DFO), a surfactant-EDTA complex, quaternary ammonium EDTA salt, benzalkonium EDTA salt, EDTA complex, salts, analogue, solvate, hydrate and derivatives thereof.

In some instances, the fifth compositions of the present disclosure further comprise at least one antiproliferative drug, such as an mTOR inhibitor.

A sixth composition for coating a surface of an implantable article in accordance with the principles of the present disclosure comprises a direct factor IIa inhibitor and one or more of (a) colchicine, (b) a first antiplatelet drug comprising a glycoprotein IIb/IIIa inhibitor, (c) a second antiplatelet drug comprising an NSAID, and (d) a third antiplatelet drug comprising an ADP inhibitor, where the direct factor Xa inhibitor and the one or more of (a) the colchicine, (b) the first antiplatelet drug, (c) the second antiplatelet drug and (d) the third antiplatelet drug are formulated to release from the surface of the article in amounts sufficient to inhibit inflammation resulting from the implantation of the article when implanted in a patient.

In some instances, the direct factor IIa inhibitor and the (a) colchicine, (b) first antiplatelet drug comprising a glycoprotein IIb/IIIa inhibitor, (c) second antiplatelet drug comprising an NSAID, and (d) third antiplatelet drug comprising an ADP inhibitor of the sixth compositions of the present disclosure are formulated to treat an injury that resulted from implantation of the article.

In some instances, the direct factor IIa inhibitor and the (a) colchicine, (b) first antiplatelet drug comprising a glycoprotein IIb/IIa inhibitor, (c) second antiplatelet drug comprising an NSAID, and (d) third antiplatelet drug comprising an ADP inhibitor of the sixth compositions of the present disclosure are formulated to treat the injury without causing any one or more of bleeding, a risk of bleeding risk, and bleeding associated with an administration of a systemic antithrombotic drug.

In some instances, the direct factor IIa inhibitor of the sixth compositions of the present disclosure may be selected from a group consisting of argatroban, dabigatran, ximelagatran, melagatran, efegatran, inogatran, atecegatran metoxil (AZD-0837), hirudin, hirudin analogs, bivalirudin, desirudin, and lepirudin, typically comprising argatroban, or a salt, isomer, solvate, analog, derivative, metabolite, or prodrugs thereof.

In some instances, the sixth compositions of the present disclosure further comprise a direct factor Xa inhibitor selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1 h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052), wherein the direct factor Xa inhibitor typically comprises (a) apixaban, or a salt, isomer, solvate, analog, derivative, metabolite, or prodrugs thereof or (b) rivaroxaban, or a salt, isomer, solvate, analog, derivative, metabolite, or prodrugs thereof.

In some instances, the glycoprotein IIb/IIIa inhibitor of the sixth compositions of present may be selected from a group consisting of abciximab, eptifibatide, orbofiban, roxifiban, sibrafiban, and tirofiban.

In some instances, the ADP receptor/P2Y12 inhibitors of the sixth compositions of the present disclosure may be selected from a group consisting of clopidogrel, ticlopidine, prasugrel, cangrelor, elinogrel, and ticagrelor.

In some instances, the sixth compositions of the present disclosure further comprise at least a fourth antiplatelet drug different than the first and second antiplatelet drugs, where the fourth antiplatelet drug may comprise one or more of a prostaglandin analogue, a thromboxane inhibitor, and a phosphodiesterase inhibitors.

In some instances, the sixth compositions of the present disclosure further comprise a chelating agent, typically being selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA). 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), deferoxamine (DFO), a surfactant-EDTA complex, quaternary ammonium EDTA salt, benzalkonium EDTA salt, EDTA complex, salts, analogue, solvate, hydrate and derivatives thereof.

In some instances, the sixth compositions of the present disclosure further comprise at least one antiproliferative drug, typically comprising an mTOR inhibitor.

A seventh composition for coating a surface of an implantable article in accordance with the principles of the present disclosure comprising (a) at least one of a direct factor IIa inhibitor and a direct factor Xa inhibitor, (b) at least one antiplatelet drug selected from (i) a first antiplatelet drug comprising a COX-1 inhibitor and (ii) a second antiplatelet drug comprising a glycoprotein IIb/IIa (2a/3b) inhibitor, and (iii) a third antiplatelet drug comprising an adenosine diphosphate (ADP)/P2Y12 inhibitor, where the (a) at least one of the direct factor IIa inhibitor and the direct factor Xa inhibitor, and (b) at least one antiplatelet drug are formulated to release from the surface of the article in amounts sufficient to inhibit inflammation resulting from the implantation of the article when implanted in a patient.

In some instances, the seventh compositions of the present disclosure comprise a direct factor IIa inhibitor selected from a group consisting of argatroban, dabigatran, ximelagatran, melagatran, efegatran, inogatran, atecegatran metoxil (AZD-0837), hirudin, hirudin analogs, bivalirudin, desirudin, and lepirudin, where the direct factor IIa inhibitor typically comprises argatroban, or a salt, isomer, solvate, analog, derivative, metabolite, or prodrugs thereof.

In some instances, the seventh compositions of the present disclosure comprise a direct factor Xa inhibitor selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052), wherein the direct factor Xa inhibitor typically comprises (a) apixaban, or a salt, isomer, solvate, analog, derivative, metabolite, or prodrugs thereof or (b) rivaroxaban, or a salt, isomer, solvate, analog, derivative, metabolite, or prodrugs thereof.

In some instances, the seventh compositions of the present disclosure comprise a COX-1 inhibitor, typically a salicylate or other NSAID.

In some instances, the seventh compositions of the present disclosure comprise a glycoprotein IIb/IIa inhibitor selected from a group consisting of abciximab, eptifibatide, orbotiban, roxifiban, sibratiban, and tirotiban.

In some instances, the seventh compositions of the present disclosure comprise an ADP receptor/P2Y12 inhibitors is selected from a group consisting of clopidogrel, ticlopidine, prasugrel, cangrelor, elinogrel, and ticagrelor.

In some instances, the seventh compositions of the present disclosure comprise at least one of a prostaglandin analogue, a thromboxane inhibitor, and a phosphodiesterase inhibitors.

In some instances, the seventh compositions of the present disclosure comprise a chelating agent, typically being selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), deferoxamine (DFO), a surfactant-EDTA complex, quaternary ammonium EDTA salt, benzalkonium EDTA salt, EDTA complex, salts, analogue, solvate, hydrate and derivatives thereof.

In some instances, the seventh compositions of the present disclosure comprise at least one antiproliferative drug, typically an mTOR inhibitor.

An eighth composition for coating a surface of an implantable article, in accordance with the principles of the present disclosure comprising (a) at least one of a direct factor IIa inhibitor and a direct factor Xa inhibitor, (b) at least one of (i) colchicine, (ii) a first antiplatelet drug, and (iii) a chelating agent, where the (a) at least one of a direct factor IIa inhibitor and a direct factor Xa inhibitor and (b) the at least one of (i) colchicine (ii) the first antiplatelet drug, and the chelating agent are formulated to release from the surface of the article in amounts sufficient to inhibit inflammation resulting from the implantation of the article when implanted in a patient.

In some instances, the eighth compositions of the present disclosure comprise a direct factor IIa inhibitor selected from a group consisting of argatroban, dabigatran, ximelagatran, melagatran, efegatran, inogatran, atecegatran metoxil (AZD-0837), hirudin, hirudin analogs, bivalirudin, desirudin, and lepirudin, where the direct factor IIa inhibitor comprises argatroban, or a salt, isomer, solvate, analog, derivative, metabolite, or prodrugs thereof.

In some instances, the eighth compositions of the present disclosure comprise a direct factor Xa inhibitor selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052), where the direct factor Xa inhibitor typically comprises (a) apixaban, or a salt, isomer, solvate, analog, derivative, metabolite, or prodrugs thereof or (b) rivaroxaban, or a salt, isomer, solvate, analog, derivative, metabolite, or prodrugs thereof.

In some instances, the eighth compositions of the present disclosure comprises colchicine.

In some instances, the eighth compositions of the present disclosure comprise one or more antiplatelet drugs selected from the group consisting of a glycoprotein IIb/IIIa inhibitor, an ADP receptor/P2Y12 inhibitor, a COX-1 inhibitor, a prostaglandin analogue, a thromboxane inhibitor, and a phosphodiesterase inhibitor.

In some instances, the eighth compositions of the present disclosure comprise a chelating agent selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), deferoxamine (DFO), a surfactant-EDTA complex, quaternary ammonium EDTA salt, benzalkonium EDTA salt, EDTA complex, salts, analogue, solvate, hydrate and derivatives thereof.

In some instances, the eighth compositions of the present disclosure comprise at least one antiproliferative drug, typically an mTOR inhibitor.

In still other examples, the therapeutic composition comprises one or more agents selected from the group consisting of direct factor Xa inhibitor, direct factor IIa inhibitor, in combination with one or two antiplatelet selected from glycoprotein IIb/IIIa inhibitor, P2Y12 inhibitor and aspirin, and additional optional an anti-inflammatory selected from colchicine, and optionally an anti-proliferation such as mTOR, or a microtubule inhibitor such as Paclitaxel in implantable devices where the combination achieves a synergistic inhibitory effect greater than achieved by coatings employing either drug alone.

In some examples, the therapeutic composition is formulated to combine colchicine with other drug(s) such as rivaroxaban, argatroban, tirofiban and the combination achieved a synergistic inhibitory effect greater than achieved by either drug alone.

In some examples, the therapeutic composition is further formulated to combine of colchicine with other drug(s) such as rivaroxaban, argatroban, tirofiban, ticagrelor and mTOR, and the combination achieved a synergistic inhibitory effect greater than achieved by either drug alone.

In still other examples, the therapeutic composition comprises a combination of direct factor Xa inhibitor with two, three or more other therapeutic agent selected from the group consisting antiplatelets selected from glycoprotein IIb/IIIa inhibitor, P2Y12 inhibitor and aspirin, and anti-inflammatory agents selected from colchicine in drug coatings for stents and other implantable devices where the combination achieves a synergistic inhibitory effect greater than achieved by coatings employing either drug alone.

In still other examples, the therapeutic composition may comprise a combination of direct factor IIa inhibitor with two, three or more other therapeutic agent selected from the group consisting of antiplatelets selected from glycoprotein IIb/IIIa inhibitor, P2Y12 inhibitor and aspirin, and anti-inflammatory agents selected from colchicine in drug coatings for stents and other implantable devices where the combination achieves a synergistic inhibitory effect greater than achieved by coatings employing either drug alone.

In still other examples, the therapeutic composition may comprise a combination of direct factor Xa inhibitor, antiplatelets selected from glycoprotein IIb/IIIa inhibitor, P2Y12 inhibitor and aspirin, in drug coatings for stents and other implantable devices where the combination achieves a synergistic inhibitory effect greater than achieved by coatings employing either drug alone.

In still other examples, the therapeutic composition may comprise a combination of direct factor IIa inhibitor, antiplatelets selected from glycoprotein IIb/IIa inhibitor, P2Y12 inhibitor and aspirin, in drug coatings for stents and other implantable devices where the combination achieves a synergistic inhibitory effect greater than achieved by coatings employing either drug alone.

The compositions of the present disclosure are typically coated on articles configured to be implanted in a patient's body. For example, the implantable article may comprise a body having a surface configured for implantation in a patient, wherein the surface is at least partially coated with any of compositions described above.

In some instances, the article comprises an expandable scaffold configured for implantation in the patient's vascular system.

In some instances, the article comprises a non-expandable body.

In some instances, the composition is formulated to be released from the article over a period of at least of at least one week, one month, or one year.

In some instances, at least of the components of the composition may be formulated in one or more carriers which degrade when implanted in the patient.

In some instances, the composition may be formulated to release no greater than 90%, 75%, 50%, 25%, or 10% of its weight of within one week, one month, three months, six months, or one year of implantation.

Exemplary chelating agents in the therapeutic composition may be formulated to deplete calcium in the environment surrounding the scaffold upon implantation of the scaffold structure in said environment.

In specific instances, the therapeutic composition may be formulated to release at least 50%, preferably at least 75%, by weight of the at least one chelating agent into the vascular environment within 72 hours of implantation, preferably within 24 hours of implantation, more preferably within 6 hours of implantation, and even more preferably within 4 hour of implantation.

In additional instances, the therapeutic composition may be formulated to release additional amounts of the at least one chelating agent into the environment for a period of at least 3 days, preferably at least 7 days, more preferably 21 days, still more preferably at least 28 days, even more preferably at least 3 months, and often 6 months or more after implantation. Exemplary chelating agents may be selected from a group consisting of ethylenediaminetetraacetic acid (EDTA), calcium disodium edetate, magnesium dipotassium edetate, and magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), deferoxamine (DFO), a surfactant-EDTA complex, quaternary ammonium EDTA salt, benzalkonium EDTA salt, EDTA complex, salts, analogue, solvate, hydrate and derivatives thereof.

In particular instances, the chelating agent consists essentially of ethylenediaminetetraacetic acid (EDTA).

The therapeutic composition that may comprise, consist essentially of, or consist of chelating agent, where the chelating agent may be present in the therapeutic composition at a weight percent from 10% to 100%. In specific instances, the therapeutic composition consists essentially of chelating agent, where the only active ingredient in the therapy composition will be the chelating agent optionally present with other inactive components and ingredients. In other instances, the therapeutic composition comprises the chelating agent in combination with additional active and/or inactive substances. In such instances, the additional active and/or inactive substances may be present in the therapeutic composition at a weight percent from 20% to 90%.

In some instances, the therapeutic compositions of the present disclosure may further comprise a cationic anti-coagulation enhancer, where the cationic anti-coagulation enhancer may selected from a group consisting of magnesium stearate and other magnesium salts, monoammonium salts, diammonium salts, triammonium salts, benzyldimethyltetradecylammonium salts, tridodecylmethylammonium salt, other benzalkonium, analogue, solvate, hydrate and derivatives thereof. Alternatively or additionally, the cationic anti-coagulation enhancer may be selected from a group consisting of cationic polymer or compounds including but not limit to poly(L-lysine) (PLL), linear polyethyleneimine (PEI), branch polyethyleneimine (PEI), chitosan, PAMAM dendrimers, and poly(2-dimethylamino)ethyl methacrylate (pDMAEMA), protamine, polylysine, a polybetaaminoester (PBAE), Histone, ethylenediamine, methylenediamine, ammonium chloride, melamine, histamine, histidine, analogue, solvate, hydrate and derivatives thereof.

In specific examples, the cationic anti-coagulation enhancer may comprise, consist essentially of, or consist of benzyldimethyltetradecylammonium chloride.

In specific examples, the cationic anti-coagulation enhancer may comprise, consist essentially of, or consist of linear polyethyleneimine (PEI).

Any of the above-described therapeutic compositions may further comprise at least one anti-coagulant. In such instances, the therapeutic composition may be formulated to release at least one anti-coagulant at a rate equal to that of the chelating agent. Alternatively, the therapeutic composition may be formulated to release at least one anti-coagulant at a rate slower than that of the chelating agent. Alternatively, the therapeutic composition may be formulated to release at least one anti-coagulant at a rate faster than that of the chelating agent.

In specific examples, the anti-coagulant may be selected from the group consisting of a direct factor IIa inhibitor and a direct factor Xa inhibitor. Exemplary direct factor IIa inhibitors may be selected from the group consisting of argatroban, dabigatran, ximelagatran, melagatran, efegatran, inogatran, atecegatran metoxil (AZD-0837), hirudin, hirudin analogs, bivalirudin, desirudin, and lepirudin. A preferred, direct factor IIa inhibitor comprises argatroban, or a salt, isomer, solvate, analog, derivative, metabolite, or prodrugs thereof.

Exemplary direct factor Xa inhibitors may be selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1 h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or cribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052). A first preferred direct factor Xa inhibitor comprises apixaban, or a salt, isomer, solvate, analog, derivative, metabolite, or prodrugs thereof. A second preferred direct factor Xa inhibitor comprises rivaroxaban, or a salt, isomer, solvate, analog, derivative, metabolite, or prodrugs thereof.

Any of the above-described therapeutic compositions may further comprise an mTOR inhibitor selected from a group consisting of sirolimus, biolimus, everolimus, myolimus, novolimus, ridaforolimus, temsirolimus, zotarolimus, or salts, isomers, solvates, analogs, derivatives, metabolites, or prodrugs thereof. A preferred MTOR inhibitor comprises sirolimus, or a salt, isomer, solvate, analog, derivative, metabolite, or prodrugs thereof.

Any of the above-described therapeutic compositions may further comprise paclitaxel, or a salt, isomer, solvate, analog, derivative, metabolite, or prodrug thereof.

Any of the above-described therapeutic compositions may further comprise an antiplatelet drug. Any of the above-described therapeutic compositions may further comprise an antiproliferative agent selected from the group consisting of mycophenolate mofetil, mycophenolate sodium, azathioprine.

The implantable scaffolds of present disclosure may have anyone of a wide variety of known structures suitable for implantation and expansion at a target site in the patient's body. Often, the scaffold will have at least an outer surface, an inner surface, and one or more edge surfaces between the outer and inner surfaces. In such instances, at least a portion of the outer surface may be coated with the therapeutic compositions. In other instances, at least a portion of the inner surface may be coated with the therapeutic compositions. In still other instances, a portion of the edge surfaces may be coated with the therapeutic compositions. As an alternative or in addition to surface coating, at least some of the surfaces may have receptacles formed therein and at least some of said receptacles have therapeutic agent therein. For example, the receptacles comprise one or more of wells, channels, holes, and surface texture.

In a second aspect, the present disclosure provides a method for treating a vascular tissue injury in a patient. The method comprises implanting a scaffold structure at a target location in the patient's vasculature proximate the tissue injury and releasing a drug composition including at least one chelating agent from the implanted scaffold structure into the vasculature, wherein the chelating agent is released sufficiently rapidly into the vasculature to prevent blood clotting and inhibit the fibrin formation.

In particular examples, at least 75% by weight of the at least one chelating agent is released into the vasculature within 72 hours of implantation, preferably within 24 hours of implantation, more preferably within 6 hours of implantation, and even more preferably within 4 hour of implantation, and usually between 10 minutes and 4 hours.

In the further examples of the methods herein, the chelating agent may be selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), deferoxamine (DFO), a surfactant-EDTA complex, quaternary ammonium EDTA salt, benzalkonium EDTA salt, EDTA complex, salts, analogue, solvate, hydrate and derivative thereof. In one specific instance, the chelating agent consists essentially of ethylenediaminetetraacetic acid (EDTA).

In some instances, therapeutic composition further comprises a cationic anti-coagulation enhancer. For example, the cationic anti-coagulation enhancer may be selected from a group consisting of magnesium stearate and other magnesium salts, monoammonium salts, diammonium salt, triammonium salt, benzyldimethyltetradecylammonium salt, tridodecylmethylammonium salt, other benzalkonium, analogue, solvate, hydrate and derivatives thereof. In another example,

the cationic anti-coagulation enhancer may be selected from a group consisting of cationic polymer or compounds including but not limit to poly(L-lysine) (PLL), linear polyethyleneimine (PEI), branch polyethyleneimine (PEI), chitosan, PAMAM dendrimers, and poly(2-dimethylamino)ethyl methacrylate (pDMAEMA), protamine, polylysine, a polybetaaminoester (PBAE), Histone, ethylenediamine, methylenediamine, ammonium chloride, melamine, histamine, histidine, analogue, solvate, hydrate and derivatives thereof. In a specific instance, the cationic anti-coagulation enhancer consists essentially of benzyldimethyltetradecylammonium chloride. In another specific instance, the cationic anti-coagulation enhancer consists essentially of linear polyethyleneimine (PEI).

The therapeutic composition delivered by the methods of the present disclosure may further comprise at least one anti-coagulant. For example, the at least one anti-coagulant may be selected from the group consisting of a direct factor IIa inhibitor and a direct factor Xa inhibitor. Alternatively, the at least one anti-coagulant may comprise a direct factor IIa inhibitor selected from the group consisting of argatroban, dabigatran, ximelagatran, melagatran, efegatran, inogatran, atecegatran metoxil (AZD-0837), hirudin, hirudin analogs, bivalirudin, desirudin, and lepirudin. An exemplary direct factor IIa inhibitor may comprises argatroban, or a salt, isomer, solvate, analog, derivative, metabolite, or prodrugs thereof.

In other examples of the methods herein, the at least one anti-coagulant may comprise a direct factor Xa inhibitor selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052). In one instance, the direct factor Xa inhibitor may comprise apixaban, or a salt, isomer, solvate, analog, derivative, metabolite, or prodrugs thereof. In another instance, the direct factor Xa inhibitor may comprise rivaroxaban, or a salt, isomer, solvate, analog, derivative, metabolite, or prodrugs thereof.

The therapeutic composition delivered by the methods of the present disclosure may further comprise an mTOR inhibitor selected from a group consisting of sirolimus, biolimus, everolimus, myolimus, novolimus, ridaforolimus, temsirolimus, zotarolimus, or salts, isomers, solvates, analogs, derivatives, metabolites, or prodrugs thereof. An exemplary mTOR inhibitor comprises sirolimus, or a salt, isomer, solvate, analog, derivative, metabolite, or prodrugs thereof.

The therapeutic composition delivered by the methods of the present disclosure may further comprise paclitaxel, or a salt, isomer, solvate, analog, derivative, metabolite, or prodrug thereof.

The therapeutic composition delivered by the methods of the present disclosure may further comprise further comprises an antiplatelet drug.

In all of the methods described herein, the therapeutic composition may be positioned on at least one of an internal surface and an external surface of the implantable scaffold and/or may be positioned on both an external and an internal surface of the implantable scaffold.

The methods of the present disclosure are generally suitable for treating tissue injury caused by expanding the scaffold at the location but are also useful for treating tissue injury that preexists deploying the structure at the location.

In a third aspect, the present disclosure provides an implant comprising a body structure having a surface configured to be implanted in a patient's body. A therapeutic composition is present on a surface of the body structure, where the therapeutic composition comprises at least one drug selected from the group consisting of a chelating agent, a direct factor IIa inhibitor, a direct factor Xa inhibitor, wherein the therapeutic composition is formulated for a delayed release into an environment surrounding the body structure upon implantation of the body structure into said environment.

In some examples, the therapeutic composition may be formulated for a rapid release into the environment surrounding the body structure a preselected time period after implantation of the body structure into said environment.

Implants may be any type of therapeutic, diagnostic, or other structure intended for implantation in the patient's body, typically being an expandable scaffold, such as a vascular stent, a prosthetic heart valve, a patent foramen ovale (PFO) occlusion device, an atrial septal defect (ASD) occlusion device, a left atrial appendage (LAA) occlusion device, or similar expandable structure, or being an orthopedic implant.

In such instances, the therapeutic composition may be present at least partly on the surface of the body structure. Alternatively or additionally, the therapeutic composition may be present at least partly within a cavity or reservoir within the body structure.

The therapeutic composition may be formulated to inhibit release of the at least one drug into the environment surrounding the body structure for a time period in a range having a lower time limit selected from 5 minutes, 10 minutes, 15 minutes, 30 minutes, and 45 minutes and an upper time limit selected from 1 hour, 2 hours, 3 hours, and 4 hours, and all combinations thereof.

The therapeutic composition may be formulated to release at least 50% by weight, preferably at least 75% by weight of the at least one drug into the environment surrounding the body structure within 72 hours of implantation, preferably within 24 hours of implantation, more preferably within 6 hours of implantation, and even more preferably within 4 hour of implantation.

The therapeutic composition may be formulated to release additional amounts of the at least one drug into the environment for a period of at least 3 days, preferably at least 7 days, more preferably 21 days, still more preferably at least 28 days, even more preferably at least 3 months, and often 6 months or more after implantation.

In some examples, the drug in the therapeutic composition may comprise at least a chelating agent in the therapeutic composition is formulated to deplete calcium in the environment surrounding the body structure upon implantation of the body structure in said environment. Such a chelating agent may be selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), deferoxamine (DFO), a surfactant-EDTA complex, quaternary ammonium EDTA salt, benzalkonium EDTA salt, EDTA complex, salts, analogue, solvate, hydrate and derivatives thereof. A specific instance, the chelating agent consists essentially of ethylenediaminetetraacetic acid (EDTA).

In some instances, the therapeutic composition may further comprise a cationic anti-coagulation enhancer. For example, the cationic anti-coagulation enhancer may be selected from a group consisting of magnesium stearate and other magnesium salts, monoammonium salts, diammonium salts, triammonium salts, benzyldimethyltetradecylammonium salts, tridodecylmethylammonium salts, other benzalkonium, analogue, solvate, hydrate and derivatives thereof. Alternatively or additionally, the cationic anti-coagulation enhancer may be selected from a group consisting of cationic polymer or compounds including but not limit to poly(L-lysine) (PLL), linear polyethyleneimine (PEI), branch polyethyleneimine (PEI), chitosan, PAMAM dendrimers, and poly(2-dimethylamino)ethyl methacrylate (pDMAEMA), protamine, polylysine, a polybetaaminoester (PBAE), Histone, ethylenediamine, methylenediamine, ammonium chloride, melamine, histamine, histidine, analogue, solvate, hydrate and derivatives thereof. In a specific instance, the cationic anti-coagulation enhancer consists essentially of benzyldimethyltetradecylammonium chloride. In another specific instance, the cationic anti-coagulation enhancer consists essentially of linear polyethyleneimine (PEI).

In some instances, the at least one drug may comprise a direct factor IIa inhibitor selected from the group consisting of argatroban, dabigatran, ximelagatran, melagatran, efegatran, inogatran, atecegatran metoxil (AZD-0837), hirudin, hirudin analogs, bivalirudin, desirudin, and lepirudin. For example, the at least one direct factor IIa inhibitor may comprise argatroban, or a salt, isomer, solvate, analog, derivative, metabolite, or prodrugs thereof.

In some examples, the at least one drug may comprise a direct factor Xa inhibitor selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052). For example, the direct factor Xa inhibitor may comprise apixaban, or a salt, isomer, solvate, analog, derivative, metabolite, or prodrugs thereof. In another example, the direct factor Xa inhibitor may comprise rivaroxaban, or a salt, isomer, solvate, analog, derivative, metabolite, or prodrugs thereof.

In some examples, the at least one drug may comprise a factor XI/XIa inhibitor selected from the group consisting of antisense oligonucleotides including but not limit to IONIS-FXI and fesomersen; monoclonal antibodies (mAbs) including but not limit to osocimab, abelacimab, xisomab (AB023), REGN9933 and MK-2060; small molecules including but not limit to milvexian, asundexian, SHR2285, BMS-262084, BMS-654457, BMS-986177, BMS-962212, ONO-5450598, ONO-7684, and BAY 2433334 and frunexian (EP-7041) and a protein Z-dependent protease inhibitors (ZPI), and all above analogue, solvate, hydrate or derivatives thereof.

In specific examples, a ZPI-containing cationic anti-coagulation enhancer may comprise, consist essentially of, or consist of Asundexian, or milvexian, a small molecule factor XIa inhibitor having the formula [(6r,10s)-10-{4-[5-chloro-2-(4-chloro-1h-1,2,3-triazol-1-yl)phenyl]-6-oxo-1(6h)-pyrimidinyl}-1-(difluoromethyl)-6-methyl-1,4,7,8,9,10-hexahydro-11,15-(metheno)pyrazolo [4,3-b][1,7] diazacyclotetradecin-5(6h)-one] and described in WO2020/210613 and WO20201210629, the full disclosures of which are incorporated herein by reference.

In some examples, the therapeutic composition is formulated to release the one or more active substances at a rate sufficient to generate a tissue concentration of at least one active ingredient at the injury site within a range of at least about 0.05 ng/mg of tissue, at least about 0.15 ng/mg of tissue, at least about 0.3 ng/mg of tissue within about 3 hours, 6 hours, 12 hours, one day, 3 days, 7 days, 30 days, or 6 months. The resulting concentration of the at least one active ingredient can be found at the injury site, adjacent to the implantable device containing the active ingredients, at the ends of the implantable device and on the body lumen sides of the implantable device.

In some examples, the therapeutic composition is formulated to release at least one or more active substances at a rate sufficient to generate a tissue concentration at the injury site of the at least one active ingredients within a range of about 0.05 ng/mg of tissue to about 100 ng/mg of tissue, about 0.05 ng/mg of tissue to about 50 ng/mg of tissue, about 0.05 ng/mg of tissue to about 25 ng/mg of tissue, about 0.05 ng/mg of tissue to about 10 ng/mg of tissue, about 0.05 ng/mg of tissue to about 7 ng/mg of tissue, about 0.05 ng/mg of tissue to about 5 ng/mg of tissue, about 0.05 ng/mg of tissue to about 2 ng/mg of tissue, about 0.05 ng/mg of tissue to about 1 ng/mg of tissue, about 0.05 ng/mg of tissue to about 0.75 ng/mg of tissue, about 0.15 ng/mg of tissue to about 100 ng/mg of tissue, about 0.15 ng/mg of tissue to about 50 ng/mg of tissue, about 0.15 ng/mg of tissue to about 25 ng/mg of tissue, about 0.15 ng/mg of tissue to about 10 ng/mg of tissue, about 0.15 ng/mg of tissue to about 1 ng/mg of tissue, about 0.3 ng/mg of tissue to about 100 ng/mg of tissue, about 0.3 ng/mg to about 50 ng/mg of tissue, about 0.3 ng/mg of tissue to about 25 ng/mg of tissue, about 0.3 ng/mg of tissue to about 10 ng/mg of tissue, about 0.3 ng/mg of tissue to about 5 ng/mg of tissue, about 0.3 ng/mg of tissue to about 1 ng/mg of tissue within about 3 hours, 6 hours, 12 hours, one day, 3 days, 7 days, 30 days, or 6 months.

The active ingredients of the therapeutic compositions may be formulated in varying amounts relative each other. In some examples, the active ingredients are formulated to provide equal tissue concentrations of each active ingredients, amounts to provide double tissue concentrations of some active ingredients compared to the other active ingredients, amounts to provide triple tissue concentrations of some active ingredients compared to the other active ingredients, or amounts provide four times the tissue concentrations of some active ingredients compared to the other active ingredients within 3 hours, 6 hours, 7 hours, 12 hours, one day, 3 days, 7 days, 30 days, or 6 months. Utilizing these ranges of active ingredients to provide the tissue concentrations disclosed herein and the proportions of each active ingredient as described, surprisingly results in a synergistic result of increased blood clotting time.

The amounts of active ingredients can vary from one to another. It is possible within the disclosure to use two, three, four or more active ingredients and achieve synergistic results in a decreased clotting time. For example, combinations or anticoagulant(s) and anti-platelet drugs result in synergism. Combinations of anticoagulant(s) and colchicine result in synergism. Combinations of anticoagulant(s), anti-platelet drug and anti-inflammatory drug, such as colchicine result in synergism. When utilizing anticoagulants, such as direct Factor Xa inhibitor and direct Factor IIa inhibitor, one can use either inhibitor in relationship to the other inhibitor in a weight compositional ratio of about 0.5:1 to about 5:1. When utilizing an anticoagulant, such as direct Factor Xa inhibitor or direct Factor IIa inhibitor alone or together, and an anti-platelet drug, such as tirofiban or ticagrelor, the weight compositional ratios may be about 0.5:1 to about 5:1, of either or both of the anticoagulants compared to a weight compositional ratio of about 0:5:1 to about 5:1, or about 0:5:1 to about 2:1, or about 0.5:1 to about 3:1 of the anti-platelet drug compared to either or both anticoagulants. When utilizing an anticoagulant, such as direct Factor Xa inhibitor or direct Factor IIa inhibitor and an anti-inflammatory drug, such as colchicine, the weight compositional ratios may be about 0.5:1 to about 5:1, of either or both of the anticoagulants compared to a weight compositional ratio of about 0:5:1 to about 5:1, or about 0:5:1 to about 2:1, or about 0.5:1 to about 3:1 of the anti-inflammatory drug compared to either or both anticoagulants. These weight compositional ratios may be used for compositions comprising direct Factor Xa inhibitor, direct Factor IIa inhibitor, an anti-platelet drug and an anti-inflammatory drug, such as colchicine. Surprisingly, synergism results in a decrease in clotting time compared to a control from these compositions.

In some embodiments, there may be two, three, four, or more active ingredients in the therapeutic compositions of the present disclosure. In some examples, the active ingredients are formulated in varying amounts relative to one another. For example, a first active ingredient may be defined by a weight compositional ratio of the first active ingredient relative to the rest of the active ingredients. In some cases, the ratio may be about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 30:1, about 40:1, about 50:1, about 60:1, about 70:1, about 80:1, about 90:1, about 100:1, or any ratio within a range between any two preceding values.

For example, a first active ingredient and a second active ingredient may be defined by a weight compositional ratio of the first and the second active ingredient relative to the rest of the active ingredients. In some cases, the ratio may be about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 30:1, about 40:1, about 50:1, about 60:1, about 70:1, about 80:1, about 90:1, about 100:1, or any ratio within a range between any two preceding values.

For example, a first active ingredient, a second active ingredient, and a third active ingredient may be defined by a weight compositional ratio of the first, the second, and the third active ingredient relative to the rest of the active ingredients. In some cases, the ratio may be about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 30:1, about 40:1, about 50:1, about 60:1, about 70:1, about 80:1, about 90:1, about 100:1, or any ratio within a range between any two preceding values.

For example, a first active ingredient, a second active ingredient, a third active ingredient, and a fourth active ingredient may be defined by a weight compositional ratio of the first, the second, the third, and the fourth active ingredient relative to the rest of the active ingredients. In some cases, the ratio may be about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 30:1, about 40:1, about 50:1, about 60:1, about 70:1, about 80:1, about 90:1, about 100:1, or any ratio within a range between any two preceding values.

In other examples, therapeutic composition may further comprise an mTOR inhibitor selected from a group consisting of sirolimus, biolimus, everolimus, myolimus, novolimus, ridaforolimus, temsirolimus, zotarolimus, or salts, isomers, solvates, analogs, derivatives, metabolites, or prodrugs thereof. The particular instance, the mTOR inhibitor comprises sirolimus, or a salt, isomer, solvate, analog, derivative, metabolite, or prodrugs thereof.

In still other examples, the composition may further comprise paclitaxel, or a salt, isomer, solvate, analog, derivative, metabolite, or prodrug thereof. In still other examples, the therapeutic composition may further comprise an antiplatelet drug. In still other examples, the therapeutic composition may further comprise an antiproliferative agent selected from the group consisting of mycophenolate mofetil, mycophenolate sodium, azathioprine.

In preferred embodiments, the therapeutic composition is formulated to release no greater than 90%, 75%, 50%, 25%, or 10% by weight of the colchicine and/or tirofiban from the scaffold into a vascular environment within one week, one month, three months, six months, or one year of implantation.

In preferred embodiments, the therapeutic composition is formulated to release no greater than 900%, 75%, 50%, 25%, or 10% by weight of the antiproliferative from the scaffold into the vascular environment within one week, one month, three months, six months, or one year of implantation.

In preferred embodiments, the scaffold structure is loaded with an amount of the colchicine and/or tirofiban in a range from 1.7 μg to 170 μg, 1.7 μg to 85 μg, 2.5 μg to 40 μg, or 3.4 μg to 20 μg per mm of length of the scaffold structure.

In preferred embodiments, the scaffold structure is loaded with an amount of the antiproliferative drug in a range from 2 μg to 200 μg, 2 μg to 100 μg, 3 μg to 50 μg, or 4 μg to 20 μg per mm of length of the scaffold structure.

In preferred embodiments, the carrier comprises a biodegradable polymer. For example, the biodegradable polymer may be selected from a group consisting of polyesters, including polylactic acids, polyglycolic acids, polylactic acid-co-glycolic acids, polylactic acid-co-caprolactones, polyethylene glycol-block-poly caprolactone, and polyurethanes; poly(methyl methacrylate) (PMMA); poly N-(2-Hydroxypropyl) methacrylamides; polyethylenimine (PEI), dextran, dextrin, chitosans, poly(L-lysine); poly(aspartamides), polyethylenes; polypropylenes; polyamides; polyethylene glycols (PEG); silicones; poly(anhydrides); and poly ortho esters.

In some instances, the therapeutic composition may further comprise at least one anti-coagulant agent. For example, the at least one anti-coagulant may be a direct factor IIa inhibitor selected from the group consisting of argatroban, dabigatran, ximelagatran, melagatran, efegatran, inogatran, atecegatran metoxil (AZD-0837), hirudin, hirudin analogs, bivalirudin, desirudin, and lepirudin.

In other examples, the at least one anti-coagulant may be a direct factor Xa inhibitor selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or cribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052).

In some instances, the therapeutic composition may further comprise an antiplatelet drug. For example, the antiplatelet drug may be selected from a group consisting of Glycoprotein III/IIIa inhibitors including but not limit to abciximab, eptifibatide, orbofiban, roxifiban, sibrafiban, tirofiban; ADP receptor/P2Y12 inhibitors including but not limit to clopidogrel, prasugrel, ticlopidine, cCangrelor, elinogrel, ticagrelor; prostaglandin analogue (PGI2) inhibitors including but not limit to beraprost, iloprost, prostacyclin, treprostinil: COX inhibitors including but not limit to acetylsalicylic acid/aspirin, aloxiprin, eCarbasalate calcium, indobufen, tTriflusal, dipyridamole; thromboxane inhibitors including but not limit to picotamide, terbogrel, Terutroban, aspirin, ridogrel, ramatroban; phosphodiesterase inhibitors including but not limit to cilostazol, dipyridamole, triflusal, milrinone; other inhibitors including but not limit to cloricromen, ditazole, vorapaxar; PAR/PAR-1 antagonists such as SCH530348, atopaxar, vorapaxar and E-5555; GPIa/IIa inhibitor such as EMS16; adhesion antagonists such as DZ-697b and RG12986; or a derivative, analogue, or salt thereof.

In a preferred instance, the antiplatelet drug will be a glycoprotein IIb/IIIa platelet inhibitor, such as tirotiban or a derivative, analogue, or salt thereof. Particularly useful formulations will comprise colchicine and/or tirofiban and sirolimus or another mTOR inhibitor in combination with using.

In some instances, the therapeutic composition may further comprise a second antiproliferative drug. For example, the second antiproliferative drug may be selected from the group consisting of mycophenolate mofetil, mycophenolate sodium, azathioprine.

In some instances, the therapeutic composition may further comprise a factor XI/XIa inhibitor. Examples of factor XI/XIa inhibitors are selected from antisense oligonucleotides including but not limit to IONIS-FXI and fesomersen; monoclonal antibodies (mAbs) including but not limit to osocimab, abelacimab, xisomab (AB023), REGN9933 and MK-2060; small molecules including but not limit to milvexian, asundexian, SHR2285, BMS-262084, BMS-654457, BMS-986177, BMS-962212, UNO-5450598, ONO-7684, and BAY 2433334 and frunexian (EP-7041); a protein Z-dependent protease inhibitor, and all above analogue, solvate, hydrate or derivatives thereof.

In some instances, the therapeutic composition may further comprise specific inhibitors of thrombin, thrombotic agents, factor IXa, factor XI, factor XIa, factor XII, factor XIIa, factor XIII, factor XIIIa, factor VIIa or factor VIIIa.

In some instances, the therapeutic composition may further comprise a chelating agent formulated to deplete calcium in the environment surrounding the scaffold upon implantation of the scaffold structure in said environment.

The delayed composition release implantable scaffolds may have at least an outer surface, an inner surface, and one or more edge surfaces between the outer and inner surfaces, where at least a portion of the outer surface may be coated with the therapeutic compositions. Additionally or alternatively, at least a portion of the inner surface may be coated with the therapeutic compositions. Additionally or alternatively, at least a portion of the edge surfaces may be coated with the therapeutic compositions. Additionally or alternatively, at least some of the surfaces may have receptacles formed therein and at least some of said receptacles have therapeutic agent therein. For example, the receptacles comprise one or more of wells, channels, holes, and surface texture.

In another aspect, the present disclosure provides an implantable scaffold comprising a scaffold structure having a surface configured to be expanded in the patient's body. A first therapeutic composition is coated, layered, bonded, or otherwise affixed to the scaffold and comprises a first drug formulation including at least one drug selected from the group consisting of a calcium chelating agent, a direct factor IIa inhibitor and a direct factor Xa inhibitor. A second therapeutic composition is also coated, layered, bonded, or otherwise affixed to the scaffold structure and or the first therapeutic composition and comprises a second drug formulation including at least one drug selected from the group consisting of a calcium chelating agent, a direct factor IIa inhibitor and a direct factor Xa inhibitor. The first therapeutic composition is formulated for a rapid release of the first drug formulation into a vascular environment and the second therapeutic composition is formulated for an extended release of the second drug formulation into the vascular environment.

The implantable scaffold may have any conventional or novel structure intended for implantation in a patient's vasculature, including, the arterial and venous coronary, peripheral and cerebral vasculature. The scaffolds may be intended for direct implantation, for example comprising or consisting of vascular stents intended to maintain patency in in a vascular lumen. Additionally, the scaffolds may be part of assemblies including additional components, such as vascular grafts, prosthetic valves, and the like. Depending on the intended purpose, the scaffold may be non-degradable in the vascular environment, for example being formed from or otherwise comprising a metal or a polymer which is non-degradable in the vascular environment. In other instances, the scaffold may be degradable in the vascular environment, for example being formed from or otherwise comprising a metal or polymer which is degradable in the vascular environment.

In addition to such implantable scaffolds, the therapeutic compositions and drug formulations described below may also find use with a wide variety of other implantable and non-implantable devices and tools which may be subject to unwanted clotting, as described elsewhere herein.

The rapid release of the first drug formulation and extended release of the second drug formulation will typically act in combination to accelerate dissolution of one or more of inflammation, cell proliferation, internal elastic lamina (IEL) injury, thrombin, fibrin formation, platelet aggregation, platelet activation, and clot or thrombus formation; and/or inhibit one or more of inflammation, cell proliferation, internal elastic lamina (IEL) injury, thrombin, fibrin formation, platelet aggregation, platelet activation, and clot or thrombus formation; and/or increase or prolong time before blood forms clot or thrombus.

In specific instances, at least one of the first drug formulation, the second drug formulation, and the third drug formulation may comprise a calcium chelating agent, a direct factor IIa inhibitor and a direct factor Xa inhibitor. In other instances, the first drug formulation, the second drug formulation, and the third drug formulation may each comprise a calcium chelating agent, a direct factor IIa inhibitor, and a direct factor Xa inhibitor.

In specific instances, the at least one drug of the first (rapid release) drug formulation is released from the first therapeutic composition over a first time period (duration) is in a range from 5 minutes to 28 days after implantation, usually from 5 minutes to 3 days after implantation, and preferably from 5 minutes to 1 days after implantation. The first therapeutic composition is typically configured to release the at least one drug of the first drug formulation at a mean rate in the range from 2 μg/hour to 40 μg/hour, usually from 2 μg/hour to 30 μg/hour, preferably from 2 μg/hour to 10 μg/hour over a 24-hour period following exposure to the vascular environment, where the mean rate may be determined based on the amount (weight) of drug released over the total duration of the release.

In specific instances, the at least one drug of the second drug formulation (sustained release) is released from the second therapeutic composition over a second time period is in a range from 30 days to 12 months after implantation, usually from 30 days to 9 months after implantation, and preferably from 30 days to 6 months after implantation. The second therapeutic composition is typically configured to delay release the at least one drug of the second drug formulation for at least one 24-hour period following exposure to the vascular environment. The second therapeutic composition is typically configured to release the at least one drug of the second drug formulation at a mean rate not exceeding 2 μg/hour, usually 1 μg/hour, preferably 0.5 μg/hour, and more preferably 0.1 μg/hour after the 24-hour period following exposure to the vascular environment, where the mean rate may be determined based on the amount (weight) of drug released over the total duration of the release.

The first and second therapeutic composition will typically but not necessarily comprise a carrier, matrix, or coating, usually but not always including a polymer, to sequester and control the release rate and duration of the drugs. In some instances, the drugs may be coated, layered, or otherwise deposited on or in surfaces or receptacles on the implantable structure without a polymer or other carrier but optionally with excipients, coating agents, and other conventional drug coating materials.

In some instances, one of the first and second therapeutic compositions may comprise a polymer while the other is free from polymer. For example, the first therapeutic (rapid release) composition may free from polymer and the second (sustained release) therapeutic composition may comprises a polymer to maintain or control the release rate and duration. For example, the first therapeutic composition may be coated on the scaffold structure or over the second therapeutic composition to affect a burst release.

In instances where the first and second therapeutic compositions each comprise a polymer, the first therapeutic composition will have a first drug-to-polymer weight ratio and the second therapeutic composition will a second drug-to-polymer weight ratio. The ratios may be the same but will more often be different. For example, the first drug-to-polymer weight ratio may be in a range from 5:1 to 1:3, usually from 5:2 to 1:2, and preferably from 5:3 to 1:1, and the second drug-to-polymer weight ratio may in a range from 5:2 to 1:5, usually from 5:3 to 2:5, and preferably from 1:1 to 1:2. The first drug-to-polymer weight ratio is usually greater than the second drug-to-polymer weight ratio (greater loading can enhance the burst effect in the first therapeutic composition), but in some instances the first drug-to-polymer weight ratio may less than the second drug-to-polymer weight ratio (greater loading can also enhance duration of release).

While drug release from the first and second therapeutic compositions may commence simultaneously, in many instances the first therapeutic composition and the second therapeutic composition are configured to delay start of release of the second drug formulation for a time period after release of the first drug formulation has started. For example, the first therapeutic composition may be layered over the second therapeutic composition to delay release of the second drug formulation, e.g., the first therapeutic composition may initially cover at least a portion of the second therapeutic composition and may be configured to dissolve over the time period in the vascular environment to expose the second therapeutic composition and allow release of the second drug formulation.

Alternatively, a sacrificial layer may present over at least one of the first therapeutic composition and the second therapeutic composition or between the first therapeutic composition and the second therapeutic composition to delay release of one or more drugs from either or both of the first therapeutic composition and the second therapeutic compositions.

Alternatively, a diffusion-rate controlling layer may be present over at least one of the first therapeutic composition and the second therapeutic composition or between the first therapeutic composition and the second therapeutic composition to control a release rate of one or more drugs from either or both of the first therapeutic composition and the second therapeutic compositions.

The polymer(s) may be configured to release the first and/or second drug formulation at least partly by dissolution of the polymer when exposed to the vascular environment. For example, the polymer of the first therapeutic composition may dissolve at a faster rate than dissolution of the second therapeutic composition in the vascular environment. Alternatively, the polymer may be configured to release the first and/or second drug formulation at least partly by a diffusion mechanism through the polymer when exposed to the vascular environment. Alternatively, the polymer may be configured to release the first and/or second drug formulation through a combination of dissolution of and diffusion through the polymer when exposed to the vascular environment.

Usually, but not necessarily, one or more polymers will be porous where the first and/or second drug formulation are sequestered in pores of the polymer(s). Often, a release rate of the first and/or second drug formulation may at least partly be determined by a pore size of the polymer. In some instances, the polymers of the first and second drug formulations may have different pore sizes which provide different release rates.

In other instances, the first and second drug formulations may be at least partially separated in different regions within the porous polymer. Alternatively or additionally, the first and second drug formulations may at least partially present in overlapping regions of the porous polymer.

In preferred instances, the implantable scaffold of the present disclosure will further comprise an anti-proliferative drug. The anti-proliferative drug is present in either or both of the first and second drug formulations or may be present in a third drug formulation or may be separately coated, coupled, bonded, or attached to the scaffold. For example, the anti-proliferative drug may present in a third therapeutic composition formulated to release the anti-proliferative drug into a vascular environment when the scaffold is present in the vascular environment.

The first, second, and optionally third or additional therapeutic compositions of the present disclosure may be positioned on an external, internal, edge, and/or other surface of the implantable scaffold. Optionally but not necessarily, the scaffold surfaces will be roughened, scored, etched, or otherwise treated to enhance attachment of the therapeutic compositions. In some instances, the therapeutic compositions may be sequestered in wells, indentations or other receptacles formed on or in the scaffold surfaces.

Exemplary calcium chelating agents of the present disclosure is selected from a group consisting of ethylenediaminetetraacetic acid (EDTA), calcium disodium edetate to calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetrmacetic acid (BAPTA), citrate, oxalate, the surfactant-EDTA complex, quaternary ammonium EDTA salt, benzalkonium EDTA salt, EDTA complex, salts, analogue, solvate, hydrate and derivative thereof.

Exemplary of EDTA complex the present disclosure include monoammonium EDTA salt, diammonium EDTA salt, triammonium EDTA salt, benzyldimethyltetradecylammonium EDTA salt, tridodecylmethylammonium EDTA salt, other benzalkonium EDTA salt, and any chemical could complex with EDTA.

Exemplary direct factor IIa inhibitors of the present disclosure include argatroban, dabigatran, ximelagatran, melagatran, efegatran, inogatran, atecegatran metoxil (AZD-0837), hirudin, hirudin analogs, bivalirudin, desirudin, and lepirudin, which may be used individually or in combination. Preferred direct factor IIa inhibitors comprise argatroban, or a salt, isomer, solvate, analog, derivative, metabolite, or prodrugs thereof.

Exemplary direct factor Xa inhibitors of the present disclosure include apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052), which may be used individually or in combination. Preferred direct factor Xa inhibitor comprise (1) apixaban, or a salt, isomer, solvate, analog, derivative, metabolite, or prodrugs thereof and (2) rivaroxaban, or a salt, isomer, solvate, analog, derivative, metabolite, or prodrugs thereof.

Exemplary anti-proliferative agents of the present disclosure include mycophenolate mofetil, mycophenolate sodium, azathioprine, mTOR inhibitors selected from a group consisting of sirolimus, biolimus, everolimus, myolimus, novolimus, ridaforolimus, temsirolimus, zotarolimus, or salts, isomers, solvates, analogs, derivatives, metabolites, or prodrugs thereof, which may be used individually or in combination. Preferred anti-mTOR proliferative agents comprise sirolimus, or a salt, isomer, solvate, analog, derivative, metabolite, or prodrugs thereof.

Exemplary anti-proliferative agents of the present disclosure also include paclitaxel, or a salt, isomer, solvate, analog, derivative, metabolite, or prodrug thereof.

In addition to the first, second, and optional third therapeutic compositions as discuss above, the implantable scaffolds of the present disclosure may further comprise at least one additional drug, typically an antiplatelet drug. The additional drug will not necessarily be incorporated as a drug formulation or as part of a therapeutic composition.

In specific examples of the present disclosure, the direct factor IIa inhibitor comprises argatroban and the direct factor Xa inhibitor comprises apixaban or rivaroxaban. In other specific examples of the present disclosure, the direct factor IIa inhibitor comprises argatroban or an analogue of argatroban, the direct factor Xa inhibitor comprises apixaban or rivaroxaban or an analogue of apixaban or rivaroxaban, and the anti-proliferative agent comprises sirolimus or an analogue of sirolimus.

In some instances, at least one of the therapeutic compositions may comprises an excipient, an adjuvant, a carrier, a wetting agent. In some instances, the first and second therapeutic compositions may be formed contiguously. In some instances, the first and second therapeutic compositions are separated by barrier, for example a polymer layer.

In some examples, a third therapeutic composition comprises a third drug formulation including at least one drug selected from the group consisting of a calcium chelating agent, a direct factor IIa inhibitor and a direct factor Xa inhibitor. The third drug formulation may comprise any one of the previously discussed drugs and/or an additional drug. The third therapeutic composition may be disposed at least partially over the first therapeutic composition which may disposed at least partially over the second therapeutic composition, where the third therapeutic composition may be configured to effect a burst release which is more rapid than the release of either the first or second therapeutic compositions.

In specific examples, the first and second therapeutic compositions may comprise polymer and the third therapeutic composition may be free from polymer and coated or otherwise deposited over at least a portion of the first therapeutic composition.

In other examples, the third drug formulation may comprise at least one polymer, where at least one polymer in the third formulation may be the same and/as or different from at least one polymer in the first and second drug formulations. For example, the at least one polymer in the third formulation may provide a different release rate than provided by at least one polymer in the first and second drug formulations. In other examples, the at least one polymer in the third formulation provides substantially the same release rate as provided by at least one polymer in the first and second drug formulations.

In specific instances, the first, second, or optional third therapeutic compositions may comprise a plurality of drug different formulations for at least one drug. For example, a single drug type may be sequestered in formulations with polymers have different release rates and/or drug loadings, allowing further control of the drug release characteristics.

In yet another aspect, the present disclosure provides an implantable scaffold comprising a scaffold structure having a surface configured to be expanded in the patient's body. A first therapeutic composition comprising a first drug formulation including at least of a calcium chelating agent or EDTA complex, argatroban, at least one of apixaban and rivaroaxaban, and sirolimus is present in a polymer configured to rapidly release the first drug formulation into a vascular environment. A second therapeutic composition comprises a second drug formulation including at least argatroban, at least one of apixaban and rivaroaxaban, and sirolimus present in a polymer or other carrier configured for extended release of the second drug formulation into the vascular environment.

Carriers will typically comprise polymers, usually biodegradable polymers, more usually but not always being synthetic polymers synthesized from petroleum and other hydrocarbon feedstocks. Exemplary biodegradable, synthetic polymers are selected from the group consisting of polyesters, including polylactic acids, polyglycolic acids, polylactic acid-co-glycolic acids, polylactic acid-co-caprolactones, polyethylene glycol-block-poly caprolactone, and polyurethanes: poly(methyl methacrylate) (PMMA); poly N-(2-Hydroxypropyl) methacrylamides; polyethylenimine (PEI), dextran, dextrin, chitosans, poly(L-lysine); poly(aspartamides), polyethylenes; polypropylenes; polyamides; polyethylene glycols (PEG); silicones; poly(anhydrides); and poly ortho esters.

An exemplary biodegradable polymer comprises poly(lactic-co-glycolic acid) (PLGA), where the PLGA is present at 5 μg to 15 μg per mm of scaffold structure length in the first therapeutic composition and from and from 5 μg to 20 μg per mm of scaffold structure length in the second therapeutic composition.

Alternatively, the polymer may comprises a non-degradable polymer, for example being selected from the group consisting of polyacrylates, polymethacrylates, poly(n-butyl methacrylate), poly(hydroxyethylmethacrylate), polyamides, nylons, nylon 12, Dacron, polyethylene terephthalate, poly(ethylene glycol), polyethylene oxide (PEO), polydimethylsiloxane, polyvinylpyrrolidone, ethylene-vinyl acetate, phosphorylcholine-containing polymers, poly(2-methacryloyloxyethylphosphorylcholine), poly(2-methacryloyloxyethylphosphorylcholine-co-butyl methacrylate), and copolymers thereof.

In specific examples, the argatroban, at least one of apixaban and rivaroaxaban, and the sirolimus may be sequestered in a porous structure of the PLGA, and the release of the argatroban, the direct factor Xa inhibitor including at least one of apixaban and rivaroaxaban, and the sirolimus into the vascular environment occurs through a combination of diffusion and dissolution.

In a particular example, (1) a calcium chelating agent may be present in the first therapeutic composition at a concentration in a range from 2 μg to 15 μg per mm of scaffold structure length, the argatroban may be present in the first therapeutic composition at a concentration in a range from 0.5 μg to 3 μg per mm of scaffold structure length, the direct factor Xa inhibitor including at least one of apixaban and rivaroaxaban may be present at a concentration in a range from 0.5 μg to 3 μg per mm of scaffold structure length, and the sirolimus may be present at a concentration in a range from 0.5 μg to 3 μg per mm of scaffold structure length in the first therapeutic composition and (2) the argatroban may be present at a concentration in a range from 2 μg to 10 μg per mm of scaffold structure length, the direct factor Xa inhibitor including at least one of apixaban and rivaroxaban may be present at a concentration in a range from 2 μg to 10 μg per mm of scaffold structure length, and the sirolimus may be present at a concentration in a range from 2 μg to 10 μg per mm of scaffold structure length in the second therapeutic composition.

In a particular example, (1) the argatroban may be present in the first therapeutic composition at a concentration in a range from 0.5 μg to 3 μg per mm of scaffold structure length, the direct factor Xa inhibitor including at least one of apixaban and rivaroaxaban may be present at a concentration in a range from 0.5 μg to 3 μg per mm of scaffold structure length, and the sirolimus may be present at a concentration in a range from 0.5 μg to 3 μg per mm of scaffold structure length in the first therapeutic composition and (2) a calcium chelating agent may be present in the first therapeutic composition at a concentration in a range from 2 μg to 15 μg per mm of scaffold structure length, the argatroban may be present at a concentration in a range from 2 μg to 10 μg per mm of scaffold structure length, the direct factor Xa inhibitor including at least one of apixaban and rivaroxaban may be present at a concentration in a range from 2 μg to 10 μg per mm of scaffold structure length, and the sirolimus may be present at a concentration in a range from 2 μg to 10 μg per mm of scaffold structure length in the second therapeutic composition.

In other examples, the first therapeutic composition may be coated on one or more surfaces of the scaffold structure and the second therapeutic composition may be coated over at least a portion of the first therapeutic composition. For example, the first and second therapeutic compositions cover at least 75% of the area of inner and outer surfaces of the scaffold structure.

In a still further aspect, the present disclosure comprises a method for treating a vascular tissue injury in a patient. The method comprises expanding a scaffold structure at a target location in the patient's vasculature proximate a tissue injury. A first drug formulation including at least one of a drug selected from the group consisting of a calcium chelating agent, a direct factor IIa inhibitor and a direct factor Xa is released from a first therapeutic composition on the scaffold, and a second drug formulation including at least one drug selected from the group consisting of a calcium chelating agent, a direct factor IIa inhibitor and a direct factor Xa is released from a second therapeutic composition on the scaffold to the location of injury. The first therapeutic composition may be formulated to rapidly release the first drug formulation into a vascular environment, and the second therapeutic composition may be formulated to provide an extended release of the second drug formulation into the vascular environment.

In different instances of the methods of the present disclosure, the therapeutic compositions may be positioned on an external surface of the implantable scaffold, on an internal surface of the implantable scaffold, or on both external and internal surfaces of the implantable scaffold.

While the tissue injury will frequently be caused by expanding the scaffold at the location, in other cases the tissue injury may preexist, deploying the structure at the location.

In specific instances, at least one of the first drug formulation and the second drug formulation may comprise either or both or three of a calcium chelating agent, a direct factor IIa inhibitor and a direct factor Xa inhibitor.

In specific instances, the first (rapid release) drug formulation may release drug from the first therapeutic composition over a first time period is in a range from 3 hours to 28 days after implantation, usually from 3 hours to 7 days after implantation, preferably from 3 hours to 3 days after implantation, where the at least one drug of the first drug formulation is typically at a mean rate in the range from 1 μg/hour to 10 μg/hour, usually from 1 μg/hour to 5 μg/hour, preferably from 2 μg/hour to 4 μg/hour over a 24 hour period following exposure to the vascular environment, where the mean rate may be determined based on the amount (weight) of drug released over the total duration of the release.

In specific instances, the at least one drug of the second drug formulation is released from the second (sustained release) therapeutic composition over a second time period is in a range from 30 days to 12 months after implantation, usually from 30 days to 9 months after implantation, preferably from 30 days to 6 months after implantation, where the second therapeutic composition is typically configured to release the at least one drug of the second drug formulation for at a mean rate not exceeding 2 μg/hour, usually 1 μg/hour, preferably 0.5 μg/hour, and more preferably 0.1 μg/hour after the 24 hour period following exposure to the vascular environment, where the mean rate may be determined based on the amount (weight) of drug released over the total duration of the release.

In preferred instances, the therapeutic compositions are formulated to locally release the first and second drug formulation of a calcium chelating agent to the injury site at a rate or a concentration sufficient to begin to inhibit one or more of inflammation, cell proliferation, internal elastic lamina (IEL) injury, fibrin formation, and clot formation within about 1 hours to about 7 days after the structure is deployed.

In preferred instances, the therapeutic compositions are formulated to locally release the first and second drug formulation Xa to the injury site at a rate or a concentration sufficient to begin to inhibit one or more of inflammation, cell proliferation, internal elastic lamina (IEL) injury, fibrin formation, and clot formation within about 3 hours to about 7 days after the structure is deployed.

In other instances, the methods may further comprise releasing an antiplatelet drug from at least one of the first and second therapeutic compositions.

In another aspect, the present disclosure provides an implantable scaffold comprising a scaffold structure having a surface configured to be expanded in the patient's body. At least one therapeutic composition comprising a drug formulation including at least one drug selected from the group consisting of a calcium chelating agent, a direct factor IIa inhibitor and a direct factor Xa inhibitors coated, layered, or otherwise bonded or affixed to the scaffold, wherein the therapeutic composition is formulated for an extended release of the drug formulation into a vascular environment.

Often, the drug formulation includes a calcium chelating agent, a direct factor IIa inhibitor and a direct factor Xa inhibitor, and in some instances, the drug formulation may include one or more additional drugs as described elsewhere herein.

Usually, the drug formulation comprises a polymer, and the drug(s) are incorporated into the polymer. The polymer is typically non-degradable in the vascular environment, where the drugs are loaded into a porous structure of the polymer and released by diffusion over an extended period. Alternatively, the polymer may be degradable in the vascular environment, and the drugs may be released by a combination of diffusion through and dissolution of the polymer.

Typically, the scaffold comprises a metal or a polymer which is non-degradable in the vascular environment, but in other instances the scaffold may be partly of wholly degradable, particularly when the polymer of the drug formulation is also degradable.

In specific examples, at least one drug of the drug formulation is released from the therapeutic composition over a time period of at least 28 days after implantation, usually at least 3 months after implantation, and preferably at least one year after implantation.

In other examples the therapeutic composition may be configured to release the at least one drug of the drug formulation at a mean rate not exceeding 2 μg/hour, usually 1 μg/hour, preferably 0.5 μg/hour, and more preferably 0.1 μg/hour following exposure to the vascular environment.

In many or all instances, the extended release of the drug formulation acts to accelerate dissolution of one or more of inflammation, cell proliferation, internal elastic lamina (IEL) injury, thrombin, fibrin formation, platelet aggregation, platelet activation, and clot or thrombus formation; and/or inhibit one or more of inflammation, cell proliferation, internal elastic lamina (IEL) injury, thrombin, fibrin formation, platelet aggregation, platelet activation, and clot or thrombus formation; and/or increase or prolong time before blood forms clot or thrombus.

In one aspect, a medical device may comprise a structure having at least one surface configured for internal use within a patient's body and a therapeutic composition comprising one or more active substances. These active substances include but not limited to a direct factor Xa inhibitor such as Apixaban, Betrixaban, Edoxaban. Otamixaban, Rivaroxaban, Razaxaban, (r)-n-(2-(4-(1-methylpiperidin-4-yl)piperazin-1-yl)-2-oxo-1-phenylethyl)-1 h-indole-6-carboxamide (LY-517717), Daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or cribaxaban (PD 0348292), or 2-(5-carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052) or others; and/or a direct IIa inhibitor such as Hirudin, Bivalirudin such as Angiomax, Desirudin, Lepirudin, atecegatran metoxil (AZD-0837), Argatroban, Dabigatran, Efegatran, Inogatran, Melagatran, Ximelagatran, or others; Vitamin K antagonist such as Acenocoumarol, Coumatetralyl, Dicoumarol, Ethyl biscoumacetate, Phenprocoumon, Warfarin, Clorindione, Diphenadione, Phenindione, Tioclomarol, or others; and/or other anti-coagulant drug such as Antithrombin III, Defibrotide, Protein C (Drotrecogin alfa). Ramatroban, REG1, or others; and/or an antiplatelet drug such as Abciximab, Eptifibatide, Orbofiban, Roxifiban, Sibrafiban, Tirofiban, Clopidogrel, Prasugrel, Cangrelor. Elinogrel. Ticagrelor, Beraprost, Iloprost, Prostacyclin, Treprostinil, Acetylsalicylic acid/Aspirin, Aloxiprin, Carbasalate calcium, Indobufen, Triflusal, Dipyridamole/aspirin, Picotamide, Terbogrel, Terutroban, Cilostazol, Dipyridamole, Triflusal, Cloricromen, Ditazole, Vorapaxar, Ticlopidine, or others; and/or thrombolytic drugs/fibrinolytics drug such as Plasminogen activators r-tPA, Alteplase, Reteplase, Tenecteplase, Desmoteplase, Saruplase, Urokinase, Anistreplase, Monteplase, Streptokinase, Ancrod, Brinase, Fibrinolysin, or others; and/or Ethylenediaminetetraacetic acid (EDTA), calcium disodium edetate, magnesium dipotassium edetate, magnesium disodium edetate, Disodium edetate, Tetrasodium edetate, Trisodium edetate, monoammonium EDTA salt, Diammonium EDTA salt, Triammonium EDTA salt, Benzyldimethyltetradecylammonium EDTA salt, Tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), citrate, oxalate, the surfactant-EDTA complex, quaternary ammonium EDTA salt, benzalkonium EDTA salt, EDTA complex, salts, analogue, solvate, hydrate and derivative thereof; and/or an inhibitor for intrinsic pathway of coagulation and thrombosis such as a FXI/FXIa inhibitor, protein Z-dependent protease inhibitor; and/or anti-proliferative drug such as Paclitaxel (Taxol), or salts, isomers, solvates, analogs, derivatives, metabolites, or prodrugs, and/or an m-TOR inhibitor such as sirolimus, biolimus, everolimus, myolimus, novolimus, ridaforolimus, temsirolimus, zotarolimus, or salts, isomers, solvates, analogs (including deuterated analogs), derivatives, metabolites, or prodrugs, and combinations thereof. In a preferred example, a medical device comprising a structure having at least one surface configured for internal use within a patient's body and a therapeutic composition comprising one or more active substances, wherein the one or more active substances comprises one of apixaban, rivaroxaban, or argatroban. In a preferred example, a medical device comprising a structure having at least one surface configured for internal use within a patient's body and a therapeutic composition comprising one or more active substances, wherein the one or more active substances comprises apixaban and argatroban, apixaban and an anti-platelet agent, Rivaroxaban, and an anti-platelet agent, or argatroban and an anti-platelet agent. In a preferred example, a medical device comprising a structure having at least one surface configured for internal use within a patient's body and a therapeutic composition comprising one or more active substances, wherein the one or more active substances comprises one of apixaban or rivaroxaban or an analogue thereof, and argatroban or an analogue of it. In another preferred example, a medical device comprising a structure having at least one surface configured for internal use within a patient's body and a therapeutic composition comprising one or more active substances, wherein the one or more active substances comprises one of apixaban or rivaroxaban or an analogue thereof, argatroban or its analogue, and one of taxol or sirolimus or an analogue thereof analogues.

In one aspect, a medical device may comprise a structure having an external surface configured for internal use within a patient's body and a therapeutic composition comprising one or more active substances including a calcium chelating agent disposed on at least one surface, preferably disposed on the entire external surface of the structure. In some examples, the external surface of the structure is configured to be positioned adjacent to an injury site in the patient's body, preferably expanding such site to a larger configuration.

In one aspect, a medical device may comprise a structure having an external surface configured for internal use within a patient's body and a therapeutic composition comprising one or more active substances including a direct factor IIa inhibitor disposed on at least one surface, preferably disposed on the entire external surface of the structure. In some examples, the external surface of the structure is configured to be positioned adjacent to an injury site in the patient's body, preferably expanding such site to a larger configuration.

In some examples, the therapeutic composition is formulated to reduce, inhibit, and/or maintain reduced cell inflammatory at implantation injury site for an extended period, usually at least about 28 days after the external surface of the structure is positioned adjacent the injury site, typically from 1 month to about 12 months.

While the drug carriers of the present disclosure will often be synthetic, biodegradable polymers, as described above, in other examples, the drug carrier may be formulated with other biocompatible, biodegradable, or non-biodegradable materials, including non-synthetic polymers, such as biological polymers, such as proteins, polypeptides, nucleic acids, carbohydrates, and the like, formulated in coating or other sequestration materials.

Such biological and other biocompatible coatings can reduce the foreign body inflammatory response induced by the intraluminal device. In some examples, such biocompatible drug carriers can deliver effective drug concentration within the vessel walls, provide a reservoir for abluminal drug elution, direct a drug toward a vessel wall, enable enhanced drug-tissue permeation to achieve enhanced drug bioavailability, improve homogeneous drug distribution, and/or improve drug stability.

In some examples, the therapeutic composition is formulated to improve drug delivery to target cells, such as diabetic cells.

In some examples, the therapeutic composition is formulated to improve drug lipophilicity and carrier hydrophobicity to facility drug effective delivery to vessel wall and extend drug release rate.

In some examples, instead of being a component of the coating, the therapeutic agent may also be chemically combined with the coating, carrier, or matrix by any chemical combination technique.

In some examples, the therapeutic composition is formulated with a biocompatible carrier having a surface-binding cell adhesion polypeptide deposited on the stent surface forming an amino-containing hydrophobic bond by binding moiety included but not limit to 3,4-dihydroxyphenylalanine (DOPA) or having adhesives peptide or polypeptide such as in the form of L-DOPA-containing proteins. These positive charged amino-terminal region polypeptides inhibit platelet activation and degranulation and limit platelet adhesion in the stent surface.

In some examples, the therapeutic composition is formulated with a biocompatible carrier deposited on the surface, forming a hydrophobic coating to enhance the corrosion resistance, avoiding the aggregation of platelets in the blood vessels and appropriate proliferation of endothelial cells and controlled proliferation of smooth muscle cells, which reduces the development of pathology, such as neointimal hyperplasia, thrombosis, and restenosis.

In some examples, the therapeutic composition is formulated with a biocompatible carrier with “mussel-inspired,” catechol-functionalized hydrogels. In some examples, the therapeutic composition is formulated with a biocompatible hydrogel or soft gel carrier when it contacted with body fluid, the coating will reduce, inhibit, and/or maintain reduced cell inflammatory at the injury site and extend drug release rate. In some examples, the therapeutic composition is formulated with polyunsaturated fatty acids (PUFAs) such as Omega-3 or Omega-6 to modify platelet responsiveness with anticoagulants.

The biocompatible drug carriers include but not limit to low water solubility amino acid, peptide, polypeptide, modified peptide conjugated with a linker or a spacer, modified polypeptide conjugated with a linker or a spacer, fatty acid including Omega-3 or Omega-6 polyunsaturated fatty acids, crosslinked fatty acid, crosslinked oil, including fish oil, Vitamin E, hazelnut oil, avocado oil, macadamia nut oil, grapeseed oil, groundnut oil (peanut oil), sesame oil, corn oil, almond oil, sunflower oil, hemp oil, tea-oil camellia, pectin and gelatin. Exemplary amino acids are selected from the group consisting of phenylalanine, valine, threonine, tryptophan, methionine, leucine, isoleucine, lysine, histidine, arginine, cysteine, glycine, glutamine, proline, tyrosine, alanine, aspartic acid, asparagine, glutamic acid, serine, and selenocysteine, and derivatives and combinations thereof. Exemplary low-solubility amino acid having a solubility in unbuffered water of less than 40 mg/mL are selected from the group consisting of asparagine, aspartic acid, cystine, eptifibatide, isoleucine, leucine, methionine, phenylalanine, tryptophan, tyrosine, and combinations thereof. Exemplary of peptides include but not limit to lysine, ornithine, arginine, histidine, glutamic acid, aspartic acid, histidine, polyornithine, serine, threonine, tyrosine, leucine, analogues including D and L isomers, oligomers, copolymers, block polymers, derivatives, and any peptide with different amino acid sequence. Exemplary of peptides include but not limit to signaling peptides, carrier peptides, enzyme-inhibiting peptides, neurotransmitter-inhibiting peptides, antimicrobial peptides, analogs, derivatives, and combinations thereof. Exemplary of polypeptides include but not limit to poly(lysine), poly(ornithine), poly(arginine), poly(histidine), poly(glutamic acid), poly(aspartic acid), poly(histidine), poly(ornithine), poly(serine), poly(threonine), poly (tyrosine), poly(leucine), analogues including D and L isomers, copolymers, block polymers, derivatives, and combinations thereof. Exemplary of cell adhesion polypeptides include but not limit to fibronectin, vitronectin, laminin, elastin, fibrinogen, and collagens, such as types I, II, and V, and any peptide derived from any of the peptides, including a cell adhesive peptide fragment having the amino acid sequence, and any peptide with different amino acid sequence. Exemplary of drug carries are containing at least one multiple bonds, i.e. preferably one unsaturated fatty acid moiety, fatty acids, cross-linked fatty acid, fatty acid esters, fatty acid derivatives, ethers, diethers, tetraethers, lipids, oils, fats, glycerides, tri-glycerides, glycol esters, glycerin esters, fish oil or derivatives thereof, vitamin E or derivatives thereof, peanut oil, cotton-seed oil, oleic acid or combinations thereof as well as mixtures of the aforementioned substances. Suitable saturated fatty acids include but not limited to, those selected from the group consisting of butyric acid, valeric acid caproic acid, enanthic acid, caprylic acid, pelargonic acid, capric acid, lauric acid, myristic acid, pentadecanoic acid, palmitic acid, margaric acid, stearic acid, arachidic acid, behenic acid, lignoceric acid, cerotic acid, linoleic acid, oleic acid, and stearic acid. Suitable unsaturated fatty acids include but not limited to those selected from the group consisting of arachidonic acid, oleic acid, erucic acid, nervonic acid, linolenic acid, arachidonic acid, eicosapentanoic acid (EPA), docosahexanoic acid (DHA), palmitoleic acid, and myristoleic acid.

In some examples, the biocompatible carrier comprises polypeptides conjugated with an adhesive moiety and/or a linker to enhance hydrophobicity and ease drug delivery. Adhesive moieties, including but not limited to catechol moieties and L-DOPA-containing proteins, may be joined to each other and to cell adhesive polypeptides by linkers including but not limit to hyaluronic acid, polyethylene glycol/poly lysine, dopamine, 1,6-diaminohexane, 1,5-diaminopentane, 1,4-diaminobutane, or 1,3-diaminopropane or any compound having at least two hydroxyl group or two amine group that could reacted with polypeptide amnio acid group. In some examples, the biocompatible carrier is peptides and conjugated with a spacer and/or a linker to make peptide more hydrophobic and having controlled delivery of therapeutic compounds or an extended-release rate of therapeutic compounds. In some examples, the biocompatible carrier is a controlled release layer contains one or more matrix forming gelling agents selected from group consisting of hydroxypropyl methylcellulose, methylcellulose, hydroxypropyl cellulose, carbomer, carboxy methylcellulose, gum tragacanth, gum acacia, guar gum, pectin, modified starch derivatives, xanthan gum, locusta bean gum, sodium alginate, which on contact with gastric fluid swells and gels, forming matrix structure that entraps the gas released and also releases the active agent in a controlled manner. In some examples, the biocompatible carrier is an absorption promoter selected from the group consisting of propylene glycol, propylene glycol monolaurate, isopropyl palmitate, 1,2,6-hexanetriol, polyethylene glycol, diisopropyl adipate, polyethylene glycol 400 acetate, and ethylene glycol monoether. In some examples, the therapeutic composition comprises a coating disposed on the external surface of the structure, and the coating comprises plasmid DNA loaded biodegradable polymer such as Polylactic-polyglycolic acid (PLGA) as stent coating. This gene therapy on the vessel wall by effective transfection of neointimal cells by local delivery of DNA. The carriers include but not limit to DNA fragments, nucleic acids, genetic material, oligonucleotides, radioisotopes, or combinations of these classes of compounds.

In some examples, the therapeutic composition is formulated to release the one or more agents, configured to release the two or more agents, or is configured to release the three or more agents, in one or more of the following: a burst release phase and an extended-release phase, wherein the release of a first phase comprises a faster release rate than a second release phase, or other.

In some examples, the therapeutic composition is formulated to release the one or more agents, wherein the therapeutic composition comprises a first therapeutic composition formulated to release said agents at a faster rate, and a second therapeutic composition formulated to release said agents at a slower release rate.

In some examples, the device comprises one therapeutic composition formulated to release one or more of calcium chelating agent such as of a calcium chelating agent, direct factor Xa inhibitor, direct factor IIa inhibitor, and/or an anti-proliferative, wherein the formulation formulated to release the drugs over an extended period ranging from 7 days to 6 months, preferably ranging from 14 days to 6 months, more preferably ranging from 21 days to 6 months, and most preferably ranging from 30 days to 1 year from exposure to vascular environment. Optionally, the formulation is configured to have a bolus drug release rate within the first 1 hour, 3 hours, or first 24 hours, from exposure to vascular environment.

In some examples, the therapeutic composition comprises at least three therapeutic active substances comprising a calcium chelating agent, a direct factor Xa inhibitor and a direct factor IIa inhibitor.

In some examples, the therapeutic composition comprising a direct factor Xa inhibitor further comprises at least one additional therapeutically active substance. In some examples, the at least one additional therapeutically active substance comprises a direct factor IIa inhibitor selected from the group consisting of argatroban, dabigatran, ximelagatran, melagatran, efegatran, inogatran, atecegatran metoxil (AZD-0837), hirudin, hirudin analogs, bivalirudin, desirudin, or lepirudin. In some examples, the direct factor IIa inhibitor comprises argatroban, or a salt, isomer, solvate, analog, derivative, metabolite, or prodrugs thereof. In some examples, the direct factor Xa inhibitor comprises apixaban and the direct factor IIa inhibitor comprises argatroban. In some examples, the therapeutically effective dose of the direct factor IIa inhibitor is within a range of about 50 micrograms to about 10 mg. In some examples, the therapeutically effective dose is sufficient to generate a blood concentration of the direct factor IIa inhibitor which is smaller than a median maximum serum concentration (Cmax) of the direct factor IIa inhibitor generated by systemic delivery of the direct factor IIa inhibitor to achieve the same tissue concentration at the site of the inflammatory ophthalmic condition or disease. In some examples, the therapeutically effective dose is sufficient to generate a blood concentration of the direct factor IIa inhibitor which does not exceed a median maximum serum concentration (Cmax) of the direct factor IIa inhibitor generated by systemic delivery of the direct factor IIa inhibitor to achieve the same tissue concentration at the site of the inflammatory ophthalmic condition or discase for more than about 6 hours to about 3 days. In some examples, the therapeutically effective dose is sufficient to maintain a tissue concentration of the direct factor IIa inhibitor of about 0.1 ng % g tissue to about 100 mg/g tissue for about 1 day to about 1 year, 30 days to about 1 year, 3 months to about 1 year, or 6 months to about 1 year. In some examples, the weight compositional ratio of the direct factor Xa inhibitor to the direct factor IIa inhibitor in the therapeutic composition is within a range of about 3:1 to about 1:3. For example, the weight compositional ratio of the direct factor Xa inhibitor to the direct factor IIa inhibitor in the therapeutic composition may be about 1:1.

In some examples, the therapeutic composition comprises one or more anticoagulant agents that has an IC50 to inhibit factor Xa and factor 11 at a dose ranging from 0.0001 nM to 1000 nM, preferably at a dose ranging from 0.0001 nM to 100 nM, more preferably at a dose ranging from 0.0001 nM to 10 nM, and most preferably at a dose ranging from 0.0001 nM to 1 nM.

In some examples, the therapeutic composition is formulated to release the direct factor Xa inhibitor and the anti-proliferative agent at the same rate. In some examples, the therapeutic composition is formulated to release the direct factor Xa inhibitor, and the anti-proliferative agent at different rates. In other examples, the therapeutic composition is formulated to release the direct factor Xa inhibitor at a faster rate than the anti-proliferative agent within the first 3 hours, 1 day, or 72 hour. In yet another example, the therapeutic composition is formulated to release the direct factor Xa inhibitor at a slower rate than the anti-proliferative agent within the first 3 hours, 1 day, or 72 hour.

In some examples, the release rate ratio of the direct factor Xa inhibitor to the anti-proliferative agent is within a range of about 1:6 to about 6:1, or about 1:3 to about 6:1, or about 2.5:2 to about 6:1. In some examples, the release rate ratio of the direct factor Xa inhibitor to the anti-proliferative agent is within a range of about 1:2 to about 6:1, about 1:3 to about 6:1, or about 2.5:2 to about 6:1 within about 3 hours, about 24 hours, about 7 days, or about 28 days. In some examples, the release rate ratio of the direct factor Xa inhibitor to the anti-proliferative agent is within a range of about 3:2 to about 6:1, or about 2.2:2 to about 6:1, or about 2.5:2 to about 6:1. In some examples, the release rate ratio of the direct factor Xa inhibitor to the anti-proliferative agent is within a range of about 3:2 to about 6:1, about 2.2:2 to about 6:1, or about 2.5:2 to about 6:1 within about 3 hours, about 24 hours, about 7 days, or about 28 days. In some other examples, the release rate ratio of the direct factor Xa inhibitor to the anti-proliferative agent is within a range of about 1:1 to about 2:1 within about 3 hours, 1 day, about 3 days, about 7 days, or about 28 days.

In some examples, the therapeutic composition is formulated to release the anti-proliferative agent at a rate of about 1 μg/second/mm device to about 50 μg/day/mm device, of about 1 μg/min/mm device to about 10 μg/day/mm device, or of about 1 μg/hour/mm device to about 7 μg/day/mm device within about 3 hours, about 1 day, or about 3 days.

In some examples, the therapeutic composition is formulated to release the anti-proliferative agent at a rate of about 1 μg/hour/mm device to about 4 μg/day/mm device.

In some examples, the weight compositional ratio of the direct factor Xa inhibitor to the anti-proliferative agent in the therapeutic composition is about 5:2, about 2:1, about 1.25:1, or about 1:1. In some examples, the weight compositional ratio of the direct factor Xa inhibitor to the anti-proliferative agent in the therapeutic composition is within a range of about 5:1 to about 3:1 or about 5:1 to about 1:1.

In some examples, the therapeutic composition comprises a coating disposed on one or more surfaces of the device structure, and the coating comprises a first layer and a second layer. In some examples, the first layer comprises the direct factor Xa inhibitor. In some examples, the first layer comprises the anti-proliferative agent and the second layer comprises the direct factor Xa inhibitor. In some examples, the therapeutic composition further comprises a top layer or coat of the same or different material as the first layer or the second layer. In some examples, the first layer comprises the direct factor Xa inhibitor and the anti-proliferative agent. In some examples, the second layer comprises a top layer or coat of the same or different material as the first layer. In some examples, the therapeutic composition comprises a coating disposed on one or more surfaces the device structure, and the coating further comprises a biodegradable polymer carrier. In some examples, the first and/or second layer comprise a drug/polymer matrix of the one or more agents. In one example, the first layer is configured for a burst release of the one or more agents, while the second layer is configured for an extended release of the one or more agents. In yet another example, the first and/or second layer are topcoat covering one or more drug agents wherein the one or more drug agents are formulated with an excipient or are formulated in a drug polymer matrix under said first and/or second layer coating. The coating of the matrix and the first or second layers maybe the same or different.

In some examples, the weight compositional ratio of the biodegradable polymer carrier to the one or more active substances is about 1:5 to about 3:2, about 0.5:1 to about 1:1, or about 1:5 to about 1.25:1. In a preferred example, the polymer is biodegradable.

In some other examples, the weight compositional ratio of the carrier to the one or more active substances is about 1:5 to about 3:2, about 0.5:1 to about 1:1, or about 1:5 to about 1.25:1. In one example the carrier is one or more excipients.

In some examples, the therapeutic composition is disposed on at least one surface of the device, preferably on at least the external and/or the inner surfaces of the structure. In some examples, the therapeutic composition is disposed on the external surface (abluminal) of the structure, on the interior surface (luminal) of the structure, and on the side surfaces of the structure. In yet other examples, the therapeutic composition is disposed on one or more surfaces of the structure. In yet other examples, the therapeutic composition is disposed on all surfaces of the structure. In yet other examples, the therapeutic composition is disposed in a reservoir on or in the structure. In some examples, the therapeutic composition is disposed on the external surface of the structure.

In some examples, the therapeutic composition comprises a coating disposed on the external surface of the structure, and the coating further comprises a non-degradable polymer carrier. In some examples, the therapeutic composition comprises a coating disposed on the external surface of the structure, and the coating comprises at least one layer of a polymeric material containing the direct factor Xa inhibitor. In some examples, the therapeutic composition comprises a coating disposed on the external surface of the structure, and the coating consists of a single layer of a polymeric material which releasably contains the direct factor Xa inhibitor. In some examples, the therapeutic composition further comprises a top layer or coat comprising the same or different polymeric material. In some examples, the direct factor Xa inhibitor is uniformly distributed in the polymeric material. In some examples, the direct factor Xa inhibitor is non-uniformly distributed in the polymeric material.

In some examples, the therapeutic composition comprises a coating disposed on at least on surface of the structure, and the coating comprises at least one layer of a polymeric material holding one or more of the direct factor Xa inhibitor and the anti-proliferative agent. In some examples, the therapeutic composition comprises a coating disposed on the external surface of the structure, and the coating consists of a single layer of a polymeric material which releasably contains the direct factor Xa inhibitor and the anti-proliferative agent. In some examples, the therapeutic composition further comprises a top layer or coat comprising the same or different polymeric material. In some examples, the direct factor Xa inhibitor, and the anti-proliferative agent are uniformly distributed in the polymeric material. In some examples, the direct factor Xa inhibitor, and the anti-proliferative agent are non-uniformly distributed in the polymeric material. In some examples, the one or more active substances is present in the polymeric material at weight ratios within a range of about 1:1 to about 6:1 of direct factor Xa inhibitor to anti-proliferative agent.

In some examples, the polymeric material is porous. In some examples, the polymeric material has a porosity within a range of about 10 nm to about 10 μm. In some examples, the polymeric material is non-degradable. In some examples, the polymeric material is biodegradable. In some examples, the polymeric material has a degradation rate within a range of about 1 month to about 36 months. In some examples, the polymeric material comprises a material selected from a group consisting of polyesters, polylactide, polyglycolide, poly(ε-caprolactone), polydioxanone, poly(hydroxyalkanoates), poly(L-lactide-co-D-lactide), poly(L-lactide-co-D,L-lactide), poly(D-lactide-co-D,L-lactide), poly(lactide-co-glycolide) (including 70:30 to 99:1 PLA-co-PGA, such as 85:15 PLA-co-PGA), poly(lactide-co-ε-caprolactone) (including 70:30 to 99:1 PLA-co-PCL, such as 90:10 PLA-co-PCL), poly(glycolide-co-ε-caprolactone), poly(lactide-co-dioxanone), poly(glycolide-co-dioxanone), poly(lactide-co-trimethylene carbonate), poly(glycolide-co-trimethylene carbonate), poly(lactide-co-ethylene carbonate), and copolymers and combinations thereof, wherein lactide includes L-lactide, D-lactide and D,L-lactide. In some examples, the polymeric material comprises a material selected from a group of non-degradable polymeric materials consisting of polyacrylates, polymethacrylates, poly(n-butyl methacrylate), poly(hydroxyethylmethacrylate), polyamides, nylons, nylon 12, Dacron, Polyethylene terephthalate, poly(ethylene glycol), polyethylene oxide (PEO), polydimethylsiloxane, polyvinylpyrrolidone, ethylene-vinyl acetate, phosphorylcholine-containing polymers, poly(2-methacryloyloxyethylphosphorylcholine), poly(2-methacryloyloxyethylphosphorylcholine-co-butyl methacrylate), and copolymers and combinations thereof.

In some examples, the therapeutic composition is disposed within a drug reservoir fluidly coupled to the external surface of the structure.

In another aspect, a medical device may comprise a structure having at least one surface configured for internal use within a patient's body and a therapeutic composition comprising two or more active substances including a calcium chelating agent, a direct factor Xa inhibitor and a direct factor IIa inhibitor. In some examples, the at least one surface of the structure is configured to be positioned adjacent an injury site in the patient's body. In some examples, the therapeutic composition is formulated to locally release the two or more active substances to the injury site at a rate or a concentration sufficient to reduce cell proliferation at the injury site within about 3 hours to about 7 days, or within about 28 days to about 12 months, after the external surface of the structure is positioned adjacent the injury site.

In some examples, the therapeutic composition further comprises an anti-proliferative agent. In some examples, the direct factor IIa inhibitor comprises argatroban, dabigatran, ximelagatran, melagatran, efegatran, inogatran, atecegatran metoxil (AZD-0837), hirudin, hirudin analogs, bivalirudin, desirudin, or lepirudin. In some examples, the direct factor IIa inhibitor comprises argatroban, or a salt, isomer, solvate, analog (including deuterated analog), derivative, metabolite, or prodrugs thereof. In some examples, the direct factor IIa inhibitor comprises dabigatran, or a salt, isomer, solvate, analog (including deuterated analog), derivative, metabolite, or prodrugs thereof.

In some examples, the direct factor Xa inhibitor comprises apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150). 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), or 2-(5-carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052). In some examples, the direct factor Xa inhibitor comprises rivaroxaban, or a salt, isomer, solvate, analog (including deuterated analog), derivative, metabolite, or prodrugs thereof. In some examples, the direct factor Xa inhibitor comprises apixaban, or a salt, isomer, solvate, analog (including deuterated analog), derivative, metabolite, or prodrugs thereof. In some examples, the anti-proliferative agent comprises Paclitaxel (Taxol), or salts, isomers, solvates, analogs, derivatives, metabolites, or prodrugs thereof. In some examples, the anti-proliferative agent comprises an m-TOR inhibitor. In some examples, the anti-proliferative agent comprises sirolimus, biolimus, everolimus, myolimus, novolimus, ridaforolimus, temsirolimus, zotarolimus, or salts, isomers, solvates, analogs (including deuterated analogs), derivatives, metabolites, or prodrugs thereof. In some examples, the anti-proliferative agent comprises sirolimus, or a salt, isomer, solvate, analog (including deuterated analog), derivative, metabolite, or prodrugs thereof.

In some examples, the direct factor IIa inhibitor comprises Argatroban and the direct factor Xa inhibitor comprises apixaban. In some examples, the direct factor IIa inhibitor comprises Argatroban, the direct factor Xa inhibitor comprises apixaban, and the anti-proliferative agent comprises sirolimus. In yet another examples the therapeutic composition comprises one of Apixaban, Rivaroxaban, or an analogue thereof, and one of Sirolimus or an analogue of Sirolimus.

In some examples, the therapeutic composition of a calcium chelating agent, a direct factor IIa inhibitor and a direct factor Xa inhibitor is formulated to reduce cell proliferation compared to either the direct factor IIa inhibitor or the direct factor Xa inhibitor alone. In some examples, the therapeutic composition is formulated to reduce, inhibit, and/or maintain reduced cell proliferation at the injury site at about 28 days after the external surface of the structure is positioned adjacent the injury site to about 12 months. In some examples, the therapeutic composition is formulated to reduce smooth muscle cell proliferation at the injury site. In some examples, the therapeutic composition of a calcium chelating agent, a direct factor IIa inhibitor and a direct factor Xa inhibitor and an antiproliferative is formulated to reduce cell proliferation compared to an antiproliferative alone. In some examples, the therapeutic composition is formulated to reduce, inhibit, and/or maintain reduced cell proliferation at the injury site at about 28 days after the external surface of the structure is positioned adjacent the injury site to about 12 months.

In other examples, the therapeutic composition comprising a calcium chelating agent, a direct factor IIa inhibitor and a direct factor Xa inhibitor is formulated to release said agents at a rate and/or concentration sufficient to accelerate dissolution or to inhibit one or more of inflammation, smooth muscle cell proliferation, cell proliferation, thrombin formation, fibrin formation, platelet aggregation, platelet activation, vessel injury, or clot formation, within about 3 hours to about 28 days or longer, or within about 3 hours to about 3 months or longer.

In other examples, the therapeutic composition comprising a calcium chelating agent, a direct factor IIa inhibitor and a direct factor Xa inhibitor is formulated to release said agents to accelerate dissolution of or to inhibit one or more of inflammation, smooth muscle cell proliferation, cell proliferation, thrombin formation, fibrin formation, platelet aggregation, platelet activation, vessel injury, or clot formation, within about 3 hours to about 28 days or longer, or within about 3 hours to about 3 months or longer.

In other examples, the therapeutic composition comprising a calcium chelating agent, a direct factor IIa inhibitor and a direct factor Xa inhibitor formulated to have a weight composition ratio of factor Xa inhibitor to factor IIa inhibitor in the ratio ranging from about 1:1:1 to about 10:1:1 In other examples, the therapeutic composition comprising a calcium chelating agent, a direct factor IIa inhibitor and a direct factor Xa inhibitor formulated to have a weight composition ratio of factor Xa inhibitor to factor IIa inhibitor in the ratio ranging from about 0.5:1 to about 5:1.

In some examples, the therapeutic composition is formulated to reduce one or more of cell proliferation or fibrin formation within 7 days or longer.

In some examples, the therapeutic composition is formulated to release the two or more active substances at a rate of 1 μg/second/mm device to about 50 μg/day/mm device, preferably at a rate of 1 μg/min/mm device to about 30 μg/day/mm device, more preferably at a rate of 1 μg/hour/mm device to about 30 μg/day/mm device. In some examples, the therapeutic composition is formulated to begin releasing the two or more active substances prior to positioning of the device adjacent to the injury site, or immediately after, or within about 5, about 15, or about 30 minutes after the at least one surface of the structure is positioned adjacent the injury site. In some examples, the therapeutic composition is formulated to begin releasing the two or more active substances before the external surface of the structure is positioned adjacent the injury site. In some examples, the therapeutic composition is formulated to release substantially all of the two or more active substances within about 1 to about 90 days or more. In some examples, the therapeutic composition is formulated to release substantially all of the two or more active substances within about 90 to about 180 days or more. In some examples, the therapeutic composition is formulated to release substantially all of the two or more active substances within about 7 days or about 28 days. In some examples, the therapeutic composition is formulated to release substantially all of the two or more active substances within about 3 hours or about 6 hours or about 12 hours or about 1 day or about 3 days. In some examples, the therapeutic composition is formulated to release at least 50% or at least 60% or at least 70% of the two or more active substances within about 3 hours or about 6 hours or about 12 hours or about 1 day or about 3 days or about 7 days or about 28 days.

In some examples, the release rate ratio of the direct factor IIa inhibitor to the direct factor Xa inhibitor to the anti-proliferative agent is within a range of about 1:1:1 to about 4:4:1. In some examples, the therapeutic composition is formulated to release the direct factor IIa inhibitor at a rate of about 4 μg/hour/mm device to about 14 μg/day/mm device. In some examples, the therapeutic composition is formulated to release the direct factor Xa inhibitor at a rate of about 4 μg/hour/mm device to about 14 μg/day/mm device. In some examples, the therapeutic composition is formulated to release the anti-proliferative agent at a rate of about 1 μg/hour/mm device to about 4 μg/day/mm device.

In some examples, the weight compositional ratio of the direct factor IIa inhibitor to the direct factor Xa inhibitor in the therapeutic composition is about 1:1. In some examples, the weight compositional ratio of the direct factor IIa inhibitor to the direct factor Xa inhibitor in the therapeutic composition is within a range of about 3:1 to about 1:3, for example about 1:1. In some examples, the weight compositional ratio of the direct factor IIa inhibitor to the direct factor Xa inhibitor to the anti-proliferative agent in the therapeutic composition is about 5:5:2. In some examples, the weight compositional ratio of the direct factor IIa inhibitor to the direct factor Xa inhibitor to the anti-proliferative agent in the therapeutic composition is within a range of about 6:6:1 to about 1:3:1.

In some other examples, the weight compositional ratio of the carrier to the two or more active substances is about 1:5 to about 3:1, about 0.5:1 to about 1:1, or about 1:5 to about 1.25:1. In one example the carrier is one or more excipients.

In some examples, the therapeutic composition is disposed on the external surface of the structure and on the internal (inner) surface of the structure. In some examples, the therapeutic composition is disposed on the external surface (abluminal) of the structure, on the interior surface (luminal) of the structure, and on the side surfaces of the structure. In yet other examples, the therapeutic composition is disposed on one or more surfaces of the structure. In yet other examples, the therapeutic composition is disposed on all surfaces of the structure. In yet other examples, the therapeutic composition is disposed in a reservoir on or in the structure. In some examples, the therapeutic composition is disposed on the external surface of the structure.

In some examples, the therapeutic composition comprises a coating disposed on at least one surface of the structure, and the coating comprises a first layer and a second layer. In some examples, the first layer comprises the direct factor IIa inhibitor and the direct factor Xa inhibitor. In sonic examples, the first layer comprises the direct factor IIa inhibitor, and the second layer comprises the direct factor Xa inhibitor. In some examples, the therapeutic composition further comprises a top layer or coat of the same or different material as the first layer or the second layer.

In some examples, the therapeutic composition comprises a coating disposed on at least one surface of the structure, and the coating comprises a first layer and a second layer. In some examples, the first layer comprises the anti-proliferative agent, the direct factor IIa inhibitor, and the direct factor Xa inhibitor. In some examples, the second layer comprises a top layer or coat of the same or different material as the first layer. In some examples, the first layer comprises the anti-proliferative agent and the second layer comprises the direct factor IIa inhibitor and the direct factor Xa inhibitor. In some examples, the first layer comprises the anti-proliferative agent and the direct factor Xa inhibitor and the second layer comprises the direct factor IIa inhibitor. In some examples, the first layer comprises the direct factor IIa inhibitor and the direct factor Xa inhibitor and the second layer comprises the anti-proliferative agent. In some examples, the first layer comprises apixaban and argatroban and the second layer comprises sirolimus. In some examples, the therapeutic composition further comprises a top layer or coat of the same or different material as the first layer or the second layer.

In some examples, the coating further comprises a third layer. In some examples, the first layer comprises the direct factor IIa inhibitor, the second layer comprises the direct factor Xa inhibitor, and the third layer comprises the anti-proliferative agent. In some examples, the therapeutic composition further comprises a top layer or coat of the same or different material as the first layer, the second layer, or the third layer.

In some examples, the therapeutic composition comprises a coating disposed on at least one surface of the structure, and the coating further comprises a biodegradable polymer carrier. In some examples, the weight compositional ratio of the biodegradable polymer carrier to the two or more active substances is about 1:5 to about 3:2. In some examples, the therapeutic composition comprises a coating disposed on the external surface of the structure, and the coating comprises at least one layer of a polymeric material holding one or more of the direct factor IIa inhibitor and the direct factor Xa inhibitor.

In some examples, the therapeutic composition comprises a coating disposed on at least one surface of the structure, and the coating consists of a single layer of a polymeric material which releasably holds each of the direct factor IIa inhibitor and the direct factor Xa inhibitor. In some examples, the therapeutic composition further comprises a top layer or coat comprising the same or different polymeric material. In some examples, the direct factor IIa inhibitor and the direct factor Xa inhibitor are uniformly distributed in the polymeric material. In some examples, the direct factor IIa inhibitor and the direct factor Xa inhibitor are non-uniformly distributed in the polymeric material.

In some examples, the therapeutic composition comprises a coating disposed on at least one surface of the structure, and the coating comprises at least one layer of a polymeric material holding one or more of the direct factor IIa inhibitor, the direct factor Xa inhibitor, and the anti-proliferative agent. In some examples, the therapeutic composition comprises a coating disposed on at least one surface of the structure, and the coating consists of a single layer of a polymeric material which releasably holds each of the direct factor IIa inhibitor, the direct factor Xa inhibitor, and the anti-proliferative agent. In some examples, the therapeutic composition further comprises a top layer or coat comprising the same or different polymeric material. In some examples, the direct factor IIa inhibitor, the direct factor Xa inhibitor, and the anti-proliferative agent are uniformly distributed in the polymeric material. In some examples, the direct factor IIa inhibitor, the direct factor Xa inhibitor, and the anti-proliferative agent are non-uniformly distributed in the polymeric material.

In some examples, the two or more active substances are present in the polymeric material at weight ratios of about 4:1:3:1; about 5:3:2:1; about 4:2:2:1; about 5:2:3:1; about 6:3:3:1; about 10:5:5:1; or about 12:6:6:1 of a calcium chelating agent, direct factor IIa inhibitor to direct factor Xa inhibitor to anti-proliferative agent.

In another aspect, a method of treating one or more of inflammation, cell proliferation, smooth muscle cell proliferation, or clotting in a patient may comprise providing a structure having an external surface; deploying the structure at a target location in the patient's body so as to cause an injury at the location; and releasing from at least one surface of the deployed structure to the location of injury in the patient's body therapeutically effective amounts of a therapeutic composition including at least a calcium chelating agent, a direct factor IIa inhibitor, a direct factor Xa inhibitor, and an anti-proliferative agent.

In other examples, the therapeutic composition comprising a calcium chelating agent, a direct factor IIa inhibitor and a direct factor Xa inhibitor and an anti-proliferative is formulated to release said agents at a rate sufficient to inhibit one or more of inflammation, smooth muscle cell proliferation, cell proliferation, thrombin formation, fibrin formation, or clot formation, within about 3 hours to about 28 days or longer, or within about 3 hours to about 3 months or longer.

In another example, the therapeutic composition is formulated to release the two or more active substances, wherein the two or more substances comprise a calcium chelating agent, a direct IIa inhibitor, a direct Xa inhibitor, and an antiproliferative, to an injury site in a body lumen. In another example, the therapeutic composition is formulated to release the two or more active substances, wherein the two or more substances include of a calcium chelating agent, a direct IIa inhibitor, a direct Xa inhibitor, and an antiproliferative, to an injury site in a body lumen. In another example, the therapeutic composition is formulated to release the two or more active substances, wherein the two or more substances include of a calcium chelating agent, a direct IIa inhibitor, and an antiproliferative, to an injury site in a body lumen. In another example, the therapeutic composition is formulated to release the two or more active substances, wherein the two or more substances include of a calcium chelating agent, a direct IIa inhibitor, a direct Xa inhibitor, to an injury site in a body lumen. In another example, the therapeutic composition is formulated to release the two or more active substances, wherein the two or more substances include of a calcium chelating agent, a direct Xa inhibitor and an anti-proliferative, to an injury site in a body lumen.

In some examples, include of a calcium chelating agent, the direct factor IIa inhibitor comprises argatroban, the direct factor Xa inhibitor comprises apixaban, and the anti-proliferative agent comprises sirolimus.

In some examples, include of a calcium chelating agent, the direct factor IIa inhibitor comprises argatroban, the direct factor Xa inhibitor comprises rivaroxaban, and the anti-proliferative agent comprises sirolimus.

In some examples, the therapeutic composition comprises a coating on the external surface of the structure or at least one surface of the structure and releasing the therapeutic composition comprises releasing the therapeutic composition from the coating. In some examples, the coating comprises one or more layers. In some examples, the coating comprises a biodegradable porous polymeric material, a degradable polymeric material, or a non-degradable polymeric material. In some examples, a calcium chelating agent, the direct factor IIa inhibitor and the direct factor Xa inhibitor are released faster than the anti-proliferative agent. In some examples, the direct factor IIa inhibitor and the direct factor Xa inhibitor enhance an anti-proliferative effect of the anti-proliferative agent. In some examples, the therapeutic composition is disposed within a drug reservoir fluidly coupled to the external surface of the structure and releasing the therapeutic composition comprises delivering the therapeutic from the drug reservoir to the external surface of the deployed structure.

In some examples, the injury is at least partially caused before deployment of the structure. In some examples, deployment of the structure causes the injury, and the therapeutic composition is formulated to release a calcium chelating agent, the direct factor IIa inhibitor, the direct factor Xa inhibitor, or the anti-proliferative agent before the injury occurs.

In some examples, the therapeutic composition comprises a first and/or second layer comprise a drug/polymer matrix of the one or more agents. In one example, the first layer is configured for a burst release of the one or more agents, while the second layer is configured for an extended release of the one or more agents. In yet another example, the first and/or second layer are topcoat covering one or more drug agents wherein the one or more drug agents are formulated with an excipient or are formulated in a drug polymer matrix under said first and/or second layer coating. The coating of the matrix and the first or second layers maybe the same or different.

In another example of any of the examples in this application, a therapeutic composition comprising two or more active substances on at least one surface of the device is configured to be positioned adjacent to an injury site in the patient's body, wherein adjacent to comprises one or more of the following: next to, touching, deployed at, expanded at, pushing against, placed against, or other. In a preferred example, the active substances are a calcium chelating agent, a direct factor IIa inhibitor and a direct factor Xa inhibitor. In another example the active substances are a calcium chelating agent, a direct factor IIa inhibitor, a direct factor Xa inhibitor and an anti-proliferative. In yet another example, the active substances are one of Argatroban, Rivaroxaban or Apixaban, and Sirolimus or Sirolimus analogue.

The illustrative examples described are not meant to be limiting. Other examples may be utilized, and other changes may be made, or combined in whole or in part, without departing from the scope of the subject matter presented herein. It will be readily understood that the aspects of the present disclosure, as generally described herein, and illustrated in the figures, and detailed description, and in the examples, can be arranged, substituted, combined, separated, and designed in a wide variety of different configurations, all of which are explicitly contemplated herein.

These and other embodiments are described in further detail in the following description related to the appended drawing figures.

In still other instances, the therapeutic compositions of the present disclosure may be formulated to commence release of a calcium chelating agent and the direct factor Fla inhibitor before commencing release of and the direct factor Xa inhibitor. For example, the release of a calcium chelating agent may commence from 1 minute to 3 days, usually 3 hours to 1 day, after release of the direct factor IIa inhibitor has commenced.

In still other instances, the therapeutic compositions of the present disclosure may be formulated to commence release of a calcium chelating agent before commencing release of the direct factor IIa inhibitor and the direct factor Xa inhibitor. For example, the release of the of the direct factor Xa inhibitor may commence from 1 minute to 3 days, usually 3 hours to 1 day, after release of the direct factor IIa inhibitor and the direct factor IIa inhibitor has commenced.

In still other instances, the therapeutic compositions of the present disclosure may be formulated to commence release of the direct factor Xa inhibitor before commencing release of the direct factor IIa inhibitor. For example, release of the of the direct factor IIa inhibitor may commence from 1 minute to 3 days, usually 3 hours to 1 day, after release of the direct factor Xa inhibitor has commenced.

Exemplary direct factor IIa inhibitors suitable for incorporation into the therapeutic compositions of the present disclosure include at least one of argatroban, dabigatran, ximelagatran, melagatran, efegatran, inogatran, atecegatran metoxil (AZD-0837), hirudin, hirudin analogs, bivalirudin, desirudin, and lepirudin. Presently preferred is argatroban, or a salt, isomer, solvate, analog, derivative, metabolite, or prodrugs thereof.

Exemplary direct factor Xa inhibitors suitable for incorporation into the therapeutic compositions of the present disclosure include at least one of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052). Presently preferred are rivaroxaban and apixaban, or a salt, isomer, solvate, analog, derivative, metabolite, or prodrugs thereof.

Exemplary anti-proliferative agents suitable for incorporation into the therapeutic compositions of the present disclosure include at least m-tor inhibitors selected from a group consisting of sirolimus, biolimus, everolimus, myolimus, novolimus, ridaforolimus, temsirolimus, zotarolimus, or salts, isomers, solvates, analogs, derivatives, metabolites, or prodrugs thereof. Preferred m-tor inhibitors include sirolimus, or a salt, isomer, solvate, analog, derivative, metabolite, or prodrugs thereof.

Exemplary anti-proliferative agents suitable for incorporation into the therapeutic compositions of the present disclosure also include paclitaxel, or a salt, isomer, solvate, analog, derivative, metabolite, or prodrug thereof, as well as antiplatelet drugs.

Preferred combinations of active agent pairs include a calcium chelating agent, argatroban as the direct factor IIa inhibitor and apixaban or rivaroxaban as the direct factor Xa inhibitor comprises.

In specific instances, the structure may comprise a scaffold having at least an outer surface, an inner surface, and one or more edge surfaces between the outer and inner surface. In such instances at least a portion of the outer surface may coated with the therapeutic composition, at least a portion of the inner surface may be coated with the therapeutic composition, at least a portion of the edge surfaces is coated with the therapeutic composition, and frequently two or three of such surfaces will be coated.

In specific instances, at least some of the surfaces, including the outer, inner, and edge surfaces, may have receptacles formed therein, and at least some of these receptacles may have therapeutic agent(s) therein. The receptacles may comprise one or more of wells, channels, holes, surface texture, and the like.

In specific instances, the therapeutic compositions may further comprise an excipient, an adjuvant, a polymeric carrier, or the like.

In specific instances, the three or more active substances may be mixed uniformly with each other. Alternatively or additionally, the three or more active substances may be layered separately from each other. Each layer may comprise an excipient mixed with the therapeutic agent, where the excipient(s) in two or more layers may the same or may be different in at least two of the three layers.

In specific instances, the, the devices may further comprise a control-release layer formed over the at least two, three or more active substances.

In specific instances, the therapeutic composition may include a base layer formed over a surface of the structure and a top layer formed over the base layer. The base layer and top layer may differ in at least some properties. For example, the base layer and top layer differ in at least one of drug dose, drug release rate, and drug release duration.

In preferred examples, the top layer of the therapeutic composition will be formulated to commence release of the active substances before commencing of the active substances from the base layer. For example, the active substances may be released from the top layer over a time period in the range from 1 hour to 7 days after the surface of the structure is positioned adjacent the injury site and/or the active substances may be released from the base layer over a time period in the range from 7 days to 12 months after the active substances have been substantially completely released from the top layer. For example, each of the base and top layers may comprise the at least three active substances are mixed in a biodegradable polymeric matrix.

In some instances, the scaffold structure may be configured to be expanded in a vascular lumen in the patient's body.

In some instances, the therapeutic composition may be coated at least partly on the surface of scaffold structure.

In some instances, the therapeutic composition is present at least partly within a cavity or reservoir within the scaffold structure.

In some instances, the implantable scaffold has at least an outer surface, an inner surface, and one or more edge surfaces between the outer and inner surfaces. For example, at least a portion of the outer surface may be coated with the therapeutic compositions, at least a portion of the inner surface may be coated with the therapeutic compositions, and/or at least a portion of the edge surfaces may be coated with the therapeutic compositions.

In some instances, at least some of the surfaces have receptacles formed therein and at least some of said receptacles have therapeutic agent therein. For example, the receptacles may comprise one or more of wells, channels, holes, and surface texture.

In a second aspect, the preset disclosure provides a method for treating a vascular tissue injury in a patient. The method comprises implanting a scaffold structure at a target location in the patient's vasculature proximate the tissue injury. A therapeutic composition on the scaffold comprises colchicine and/or tirofiban and at least one antiproliferative drug. The colchicine and/or tirofiban is released from a carrier in the therapeutic composition into the vascular tissue over a period of at least one week in an amount sufficient to inhibit inflammation resulting from the implantation of the scaffold. The at least one antiproliferative drug is released from the same or a different carrier over a period of at least one week in an amount sufficient to inhibit smooth cell muscle (SMC) proliferation.

The scaffold structures of present disclosure may have anyone of a wide variety of known structures suitable for implantation and expansion at a target site in the patient's body. Often, the scaffold will have at least an outer surface, an inner surface, and one or more edge surfaces between the outer and inner surfaces. In such instances, at least a portion of the outer surface may be coated with therapeutic compositions. In other instances, at least a portion of the inner surface may be coated with therapeutic compositions. In still other instances, a portion of the edge surfaces may be coated with therapeutic compositions. As an alternative or in addition to surface coating, at least some of the surfaces may have receptacles formed therein and at least some of said receptacles have therapeutic agent therein. For example, the receptacles comprise one or more of wells, channels, holes, and surface texture.

In all aspects and examples of the present disclosure, the therapeutic composition may be positioned on at least one of an internal surface and an external surface of the implantable scaffold and/or may be positioned on both an external and an internal surface of the implantable scaffold.

The implantable scaffolds and methods of the present disclosure are generally suitable for mitigating tissue injury caused by expanding the scaffold at the location but are also useful for treating tissue injury that preexists deploying the structure at the location.

In some examples, the therapeutic composition may be formulated for an initial rapid release into the environment surrounding the implantable scaffold a preselected time period after implantation of the body structure into said environment followed by a slower controlled release over the extended periods as described above.

The scaffolds of the present disclosure are particularly intended for use in vascular stents and grafts, but may also find use may be any type of therapeutic, diagnostic, or other structure intended for implantation in the patient's vasculature or other vascularized regions of the patient's body, such as prosthetic heart valves, patent foramen ovale (PFO) occlusion devices, atrial septal defect (ASD) occlusion devices, left atrial appendage (LAA) occlusion devices, or similar expandable structure, including an orthopedic implants.

Carriers will typically comprise polymers, usually biodegradable polymers, more usually but not always being synthetic polymers synthesized from petroleum and other hydrocarbon feedstocks. Exemplary biodegradable, synthetic polymers are selected from the group consisting of polyesters, including polylactic acids, polyglycolic acids, polylactic acid-co-glycolic acids, polylactic acid-co-caprolactones, polyethylene glycol-block-poly caprolactone, and polyurethanes; poly(methyl methacrylate) (PMMA); poly N-(2-Hydroxypropyl) methacrylamides; polyethylenimine (PEI), dextran, dextrin, chitosans, poly(L-lysine); poly(aspartamides), polyethylenes; polypropylenes; polyamides; polyethylene glycols (PEG); silicones; poly(anhydrides); and poly ortho esters.

An exemplary biodegradable polymer comprises poly(lactic-co-glycolic acid) (PLGA), where the PLGA is present at 5 μg to 15 μg per mm of scaffold structure length in the first therapeutic composition and from and from 5 μg to 20 μg per mm of scaffold structure length in the second therapeutic composition.

Alternatively, the polymer may comprises a non-degradable polymer, for example being selected from the group consisting of polyacrylates, polymethacrylates, poly(n-butyl methacrylate), poly(hydroxyethylmethacrylate), polyamides, nylons, nylon 12, Dacron, polyethylene terephthalate, poly(ethylene glycol), polyethylene oxide (PEO), polydimethylsiloxane, polyvinylpyrrolidone, ethylene-vinyl acetate, phosphorylcholine-containing polymers, poly(2-methacryloyloxyethylphosphorylcholine), poly(2-methacryloyloxyethylphosphorylcholine-co-butyl methacrylate), and copolymers thereof.

In another aspect, the present disclosure provides a method for treating tissue injury in patients. The method comprises deploying a structure at a target tissue injury location in the patient's body lumen. A therapeutic composition is released from the deployed structure to the location of injury, where the therapeutic composition comprises at least a calcium chelating agent, a direct factor IIa inhibitor, a direct factor Xa inhibitor, and optionally an anti-proliferative agent.

The therapeutic composition may be positioned on an external surface of the device, on an internal surface of the device, or on both external and internal surfaces of the device.

The tissue injury may be caused by deploying the structure at the location or may preexists deploying the structure at the location.

In specific instances, the body lumen comprises a blood vessel and the therapeutic composition is formulated to locally release the at least two, three or more active substances to the injury site at a rate or a concentration sufficient to begin to inhibit or resolve one or more of inflammation, cell proliferation, internal elastic lamina (IEL) injury, fibrin formation, platelet aggregation, platelet activation, and clot formation or dissolution within about 3 hours to about 7 days after the structure is deployed.

In specific instances, the at least two, three or more active substances may be released to the injury site at a rate or a concentration sufficient to inhibit one or more of inflammation, cell proliferation, internal elastic lamina (IEL) injury, fibrin formation, platelet aggregation, platelet activation, and clot formation or dissolution for a period of at least 1 day, for a period of at least one week, for a period of at least one month, for a period of at least three months, for a period of at least six months, or for a period of at least one year after the surface of the structure is positioned adjacent the injury site.

In specific instances, the two, three or more active substances are released substantially simultaneously.

Alternatively or additionally, the direct factor IIa inhibitor and the direct factor Xa inhibitor are released substantially simultaneously and the anti-proliferative is released after the release of the direct factor IIa inhibitor and the direct factor Xa inhibitor has commenced. For example, the release of the of the anti-proliferative agent may commence in a period of 1 minute to 3 days, usually 6 hours to 1 day, after release of the direct factor IIa inhibitor and the direct factor Xa inhibitor has commenced.

Alternatively or additionally, the therapeutic composition may commences release of the direct factor 11a inhibitor before commencing release of the direct factor Xa inhibitor. For example, the release of the of the direct factor Xa inhibitor commences from 1 minute to 3 days, usually 6 hours to 1 day, after release of the direct factor IIa inhibitor has commenced.

Alternatively or additionally, the therapeutic composition may commence release of the direct factor Xa inhibitor before commencing release of the direct factor IIa inhibitor. For example, wherein the release of the of the direct factor IIa inhibitor commences from 1 minute to 3 days, usually 6 hours to 1 day, after release of the direct factor Xa inhibitor has commenced.

In some instances, the direct factor IIa inhibitor comprises at least one of argatroban, dabigatran, ximelagatran, melagatran, efegatran, inogatran, atecegatran metoxil (AZD-0837), hirudin, hirudin analogs, bivalirudin, desirudin, and lepirudin. In presently preferred instances, the direct factor IIa inhibitor comprises argatroban, or a salt, isomer, solvate, analog, derivative, metabolite, or prodrugs thereof.

In some instances, the direct factor Xa inhibitor comprises at least one of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052). In a presently preferred example, the direct factor Xa inhibitor comprises rivaroxaban, or a salt, isomer, solvate, analog, derivative, metabolite, or prodrugs thereof. In a second preferred example, the direct factor Xa inhibitor comprises apixaban, or a salt, isomer, solvate, analog, derivative, metabolite, or prodrugs thereof.

In some instances, the anti-proliferative agent comprises an m-Tor inhibitor may be selected from a group consisting of sirolimus, biolimus, everolimus, myolimus, novolimus, ridaforolimus, temsirolimus, zotarolimus, or salts, isomers, solvates, analogs, derivatives, metabolites, or prodrugs thereof. A presently preferred anti-proliferative agent comprises sirolimus, or a salt, isomer, solvate, analog, derivative, metabolite, or prodrugs thereof.

In other instances, the anti-proliferative agent may comprise paclitaxel, or a salts, isomer, solvate, analog, derivative, metabolite, or prodrug thereof.

In still other instances, the anti-proliferative agent may comprise an antiplatelet drug.

Preferred combinations and pairings of active substances in the methods herein, comprise: a calcium chelating agent (1), the direct factor IIa inhibitor comprising (2) argatroban and the direct factor Xa inhibitor comprising apixaban and (3) the direct factor IIa inhibitor comprising argatroban, the direct factor Xa inhibitor comprises apixaban, and the anti-proliferative agent comprising sirolimus.

In some instances, the structure comprises a scaffold having at least an outer surface, an inner surface, and one or more edge surfaces between the outer and inner surfaces and wherein deploying comprises expanding the scaffold in the body lumen. Typically, at least a portion of the outer surface is coated with the therapeutic composition. Optionally, at least a portion of the inner surface is coated with the therapeutic composition. Further optionally, a portion of the edge surfaces is coated with the therapeutic composition.

In some instances, at least some of the surfaces may have receptacles formed therein and at least some of these receptacles have therapeutic agent therein wherein the receptacles may comprise one or more of wells, channels, holes, and surface texture.

In some instances, the release rate(s) of the active substances will be controlled. For example, excipients with different degradation or release rates can be added to different layers and/or combined with different active substances. Additionally or alternatively, a control-release layer may be formed over therapeutic composition to control the release of the three or more active substances.

In some, the therapeutic composition may include a base layer formed over the surface and a top layer formed over the base layer, wherein the base layer and top layer differ in at least some properties. In such instances, the therapeutic composition may be formulated to release the active substances substantially completely from the top layer before releasing the active substances from the base layer. For example, the active substances may be released from the top layer over a time period in the range from 1 hour to 7 days after the surface of the structure is positioned adjacent the injury site, and the active substances are released from the base layer over a time period in the range from 7 days to 12 months after the after the active substances have been substantially completely released from the top layer. In some instances, each of the base and top layers comprises the at least three active substances are mixed in a biodegradable polymeric matrix.

Often, the injury is at least partially caused before deployment of the structure, but more commonly deployment of the structure, e.g., stent expansion in an artery, causes the injury and wherein the therapeutic composition is formulated to release the calcium chelating agent EDTA the direct factor IIa inhibitor, the direct factor Xa inhibitor, or the anti-proliferative agent before, during, and/or following the injury occurs. Specific examples include vascular wall injury during vascular interventions, including, angioplasty, atherectomy, stent placement, graft placement, and the like.

In other instances, injury occurs during placement, implantation, or other introduction of a temporary or non-temporary device which is selected from the group consisting of access devices, infusion devices, tools, surgical instruments and tools, implants, bodily implants, hip implants, shoulder implants, knee implants, organ implants, luminal implants, vascular implants, stent-delivery systems, stents, stent-grafts, catheters, balloons, graft implants, grafts, aneurysm coils, valves, valve implants, shunts, left atrial appendage implants, foramen implants, leads, closure devices, clips, wound-closure devices and implants, sutures, patches, injection devices, needles inserted in the body, and needles inserted from outside the body.

Each class and type of anti-coagulation agent described and claimed herein, including but not limited to the inhibitors of the clotting cascade, such as the specific factor IIa inhibitor, factor Xa inhibitor, factor XI inhibitor, and factor XIa inhibitor listed herein, as well as the specific chelating agents and the specific anti-coagulation enhancers, may be combined one or more of the other classes and types of anti-coagulation agents described and claimed herein. Moreover, each class and type of anti-coagulation agent described and claimed herein may be formulated for any release profile as described and claimed such as bolus release, dual bolus release, and extended release.

In an aspect, the present disclosure provides a composition for coupling to a surface of an implantable article. The composition may comprise: an anti-coagulant comprising a direct factor Xa inhibitor and at least one of (a) an anti-platelet drug, (b) anti-coagulant comprising a direct factor IIa inhibitor, or (c) an anti-inflammatory drug comprising colchicine.

In some embodiments, the composition further comprises at least two of (a) the anti-platelet drug, (b) the direct factor IIa inhibitor, or (c) the anti-inflammatory drug comprising colchicine. In some embodiments, the composition further comprises all three of (a) an anti-platelet drug, (b) a direct factor IIa inhibitor, and (c) anti-inflammatory drug comprising colchicine.

In some embodiments, wherein the direct factor Xa inhibitor comprises at least one drug selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052), and a salt, isomer, solvate, derivative, metabolite, or pro-drug of any preceding member of the group. In some embodiments, the direct factor Xa inhibitor comprises rivaroxaban, apixaban, or a salt, isomer, solvate, derivative, metabolite, or prodrug thereof.

In some embodiments, the composition further comprises at least one an anti-platelet drug selected from the group consisting of: a Cox 1 inhibitor, or a salt, isomer, solvate, derivative, metabolite, or prodrug thereof; a Cox 2 inhibitor, or a salt, isomer, solvate, derivative, metabolite, or prodrug thereof; a glycoprotein IIIb/IIIa inhibitor, or a salt, isomer, solvate, derivative, metabolite, or prodrug thereof; and a P2Y12 inhibitor, or a salt, isomer, solvate, derivative, metabolite, or prodrug thereof. In some embodiments, the anti-platelet drug comprises tirofiban, or a salt, isomer, solvate, derivative, metabolite, or prodrug thereof. In some embodiments, the anti-platelet drug comprises ticagrelor, or a salt, isomer, solvate, derivative, metabolite, or prodrug thereof. In some embodiments, the anti-platelet drug comprises a Cox1/2 inhibitor or a salt, isomer, solvate, derivative, metabolite, or prodrug thereof; optionally, wherein the Cox1/2 inhibitor comprises acetylsalicylic acid. In some embodiments, the antiplatelet drug comprises clopidogrel, or a salt, isomer, solvate, derivative, metabolite, or prodrug thereof.

In some embodiments, the composition further comprises at least one direct factor IIa inhibitor is selected from the group consisting of argatroban, dabigatran, ximelagatran, melagatran, efegatran, inogatran, atecegatran metoxil (AZD-0837), hirudin, hirudin analogs, bivalirudin, desirudin, and lepirudin. In some embodiments, the at least one direct factor IIa inhibitor drug comprises argatroban, or a salt, isomer, solvate, derivative, metabolite, or prodrug thereof.

In another aspect, the present disclosure provides a composition for coupling to a surface of an implantable article. The composition may comprise: an anti-coagulant comprising a direct factor IIa inhibitor and at least one of (a) an anti-platelet drug, (b) an anti-coagulant comprising a direct factor Xa inhibitor, or (c) an anti-inflammatory drug comprising colchicine.

In some embodiments, the composition further comprises at least two of (a) the anti-platelet drug, (b) the anti-coagulant comprising the direct factor Xa inhibitor, or (c) the anti-inflammatory drug comprising colchicine. In some embodiments, the composition further comprises all three of (a) an anti-platelet drug, (b) the anti-coagulant comprising the direct factor Xa inhibitor, and (c) the anti-inflammatory drug comprising colchicine.

In some embodiments, the direct factor IIa inhibitor is selected from the group consisting of: argatroban, dabigatran, ximelagatran, melagatran, efegatran, inogatran, atecegatran metoxil (AZD-0837), hirudin, hirudin analogs, bivalirudin, desirudin, lepirudin, and a salt, isomer, solvate, derivative, metabolite, or pro-drug of any preceding member of the group. In some embodiments, the direct factor IIa inhibitor drug comprises argatroban, or a salt, isomer, solvate, derivative, metabolite, or prodrug thereof.

In some embodiments, the composition further comprises at least one an anti-platelet drug selected from the group consisting of: a Cox 1 inhibitor or a salt, isomer, solvate, derivative, metabolite, or prodrug thereof; a Cox 2 inhibitor or a salt, isomer, solvate, derivative, metabolite, or prodrug thereof; a glycoprotein IIb/IIIa inhibitor or a salt, isomer, solvate, derivative, metabolite, or prodrug thereof; and a P2Y12 inhibitor or a salt, isomer, solvate, derivative, metabolite, or prodrug thereof. In some embodiments, the anti-platelet drug comprises tirofiban, or a salt, isomer, solvate, derivative, metabolite, or prodrug thereof. In some embodiments, the anti-platelet drug comprises ticagrelor, or a salt, isomer, solvate, derivative, metabolite, or prodrug thereof. In some embodiments, the anti-platelet drug comprises a Cox1/2 inhibitor or a salt, isomer, solvate, derivative, metabolite, or prodrug thereof; optionally, wherein the Cox1/2 inhibitor comprises acetylsalicylic acid. In some embodiments, the antiplatelet drug comprises: clopidogrel or a salt, isomer, solvate, derivative, metabolite, or prodrug thereof; or the active metabolite of clopidogrel or a salt, isomer, solvate, derivative, metabolite, or prodrug thereof.

In some embodiments, the direct factor Xa inhibitor comprises at least one drug selected from the group consisting of: apixaban, betrixaban, edoxaban, otamixaban, razaxaban, rivaroxaban, (r)-n-(2-44-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052), and a salt, isomer, solvate, derivative, metabolite, or pro-drug of any preceding member of the group. In some embodiments, the direct factor Xa inhibitor comprises: apixaban, or a salt or an analog thereof; and/or rivaroxaban, or a salt, isomer, solvate, derivative, metabolite, or prodrug thereof.

In another aspect, the present disclosure provides a composition for coupling to a surface of an implantable article. The composition may comprise: a direct factor Xa inhibitor; and at least two of (a) a direct factor IIa inhibitor, (b) an anti-platelet drug selected from the group consisting of a Cox 1 inhibitor, a Cox 2 inhibitor, a IIb/IIIa inhibit, and a P2y12 inhibitor; or (c) an anti-inflammatory drug. In some embodiments, the composition further comprises all three of (a) the direct factor IIa inhibitor, (b) the anti-platelet drug selected from the group consisting of a Cox 1 inhibitor, a Cox 2 inhibitor, and a IIb/IIIa inhibitor; and (c) the anti-inflammatory drug.

The composition of claim 25 or 26, wherein the direct factor Xa inhibitor comprises at least one drug selected from the group consisting of: apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1 h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052), and a salt, isomer, solvate, derivative, metabolite or prodrug of any preceding member of the group.

In some embodiments, the direct factor Xa inhibitor comprises rivaroxaban, apixaban, or a salt, isomer, solvate, derivative, metabolite, or prodrug thereof. In some embodiments, the composition further comprises the direct factor IIa inhibitor comprises argatroban, or a salt, isomer, solvate, derivative, metabolite, or prodrug thereof. In some embodiments, the anti-platelet drug comprises at least one drug selected from the group consisting of tirotiban, ticagrelor, a Cox 1 inhibitor, a Cox 2 inhibitor, a IIb/IIIa inhibitor, a P2Y12 inhibitor, and a salt, analog, or metabolite of any preceding member of the group. In some embodiments, the anti-inflammatory drug comprises colchicine.

In another aspect, the present disclosure provides a composition for coating a surface of an implantable article. The composition may comprise: apixaban or a salt, analog, or metabolite thereof; rivaroxaban or a salt, analog, or metabolite thereof: argatroban or a salt, analog, or metabolite thereof: tirofiban or a salt, analog, or metabolite thereof; and colchicine or a salt, analog, or metabolite thereof.

In some embodiments, the composition of any aspect or embodiment further comprises an additional an anti-platelet drug. In some embodiments, the composition of any aspect or embodiment further comprises an additional anti-inflammatory drug.

In another aspect, the present disclosure provides a surgical article comprising a base structure, wherein the composition of any aspect or embodiment and/or one or more components thereof are coupled to a surface or interior of the base structure.

In some embodiments, the composition is coupled to the surface in a single layer. In some embodiments, the single layer is homogeneous. In some embodiments, the single layer is non-homogeneous. In some embodiments, at least two layers of the composition and/or one or more components are coupled to the surface. In some embodiments, the surgical article further comprises an intermediate layer over and/or between said at least two layers. In some embodiments, the intermediate layer comprises a drug in addition to drugs on the composition. In some embodiments, the drugs in the composition coupled to the surface have a release rate in a range from 1 day to one year. In some embodiments, the drugs in the composition coupled to the surface have a release rate in a range from 1 day to 3 days, from 1 day to 30 days, or 1 day to 90 days.

Clause 1. An implantable scaffold comprising: a scaffold structure having a surface configured to be implanted in a patient's vasculature; a therapeutic composition configured to be released from a surface of the scaffold, said therapeutic composition comprising colchicine and/or tirofiban and at least one antiproliferative drug; wherein the colchicine and/or tirofiban is present in a carrier configured to release the colchicine and/or tirofiban over a period of at least one week in an amount sufficient to inhibit inflammation resulting from the implantation of the scaffold; and wherein the at least one antiproliferative drug is present in the same or a different carrier configured to release the antiproliferative drug over a period of at least one week in an amount sufficient to inhibit smooth cell muscle (SMC) proliferation.

Clause 2. The implantable scaffold of clause 1, wherein the at least one antiproliferative drug comprises an mTOR inhibitor.

Clause 3. The implantable scaffold of clause 2, wherein the mTOR inhibitor comprises one or more drugs selected from a group consisting of sirolimus, biolimus, everolimus, myolimus, novolimus, ridaforolimus, temsirolimus, zotarolimus, and salts, isomers, solvates, analogs, derivatives, metabolites, or prodrugs thereof.

Clause 4. The implantable scaffold of clause 3, wherein the mTOR inhibitor comprises sirolimus, or a salts, isomer, solvate, analog, derivative, metabolite, or prodrug thereof.

Clause 5. The implantable scaffold of clause 1 to 4, wherein the therapeutic composition is formulated to release no greater than 90%, 75%, 50%, 25%, or 10% by weight of the colchicine and/or tirofiban from the scaffold into a vascular environment within one week, one month, three months, six months, or one year of implantation.

Clause 6. The implantable scaffold of clause 5, wherein the therapeutic composition is formulated to release no greater than 90%, 75%, 50%, 25%, or 10% by weight of the antiproliferative drug from the scaffold into the vascular environment within one week, one month, three months, six months, or one year of implantation.

Clause 7. The implantable scaffold of clause 1 to 5, wherein the scaffold structure is loaded with an amount of the colchicine in a range from 1.7 μg to 170 μg, 1.7 μg to 85 μg, 2.5 μg to 40 μg, or 3.4 μg to 20 μg per mm of the scaffold structure of the length.

Clause 8. The implantable scaffold of clause 1 to 6, wherein the scaffold structure is loaded with an amount of the antiproliferative drug in a range from 2 μg to 200 μg, 2 μg to 100 μg, 3 μg to 50 μg, or 4 μg to 20 μg per mm of the scaffold structure of the length.

Clause 9. The implantable scaffold of clause 1 to 8, wherein the carrier comprises a biodegradable polymer.

Clause 10. The implantable scaffold of clause 9, wherein the biodegradable polymer is selected from a group consisting of polyesters, including polylactic acids, polyglycolic acids, polylactic acid-co-glycolic acids, polylactic acid-co-caprolactones, polyethylene glycol-block-poly caprolactone, and polyurethanes; poly(methyl methacrylate) (PMMA); poly N-(2-Hydroxypropyl) methacrylamides; polyethylenimine (PEI), dextran, dextrin, chitosans, poly(L-lysine); poly(aspartamides), polyethylenes; polypropylenes; polyamides; polyethylene glycols (PEG); silicones; poly(anhydrides); and poly ortho esters.

Clause 11. The implantable scaffold of clause 1 to 10, wherein the therapeutic composition further comprises at least one anti-coagulant agent.

Clause 12. The implantable scaffold of clause 11, wherein the at least one anti-coagulant is selected from the group consisting of a direct factor IIa inhibitor and a direct factor Xa inhibitor.

Clause 13. The implantable scaffold of clauses 12, wherein the at least one anti-coagulant comprises a direct factor IIa inhibitor selected from the group consisting of argatroban, dabigatran, ximelagatran, melagatran, efegatran, inogatran, atecegatran metoxil (AZD-0837), hirudin, hirudin analogs, bivalirudin, desirudin, and lepirudin.

Clause 14. The implantable scaffold of clause 13, wherein the at least one direct factor 11a inhibitor comprises argatroban, or a salt, isomer, solvate, analog, derivative, metabolite, or prodrugs thereof.

Clause 15. The implantable scaffold of any one of clauses 11 to 14, wherein the at least one anti-coagulant comprises a direct factor Xa inhibitor selected from the group consisting of apixaban, betrixaban, edoxaban, otamixaban, razaxaban, rivaroxaban. (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052).

Clause 16. The implantable scaffold of clause 15, wherein the direct factor Xa inhibitor comprises apixaban, or a salt, isomer, solvate, analog, derivative, metabolite, or prodrugs thereof.

Clause 17. The implantable scaffold of clause 16, wherein the direct factor Xa inhibitor comprises rivaroxaban, or a salt, isomer, solvate, analog, derivative, metabolite, or prodrugs thereof.

Clause 18. The implantable scaffold of any one of clauses 1 to 17, wherein therapeutic composition further comprises an antiplatelet drug.

Clause 19. The method of any one of clauses 18, wherein the antiplatelet drug is selected from a group consisting of Abciximab, Eptifibatide, Orbofiban, Roxifiban, Sibrafiban, Tirofiban, Clopidogrel, Prasugrel, Cangrelor, Elinogrel, Ticagrelor, Beraprost, Iloprost, Prostacyclin, Treprostinil, Acetylsalicylic acid/Aspirin, Aloxiprin, Carbasalate calcium, Indobufen, Triflusal, Dipyridamole/aspirin, Picotamide, Terbogrel, Terutroban, Cilostazol, Dipyridamole, Triflusal, Cloricromen, Ditazole, Vorapaxar, and Ticlopidine.

Clause 20. The implantable scaffold of clause 1 to 19, wherein therapeutic composition further comprises a second antiproliferative drug.

Clause 21. The implantable scaffold of clause 20, wherein the second antiproliferative drug is selected from the group consisting of mycophenolate mofetil, mycophenolate sodium, azathioprine.

Clause 22. The implantable scaffold of clause 1 to 21, wherein therapeutic composition further comprises a cationic anti-coagulation enhancer.

Clause 23. The implantable scaffold of clause 22, wherein the cationic anti-coagulation enhancer is selected from a first group consisting of magnesium stearate and other magnesium salts, monoammonium salt, diammonium salt, triammonium salt, benzyldimethyltetradecylammonium salt, tridodecylmethylammonium salt, a benzalkonium, analogue, solvate, hydrate and derivatives thereof or from a second group consisting of poly(L-lysine) (PLL), linear polyethyleneimine (PEI), branch polyethyleneimine (PEI), chitosan, PAMAM dendrimers, and poly(2-dimethylamino)ethyl methacrylate (pDMAEMA), protamine, polylysine, a polybetaaminoester (PBAE), Histone, ethylenediamine, methylenediamine, ammonium chloride, melamine, histamine, histidine, analogue, solvate, hydrate and derivatives thereof.

Clause 24. The implantable scaffold of clause 1 to 23, wherein therapeutic composition further comprises a factor XI/XIa inhibitor.

Clause 25. The implantable scaffold of clause 24, wherein factor XI/XIa inhibitor comprises a protein Z-dependent protease inhibitor, analogue, solvate, hydrate or derivatives thereof.

Clause 26. The implantable scaffold of clause 1 to 25, wherein the therapeutic composition further comprises a chelating agent formulated to deplete calcium in the environment surrounding the scaffold upon implantation of the scaffold structure in said environment.

Clause 27. The implantable scaffold of clause 26, wherein the chelating agent is selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), deferoxamine (DFO), a surfactant-EDTA complex, quaternary ammonium EDTA salt, benzalkonium EDTA salt, EDTA complex, salts, analogue, solvate, hydrate and derivatives thereof.

Clause 28. The implantable scaffold of clause 1 to 27, wherein the scaffold structure is configured to be expanded in a vascular lumen in the patient's body.

Clause 29. The implantable scaffold of clause 1 to 28, wherein the therapeutic composition is coated at least partly on the surface of scaffold structure.

Clause 30. The implantable scaffold of clause 1 to 29, wherein the therapeutic composition is present at least partly within a cavity or reservoir within the scaffold structure.

Clause 31. The implantable scaffold of clause 1 to 30, wherein the implantable scaffold has at least an outer surface, an inner surface, and one or more edge surfaces between the outer and inner surfaces.

Clause 32. The implantable scaffold of clause 31, wherein at least a portion of the outer surface is coated with the therapeutic compositions.

Clause 33. The implantable scaffold of clause 30 or 31, wherein at least a portion of the inner surface is coated with the therapeutic compositions.

Clause 34. The implantable scaffold of any one of clauses 31 to 33, wherein a portion of the edge surfaces is coated with the therapeutic compositions.

Clause 35. The implantable scaffold of any one of clauses 31 to 34, wherein at least some of the surfaces have receptacles formed therein and at least some of said receptacles have therapeutic agent therein.

Clause 36. The implantable scaffold of clause 35, wherein the receptacles comprise one or more of wells, channels, holes, and surface texture.

Clause 37-50 intentionally left blank.

Clause 51. A method for treating a vascular tissue injury in a patient, the method comprising: implanting a scaffold structure at a target location in the patient's vasculature proximate the tissue injury; and wherein a therapeutic composition on the scaffold comprises colchicine and/or tirofiban and at least one antiproliferative drug; wherein the colchicine and/or tirofiban is released from a carrier in the therapeutic composition into the vascular tissue over a period of at least one week in an amount sufficient to inhibit inflammation resulting from the implantation of the scaffold; and wherein the at least one antiproliferative drug is released from the same or a different carrier over a period of at least one week in an amount sufficient to inhibit smooth cell muscle (SMC) proliferation.

Clause 52. The method of clause 51, wherein the at least one antiproliferative drug comprises an mTOR inhibitor.

Clause 53. The method of clause 52, wherein the mTOR inhibitor comprises one or more drugs selected from a group consisting of sirolimus, biolimus, everolimus, myolimus, novolimus, ridaforolimus, temsirolimus, zotarolimus, and salts, isomers, solvates, analogs, derivatives, metabolites, or prodrugs thereof.

Clause 54. The method of clause 53, wherein the mTOR inhibitor comprises sirolimus, or a salts, isomer, solvate, analog, derivative, metabolite, or prodrug thereof.

Clause 55. The method of clause 51 to 54, wherein the therapeutic composition is formulated to release no greater than 90%, 75%, 50%, 25%, or 10% by weight of the colchicine and/or tirofiban from the scaffold into a vascular environment within one week, one month, three months, six months, or one year of implantation.

Clause 56. The method of clause 55, wherein the therapeutic composition is formulated to release no greater than 90%, 75%, 50%, 25%, or 10% by weight of the mTOR inhibitor from the scaffold into the vascular environment within one week, one month, three months, six months, or one year of implantation.

Clause 57. The method of clause 51 to 55, wherein the scaffold structure is loaded with an amount of the colchicine and/or tirofiban in a range 1.7 μg to 170 μg, 1.7 μg to 85 μg, 2.5 μg to 40 μg, or 3.4 μg to 20 μg per mm of the length of the scaffold structure.

Clause 58. The method of clause 51 to 56, wherein the scaffold structure is loaded with an amount of the antiproliferative drug in a range from 2 μg to 200 μg, 2 μg to 100 μg, 3 μg to 50 μg, or 4 μg to 20 μg per mm of the length of the scaffold structure.

Clause 59. The method of clause 51 to 58, wherein the carrier comprises a biodegradable polymer.

Clause 60. The method of clause 59, wherein the biodegradable polymer is selected from a group consisting of polyesters, including polylactic acids, polyglycolic acids, polylactic acid-co-glycolic acids, polylactic acid-co-caprolactones, polyethylene glycol-block-poly caprolactone, and polyurethanes; poly(methyl methacrylate) (PMMA); poly N-(2-Hydroxypropyl) methacrylamides; polyethylenimine (PEI), dextran, dextrin, chitosans, poly(L-lysine); poly(aspartamides), polyethylenes; polypropylenes; polyamides; polyethylene glycols (PEG); silicones; poly(anhydrides); and poly ortho esters.

Clause 61. The method of clause Si to 60, wherein the therapeutic composition further comprises at least one anti-coagulant agent.

Clause 62. The method of clause 61, wherein the at least one anti-coagulant is selected from the group consisting of a direct factor IIa inhibitor and a direct factor Xa inhibitor.

Clause 63. The method of clauses 62, wherein the at least one anti-coagulant comprises a direct factor IIa inhibitor selected from the group consisting of argatroban, dabigatran, ximelagatran, melagatran, efegatran, inogatran, atecegatran metoxil (AZD-0837), hirudin, hirudin analogs, bivalirudin, desirudin, and lepirudin.

Clause 64. The method of clause 63, wherein the at least one direct factor IIa inhibitor comprises argatroban, or a salt, isomer, solvate, analog, derivative, metabolite, or prodrugs thereof.

Clause 65. The method of any one of clauses 61 to 64, wherein the at least one anti-coagulant comprises a direct factor Xa inhibitor selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052).

Clause 66. The method of clause 65, wherein the direct factor Xa inhibitor comprises apixaban, or a salt, isomer, solvate, analog, derivative, metabolite, or prodrugs thereof.

Clause 67. The method of clause 66, wherein the direct factor Xa inhibitor comprises rivaroxaban, or a salt, isomer, solvate, analog, derivative, metabolite, or prodrugs thereof.

Clause 68. The method of any one of clauses 51 to 67, wherein therapeutic composition further comprises an antiplatelet drug.

Clause 69. The method of any one of clauses 68, wherein the antiplatelet drug is selected from a group consisting of Abciximab, Eptifibatide, Orbofiban, Roxitiban, Sibrafiban, Tirofiban, Clopidogrel, Prasugrel, Cangrelor. Elinogrel, Ticagrelor, Beraprost, Iloprost, Prostacyclin, Treprostinil, Acetylsalicylic acid/Aspirin, Aloxiprin, Carbasalate calcium, Indobufen, Triflusal, Dipyridamolc aspirin, Picotamide, Terbogrel, Terutroban, Cilostazol, Dipyridamole, Triflusal, Cloricromen, Ditazole, Vorapaxar, and Ticlopidine.

Clause 70. The method of clause 51 to 69, wherein therapeutic composition further comprises a second antiproliferative drug.

Clause 71. The method of clause 70, wherein the second antiproliferative drug is selected from the group consisting of mycophenolate mofetil, mycophenolate sodium, azathioprine.

Clause 72. The method of clause 51 to 71, wherein therapeutic composition further comprises a cationic anti-coagulation enhancer.

Clause 73. The method of clause 72, wherein the cationic anti-coagulation enhancer is selected from a first group consisting of magnesium stearate and other magnesium salts, monoammonium salt, diammonium salt, triammonium salt, benzyldimethyltetradecylammonium salt, tridodecylmethylammonium salt, a benzalkonium, analogue, solvate, hydrate and derivatives thereof or from a second group consisting of poly(L-lysine) (PLL), linear polyethyleneimine (PEI), branch polyethyleneimine (PEI), chitosan, PAMAM dendrimers, and poly(2-dimethylamino)ethyl methacrylate (pDMAEMA), protamine, polylysine, a polybetaaminoester (PBAE), Histone, ethylenediamine, methylenediamine, ammonium chloride, melamine, histamine, histidine, analogue, solvate, hydrate and derivatives thereof.

Clause 74. The method of clause 51 to 73, wherein therapeutic composition further comprises a factor XI/XIa inhibitor.

Clause 75. The method of clause 74, wherein factor XI/XIa inhibitor comprises a protein Z-dependent protease inhibitor, analogue, solvate, hydrate or derivatives thereof.

Clause 76. The method of clause 51 to 54, wherein the therapeutic composition further comprises a chelating agent formulated to deplete calcium in the environment surrounding the scaffold upon implantation of the scaffold structure in said environment.

Clause 77. The method of clause 76, wherein the chelating agent is selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), deferoxamine (DFO), a surfactant-EDTA complex, quaternary ammonium EDTA salt, benzalkonium EDTA salt, EDTA complex, salts, analogue, solvate, hydrate and derivatives thereof.

Clause 78. The method of clause 51 to 77, wherein the scaffold structure is configured to be expanded in a vascular lumen in the patient's body.

Clause 79. The method of clause 51 to 78, wherein the therapeutic composition is coated at least partly on the surface of scaffold structure.

Clause 80. The method of clause 51 to 79, wherein the therapeutic composition is present at least partly within a cavity or reservoir within the scaffold structure.

Clause 81. The method of clause 51 to 80, wherein the scaffold has at least an outer surface, an inner surface, and one or more edge surfaces between the outer and inner surfaces.

Clause 82. The method of clause 81, wherein at least a portion of the outer surface is coated with the therapeutic compositions.

Clause 83. The method of clause 81 or 32, wherein at least a portion of the inner surface is coated with the therapeutic compositions.

Clause 84. The method of any one of clauses 81 to 83, wherein a portion of the edge surfaces is coated with the therapeutic compositions.

Clause 85. The method of any one of clauses 81 to 83, wherein at least some of the surfaces have receptacles formed therein and at least some of said receptacles have therapeutic agent therein.

Clause 86. The method of clause 85, wherein the receptacles comprise one or more of wells, channels, holes, and surface texture.

Clause 87-100 intentionally left blank.

Clause 101. A composition for coating a surface of an implantable article, said composition comprising: (a) at least one of a factor IIa inhibitor and a factor Xa inhibitor (b) colchicine; and (c) a first antiplatelet drug comprising tirofiban; wherein the (a) at least one of a factor 11a inhibitor and a factor Xa inhibitor, (b) colchicine; and (c) first antiplatelet drug are formulated to release from the surface of the article in amounts sufficient to inhibit inflammation and/or thrombosis resulting from an injury.

Clause 102. The composition of clause 101, wherein the (a) at least one of a factor IIa inhibitor and a factor Xa inhibitor, (b) colchicine; and (c) first antiplatelet drug are formulated to treat an injury that resulted from implantation of the article.

Clause 103. The composition of clause 101 or 102, wherein the (a) at least one of a factor IIIa inhibitor and a factor Xa inhibitor, (b) colchicine; and (c) first antiplatelet drug are formulated to treat the injury without causing any one or more of bleeding, a risk of bleeding risk, and bleeding associated with an administration of a systemic antithrombotic drug.

Clause 104. The composition of clause 101 to 103, comprising at least one factor IIa inhibitor selected from a group consisting of argatroban, dabigatran, ximelagatran, melagatran, efegatran, inogatran, atecegatran metoxil (AZD-0837), hirudin, hirudin analogs, bivalirudin, desirudin, and lepirudin.

Clause 105. The composition of clause 104, wherein the at least one direct factor IIa inhibitor comprises argatroban, or a salt, isomer, solvate, analog, derivative, metabolite, or prodrugs thereof.

Clause 106. The composition of clause 101 to 105, comprising at least one factor Xa inhibitor selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1 h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052).

Clause 107. The composition of clause 106, wherein the direct factor Xa inhibitor comprises apixaban, or a salt, isomer, solvate, analog, derivative, metabolite, or prodrugs thereof.

Clause 108. The composition of clause 106, wherein the direct factor Xa inhibitor comprises rivaroxaban, or a salt, isomer, solvate, analog, derivative, metabolite, or prodrugs thereof.

Clause 109. The composition of clause 101 to 108, further comprising at least a second antiplatelet drug different from the first antiplatelet drug.

Clause 110. The composition of clause 109, wherein the second antiplatelet drug comprises one or more of a glycoprotein IIb/IIa inhibitor, a COX inhibitor, an ADP receptor/P2Y12 inhibitor, a prostaglandin analogue, a thromboxane inhibitor, and a phosphodiesterase inhibitors.

Clause 111. The composition of clause 109 and 110, further comprising at least a third antiplatelet drug different than the first and second antiplatelet drugs.

Clause 112. The composition of clause 111, wherein the third antiplatelet drug comprises one or more of a factor IIb/IIIa inhibitor, a COX inhibitor, an ADP receptor/P2Y12 inhibitor, a prostaglandin analogue, a thromboxane inhibitor, and a phosphodiesterase inhibitors.

Clause 113. The composition of clause 101 to 112, further comprising a chelating agent.

Clause 114. The composition of clause 113, wherein the chelating agent is selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), deferoxamine (DFO), a surfactant-EDTA complex, quaternary ammonium EDTA salt, benzalkonium EDTA salt, EDTA complex, salts, analogue, solvate, hydrate and derivatives thereof.

Clause 115. The composition of clause 101 to 114, further comprising at least one antiproliferative drug.

Clause 116. The composition of clause 115, wherein the antiproliferative drug comprises an mTOR inhibitor.

Clause 117. The composition of clause 101 to 116, wherein individual components of the composition are configured to be coated on the surface simultaneously.

Clause 118. The composition of clause 101 to 116, wherein individual components of the composition are configured to be coated on the surface sequentially.

Clause 119 to 200 intentionally left blank.

Clause 201. A composition for coating a surface of an implantable article, said composition comprising: (a) a factor IIa inhibitor; (b) a factor Xa inhibitor; and (c) a first antiplatelet drug comprising a factor IIa/IIIb inhibitor, wherein (a) the factor IIa inhibitor, (b) the factor Xa inhibitor, and (c) the first antiplatelet drug are formulated to release from the surface of the article in amounts sufficient to inhibit inflammation and/or thrombosis resulting from an injury.

Clause 202. The composition of clause 201, wherein (a) the factor IIa inhibitor, (b) the factor Xa inhibitor, and (c) first antiplatelet drug are formulated to treat an injury that resulted from implantation of the article.

Clause 203. The composition of clause 201 or 202, wherein the (a) the factor IIa inhibitor, (b) the factor Xa inhibitor, and (c) first antiplatelet drug are formulated to treat the injury without causing any one or more of bleeding, a risk of bleeding risk, and bleeding associated with an administration of a systemic antithrombotic drug.

Clause 204. The composition of clause 201 to 203, further comprising (d) colchicine which in combination with (a) the factor IIa inhibitor, (b) the factor Xa inhibitor, and (c) first antiplatelet drug is formulated to release from the surface of the article in an amount sufficient to enhance inhibition of inflammation and/or thrombosis resulting from the injury.

Clause 205. The composition of clause 201 to 204, wherein the factor IIa inhibitor is selected from a group consisting of argatroban, dabigatran, ximelagatran, melagatran, efegatran, inogatran, atecegatran metoxil (AZD-0837), hirudin, hirudin analogs, bivalirudin, desirudin, and lepirudin.

Clause 206. The composition of clause 205, wherein the factor IIa inhibitor comprises argatroban, or a salt, isomer, solvate, analog, derivative, metabolite, or prodrugs thereof.

Clause 207. The composition of clause 201 to 206, wherein the factor Xa inhibitor is selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1 h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052).

Clause 208. The composition of clause 207, wherein the factor Xa inhibitor comprises apixaban, or a salt, isomer, solvate, analog, derivative, metabolite, or prodrugs thereof.

Clause 209. The composition of clause 20, wherein the factor Xa inhibitor comprises rivaroxaban, or a salt, isomer, solvate, analog, derivative, metabolite, or prodrugs thereof.

Clause 210. The composition of clause 201 to 209, wherein the factor IIb/IIIa inhibitor is selected from a group consisting of abciximab, eptifibatide, orbofiban, roxifiban, sibrafiban, and tirofiban.

Clause 211. The composition of clause 201 to 210, further comprising at least a second antiplatelet drug different than the first antiplatelet drug.

Clause 212. The composition of clause 211, wherein the second antiplatelet drug comprises one or more of a factor IIb/IIIa inhibitor, a COX inhibitor, an ADP receptor/P2Y12 inhibitor, a prostaglandin analogue, a thromboxane inhibitor, and a phosphodiesterase inhibitors.

Clause 213. The composition of clause 211 and 212, further comprising at least a third antiplatelet drug different than the first and second antiplatelet drugs.

Clause 214. The composition of clause 213, wherein the third antiplatelet drug comprises one or more of a factor IIb/IIIa inhibitor, a COX inhibitor, an ADP receptor/P2Y12 inhibitor, a prostaglandin analogue, a thromboxane inhibitor, and a phosphodiesterase inhibitors.

Clause 215. The composition of clause 201 to 214, further comprising a chelating agent.

Clause 216. The composition of clause 215, wherein the chelating agent is selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), deferoxamine (DFO), a surfactant-EDTA complex, quaternary ammonium EDTA salt, benzalkonium EDTA salt, EDTA complex, salts, analogue, solvate, hydrate and derivatives thereof.

Clause 217. The composition of clause 201 to 216, further comprising at least one antiproliferative drug.

Clause 218. The composition of clause 217, wherein the antiproliferative drug comprises an mTOR inhibitor.

Clause 219. The composition of clause 201 to 218, wherein individual components of the composition are configured to be coated on the surface simultaneously.

Clause 220. The composition of clause 201 to 218, wherein individual components of the composition are configured to be coated on the surface sequentially.

Clause 221 to 300 intentionally left blank.

Clause 301. A composition for coating a surface of an implantable article, said composition comprising: (a) a factor IIa inhibitor; (b) a factor Xa inhibitor, (c) a first antiplatelet drug comprising a factor IIa/IIIb inhibitor; and (d) a second antiplatelet drug comprising an NSAID: wherein (a) the factor IIa inhibitor, (b) the factor Xa inhibitor, (c) the first antiplatelet drug and (d) the second antiplatelet drug are formulated to release from the surface of the article in amounts sufficient to inhibit inflammation resulting from the implantation of the article when implanted in a patient.

Clause 302. The composition of clause 301, wherein (a) the factor IIa inhibitor, (b) the factor Xa inhibitor, (c) the first antiplatelet drug, and (d) the second antiplatelet drug are formulated to treat an injury that resulted from implantation of the article.

Clause 303. The composition of clause 301 or 302, wherein the (a) the factor IIa inhibitor, (b) the factor Xa inhibitor, (c) the first antiplatelet drug, and (d) the second antiplatelet drug are formulated to treat the injury without causing any one or more of bleeding, a risk of bleeding risk, and bleeding associated with an administration of a systemic antithrombotic drug.

Clause 304. The composition of clause 301 to 303, further comprising (e) colchicine which in combination with (a) the factor IIa inhibitor, (b) the factor Xa inhibitor, (c) the first antiplatelet drug, and (d) the second antiplatelet drug is formulated to release from the surface of the article in amounts sufficient to enhance inhibition of inflammation and/or thrombosis resulting from the injury.

Clause 305. The composition of clause 301 to 304, wherein the factor IIa inhibitor is selected from a group consisting of argatroban, dabigatran, ximelagatran, melagatran, efegatran, inogatran, atecegatran metoxil (AZD-0837), hirudin, hirudin analogs, bivalirudin, desirudin, and lepirudin.

Clause 306. The composition of clause 305, wherein the factor IIa inhibitor comprises argatroban, or a salt, isomer, solvate, analog, derivative, metabolite, or prodrugs thereof.

Clause 307. The composition of clause 301 to 306, wherein factor Xa inhibitor selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1 h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052).

Clause 308. The composition of clause 307, wherein the direct factor Xa inhibitor comprises apixaban, or a salt, isomer, solvate, analog, derivative, metabolite, or prodrugs thereof.

Clause 309. The composition of clause 30, wherein the direct factor Xa inhibitor comprises rivaroxaban, or a salt, isomer, solvate, analog, derivative, metabolite, or prodrugs thereof.

Clause 310. The composition of clause 301 to 309, wherein the factor IIb/IIIa inhibitor is selected from a group consisting of abciximab, eptifibatide, orbofiban, roxifiban, sibrafiban, and tirofiban.

Clause 311. The composition of clause 301 to 310, wherein the NSAID comprises a salicylate.

Clause 312. The composition of clause 301 to 311, further comprising at least a third antiplatelet drug different than the first and second antiplatelet drugs.

Clause 313. The composition of clause 311, wherein the third antiplatelet drug comprises one or more of a factor IIb/IIIa inhibitor, a COX inhibitor, an ADP receptor/P2Y12 inhibitor, a prostaglandin analogue, a thromboxane inhibitor, and a phosphodiesterase inhibitors.

Clause 314. The composition of clause 301 to 313, further comprising a chelating agent.

Clause 315. The composition of clause 314, wherein the chelating agent is selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), deferoxamine (DFO), a surfactant-EDTA complex, quaternary ammonium EDTA salt, benzalkonium EDTA salt, EDTA complex, salts, analogue, solvate, hydrate and derivatives thereof.

Clause 316. The composition of clause 301 to 315, further comprising at least one antiproliferative drug.

Clause 317. The composition of clause 316, wherein the antiproliferative drug comprises an mTOR inhibitor.

Clause 318. The composition of clause 301 to 317, wherein individual components of the composition are configured to be coated on the surface simultaneously.

Clause 319. The composition of clause 301 to 317, wherein individual components of the composition are configured to be coated on the surface sequentially.

Clause 320 to 350 intentionally left blank.

Clause 351. A composition for coating a surface of an implantable article, said composition comprising: (a) a factor IIa inhibitor; (b) a factor Xa inhibitor; (c) a first antiplatelet drug comprising a factor IIa/IIIb inhibitor; and (d) a second antiplatelet drug comprising an ADP receptor/P2Y12 inhibitors; wherein (a) the factor IIa inhibitor, (b) the factor Xa inhibitor, (c) the first antiplatelet drug and (d) the second antiplatelet drug are formulated to release from the surface of the article in amounts sufficient to inhibit inflammation resulting from the implantation of the article when implanted in a patient.

Clause 352. The composition of clause 351, wherein (a) the factor IIa inhibitor, (b) the factor Xa inhibitor, (c) the first antiplatelet drug, and (d) the second antiplatelet drug are formulated to treat an injury that resulted from implantation of the article.

Clause 353. The composition of clause 351 or 352, wherein the (a) the factor IIa inhibitor, (b) the factor Xa inhibitor, (c) the first antiplatelet drug, and (d) the second antiplatelet drug are formulated to treat the injury without causing any one or more of bleeding, a risk of bleeding risk, and bleeding associated with an administration of a systemic antithrombotic drug.

Clause 354. The composition of clause 351 to 353, further comprising (e) colchicine which in combination with (a) the factor 11a inhibitor, (b) the factor Xa inhibitor, (c) the first antiplatelet drug, and (d) the second antiplatelet drug is formulated to release from the surface of the article in amounts sufficient to enhance inhibition of inflammation and/or thrombosis resulting from the injury.

Clause 355. The composition of clause 351 to 354, further comprising (t) a third antiplatelet drug comprising aspirin and/or another NSAID which in combination with (a) the factor IIa inhibitor, (b) the factor Xa inhibitor, (c) the first antiplatelet drug, and (d) the second antiplatelet drug is formulated to release from the surface of the article in amounts sufficient to enhance inhibition of inflammation and/or thrombosis resulting from the injury.

Clause 356. The composition of clause 351 to 355, wherein the factor IIa inhibitor is selected from a group consisting of argatroban, dabigatran, ximelagatran, melagatran, efegatran, inogatran, atecegatran metoxil (AZD-0837), hirudin, hirudin analogs, bivalirudin, desirudin, and lepirudin.

Clause 357. The composition of clause 356, wherein the factor IIa inhibitor comprises argatroban, or a salt, isomer, solvate, analog, derivative, metabolite, or prodrugs thereof.

Clause 358. The composition of clause 351 to 357, wherein factor Xa inhibitor selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052).

Clause 359. The composition of clause 358, wherein the direct factor Xa inhibitor comprises apixaban, or a salt, isomer, solvate, analog, derivative, metabolite, or prodrugs thereof.

Clause 360. The composition of clause 359, wherein the direct factor Xa inhibitor comprises rivaroxaban, or a salt, isomer, solvate, analog, derivative, metabolite, or prodrugs thereof.

Clause 361. The composition of clause 351 to 360, wherein the factor IIb/IIa inhibitor is selected from a group consisting of abciximab, eptifibatide, orbofiban, roxifiban, sibratiban, and tirofiban.

Clause 362. The composition of clause 351 to 361, wherein the ADP receptor/P2Y12 inhibitors is selected from a group consisting of clopidogrel, ticlopidine, prasugrel, cangrelor, elinogrel, and ticagrelor. [in SLIDER #1 only]

Clause 363. The composition of clause 351 to 362, further comprising at least a third antiplatelet drug different than the first and second antiplatelet drugs.

Clause 364. The composition of clause 363, wherein the third antiplatelet drug comprises one or more of a factor IIb/IIIa inhibitor, a COX inhibitor, an ADP receptor/P2Y12 inhibitor, a prostaglandin analogue, a thromboxane inhibitor, and a phosphodiesterase inhibitors.

Clause 365. The composition of clause 364, wherein the third antiplatelet drug comprises aspirin or another NSAID.

Clause 366. The composition of clause 351 to 365, further comprising a chelating agent.

Clause 367. The composition of clause 366, wherein the chelating agent is selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), deferoxamine (DFO), a surfactant-EDTA complex, quaternary ammonium EDTA salt, benzalkonium EDTA salt, EDTA complex, salts, analogue, solvate, hydrate and derivatives thereof.

Clause 368. The composition of clause 351 to 367, further comprising at least one antiproliferative drug.

Clause 369. The composition of clause 368, wherein the antiproliferative drug comprises an mTOR inhibitor.

Clause 370. The composition of clause 351 to 369, wherein individual components of the composition are configured to be coated on the surface simultaneously.

Clause 371. The composition of clause 351 to 370, wherein individual components of the composition are configured to be coated on the surface sequentially.

Clause 372 to 400 intentionally left blank.

Clause 401. A composition for coating a surface of an implantable article, said composition comprising: a factor Xa inhibitor; and one or more of (a) colchicine, (b) a first antiplatelet drug comprising a factor IIa/IIIb inhibitor; (c) a second antiplatelet drug comprising an NSAID, and (d) a third antiplatelet drug comprising an ADP inhibitor, wherein the factor Xa inhibitor and the one or more of (a) the colchicine, (b) the first antiplatelet drug, (c) the second antiplatelet drug and (d) the third antiplatelet drug are formulated to release from the surface of the article in amounts sufficient to inhibit inflammation resulting from the implantation of the article when implanted in a patient.

Clause 402. The composition of clause 401, wherein the factor Xa inhibitor and (a) the colchicine, (b) the first antiplatelet drug comprising a factor IIa/IIIb inhibitor; (c) the second antiplatelet drug comprising an NSAID, and (d) the third antiplatelet drug comprising an ADP inhibitor are formulated to treat an injury that resulted from implantation of the article.

Clause 403. The composition of clause 401 or 402, wherein the factor Xa inhibitor and (a) the colchicine, (b) the first antiplatelet drug comprising a factor IIa IIb inhibitor, (c) the second antiplatelet drug comprising an NSAID, and (d) the third antiplatelet drug comprising an ADP inhibitor are formulated to treat the injury without causing any one or more of bleeding, a risk of bleeding risk, and bleeding associated with an administration of a systemic antithrombotic drug.

Clause 404. The composition of clause 401 to 403, further comprising a factor IIa inhibitor is selected from a group consisting of argatroban, dabigatran, ximelagatran, melagatran, efegatran, inogatran, atecegatran metoxil (AZD-0837), hirudin, hirudin analogs, bivalirudin, desirudin, and lepirudin.

Clause 405. The composition of clause 404, wherein the factor IIa inhibitor comprises argatroban, or a salt, isomer, solvate, analog, derivative, metabolite, or prodrugs thereof.

Clause 406. The composition of clause 401 to 405, wherein factor Xa inhibitor selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052).

Clause 407. The composition of clause 406, wherein the direct factor Xa inhibitor comprises apixaban, or a salt, isomer, solvate, analog, derivative, metabolite, or prodrugs thereof.

Clause 408. The composition of clause 407, wherein the direct factor Xa inhibitor comprises rivaroxaban, or a salt, isomer, solvate, analog, derivative, metabolite, or prodrugs thereof.

Clause 409. The composition of clause 401 to 408, wherein the factor IIb/IIIa inhibitor is selected from a group consisting of abciximab, eptifibatide, orbofiban, roxifiban, sibrafiban, and tirofiban.

Clause 410. The composition of clause 401 to 409, wherein the ADP receptor/P2Y12 inhibitors is selected from a group consisting of clopidogrel, ticlopidine, prasugrel, cangrelor, elinogrel, and ticagrelor. [in SLIDER #1 only]

Clause 411. The composition of clause 401 to 410, further comprising at least a fourth antiplatelet drug different than the first and second antiplatelet drugs.

Clause 412. The composition of clause 363, wherein the fourth antiplatelet drug comprises one or more of a prostaglandin analogue, a thromboxane inhibitor, and a phosphodiesterase inhibitors.

Clause 413. The composition of clause 401 to 412, further comprising a chelating agent.

Clause 414. The composition of clause 413, wherein the chelating agent is selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), deferoxamine (DFO), a surfactant-EDTA complex, quaternary ammonium EDTA salt, benzalkonium EDTA salt, EDTA complex, salts, analogue, solvate, hydrate and derivatives thereof.

Clause 415. The composition of clause 401 to 414, further comprising at least one antiproliferative drug.

Clause 416. The composition of clause 415, wherein the antiproliferative drug comprises an mTOR inhibitor.

Clause 417. The composition of clause 401 to 416, wherein individual components of the composition are configured to be coated on the surface simultaneously.

Clause 418. The composition of clause 401 to 416, wherein individual components of the composition are configured to be coated on the surface sequentially.

Clause 419 to 500 intentionally left blank.

Clause 501. A composition for coating a surface of an implantable article, said composition comprising: a factor IIa inhibitor; and one or more of (a) colchicine, (b) a first antiplatelet drug comprising a factor IIa/IIIb inhibitor. (c) a second antiplatelet drug comprising an NSAID, and (d) a third antiplatelet drug comprising an ADP inhibitor, wherein the factor Xa inhibitor and the one or more of (a) the colchicine, (b) the first antiplatelet drug, (c) the second antiplatelet drug and (d) the third antiplatelet drug are formulated to release from the surface of the article in amounts sufficient to inhibit inflammation resulting from the implantation of the article when implanted in a patient.

Clause 502. The composition of clause 501, wherein the factor IIa inhibitor and (a) the colchicine, (b) the first antiplatelet drug comprising a factor IIa/IIIb inhibitor, (c) the second antiplatelet drug comprising an NSAID, and (d) the third antiplatelet drug comprising an ADP inhibitor are formulated to treat an injury that resulted from implantation of the article.

Clause 503. The composition of clause 501 or 502, wherein the factor IIa inhibitor and (a) the colchicine, (b) the first antiplatelet drug comprising a factor IIa/IIIb inhibitor; (c) the second antiplatelet drug comprising an NSAID, and (d) the third antiplatelet drug comprising an ADP inhibitor are formulated to treat the injury without causing any one or more of bleeding, a risk of bleeding risk, and bleeding associated with an administration of a systemic antithrombotic drug.

Clause 504. The composition of clause 501 to 503, wherein the factor IIa inhibitor is selected from a group consisting of argatroban, dabigatran, ximelagatran, melagatran, efegatran, inogatran, atecegatran metoxil (AZD-0837), hirudin, hirudin analogs, bivalirudin, desirudin, and lepirudin.

Clause 505. The composition of clause 504, wherein the factor IIa inhibitor comprises argatroban, or a salt, isomer, solvate, analog, derivative, metabolite, or prodrugs thereof.

Clause 506. The composition of clause 501 to 505, further comprising a factor Xa inhibitor selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1 h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052).

Clause 507. The composition of clause 506, wherein the direct factor Xa inhibitor comprises apixaban, or a salt, isomer, solvate, analog, derivative, metabolite, or prodrugs thereof.

Clause 508. The composition of clause 507, wherein the direct factor Xa inhibitor comprises rivaroxaban, or a salt, isomer, solvate, analog, derivative, metabolite, or prodrugs thereof.

Clause 509. The composition of clause 501 to 508, wherein the factor IIb/IIIa inhibitor is selected from a group consisting of abciximab, eptifibatide, orbofiban, roxifiban, sibrafiban, and tirofiban.

Clause 510. The composition of clause 501 to 509, wherein the ADP receptor/P2Y12 inhibitors is selected from a group consisting of clopidogrel, ticlopidine, prasugrel, cangrelor, elinogrel, and ticagrelor. [in SLIDER #1 only]

Clause 511. The composition of clause 501 to 510, further comprising at least a fourth antiplatelet drug different than the first and second antiplatelet drugs.

Clause 512. The composition of clause 511, wherein the fourth antiplatelet drug comprises one or more of a prostaglandin analogue, a thromboxane inhibitor, and a phosphodiesterase inhibitors.

Clause 513. The composition of clause 501 to 512, further comprising a chelating agent.

Clause 514. The composition of clause 513, wherein the chelating agent is selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), deferoxamine (DFO), a surfactant-EDTA complex, quaternary ammonium EDTA salt, benzalkonium EDTA salt, EDTA complex, salts, analogue, solvate, hydrate and derivatives thereof.

Clause 515. The composition of clause 501 to 514, further comprising at least one antiproliferative drug.

Clause 516. The composition of clause 515, wherein the antiproliferative drug comprises an mTOR inhibitor.

Clause 517. The composition of clause 501 to 516, wherein individual components of the composition are configured to be coated on the surface simultaneously.

Clause 518. The composition of clause 5101 to 516, wherein individual components of the composition are configured to be coated on the surface sequentially.

Clause 519 to 600 intentionally left blank.

Clause 601. A composition for coating a surface of an implantable article, said composition comprising: (a) at least one of a factor IIa inhibitor and a factor Xa inhibitor, (b) at least one antiplatelet drug selected from: (i) a first antiplatelet drug comprising a COX-1 inhibitor; (ii) a second antiplatelet drug comprising a factor IIa/IIIb (2a/3b) inhibitor; and (iii) a third antiplatelet drug comprising an adenosine diphosphate (ADP)/P2Y12 inhibitor, wherein the (a) at least one of the factor IIa inhibitor and the factor Xa inhibitor and the (b) at least one antiplatelet drug are formulated to release from the surface of the article in amounts sufficient to inhibit inflammation resulting from the implantation of the article when implanted in a patient.

Clause 602. The composition of clause 601, comprising a factor IIa inhibitor selected from a group consisting of argatroban, dabigatran, ximelagatran, melagatran, efegatran, inogatran, atecegatran metoxil (AZD-0837), hirudin, hirudin analogs, bivalirudin, desirudin, and lepirudin.

Clause 603. The composition of clause 602, wherein the factor IIa inhibitor comprises argatroban, or a salt, isomer, solvate, analog, derivative, metabolite, or prodrugs thereof.

Clause 604. The composition of clause 601 to 603, comprising a factor Xa inhibitor selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or cribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052).

Clause 605. The composition of clause 604, wherein the direct factor Xa inhibitor comprises apixaban, or a salt, isomer, solvate, analog, derivative, metabolite, or prodrugs thereof.

Clause 606. The composition of clause 604 wherein the direct factor Xa inhibitor comprises rivaroxaban, or a salt, isomer, solvate, analog, derivative, metabolite, or prodrugs thereof.

Clause 607. The composition of clause 601 to 606, comprising a COX-1 inhibitor

Clause 608. The composition of clause 607, wherein the COX-1 inhibitor comprises a salicylate or other NSAID.

Clause 609. The composition of clause 601 to 607, comprising a factor IIb/IIIa inhibitor selected from a group consisting of abciximab, eptifibatide, orbofiban, roxifiban, sibrafiban, and tirofiban.

Clause 610. The composition of clause 601 to 609, comprising an ADP receptor/P2Y12 inhibitors is selected from a group consisting of clopidogrel, ticlopidine, prasugrel, cangrelor, elinogrel, and ticagrelor. [in SLIDER #1 only]

Clause 611. The composition of clause 601 to 610, further comprising at least one of a prostaglandin analogue, a thromboxane inhibitor, and a phosphodiesterase inhibitors.

Clause 612. The composition of clause 601 to 610, further comprising a chelating agent.

Clause 613. The composition of clause 612, wherein the chelating agent is selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), deferoxamine (DFO), a surfactant-EDTA complex, quaternary ammonium EDTA salt, benzalkonium EDTA salt, EDTA complex, salts, analogue, solvate, hydrate and derivatives thereof.

Clause 614. The composition of clause 601 to 613, further comprising at least one antiproliferative drug.

Clause 615. The composition of clause 614, wherein the antiproliferative drug comprises an mTOR inhibitor.

Clause 616. The composition of clause 601 to 615, wherein individual components of the composition are configured to be coated on the surface simultaneously.

Clause 617. The composition of clause 601 to 615, wherein individual components of the composition are configured to be coated on the surface sequentially.

Clause 618 to 700 intentionally left blank.

Clause 701. A composition for coating a surface of an implantable article, said composition comprising: (a) at least one of a factor IIa inhibitor and a factor Xa inhibitor (b) at least one of (i) colchicine, (ii) a first antiplatelet drug, and (iii) a chelating agent; wherein the (a) at least one of a factor IIa inhibitor and a factor Xa inhibitor and (b) the at least one of (i) colchicine (ii) the first antiplatelet drug, and the chelating agent are formulated to release from the surface of the article in amounts sufficient to inhibit inflammation resulting from the implantation of the article when implanted in a patient.

Clause 702. The composition of clause 701, comprising a factor IIa inhibitor selected from a group consisting of argatroban, dabigatran, ximelagatran, melagatran, efegatran, inogatran, atecegatran metoxil (AZD-0837), hirudin, hirudin analogs, bivalirudin, desirudin, and lepirudin.

Clause 703. The composition of clause 702, wherein the factor IIa inhibitor comprises argatroban, or a salt, isomer, solvate, analog, derivative, metabolite, or prodrugs thereof.

Clause 704. The composition of clause 701 to 703, comprising a factor Xa inhibitor selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052).

Clause 705. The composition of clause 704, wherein the direct factor Xa inhibitor comprises apixaban, or a salt, isomer, solvate, analog, derivative, metabolite, or prodrugs thereof.

Clause 706. The composition of clause 704 wherein the direct factor Xa inhibitor comprises rivaroxaban, or a salt, isomer, solvate, analog, derivative, metabolite, or prodrugs thereof.

Clause 707. The composition of clause 701 to 706, comprising colchicine.

Clause 708. The composition of clause 701 to 707, comprising one or more antiplatelet drugs selected from the group consisting of a factor IIa/IIIb inhibitor, an ADP receptor/P2Y12 inhibitor, a COX-1 inhibitor, a prostaglandin analogue, a thromboxane inhibitor, and a phosphodiesterase inhibitor.

Clause 709. The composition of clause 708, comprising a chelating agent selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), deferoxamine (DFO), a surfactant-EDTA complex, quaternary ammonium EDTA salt, benzalkonium EDTA salt, EDTA complex, salts, analogue, solvate, hydrate and derivatives thereof.

Clause 710. The composition of clause 701 to 709, further comprising at least one antiproliferative drug.

Clause 711. The composition of clause 710, wherein the antiproliferative drug comprises an mTOR inhibitor.

Clause 712. The composition of clause 701 to 711, wherein individual components of the composition are configured to be coated on the surface simultaneously.

Clause 713. The composition of clause 701 to 711, wherein individual components of the composition are configured to be coated on the surface sequentially.

Clause 714 to 800 intentionally left blank.

Clause 801. An implantable article comprises a body having a surface configured for implantation in a patient, wherein the surface is at least partially coated with a composition according to any one of clauses 101 to 713.

Clause 802. The article of clause 801, wherein the article is an expandable scaffold configured for implantation in the patient's vascular system.

Clause 803. The article of clause 801, wherein the article comprises a non-expandable body.

Clause 804. The article of clauses 801 to 803, wherein the composition is formulated to be released from the article over a period of at least of at least one week, one month, or one year.

Clause 805. The article of clauses 801 to 804, wherein at least some the components of the composition are formulated in one or more carriers which degrade when implanted in the patient.

Clause 806. The article of clauses 801 to 805, wherein the composition is formulated to release no greater than 90%, 75%, 50%, 25%, or 10% of its weight of within one week, one month, three months, six months, or one year of implantation.

The illustrative examples described are not meant to be limiting. Other examples may be utilized, and other changes may be made, or combined in whole or in part, without departing from the scope of the subject matter presented herein. It will be readily understood that the aspects of the present disclosure, as generally described herein, and illustrated in the figures, and detailed description, and in the examples, can be arranged, substituted, combined, separated, and designed in a wide variety of different configurations, all of which are explicitly contemplated herein.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A shows a plot of HAoSMC cell proliferation in the presence of rapamycin and varying concentrations of Apixaban, in accordance with example 1.

FIG. 1B shows a plot of HAoSMC cell proliferation in the presence of rapamycin and varying concentrations of Argatroban, in accordance with example 1.

FIG. 1C shows a plot of HAoSMC cell proliferation in the presence of rapamycin and varying concentrations of Apixaban and Argatroban, in accordance with example 1.

FIG. 1D shows a plot of HAoSMC cell proliferation in the presence of difference concentrations of Apixaban, in accordance with example 1.

FIG. 1E shows a plot of HAoSMC cell proliferation in the presence of difference concentrations of Argatroban, in accordance with example 1.

FIG. 2A shows a plot of activated clotting time (ACT) versus drug concentration, in accordance with examples.

FIG. 2B shows a plot of activated clotting time (ACT) versus drug concentration, in accordance with example 2.

FIG. 2C shows a plot of activated clotting time (ACT) versus drug concentration, showing the synergistic effects of Apixaban in combination with Argatroban, in accordance with example 2.

FIG. 2D shows a plot of various synergistic effects of drug combination ratios between Apixaban and Argatroban, in accordance with example 2.

FIG. 3A shows a plot of net activated clotting time (net ACT) versus drug concentration, showing the synergy effect between rivaroxaban and argatroban, in accordance with example 3.

FIG. 3B shows a plot of net activated clotting time (net ACT) versus drug concentration, showing the synergy effect between edoxaban and argatroban, in accordance with example 4.

FIG. 3C shows a plot of net activated clotting time (net ACT) versus drug concentration, showing the synergy effect between betrixaban and argatroban, in accordance with example 5.

FIG. 4A shows a plot of net activated clotting time (net ACT) versus drug concentration, showing the synergy effect between colchicine, rivaroxaban and argatroban, in accordance with example 6.

FIG. 4B shows a plot of net activated clotting time (net ACT) versus drug concentration, showing the synergy effect between tirofiban, rivaroxaban and argatroban, in accordance with example 7.

FIG. 4C shows a plot of net activated clotting time (net ACT) versus drug concentration, showing the synergy effect between ticagrelor, rivaroxaban and Argatroban, in accordance with example 8.

FIG. 4D shows a plot of net activated clotting time (net ACT) versus drug concentration, showing the synergy effect between tirofiban, colchicine, rivaroxaban and argatroban, in accordance with example 9.

FIG. 4E shows a plot of net activated clotting time (net ACT) versus drug concentration, showing the synergy effect between rivaroxaban and Tirofiban or colchicine or ticagrelor, in accordance with example 10.

FIG. 4F shows a plot of net activated clotting time (net ACT) versus drug concentration, showing the synergy effect between argatroban and tirofiban or colchicine or ticagrelor, in accordance with example 11.

FIG. 5 shows a plot of in-vivo mean tissue concentration vs time of Rapamycin, Rivaroxaban and Argatroban in stented segments, in accordance with example 27.

FIG. 6 shows a plot of in-vitro average percent of drug released vs time of Colchicine, Tirofiban, Rapamycin, Rivaroxaban and Argatroban in coated stent, in accordance with example 45.

FIG. 7 shows a plot of in-vitro average percent of drug released vs time of Colchicine, Tirofiban, Rapamycin, Rivaroxaban and Argatroban in coated stent, in accordance with example 46.

FIG. 8 shows a plot of in-vitro average percent of drug released vs time of Colchicine, Tirofiban, Rapamycin, Rivaroxaban and Argatroban in coated stent, in accordance with example 47.

FIG. 9 shows a plot of in-vitro average percent of drug released vs time of Colchicine, Tirofiban, Rapamycin, Rivaroxaban and Argatroban in coated stent, in accordance with example 48.

### DETAILED DESCRIPTION OF THE DISCLOSURE

In the following detailed description, reference is made to the accompanying figures, which form a part hereof. In the figures, similar symbols typically identify similar components, unless context dictates otherwise. The illustrative examples described in the detailed description, figures, and claims are not meant to be limiting. Other examples may be utilized, and other changes may be made, or combined in whole or in part, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain examples and examples are disclosed below, inventive subject matter extends beyond the specifically disclosed examples to other alternative examples and/or uses, and to modifications and equivalents thereof. Thus, the scope of the claims appended hereto is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples, however, the order of description should not be construed to imply that these operations are order dependent. Additionally, the structures, systems, and/or devices described herein may be embodied as integrated components or as separate components.

For purposes of comparing various examples, certain aspects and advantages of these examples are described. Not necessarily all such aspects or advantages are achieved by any particular example or embodiment. Thus, for example, various examples may be carried out in a manner that achieves or optimizes one advantage or group of advantages as taught herein without necessarily achieving other aspects or advantages as may also be taught or suggested herein.

Every example of the present disclosure may optionally be combined with any one or more of the other examples described herein. Every patent literature, and every non-patent literature, cited herein is incorporated herein by reference in its entirety.

The present disclosure is described in relation to drug-coated stents, drug-coated balloons, balloon reservoirs, heart implants, hip implants, knee implants, shoulder implants, and the like. However, one of skill in the art will appreciate that this is not intended to be limiting and the devices and methods disclosed herein may be used in other anatomical areas and in other surgical procedures or in other devices whether used as temporary devices or permanent devices.

As used herein, the term coagulation comprises one or more of thrombin formation, fibrin formation, platelet activation, platelet aggregation, and/or thrombus/clot formation. Coagulation typically arises in response to a body part injury and/or to a foreign body such as a device. This may lead to one or more of inflammation, injury, blockage of a lumen or vessel partially or fully, degradation of the device function, formation of clot, and/or adverse clinical events. In some examples, any of the devices described herein may, at least partially, cause an injury to the tissue which may initiate the coagulation cascade.

As used herein, the term anti-coagulant refers to an agent that inhibits one or more of thrombin formation, fibrin formation, platelet activation (typically indirectly), platelet aggregation (typically indirectly), thrombus (clot) formation, thrombin dissolution, fibrin dissolution, or thrombus dissolution, thereby inhibiting one or more of blockage of a lumen or vessel partially or fully, degradation of the device function, formation of clot, and/or adverse clinical events.

Inhibiting one or more of thrombin formation, fibrin formation, platelet activation, and/or platelet aggregation enables the inhibition of one or more of blockage of a lumen or vessel partially or fully, degradation of the device function, formation of thrombus (clot) formation, inflammation, and/or adverse clinical events.

Described herein are devices and methods for locally delivering a therapeutic composition to a patient. The therapeutic composition includes one or more agents which inhibit one or more of thrombin, fibrin, and/or thrombus formation or promote one or more of thrombin, fibrin, and/or thrombus dissolution. In preferred examples, the therapeutic composition includes one or more of a calcium chelating agent, a direct Xa inhibitor and a direct Fla inhibitor, in another preferred example, an anti-proliferative agent may be added to the therapeutic composition of the direct Xa inhibitor and/or the direct IIa inhibitor.

In some examples, the device or the implant comprising: a body structure having a surface configured to be implanted in a patient's body; and a therapeutic composition present on a surface of the body structure, therapeutic composition comprising at least one drug selected from the group consisting of a chelating agent, a direct factor IIa inhibitor, an a direct factor Xa inhibitor, wherein the therapeutic composition is formulated for a delayed release into an environment surrounding the body structure upon implantation of the body structure into environment.

In some examples, the implantable scaffold with the therapeutic composition is formulated for a rapid release into the environment surrounding the body structure a preselected time period after implantation of the body structure into environment.

In some examples, the implantable scaffold with the body structure comprises an expandable scaffold, an orthopedic implant or any type of therapeutic, diagnostic, or other structure intended for implantation in the patient's body, typically being an expandable scaffold, such as a vascular stent, a prosthetic heart valve, a patent foramen ovale (PFO) occlusion device, an atrial septal defect (ASD) occlusion device, a left atrial appendage (LAA) occlusion device, or similar expandable structure. Orthopedic implants include artificial joints, spinal implants, medullary screws, and the like.

In some examples, the implantable scaffold with the therapeutic composition is present at least partly on the surface of the body structure.

In some examples, the implantable scaffold with the therapeutic composition is present at least partly within a cavity or reservoir within the body structure.

In some examples, the implantable scaffold with the therapeutic composition is formulated to inhibit release of the at least one drug into the environment surrounding the body structure for a time period in a range from 5 minutes to 240 minutes, preferably from 5 minutes to 180 minutes, and more preferably from 5 minutes to 60 minutes.

In some examples, the implantable scaffold with the therapeutic composition is formulated to release at least 50% by weight, preferably at least 75% by weight of the at least one drug into the environment surrounding the body structure within 72 hours of implantation, preferably within 24 hours of implantation, more preferably within 6 hours of implantation, and even more preferably within 4 hour of implantation.

In some examples, the implantable scaffold with the therapeutic composition is formulated to release additional amounts of the at least one drug into the environment for a period of at least 3 days, preferably at least 7 days, more preferably 21 days, still more preferably at least 28 days, even more preferably at least 3 months, and often 6 months or more after implantation.

In some examples, the implantable scaffold with the drug comprises at least a chelating agent in the therapeutic composition is formulated to deplete calcium in the environment surrounding the body structure upon implantation of the body structure in environment.

In some examples, the implantable scaffold with the chelating agent is selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenctriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), the surfactant-EDTA complex, quaternary ammonium EDTA salt, benzalkonium EDTA salt, EDTA complex, salts, analogue, solvate, hydrate and derivatives thereof.

In some examples, the implantable scaffold with the cationic anti-coagulation enhancer is selected from a group consisting of cationic polymer or compounds including but not limit to poly(L-lysine) (PLL), linear polyethyleneimine (PEI), branch polyethyleneimine (PEI), chitosan, PAMAM dendrimers, and poly(2-dimethylamino)ethyl methacrylate (pDMAEMA), protamine, polylysine, a polybetaaminoester (PBAE), Histone, ethylenediamine, methylenediamine, ammonium chloride, melamine, histamine, histidine, analogue, solvate, hydrate and derivatives thereof.

In some examples, the implantable scaffold with the chelating agent consists essentially of ethylenediaminetetraacetic acid (EDTA).

In some examples, the implantable scaffold with the cationic anti-coagulation enhancer consists essentially of benzyldimethyltetradecylammonium chloride.

In some examples, the implantable scaffold with the cationic anti-coagulation enhancer consists essentially of linear polyethyleneimine (PEI).

In some examples, the implantable scaffold with the at least one drug comprises a direct factor IIa inhibitor selected from the group consisting of argatroban, dabigatran, ximelagatran, melagatran, efegatran, inogatran, atecegatran metoxil (AZD-0837), hirudin, hirudin analogs, bivalirudin, desirudin, and lepirudin.

In some examples, the implantable scaffold with the at least one direct factor IIa inhibitor comprises argatroban, or a salt, isomer, solvate, analog, derivative, metabolite, or prodrugs thereof.

In some examples, the implantable scaffold with the at least one drug comprises a direct factor Xa inhibitor selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052).

In some examples, the implantable scaffold of claim 64, wherein the direct factor Xa inhibitor comprises apixaban, or a salt, isomer, solvate, analog, derivative, metabolite, or prodrugs thereof.

In some examples, the implantable scaffold of claim 64, wherein the direct factor Xa inhibitor comprises rivaroxaban, or a salt, isomer, solvate, analog, derivative, metabolite, or prodrugs thereof.

In some examples, the implantable scaffold with therapeutic composition further comprises an mTOR inhibitor selected from a group consisting of sirolimus, biolimus, everolimus, myolimus, novolimus, ridaforolimus, temsirolimus, zotarolimus, or salts, isomers, solvates, analogs, derivatives, metabolites, or prodrugs thereof.

In some examples, the implantable scaffold of claim 14, wherein the mTOR inhibitor comprises sirolimus, or a salt, isomer, solvate, analog, derivative, metabolite, or prodrugs thereof.

In some examples, the implantable scaffold with therapeutic composition further comprises paclitaxel, or a salt, isomer, solvate, analog, derivative, metabolite, or prodrug thereof.

In some examples, the implantable scaffold with the therapeutic composition further comprises an antiplatelet drug.

In some examples, the implantable scaffold with the therapeutic composition further comprises an antiproliferative agent selected from the group consisting of mycophenolate mofetil, mycophenolate sodium, azathioprine.

In some examples, the implantable scaffold with the implantable scaffold has at least an outer surface, an inner surface, and one or more edge surfaces between the outer and inner surfaces.

In some examples, the implantable scaffold with at least a portion of the outer surface is coated with the therapeutic compositions.

In some examples, the implantable scaffold with at least a portion of the inner surface is coated with the therapeutic compositions.

In some examples, the implantable scaffold with a portion of the edge surfaces is coated with the therapeutic compositions.

In some examples, the implantable scaffold at least some of the surfaces have receptacles formed therein and at least some of receptacles have therapeutic agent therein.

In some examples, the implantable scaffold with the receptacles comprises one or more of wells, channels, holes, and surface texture.

In some examples, a therapeutic composition present on a surface of the scaffold. The therapeutic composition comprising at least one chelating agent. In some examples, the implantable scaffold the therapeutic composition is present at least partly on the surface of scaffold structure. In some examples, the implantable scaffold the therapeutic composition is present at least partly within a cavity or reservoir within the scaffold structure.

In some examples, the implantable scaffold with the chelating agent in the therapeutic composition is formulated to deplete calcium in the environment surrounding the scaffold upon implantation of the scaffold structure in environment.

In some examples, the implantable scaffold with the therapeutic composition is formulated to release at least 50%, preferably at least 75%, by weight of the at least one chelating agent into the vascular environment within 72 hours of implantation, preferably within 24 hours of implantation, more preferably within 6 hours of implantation, and even more preferably within 4 hours of implantation.

In some examples, the implantable scaffold with the therapeutic composition is formulated to release additional amounts of the at least one chelating agent into the environment for a period of at least 3 days, preferably at least 7 days, more preferably 21 days, still more preferably at least 28 days, even more preferably at least 3 months, and often 6 months or more after implantation.

In some examples, the implantable scaffold with the therapeutic composition consists essentially of chelating agent.

In some examples, the implantable scaffold with the chelating agent is selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), the surfactant-EDTA complex, quaternary ammonium EDTA salt, benzalkonium EDTA salt, EDTA complex, salts, analogue, solvate, hydrate and derivatives thereof.

In some examples, the implantable scaffold with the cationic anti-coagulation enhancers is selected from a group consisting of cationic polymer or compounds including but not limit to poly(L-lysine) (PLL), linear polyethyleneimine (PEI), branch polyethyleneimine (PEI), chitosan, PAMAM dendrimers, and poly(2-dimethylamino)ethyl methacrylate (pDMAEMA), protamine, polylysine, a polybetaaminoester (PBAE), Histone, ethylenediamine, methylenediamine, ammonium chloride, melamine, histamine, histidine, analogue, solvate, hydrate and derivatives thereof.

In some examples, the implantable scaffold with the chelating agent consists essentially of ethylenediaminetetraacetic acid (EDTA).

In some examples, the implantable scaffold with the cationic anti-coagulation enhancer consists essentially of benzyldimethyltetradecylammonium chloride.

In some examples, the implantable scaffold with the cationic anti-coagulation enhancer consists essentially of linear polyethyleneimine (PEI).

In some examples, the implantable scaffold with the chelating agent is present in the therapeutic composition at a weight percent from 10% to 100%.

In some examples, the implantable scaffold with therapeutic composition comprises additional active and/or inactive substances.

In some examples, the implantable scaffold with the additional active and/or inactive substances are present in the therapeutic composition at a weight percent from 20% to 90%.

In some examples, the implantable scaffold with the therapeutic composition further comprises at least one anti-coagulant.

In some examples, the implantable scaffold with the therapeutic composition is formulated to release at least one anti-coagulant at a rate equal to that of the chelating agent.

In some examples, the implantable scaffold with the therapeutic composition is formulated to release at least one anti-coagulant at a rate slower than that of the chelating agent.

In some examples, the implantable scaffold with the therapeutic composition is formulated to release at least one anti-coagulant at a rate faster than that of the chelating agent.

In some examples, the implantable scaffold with the at least one anti-coagulant is selected from the group consisting of a direct factor IIa inhibitor and a direct factor Xa inhibitor.

In some examples, the implantable scaffold with the at least one anti-coagulant comprises a direct factor IIa inhibitor selected from the group consisting of argatroban, dabigatran, ximelagatran, melagatran, efegatran, inogatran, atecegatran metoxil (AZD-0837), hirudin, hirudin analogs, bivalirudin, desirudin, and lepirudin.

In some examples, the implantable scaffold with the at least one direct factor IIa inhibitor comprises argatroban, or a salt, isomer, solvate, analog, derivative, metabolite, or prodrugs thereof.

In some examples, the implantable scaffold with any one of the at least one anti-coagulant comprises a direct factor Xa inhibitor selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150). 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052).

In some examples, the implantable scaffold with the direct factor Xa inhibitor comprises apixaban, or a salt, isomer, solvate, analog, derivative, metabolite, or prodrugs thereof.

In some examples, the implantable scaffold with the direct factor Xa inhibitor comprises rivaroxaban, or a salt, isomer, solvate, analog, derivative, metabolite, or prodrugs thereof.

In some examples, the implantable scaffold with the therapeutic composition further comprises an mTOR inhibitor selected from a group consisting of sirolimus, biolimus, everolimus, myolimus, novolimus, ridaforolimus, temsirolimus, zotarolimus, or salts, isomers, solvates, analogs, derivatives, metabolites, or prodrugs thereof.

In some examples, the implantable scaffold with the mTOR inhibitor comprises sirolimus, or a salt, isomer, solvate, analog, derivative, metabolite, or prodrugs thereof.

In some examples, the implantable scaffold with therapeutic composition further comprises paclitaxel, or a salt, isomer, solvate, analog, derivative, metabolite, or prodrug thereof.

In some examples, the implantable scaffold with therapeutic composition further comprises an antiplatelet drug.

In some examples, the implantable scaffold with therapeutic composition further comprises an antiproliferative agent selected from the group consisting of mycophenolate mofetil, mycophenolate sodium, azathioprine.

In some examples, the implantable scaffold with at least a portion of the outer surface is coated with the therapeutic compositions.

In some examples, the implantable scaffold with at least a portion of the inner surface is coated with the therapeutic compositions.

In some examples, the implantable scaffold with a portion of the edge surfaces is coated with the therapeutic compositions.

In some examples, the implantable scaffold with at least some of the surfaces have receptacles formed therein and at least some of receptacles have therapeutic agent therein.

In some examples, the implantable scaffold with the receptacles comprises one or more of wells, channels, holes, and surface texture.

In some examples, a method for treating a vascular tissue injury in a patient, the method comprising: implanting a scaffold structure at a target location in the patient's vasculature proximate the tissue injury; and releasing a drug composition including at least one chelating agent from the implanted scaffold structure into the vasculature, wherein the chelating agent is released sufficiently rapidly into the vasculature to prevent blood clotting and inhibit the fibrin formation.

In some examples, the method with at least 75% by weight of the at least one chelating agent is released into the vasculature within 72 hours of implantation, preferably within 24 hours of implantation, more preferably within 6 hours of implantation, and even more preferably within 4 hour of implantation, and usually between 10 minutes and 4 hours.

In some examples, the implantable scaffold with the therapeutic composition consists essentially of chelating agent.

In some examples, the implantable scaffold with the chelating agent is selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy)ethane-N,N,N′,N′-tetraacetic acid (BAPTA), the surfactant-EDTA complex, quaternary ammonium EDTA salt, benzalkonium EDTA salt, EDTA complex, salts, analogue, solvate, hydrate and derivatives thereof.

In some examples, the implantable scaffold with the cationic anti-coagulation enhancer is selected from a group consisting of cationic polymer or compounds including but not limit to poly(L-lysine) (PLL), linear polyethyleneimine (PEI), branch polyethyleneimine (PEI), chitosan, PAMAM dendrimers, and poly(2-dimethylamino)ethyl methacrylate (pDMAEMA), protamine, polylysine, a polybetaaminoester (PBAE), Histone, ethylenediamine, methylenediamine, ammonium chloride, melamine, histamine, histidine, analogue, solvate, hydrate and derivatives thereof.

In some examples, the implantable scaffold with the chelating agent consists essentially of ethylenediaminetetraacetic acid (EDTA).

In some examples, the implantable scaffold with the cationic anti-coagulation enhancer consists essentially of benzyldimethyltetradecylammonium chloride.

In some examples, the implantable scaffold with the cationic anti-coagulation enhancer consists essentially of linear polyethyleneimine (PEI).

In some examples, the implantable scaffold with the chelating agent is present in the therapeutic composition at a weight percent from 10% to 100%.

In some examples, the implantable scaffold with therapeutic composition comprises additional active and/or inactive substances.

In some examples, the implantable scaffold with the additional active and/or inactive substances are present in the therapeutic composition at a weight percent from 20% to 90%.

In some examples, the implantable scaffold with the therapeutic composition further comprises at least one anti-coagulant.

In some examples, the implantable scaffold with the therapeutic composition is formulated to release at least one anti-coagulant at a rate equal to that of the chelating agent.

In some examples, the implantable scaffold with the therapeutic composition is formulated to release at least one anti-coagulant at a rate slower than that of the chelating agent.

In some examples, the implantable scaffold with the therapeutic composition is formulated to release at least one anti-coagulant at a rate faster than that of the chelating agent.

In some examples, the implantable scaffold with the at least one anti-coagulant is selected from the group consisting of a direct factor IIa inhibitor and a direct factor Xa inhibitor.

In some examples, the implantable scaffold with the at least one anti-coagulant comprises a direct factor IIa inhibitor selected from the group consisting of argatroban, dabigatran, ximelagatran, melagatran, efegatran, inogatran, atecegatran metoxil (AZD-0837), hirudin, hirudin analogs, bivalirudin, desirudin, and lepirudin.

In some examples, the implantable scaffold with the at least one direct factor IIa inhibitor comprises argatroban, or a salt, isomer, solvate, analog, derivative, metabolite, or prodrugs thereof.

In some examples, the implantable scaffold with any one of the at least one anti-coagulant comprises a direct factor Xa inhibitor selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1 h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or cribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052).

In some examples, the implantable scaffold with the direct factor Xa inhibitor comprises apixaban, or a salt, isomer, solvate, analog, derivative, metabolite, or prodrugs thereof.

In some examples, the implantable scaffold with the direct factor Xa inhibitor comprises rivaroxaban, or a salt, isomer, solvate, analog, derivative, metabolite, or prodrugs thereof.

In some examples, the implantable scaffold with the therapeutic composition further comprises an mTOR inhibitor selected from a group consisting of sirolimus, biolimus, everolimus, myolimus, novolimus, ridaforolimus, temsirolimus, zotarolimus, or salts, isomers, solvates, analogs, derivatives, metabolites, or prodrugs thereof.

In some examples, the implantable scaffold with the mTOR inhibitor comprises sirolimus, or a salt, isomer, solvate, analog, derivative, metabolite, or prodrugs thereof.

In some examples, the implantable scaffold with therapeutic composition further comprises paclitaxel, or a salt, isomer, solvate, analog, derivative, metabolite, or prodrug thereof.

In some examples, the implantable scaffold with therapeutic composition further comprises an antiplatelet drug.

In some examples, the implantable scaffold with therapeutic composition further comprises an antiproliferative agent selected from the group consisting of mycophenolate mofetil, mycophenolate sodium, azathioprine.

In some examples, the implantable scaffold with at least a portion of the outer surface is coated with the therapeutic compositions.

In some examples, the implantable scaffold with at least a portion of the inner surface is coated with the therapeutic compositions.

In some examples, the implantable scaffold with a portion of the edge surfaces is coated with the therapeutic compositions.

In some examples, the implantable scaffold with at least some of the surfaces have receptacles formed therein and at least some of receptacles have therapeutic agent therein.

In some examples, the implantable scaffold with the receptacles comprises one or more of wells, channels, holes, and surface texture.

In some examples, a method for treating a vascular tissue injury in a patient, the method comprising: implanting a scaffold structure at a target location in the patient's vasculature proximate the tissue injury; and releasing a drug composition including at least one chelating agent from the implanted scaffold structure into the vasculature, wherein the chelating agent is released sufficiently rapidly into the vasculature to prevent blood clotting and inhibit the fibrin formation.

In some examples, the method with at least 75% by weight of the at least one chelating agent is released into the vasculature within 72 hours of implantation, preferably within 24 hours of implantation, more preferably within 6 hours of implantation, and even more preferably within 4 hour of implantation, and usually between 10 minutes and 4 hours.

In some examples, the method with the therapeutic composition is positioned on at least one of an internal surface and an external surface of the implantable scaffold.

In some examples, the method with the therapeutic composition is positioned on both an external and an internal surface of the implantable scaffold.

In some examples, the method with the tissue injury is caused by expanding the scaffold at the location.

In some examples, the method with the tissue injury pre-exists deploying the structure at the location.

In some examples, the devices described herein can be configured to release a direct factor Xa inhibiting agent to a mammalian body, lumen, tissue, and/or device surface prior to an injury to said tissue, concurrent with injury to said tissue, or after an initial injury to said tissue. The device is introduced into said mammalian body and advanced to said tissue site or body lumen. In some specific examples, the device is expanded against said tissue to release said agent. In other examples, the device is expanded against said tissue to perform a function such as opening up a vessel or lumen and to release said agent. In specific examples, the device is a stent or a balloon catheter. In yet another example, the device is placed adjacent to said tissue.

In some examples, the direct factor Xa inhibitor is selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), cribaxaban (PD 0348292), 2-(5-carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052), (S)-3-(7-carbamimidoylnaphthalen-2-yl)-2-(4-(((S)-1-(1-iminoethyl)pyrrolidin-3-yl)oxy)phenyl)propanoic acid hydrochloride pentahydrate (DX9065a), letaxaban (TAK-442), GW813893, JTV-803, KFA-144, DPC-423, RPR-209685, MCM-09, or antistasin. For example, the direct factor Xa inhibitor may comprise rivaroxaban, or a salt, isomer, solvate, analog, derivative, metabolite, or prodrugs thereof. Alternatively, or in combination, the direct factor Xa inhibitor may comprise apixaban.

In some examples, an IC50 of the direct factor Xa inhibitor is within a range of about 0.01 nanomolar (nM) to about 1000 nM. For example, the IC50 of the direct factor Xa inhibitor is within a range of about 0.1 nM to about 1000 nM, about 1 nM to about 1000 nM, or about 10 nM to about 1000 nM.

In some examples, the therapeutically effective dose of the direct factor Xa inhibitor is within a range of about 1 microgram to 10 mg, more preferably is within a range of about 50 micrograms to about 10 mg. For example, the therapeutically effective dose of the direct factor Xa inhibitor may be within a range of about 0.1 mg to about 10 mg or about 1 mg to about 5 mg.

In some examples, the therapeutically effective dose is sufficient to generate a tissue concentration of the direct factor Xa inhibitor of about 0.001 ng/g tissue to about 100 mg/g tissue, preferably, the therapeutically effective dose is sufficient to generate a tissue concentration of the direct factor Xa inhibitor of about 0.01 ng/g tissue to about 100 mg/g tissue, more preferably, the therapeutically effective dose is sufficient to generate a tissue concentration of the direct factor Xa inhibitor of about 0.1 ng/g tissue to about 100 mg/g tissue. For example, the therapeutically effective dose may be sufficient to generate a tissue concentration of the direct factor Xa inhibitor of about 0.2 ng/g tissue to about 100 mg/g tissue, about 0.5 ng/g tissue to about 100 mg/g tissue, about 1 ng/g tissue to about 100 mg/g tissue, about 10 ng/g tissue to about 100 mg/g tissue, or about 100 ng/g tissue to about 100 mg/g tissue; in about 1 day, 30 days, 60 days, 90 days, or 120 days after introducing the therapeutically effective dose. In some examples, the therapeutically effective dose is sufficient to generate a blood concentration of the direct factor Xa inhibitor which is less than about 200 ng/ml, 100 ng/ml, 50 ng/ml 25 ng/ml, or 10 ng/ml. In some examples, the therapeutically effective dose is sufficient to generate a blood concentration of the direct factor Xa inhibitor which is less than a systemic therapeutic concentration of the direct factor Xa inhibitor for any systemic indication. In some examples, the therapeutically effective dose is sufficient to generate a blood concentration of the direct factor Xa inhibitor which is smaller than a median maximum serum concentration (Cmax) of the direct factor Xa inhibitor generated by systemic delivery of the direct factor Xa inhibitor to achieve the same tissue concentration at the site of the inflammatory ophthalmic condition or disease. In some examples, the therapeutically effective dose is sufficient to generate a blood concentration of the direct factor Xa inhibitor which does not exceed a median maximum serum concentration (Cmax) of the direct factor Xa inhibitor generated by systemic delivery of the direct factor Xa inhibitor to achieve the same tissue concentration at the site of the inflammatory ophthalmic condition or disease for more than about 6 hours to about 3 days.

In some examples, the therapeutically effective dose is sufficient to maintain a tissue concentration of the direct factor Xa inhibitor of about 0.1 ng/g tissue to about 100 mg/g tissue for about 1 day to about 120 days, for about 1 day to about 1 year, 30 days to about 1 year, 3 months to about 1 year, or 6 months to about 1 year. In some other examples, the device releases said agent prior to engaging (or coupling or contacting) of the device to the tissue site.

In a preferred example, the tissue concentration in the tissue segment at 3 hours after injury and/or release of said agent to the tissue segment ranges from about 100,000 times the IC50 of factor Xa inhibition to 10,000,000 times the IC50 of factor Xa inhibition, preferably ranges from 500.000 times to 5,000,000 times the IC50 of factor Xa inhibition. The tissue concentration in the adjacent tissue segment (e.g., 5 mm) at 3 hours after release of said agent to the tissue segment ranges from 100 times the IC50 of factor Xa inhibition to 1,000,000 times the IC50 of factor Xa inhibition, preferably ranges from 1,000 times to 100,000 times the IC50 of factor Xa inhibition. In a preferred example, the tissue concentration in the tissue segment at about 24 hours after injury and/or release of said agent to the tissue segment ranges from 100,000 times the IC50 of factor Xa inhibition to 1000,000 times the IC50 of factor Xa inhibition, preferably ranges from 1000 times to 20,000 times the IC50 of factor Xa inhibition. In a preferred example, the agent is rivaroxaban, apixaban, and/or analogs, derivatives, or salts thereof. In a most preferred example, the agent is apixaban.

In another example, the combination of apixaban and argatroban have an additive effect on thrombin formation inhibition or dissolution.

In some examples, a combination of factor IIa inhibitor and factor Xa inhibitor are released from a device to a mammalian body, lumen, tissue, and/or device surface after injury at sufficient concentrations in the tissue segment and adjacent tissue segments within about 3 hours after injury to inhibit thrombus (clot) formation. In a preferred example, the agents are apixaban and argatroban.

In some examples, the combination of apixaban and argatroban released from a device containing an mTOR inhibitor such as sirolimus maintains or enhances the antiproliferative effect of said mTOR at the tissue segment site while inhibiting thrombus formation at the said tissue segment site.

In some examples, the combination of apixaban and argatroban released from a device containing an mTOR inhibitor inhibits thrombus formation on the device surface.

In some examples, the device is coated or loaded with one or more agents comprising apixaban, argatroban and an mTOR inhibitor. The coating coats one or more surfaces of the device, preferably coating all surfaces of the device including the abluminal and luminal surfaces of the device. Alternatively, or in combination, structural elements of the device are loaded with the one or more agents. In a specific example, the one or more agents are contained in a drug polymer matrix, or contained in a polymer top layer or coat, or is coated as a top layer or coat. In a preferred example of a device configured to release two or more agents, the agents are contained in the same polymer matrix or a different polymer matrix, or one agent is in a polymer matrix while the other agent is under a top polymer coat. In yet another preferred example, the device contains three agents in the same polymer matrix. In another example, each of the drugs is contained in a separate polymer matrix. In yet another example, two of the agents are contained in one polymer matrix while the third agent is contained in a separate polymer matrix or a top layer or coat. In yet another example, the one or more agents are contained in the same polymer matrix and a top layer or coat of a polymer material covers the surface of the device.

While many of the examples described herein depict one or more active substances being coated on a device for local delivery of the one or more active substances, it will be understood by one of ordinary skill in the art that any of the devices described herein may locally delivery one or more of the active substances through any other means. For examples, one or more of the active substances may be coated, dipped, printed, deposited, painted, brushed, loaded, or otherwise disposed on one or more surfaces of the device for local delivery. In some examples, one or more of the active substances may be incorporated into the backbone structure of the device. Alternatively, or in combination, one or more of the active substances may be locally delivered via a drug reservoir coupled to the device. In some examples, one or more or the active substances may be coated or otherwise disposed directly onto one or more surfaces of the device. In some examples, one or more or the active substances may be coated or otherwise disposed on one or more surfaces of the device in a carrier such as a polymer matrix. In some examples, one or more of the active substances may be cross-linked with a polymer, or to itself, or to another drug (in order to be another active substance, e.g., after the links are broken in vivo). In some examples, the carrier may be an excipient, a polymer, or other types of material to facilitate applying or controlling the drug onto the device or controlling release of the drug from the device or protecting the drug from washing out during entry or deployment into the body. In some examples, the carrier may comprise a microsphere or a nanosphere.

When the therapeutic composition comprises a calcium chelating agent, a direct factor IIa inhibitor, a direct factor Xa inhibitor, and an anti-proliferative agent, the therapeutic composition may be present in the carrier material at weight ratios of 1:3:1, 3:2:1, 2:2:1, 2:3:1, 3:3:1, 5:5:1, or 6:6:1, respectively. In some examples, the weight compositional ratio of the direct factor IIa inhibitor to the direct factor Xa inhibitor to the anti-proliferative agent in the therapeutic composition may be about 5:5:2. In some examples, the weight compositional ratio of the direct factor IIa inhibitor to the direct factor Xa inhibitor to the anti-proliferative agent in the coating may be within a range of about 6:6:1 to 1:3:1.

When the therapeutic composition comprises a calcium chelating agent, a direct factor IIa inhibitor, a direct factor Xa inhibitor, and an anti-proliferative agent, the release rate ratio of the direct factor IIa inhibitor to the direct factor Xa inhibitor to the anti-proliferative agent may be about 1:1:1 to about 4:4:1. In some examples, the coating may be configured to release the direct factor IIa inhibitor, the direct factor Xa inhibitor, and the anti-proliferative agent at the same rate. In other examples, the coating may be configured to release the direct factor IIa inhibitor, the direct factor Xa inhibitor, and the anti-proliferative agent at different rates.

In some examples, the coating may be configured to release the direct factor IIa inhibitor at a rate of about 4 μg/hour/mm to about 14 μg/day/mm per device.

In some examples, the coating may be configured to release the direct factor Xa inhibitor at a rate of about 4 μg/hour/mm to about 14 μg/day/mm per device.

In some examples, the coating may be configured to release the anti-proliferative agent at a rate of about 1 μg/hour/mm to about 4 μg/day/mm per device.

In some examples, the direct factor IIa inhibitor may have an inhibition potency for factor IIa ranging from about 0.001 nM to about 100 nM.

In some examples, the direct factor Xa inhibitor may have an inhibition potency for factor Xa ranging from about 0.001 nM to about 50 nM.

In some examples, the direct factor IIa inhibitor may comprise argatroban, or a salt, isomer, solvate, analog (including deuterated analog), derivative, metabolite, or prodrugs thereof.

In some examples, the direct factor Xa inhibitor may comprise apixaban, or a salt, isomer, solvate, analog (including deuterated analog), derivative, metabolite, or prodrugs thereof.

In some examples, the direct factor Xa inhibitor may comprise rivaroxaban, or a salt, isomer, solvate, analog (including deuterated analog), derivative, metabolite, or prodrugs thereof.

In some examples, the anti-proliferative agent may comprise rapamycin, or a salt, isomer, solvate, analog (including deuterated analog), derivative, metabolite, or prodrugs thereof.

In some examples, the direct factor Xa inhibitor may comprise apixaban, the direct factor IIa inhibitor may comprise argatroban, and the anti-proliferative agent may comprise rapamycin.

In some examples, the therapeutic composition is disposed on the external surface of the structure and on the internal surface of the structure. In some examples, the therapeutic composition is disposed on the external surface (abluminal) of the structure, on the interior surface (luminal) of the structure, and on the side surfaces of the structure. In yet other examples, the therapeutic composition is disposed on one or more surfaces of the structure. In yet other examples, the therapeutic composition is disposed on all surfaces of the structure. In yet other examples, the therapeutic composition is disposed in a reservoir on or in the structure. In some examples, the therapeutic composition is disposed on the external surface of the structure.

In some examples, the first layer or the second layer may comprise one or more bioactive agents and the other layer may not comprise a bioactive agent.

In some examples, the first layer may comprise one or more bioactive agents and the second layer may comprise one or more bioactive agents. The first layer and the second layer may be configured to release their respective bioactive agents at the same rate. Alternatively, the first layer and the second layer may be configured to release their respective bioactive agents at different rates.

In some examples, the first layer may comprise a calcium chelating agent, a direct factor IIa inhibitor and the second layer may comprise a direct factor Xa inhibitor. In some examples, the first layer may comprise a direct factor Xa inhibitor, and the second layer may comprise a direct factor IIa inhibitor.

In some examples, the first layer may comprise an anti-proliferative agent and the second layer may comprise a calcium chelating agent, a direct factor IIa inhibitor and a direct factor Xa inhibitor.

In some examples, the first layer may comprise a calcium chelating agent, a direct factor IIa inhibitor and a direct factor Xa inhibitor and the second layer may comprise an anti-proliferative agent.

In some examples, one or both of the layers may comprise a polymeric material as described herein. For example, the first layer may comprise a polymeric material and the second layer may not comprise a polymeric material. Alternatively, the first layer may not comprise a polymeric material and the second layer may comprise a polymeric material. Alternatively, both the first layer and the second layer may comprise the same or a different polymeric material and/or polymeric material concentration and/or formulation.

The first layer and/or second layer may be disposed on an external surface of the stent (e.g., on the abluminal surface of one or more filaments of the stent), on an internal surface of the stent (e.g., on the luminal surface of one or more filaments of the stent), or on both the external surface and the internal surface of the stent (e.g., partially or fully surround one or more filaments of the stent). The first layer and the second layer of the coating are shown fully coating each of the filaments of the stent. However, it will be understood by one of ordinary skill in the art that first layer and the second layer may coat the stent differently. For example, the first layer of the coating may fully surround each of the filaments of the stent while the second layer may be applied only on one surface (e.g., luminal or abluminal) of the stent. In some examples, both layers are not on an external surface of the stent.

In some examples, the first layer, the second layer, and/or the third layer may comprise one or more bioactive agents and one or more of the other layers may not comprise a bioactive agent.

In some examples, the first layer may comprise one or more bioactive agents, the second layer may comprise one or more bioactive agents, and the third layer may comprise one or more bioactive agents. The first layer, the second layer, and the third layer may be configured to release their respective bioactive agents at the same rate. Alternatively, two or more of the first layer, the second layer, or the third layer may be configured to release their respective bioactive agents at different rates.

In some examples, the first layer may comprise a calcium chelating agent, a direct factor IIa inhibitor, the second layer may comprise a direct factor Xa inhibitor, and the third layer may comprise an anti-proliferative agent. In some examples, the first layer may comprise a calcium chelating agent, the second layer may comprise a direct factor Xa/IIa inhibitor, and the third layer may comprise an anti-proliferative agent.

In some examples, the first layer may comprise an anti-proliferative agent, the second layer may comprise a calcium chelating agent, a direct factor IIa inhibitor, and a direct factor Xa inhibitor. In some examples, the first layer may comprise an anti-proliferative agent, the second layer may comprise a calcium chelating agent, and the third layer may comprise a direct factor IIa inhibitor and a direct factor IIa inhibitor.

In some examples, one, two, or three of the layers may comprise a polymeric material as described herein. For example, the first layer may comprise a polymeric material, the second layer may comprise a polymeric material, and the third layer may not comprise a polymeric material. The layers may comprise the same or a different polymeric material and/or polymeric material concentration and/or formulation.

The first layer, second layer, and/or third layer may be disposed on an external surface of the stent (e.g., on the abluminal surface of one or more filaments of the stent), on an internal surface of the stent (e.g., on the luminal surface of one or more filaments of the stent), or on both the external surface and the internal surface of the stent (e.g., partially or fully surround one or more filaments of the stent). The first layer, the second layer, and third layer of the coating are shown fully coating each of the filaments of the stent. However, it will be understood by one of ordinary skill in the art that one or more of the first layer, the second layer, and the third layer may coat the stent differently. For example, the first layer of the coating may fully surround each of the filaments of the stent while the second layer may be applied to a first surface (e.g., luminal) and the third layer may be applied to a second surface (e.g., abluminal) of the stent. In some examples, the layers are not on an external surface of the stent.

It will be understood by one of ordinary skill in the art based on the description herein that any of the coating described herein may comprise any number of layers desired and that the layers may comprise any number and combination of bioactive agents, carrier materials, etc. desired.

In some examples, the therapeutic coating may comprise a single layer comprising a plurality of bioactive agents. For example, the single layer may comprise a therapeutic composition of a calcium chelating agent, an anti-proliferative agent, a direct factor Xa inhibitor, and/or a direct factor IIa inhibitor as described herein. The bioactive agents may be uniformly distributed in a carrier material of the single layer. Alternatively, the bioactive agents may be non-uniformly distributed in a carrier material of the single layer. In some examples, the carrier material is a polymeric material.

In some examples, the therapeutic composition may comprise a calcium chelating agent, a direct factor IIa inhibitor, a direct factor Xa inhibitor, and/or an anti-proliferative agent as described herein.

In still further examples, a substantial amount, or substantially all, of each of the fibrin formation inhibition, thrombus formation-inhibiting or fibrin or thrombus dissolution-promoting agent(s) is released from the device within about 1 minute(s) (min), 15 min, 30 min, 1 hour(s) (hr), 6 hr, 12 hr, 1 day, 2 days, 3 days, 1 week, 2 weeks, 3 weeks, 1 month, 2 months, 3 months, 4 months, 5 months, 6 months, 9 months, or 1 years. In a preferred example, the one or more agents comprising a calcium chelating agent, a direct factor IIa inhibitor or factor Xa inhibitor are configured to substantially release over at least 28 days, preferably over at least 90 days, over at least 6 months, or over at least 1 year.

In some examples, the therapeutic coating may locally release the one or more cationic anti-coagulation enhancers to the device surface, which may be disposed adjacent a tissue segment of interest. The coating may release the one or more cationic anti-coagulation enhancers to the device surface in sufficient dose/concentrations to inhibit platelet aggregation, thrombus, thrombin, and/or clot formation. The vicinity to the wall or tissue contacting blood should have sufficient drug concentration to inhibit platelet aggregation, fibrin, thrombin, and/or clot formation.

In some examples, the therapeutic coating may locally release the one or more cationic anti-coagulation enhancers agents into the blood adjacent the tissue segment.

In some examples, the therapeutic coating may locally release the one or more bioactive agents to the device surface, which may be disposed adjacent a tissue segment of interest. The coating may release the one or more bioactive agents to the device surface in sufficient dose/concentrations to inhibit platelet aggregation, thrombus, thrombin, and/or clot formation. The vicinity to the wall or tissue contacting blood should have sufficient drug concentration to inhibit platelet aggregation, fibrin, thrombin, and/or clot formation.

In some examples, the therapeutic coating may locally release the one or more bioactive agents into the blood adjacent the tissue segment.

As used herein, the term coating refers to one or more layers disposed on a surface of a device. In some examples, a single coating is applied to the device. In other examples, one or more coatings are used. In some examples, each component of the therapeutic composition is disposed within the same layer of the coating. In some examples, at least one component of the therapeutic composition is disposed in a different layer of the coating. In some examples, one or more component of the therapeutic composition is coated on all surfaces of the device. In some examples, one or more component of the therapeutic composition is coated on a single surface of the device. In some examples, one or more component of the therapeutic composition is coated on two or more surfaces of the device. In some examples, one or more component of the therapeutic composition is coated on at least four surfaces (e.g., an inner surface, an outer surface, a first side, and a second side) of the device.

In addition to, the coatings described herein. For example, in some instances, the device can comprise one or more components of the therapeutic composition in a reservoir on or in the device. Alternatively, or in combination, the device can comprise one or more components of the therapeutic composition dispersed within the device structure.

In some examples, the therapeutic composition may be formulated to release one or more of the agents at a dose substantially below a systemic therapeutic dose of each agent to minimize off-target effects. Preferably, the dose is at least about 5 times lower than the systemic dose or more preferably about 10 times lower than the systemic dose.

In many examples, a tissue segment is composed of the tissue segment coupled to the device releasing agent. For example, if the stent is 20 mm in length, the tissue segment is 20 mm in length. In another specific example, the agent is released beyond the tissue segment. For example, when the tissue segment coupled to a device is 20 mm in length, the tissue adjacent to the tissue segment is called the adjacent tissue segment. In many cases the adjacent tissue segment ranges from 1 mm to 10 mm, preferably within a range from 1 mm to 5 mm, more preferably about 5 mm proximal and/or distal to the tissue segment, and most preferably is about 5 mm proximal and distal to the tissue segment.

As used herein, the term “coating” refers to a layer of polymer and/or drug (or therapeutic agent or active agent) disposed on a surface of a device structure. The layer may comprise a polymer, a drug, or a combination of a drug and a polymer.

As used herein, the term “top layer or coat” refers to an outer-most layer of a coating. The top layer or coat may comprise a polymer, a drug (or therapeutic agent or active agent), or a combination of a drug and a polymer. The top layer or coat may comprise the same polymer or a different polymer as layers of coating disposed there below. The top layer or coat may comprise the same drug or a different drug(s) as layers of coating disposed there below.

As used herein, the term “matrix” refers to a mixture of a drug (or therapeutic agent or active agent) and a polymer.

The terms anti-thrombin, thrombin inhibiter, and thrombin formation inhibitor are used interchangeably herein. Also, the terms anti-fibrin, fibrin inhibitor, and fibrin formation inhibitor are used interchangeably herein.

As used herein, a calcium chelating agent refer to any agent that could chelate calcium.

As used herein, a direct factor Xa inhibitor refers to a direct, selective inhibitor of factor Xa that acts directly on factor Xa without using antithrombin as a mediator. The term “direct factor Xa inhibitor” is used herein interchangeably with the term “factor Xa inhibitor” or “anti-factor Xa”. Direct factor Xa inhibitors inhibit thrombin formation and/or fibrin formation, thereby inhibiting clot formation. Direct factor Xa inhibitors include, but are not limited to, apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), or 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), or 2-(5-carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052). Preferred direct Xa inhibitors include apixaban and rivaroxaban.

As used herein, a direct factor IIa inhibitor refers to a direct, selective inhibitor of factor IIa (also referred to herein as thrombin) which acts directly on factor IIa/thrombin. The term “direct factor IIa inhibitor” is used herein interchangeably with the term “factor IIa inhibitor” or “anti-factor IIa”. Direct factor IIa inhibitors inhibit thrombin formation and/or fibrin formation, thereby inhibiting clot formation. Direct thrombin/factor IIa inhibitors include, but are not limited to, argatroban, dabigatran, ximelagatran, melagatran, efegatran, inogatran, atecegatran metoxil (AZD-0837), hirudin, hirudin analogs, bivalirudin, desirudin, and lepirudin. Preferred direct factor IIa inhibitors include argatroban.

As used herein, the term “inflammation” refers to inflammation of a patient's vasculature resulting from injury resulting from performance of a vascular intervention, for example, angioplasty, stenting, graft implantation, cardiac valve replacement, valvuloplasty, and the like. Injury to a vascular wall can activate of inflammatory cells, increase thrombogenicity and reduce efflux of β-lipoprotein, which then accumulates within the neointima in a process referred to as “hyperplasia.” While a number of immunosuppressive agents have been proposed for reducing inflammation and limiting hyperplasia, the present disclosure relies specifically on the local release of colchicine and/or tirofiban in combination with at least one antiproliferative drug, as described in more detail below.

As used herein, the phrase “antiproliferative drug” refers generally to a class of agents which inhibit cell-cycle pathways to reduce the cytotoxic response directed toward the stent or other implant or interventional device, typically be anti-mitotic agents, cytostatic agents and anti-migratory agents which suppress cell growth, proliferation, and/or metabolism. Preferred antiproliferative drugs are those which specifically inhibit deregulated smooth muscle cell (SMC) growth and proliferation, such as mTOR inhibitors. Examples of anti-proliferative agents include without limitation inhibitors of mammalian target of rapamycin (mTOR), rapamycin (also called sirolimus), deuterated rapamycin, rapamycin prodrug TAFA93, 40-O-alkyl-rapamycin derivatives, 40-O-hydroxyalkyl-rapamycin derivatives, everolimus {40-O-(2-hydroxyethyl)-rapamycin), 40-O-(3-hydroxy)propyl-rapamycin, 40-O-[2-(2-hydroxy)ethoxy]ethyl-rapamycin, 40-O-alkoxyalkyl-rapamycin derivatives, biolimus (40-O-(2-ethoxyethyl)-rapamycin}, 40-O-acyl-rapamycin derivatives, temsirolimus {40-(3-hydroxy-2-hydroxymethyl-2-methylpropanoate)-rapamycin, or CCI-779 (temsirolimus), 40-O-phospho-containing rapamycin derivatives, ridaforolimus (40-dimethylphosphinate-rapamycin, or AP23573 (ridaforolimus, formerly known as deforolimus), 40(R or S)-heterocyclyl- or heteroaryl-containing rapamycin derivatives, zotarolimus (40-epi-(N1-tetrazolyl)-rapamycin, or ABT-578 (zotarolimus), 40-epi-(N2-tetrazolyl)-rapamycin, 32(R or S)-hydroxy-rapamycin, myolimus (32-deoxo-rapamycin), novolimus (16-O-desmethyl-rapamycin), taxanes, paclitaxel, docetaxel, cytochalasins, cytochalasins A through J, latrunculins, and salts, isomers, solvates, analogs (including deuterated analogs), derivatives, metabolites, and prodrugs thereof. The IUPAC numbering system for rapamycin is used herein. Preferred anti-proliferative agents include mTOR inhibitors and/or taxanes, or salts, isomers, solvates, analogs, derivatives, metabolites, or prodrugs thereof.

Table 1A provides non-limiting examples of derivatives of each of rapamycin, everolimus, biolimus, temsirolimus, ridaforolimus, zotarolimus, myolimus and novolimus.

TABLE 1A
Derivatives of rapamycin-type compounds
Derivatives of Each of Rapamycin, Everolimus, Biolimus, Temsirolimus,
Ridaforolimus, Zotarolimus, Myolimus and Novolimas
N7-oxide
2-hydroxy
3-hydroxy
4-hydroxy
S-hydroxy
6-hydroxy
11-hydroxy
12-bydroxy
13-hydroxy
14-hydroxy
23-bydroxy
24-hydroxy
25-hydroxy
31-bydroxy
35-hydroxy
43-hydroxy (11-hydroxymethyl)
44-hydroxy (17-hydroxymethyl)
45-hydroxy (23-hydroxymethyl)
46-hydroxy (25-hydroxymethyl)
47-bydroxy (29-hydroxymethyl)
48-hydroxy (3 1-hydroxymethyl)
49-hydroxy (35-hydroxymethyl)
17,18-dibydroxy
19,20-dihydroxy
21,22-dihydroxy
29,30-dihydroxy
10-phosphate
28-phosphate
40-phosphate
16-O-desmethyl
27-O-desmethyl
39-O-desmethyl
16,27-bis(O)-desmethyl)
16,39-bis(O-desmethyl)
27,39-bis(O-desmethyl)
16,27,39-tris(O)-desmethyl)
16-desmethoxy
27-desmethoxy
39-O-desmethyl-14-hydroxy
17,18-epoxide
19,20-epoxide
21,22-epoxide
29,30-epoxide
17,18-29,30-bis-epoxide
17,18-19,20-21,22-tris-epoxide
19,20-21,22-29,30-tris-epoxide
16-O-desmethyl-17,18-19,20-bis-epoxide
16-O-desmethyl-17,18-29,30-bis-epoxide
16-O-desmethyl-17,18-19,20-21,22-tris-epoxide
16-O-desmethyl-19,20-21,22-29,30-tris-epoxide
27-O-desmethyl-17,18-19,20-21,22-tris-epoxide
39-O-desmethyl-17,18-19,20-21,22-tris-epoxide
16,27-bis(O-desmethyl)-17,18-19,20-21,22-tris-epoxide
16-O-desmethyl-24-hydroxy-17,18-19,20-bis-epoxide
16-O-desmethyl-24-hydroxy-17, 18-29,30-bis-epoxide
12-hydroxy and opened hemiketal ring

As used herein, the term “coagulation” comprises one or more of thrombin formation, fibrin formation, platelet activation, platelet aggregation, and/or thrombus/clot formation. Coagulation typically arises in response to a body part injury and/or to a foreign body such as a device. This may lead to one or more of inflammation, injury, blockage of a lumen or vessel partially or fully, degradation of the device function, formation of clot, and/or adverse clinical events. In some examples, any of the devices described herein may, at least partially, cause an injury to the tissue which may initiate the coagulation cascade.

As used herein, the term anti-coagulant refers to an agent that inhibits one or more of thrombin formation, fibrin formation, platelet activation (typically indirectly), platelet aggregation (typically indirectly), thrombus (clot) formation, thrombin dissolution, fibrin dissolution, or thrombus dissolution, thereby inhibiting one or more of blockage of a lumen or vessel partially or fully, degradation of the device function, formation of clot, and/or adverse clinical events.

As used herein, the term “top layer or coat” refers to an outer-most layer of a coating. The top layer or coat may comprise a polymer, a drug (or therapeutic agent or active agent), or a combination of a drug and a polymer. The top layer or coat may comprise the same polymer or a different polymer as layers of coating disposed there below. The top layer or coat may comprise the same drug or a different drug(s) as layers of coating disposed there below.

The terms anti-thrombin, thrombin inhibiter, and thrombin formation inhibitor are used interchangeably herein. Also, the terms anti-fibrin, fibrin inhibitor, and fibrin formation inhibitor are used interchangeably herein.

As used herein, a direct factor Xa inhibitor refers to a direct, selective inhibitor of factor Xa that acts directly on factor Xa without using antithrombin as a mediator. The term “direct factor Xa inhibitor” is used herein interchangeably with the term “factor Xa inhibitor” or “anti-factor Xa”. Direct factor Xa inhibitors inhibit thrombin formation and/or fibrin formation, thereby inhibiting clot formation. Direct factor Xa inhibitors include, but are not limited to, apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), or 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), or 2-(5-carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052). Preferred direct Xa inhibitors include apixaban and rivaroxaban.

As used herein, a direct factor IIa inhibitor refers to a direct, selective inhibitor of factor IIa (also referred to herein as thrombin) which acts directly on factor IIa/thrombin. The term “direct factor IIa inhibitor” is used herein interchangeably with the term “factor IIa inhibitor” or “anti-factor IIa”. Direct factor IIa inhibitors inhibit thrombin formation and/or fibrin formation, thereby inhibiting clot formation. Direct thrombin/factor IIa inhibitors include, but are not limited to, argatroban, dabigatran, ximelagatran, melagatran, efegatran, inogatran, atecegatran metoxil (AZD-0837), hirudin, hirudin analogs, bivalirudin, desirudin, and lepirudin. Preferred direct factor IIa inhibitors include argatroban.

As used herein, anti-platelet agent refers to any agent which, irrespective of its specific mechanism of action, blocks, reduces or inhibits platelet aggregation and prevents blood clots from forming. Anti-platelet agent include, but are not limited to Glycoprotein IIb/IIIa inhibitors including but not limit to Abciximab, Eptifibatide, Orbofiban, Roxifiban, Sibrafiban, Tirofiban; ADP receptor/P2Y12 inhibitors including but not limit to Clopidogrel, Prasugrel, Ticlopidine, Cangrelor, Elinogrel. Ticagrelor; Prostaglandin analogue (PGI2) inhibitors including but not limit to Beraprost, Iloprost, Prostacyclin, Treprostinil; COX inhibitors including but not limit to Acetylsalicylic acid/Aspirin, Aloxiprin, Carbasalate calcium, Indobufen, Triflusal, Dipyridamole aspirin; Thromboxane inhibitors including but not limit to Picotamide, Terbogrel, Terutroban, Aspirin, Ridogrel, Ramatroban; Phosphodiesterase inhibitors including but not limit to Cilostazol, Dipyridamole, Triflusal, Milrinone; other inhibitors including but not limit to Cloricromen, Ditazole, Vorapaxar; PAR/PAR-1 antagonists such as SCH530348, Atopaxar, Vorapaxar and E-5555; GPIa/IIa inhibitor such as EMS16; adhesion antagonists such as DZ-697b and RG12986; or a derivative, analogue, or salt thereof.

It will be understood by one of ordinary skill in the art that the devices and methods described herein may be used in combination with one or more additional bioactive agents. Such agents optionally include anti-mitotic agents, cytostatic agents, anti-migratory agents, immunomodulators, immunosuppressants, anti-inflammatory agents, anti-ischemia agents, anti-hypertensive agents, vasodilators, anti-hyperlipidemia agents, anti-diabetic agents, anti-cancer agents, anti-tumor agents, anti-angiogenic agents, angiogenic agents, anti-chemokine agents, healing-promoting agents, anti-bacterial agents, anti-fungal agents, and combinations thereof. It is understood that a bioactive agent may exert more than one biological effect.

Using colchicine and/or tirofiban to inhibit vascular inflammation in combination with inhibiting SMC and/or other vascular cell proliferation has been found to reduce one or more of blockage of a lumen or vessel partially or fully, degradation of the device function, formation of thrombus (clot) formation, inflammation, and/or adverse clinical events.

Described herein are devices and methods for locally delivering a therapeutic composition to a patient. The therapeutic composition includes colchicine and/or tirofiban as an anti-inflammatory agent and one or more antiproliferative agents. Optionally, the therapeutic compositions may further include agents which inhibit one or more of thrombin, fibrin, and/or thrombus formation or promote one or more of thrombin, fibrin, and/or thrombus dissolution. In other examples, the therapeutic composition further includes one or more of a calcium chelating agent, a direct Xa inhibitor and a direct IIa inhibitor.

While in some examples, the implantable scaffold with the therapeutic composition may be formulated for a rapid release into the environment surrounding the body structure, more usually the drugs will be released over an extended time period, usually at least one week, more usually at least one months, still more usually at least three months, at least six months, and at least one year after implantation of the body structure into environment.

In some examples, the colchicine and/or tirofiban and the antiproliferative drug(s) of the therapeutic composition are disposed on the external surface of the structure and on the internal surface of the structure. In some examples, the therapeutic composition is disposed on the external surface (abluminal) of the structure, on the interior surface (luminal) of the structure, and on the side surfaces of the structure. In yet other examples, the therapeutic composition is disposed on one or more surfaces of the structure. In yet other examples, the therapeutic composition is disposed on all surfaces of the structure. In yet other examples, the therapeutic composition is disposed in a reservoir on or in the structure. In some examples, the therapeutic composition is disposed on the external surface of the structure.

In some examples, the implantable scaffold with the therapeutic composition is formulated to inhibit release of the at least one drug into the environment surrounding the body structure for a time period in a range from 5 minutes to 240 minutes, preferably from 5 minutes to 180 minutes, and more preferably from 5 minutes to 60 minutes.

In some examples, the devices described herein can be configured to release colchicine and/or tirofiban and the antiproliferative drug(s) to a mammalian body, lumen, tissue, and/or device surface prior to an injury to said tissue, concurrent with injury to said tissue, or after an initial injury to said tissue. The device is introduced into said mammalian body and advanced to said tissue site or body lumen. In some specific examples, the device is expanded against said tissue to release said agent. In other examples, the device is expanded against said tissue to perform a function such as opening up a vessel or lumen and to release said agent. In specific examples, the device is a stent or a balloon catheter. In yet another example, the device is placed adjacent to said tissue.

The therapeutic compositions of the present disclosure may be coated on a device for local delivery of the optionally additional active substances. For example, one or more of the colchicine and/or tirofiban, antiproliferative drug(s), and other active substances may be coated, dipped, printed, deposited, painted, brushed, loaded, or otherwise disposed on one or more surfaces of the device for local delivery. In some examples, one or more of the active substances may be incorporated into the backbone structure of the device. Alternatively, or in combination, one or more of the active substances may be locally delivered via a drug reservoir coupled to the device. In some examples, one or more or the active substances may be coated or otherwise disposed directly onto one or more surfaces of the device. In some examples, one or more or the active substances may be coated or otherwise disposed on one or more surfaces of the device in a carrier such as a polymer matrix. In some examples, one or more of the active substances may be cross-linked with a polymer, or to itself, or to another drug (in order to be another active substance, e.g., after the links are broken in vivo). In some examples, the carrier may be an excipient, a polymer, or other types of material to facilitate applying or controlling the drug onto the device or controlling release of the drug from the device or protecting the drug from washing out during entry or deployment into the body. In some examples, the carrier may comprise a microsphere or a nanosphere.

In some examples, the colchicine and/or tirofiban and the antiproliferative drug(s) of the therapeutic composition may be disposed in two or more discrete or blended layers on a surface of the device, for example a first layer, a second layer, or an additional layer may comprise one or more of colchicine and/or tirofiban, the antiproliferative drug(s), and optional agents, while other layer may not comprise a bioactive agent. Some layers may be present for the purpose of rate control of the drugs and agents.

In some examples, a first layer may comprise one or more of colchicine and/or tirofiban and the antiproliferative drug(s) of the therapeutic composition and the second layer may comprise one or more of colchicine and/or tirofiban and the antiproliferative drug(s) of the therapeutic composition. The first layer and the second layer may be configured to release their respective active agents at the same rate. Alternatively, the first layer and the second layer may be configured to release their respective bioactive agents at different rates.

In some examples, one, two, or more discrete or blended layers may comprise a polymeric material as described herein. For example, the first layer may comprise a polymeric material and the second layer may not comprise a polymeric material. Alternatively, the first layer may not comprise a polymeric material and the second layer may comprise a polymeric material. Alternatively, both the first layer and the second layer may comprise the same or a different polymeric material and/or polymeric material concentration and/or formulation.

Colchicine could be combined with such as rivaroxaban, argatroban and tirofiban in drug coatings for stents and other implantable devices where the combination achieves a synergistic inhibitory effect greater than achieved by coatings employing either drug alone.

Discrete layers of the therapeutic compositions may be disposed on an external surface of the stent (e.g., on the abluminal surface of one or more filaments of the stent), on an internal surface of the stent (e.g., on the luminal surface of one or more filaments of the stent), or on both the external surface and the internal surface of the stent (e.g., partially or fully surround one or more elements of the stent). However, it will be understood by one of ordinary skill in the art that different layers of ten therapeutic composition may coat the stent or other implant or device differently. For example, a first layer of the coating may fully surround each element of a stent while the second layer may be applied only on one surface (e.g., luminal or abluminal) of the stent. In some examples, only internal layers of a device are coated (not external surfaces).

It will be understood by one of ordinary skill in the art that the devices and methods described herein may be used in combination with one or more additional bioactive agents. Such agents optionally include anti-mitotic agents, cytostatic agents, anti-migratory agents, immunomodulators, immunosuppressants, anti-inflammatory agents, anti-ischemia agents, anti-hypertensive agents, vasodilators, anti-hyperlipidemia agents, anti-diabetic agents, anti-cancer agents, anti-tumor agents, anti-angiogenic agents, angiogenic agents, anti-chemokine agents, healing-promoting agents, anti-bacterial agents, anti-fungal agents, and combinations thereof. It is understood that a bioactive agent may exert more than one biological effect.

In a preferred example, a device releasing one or more calcium chelating agent, factor Xa inhibitors, and/or one or more factor IIa inhibitors, and/or one or more antiproliferative agents, wherein said one or more agents inhibit thrombin formation and/or fibrin formation thereby inhibiting clot formation and smooth muscle cell proliferation.

In a preferred example, a device releasing one or calcium chelating agent of EDTA ammonium slat complex, factor Xa inhibitors, and/or one or more factor IIa inhibitors, and/or one or more antiproliferative agents, wherein said one or more agents inhibit thrombin formation and/or fibrin formation thereby inhibiting clot formation and smooth muscle cell proliferation.

In a preferred example, a device releasing one or more cationic anti-coagulation enhancer, factor Xa inhibitors, and/or one or more factor IIa inhibitors, and/or one or more antiproliferative agents, wherein said one or more agents inhibit thrombin formation and/or fibrin formation thereby inhibiting clot formation and smooth muscle cell proliferation.

In a preferred example, a device releasing one or more calcium chelating agent, one or more a calcium chelating agent of EDTA ammonium slat complex, factor Xa inhibitors, and/or one or more factor IIa inhibitors, and/or one or more antiproliferative agents, wherein said one or more agents inhibit thrombin formation and/or fibrin formation thereby inhibiting clot formation and smooth muscle cell proliferation.

In a preferred example, a device releasing one or more a calcium chelating agent, a direct factor Xa inhibitors, and/or one or more factor IIa inhibitors, and/or one or more antiproliferative agents, wherein said one or more agents inhibit thrombin formation and/or fibrin formation thereby inhibiting clot formation and smooth muscle cell proliferation.

In some examples, the injury to a tissue, surface, vessel/lumen wall, or other body part is the first substantial injury resulting from a surgery or intervention. In certain examples, the surgery or intervention is selected from the group consisting of vascular surgeries and interventions, cardiovascular surgeries and interventions, peripheral vascular surgeries and interventions, vascular grafting, vascular replacement, vascular angioplasty, thrombectomy, vascular stent placement, vascular laser therapy, coronary by-pass surgery, coronary angiography, coronary stent placement, carotid artery procedures, peripheral stent placement, organ transplants, artificial heart transplant, and plastic and cosmetic surgeries and interventions. In additional examples, the injury is the first substantial injury caused by the device delivering the one or more active substances, and optionally one or more other kinds of bioactive agents (e.g., anti-proliferative agents, anti-inflammatory agents, etc.). In some examples, a substantial injury to a tissue, surface, vessel/lumen wall or other body part results from contact of a device with the tissue, surface, vessel/lumen wall or other body part in a surgery or intervention (e.g., contact of the device causing damage to the endothelium lining a blood vessel, a surgical cutting instrument cutting a tissue, a deployed stent embedding into the wall of a blood vessel, etc.). In further examples, a substantial injury to a tissue, surface, vessel/lumen wall or other body part has a potential to elicit fibrin/thrombus formation, cell migration, cell proliferation or inflammation, or a combination thereof, at the site of injury or at an area adjacent thereto.

In some examples, the therapeutic composition is formulated to release the one or more active substances at a rate of 1 μg/second/mm device to about 50 μg/day/mm device, preferably at a rate of 1 μg/min/mm device to about 30 μg/day/mm device, more preferably at a rate of 1 μg/hour/mm device to about 30 μg/day/mm device.

In some examples, each of the one or more active substances is released from a temporary or non-temporary device at a rate within a range of about 1 μg/hour/mm device length to about 30 μg/day/mm device length, for example about 1 μg/hour/mm device length to about 20 μg/day/mm device length. In some examples, the therapeutic composition is formulated to release the one or more active substances at a rate of 1 μg/hour/mm device to about 20 μg/day/mm device. In some examples, the therapeutic composition may be formulated to release the one or more active substances at a rate within a range of about 1 μg/hour/mm device length to about 14 μg/hour/mm device length. In some examples, the therapeutic composition may be formulated to release the one or more active substances at a rate within a range bounded by any two of the following values: about 1 μg/hour/mm device length, about 2 μg/hour/mm device length, about 3 μg/hour/mm device length, about 4 μg/hour/mm device length, about 5 μg/hour/mm device length, about 6 μg/hour/mm device length, about 7 μg/hour/mm device length, about 8 μg/hour/mm device length, about 9 μg/hour/mm device length, about 10 μg/hour/mm device length, about 1 μg/hour/mm device length, about 12 μg/hour/mm device length, about 13 μg/hour/mm device length, about 14 μg/hour/mm device length, about 15 μg/hour/mm device length, about 16 μg/hour/mm device length, about 17 μg/hour/mm device length, about 18 μg/hour/mm device length, about 19 μg/hour/mm device length, about 20 μg/hour/mm device length, about 21 μg/hour/mm device length, about 22 μg/hour/mm device length, about 23 μg/hour/mm device length, about 24 μg/hour/mm device length, about 25 μg/hour/mm device length, about 26 μg/hour/mm device length, about 27 μg/hour/mm device length, about 28 μg/hour/mm device length, about 29 μg/hour/mm device length, or about 30 μg/hour/mm device length.

In some examples, the therapeutic composition is formulated to begin releasing the one or more active substances within about 1 minute, 5, 10, 15, 20, 25, or 30 minutes after the external surface of the structure is positioned adjacent the injury site.

In some examples, substantially all of each of the one or more active substances is released from a temporary or non-temporary device within about 1 day to about 180 days or more, for example within about 1 day to about 90 days. In some examples, the therapeutic composition is formulated to release substantially all of the one or more active substances within about 7 days or about 28 days. In some examples, the therapeutic composition may be formulated to release substantially all of the one or more active substances within a range bounded by any two of the following values: 1 day, 3 days, 7 days, 14 days, 21 days, 28 days, 45 days, 90 days, 180 days, or more.

In some examples, the therapeutic composition is formulated to release substantially all of the one or more active substances within about 3 hours, about 6 hours, about 12 hours, about 1 day, or about 3 days. In some examples, the therapeutic composition is formulated to release at least 50%, at least 60%, or at least 70% of the one or more active substances within about 3 hours, about 6 hours, about 12 hours, about 1 day, about 3 days, about 7 days, or about 28 days.

In another example, the device releases the one or more active substances from 1 microgram per mm of device length to 25 micrograms per mm of device length, and preferably releases said agent from 5 micrograms per mm of device length to 20 micrograms per mm of device length.

In some examples, the therapeutic composition is formulated to release the one or more active substances at a rate within a range of about 2 μg/mm device to about 100 μg/mm device, about 5 μg/mm device to about 100 μg/mm device, about 7 μg/mm device to about 100 μg/mm device, or about 10 μg/mm device to about 100 μg/mm device within about 3 hours, 12 hours, 1 day, 3 days, 7 days, 28 days, 90 days, or 180 days. In some examples, the therapeutic composition is formulated to release the one or more active substances at a rate within a range of about 5 μg/mm device to about 100 μg/mm device within about 3 hours. In some examples, the therapeutic composition is formulated to release the one or more active substances at a rate within a range of about 5 μg/mm device to about 100 μg/mm device within about 12 hours. In some examples, the therapeutic composition is formulated to release the one or more active substances at a rate within a range of about 5 μg/mm device to about 100 μg/mm device within about 7 days. In some examples, the therapeutic composition is formulated to release the one or more active substances at a rate within a range of about 5 μg/mm device to about 100 μg/mm device within about 28 days.

In some examples, the therapeutic composition is formulated to release the one or more active substances at a dose within a range of about 0.5 μg/mm2 device to about 15 μg/mm2 device, or of about 1 μg/mm2 device to about 12 μg/mm2 device, or of about 2 μg/mm2 device to about 12 μg/mm2 device, or of about 5 μg/mm2 device to about 12 μg/mm2 device, or of about 7 μg/mm2 device to about 12 μg/mm2 device, within about 3 hours or about 12 hours or about 1 day or about 3 days or about 7 days. In some examples, the therapeutic composition is formulated to release the one or more active substances at a dose within a range of about 1 μg/mm2 device to about 12 μg/mm2 device within about 3 hours. In some examples, the therapeutic composition is formulated to release the one or more active substances at a dose within a range of about 1 μg/mm2 device to about 12 μg/mm2 device within about 12 hours. In some examples, the therapeutic composition is formulated to release the one or more active substances at a dose within a range of about 1 μg/mm2 device to about 12 μg/mm2 device within about 7 days. In some examples, the therapeutic composition is formulated to release the one or more active substances at a dose within a range of about 1 μg/mm2 device to about 12 μg/mm2 device within about 28 days, about 90 days, or about 180 days.

In some examples, each of the one or more agents is released from a temporary or non-temporary device at the same rate. In other examples, one or more of the one or more agents that inhibit fibrin/thrombus formation or promote fibrin/thrombus dissolution and/or other bioactive agents is released from a temporary or non-temporary device at a different rate.

In some examples, the therapeutic composition is formulated to release the calcium chelating agent, direct factor Xa inhibitor and/or the direct factor IIa inhibitor faster than the anti-proliferative agent.

In some examples, the therapeutic composition is formulated to release a larger dose of a calcium chelating agent, the direct factor Xa inhibitor than the anti-proliferative agent. In some examples, the dose of a calcium chelating agent, the direct factor Xa inhibitor is about 1.25 to about 5 times larger, about 1.5 to about 3 times larger, or about 1.5 to about 2.5 times larger than a dose of the anti-proliferative agent.

In some examples, the therapeutic composition is formulated to release a dose of colchicine and/or tirofiban sufficient to generate a blood concentration of the colchicine and/or tirofiban which is smaller than a median maximum serum concentration (Cmax) of the colchicine and/or tirofiban inhibitor generated by systemic delivery of the colchicine and/or tirofiban to achieve the same tissue concentration from 5 to 20 picograms per milliliter (pg/ml) at the injury site. In other examples, the therapeutic composition is formulated to release a dose of the colchicine and/or tirofiban sufficient to generate a blood concentration of the colchicine and/or tirofiban which is smaller than a median maximum serum concentration (Cmax) of the colchicine and/or tirofiban generated by systemic delivery of the colchicine and/or tirofiban when taking one or more oral dose of up to 2 mg of colchicine and/or tirofiban. In some examples, the systemic delivery comprises a single oral dose, a daily oral dose, or a smallest oral dose of the colchicine and/or tirofiban. In some examples, the blood concentration is larger than a median minimum serum concentration (Cmin) of the colchicine and/or tirofiban generated by systemic delivery. In some examples, the blood concentration is smaller than a median minimum serum concentration (Cmin) of the colchicine and/or tirofiban generated by systemic delivery. In some examples, the Cmax is measured using one of plasma blood, serum blood, or whole blood. In other examples, the median Cmax is 1 ng/ml, or 2 ng/ml, or 4 ng/ml, or 6 ng/ml, or 8 ng/ml of blood.

In some examples, the therapeutic composition is formulated to release a dose of the direct factor Xa inhibitor sufficient to generate a blood concentration of the direct factor Xa inhibitor which is smaller than a median maximum serum concentration (Cmax) of the direct factor Xa inhibitor generated by systemic delivery of the direct factor Xa inhibitor to achieve the same tissue concentration at the injury site. In other examples, the therapeutic composition is formulated to release a dose of the direct factor Xa inhibitor sufficient to generate a blood concentration of the direct factor Xa inhibitor which is smaller than a median maximum serum concentration (Cmax) of the direct factor Xa inhibitor generated by systemic delivery of the direct factor Xa inhibitor when taking one or more oral dose of said factor Xa inhibitor. In some examples, the systemic delivery comprises a single oral dose, a daily oral dose, or a smallest oral dose of the direct factor Xa inhibitor. In some examples, the blood concentration is larger than a median minimum serum concentration (Cmin) of the direct factor Xa inhibitor generated by systemic delivery. In some examples, the blood concentration is smaller than a median minimum serum concentration (Cmin) of the direct factor Xa inhibitor generated by systemic delivery. In some examples, the Cmax is measured using one of plasma blood, serum blood, or whole blood. In other examples, the median Cmax is 80 ng/ml, or 123 ng/ml, or 171 ng/ml, or 321 ng/ml, or 480 ng/ml of blood.

In some examples, the therapeutic composition is formulated to release a dose of the direct factor Xa inhibitor sufficient to generate a plasma drug level area under the curve (AUC (0-24) or AUC (0-∞)) in ng·h/ml which is smaller than a median (AUC (0-24) or AUC (0-∞)) in ng·h/ml of the direct factor Xa inhibitor generated by systemic delivery of the direct factor Xa inhibitor to achieve the same tissue concentration at the injury site. In other examples, the therapeutic composition is formulated to release a dose of the direct factor Xa inhibitor sufficient to generate a plasma drug level area under the curve (AUC (0-24) or AUC (0-∞)) in ng·h/ml which is smaller than a median (AUC (0-24) or AUC (0-∞)) in ng·h/ml of the direct factor Xa inhibitor generated by systemic delivery of the direct factor Xa inhibitor when taking one or more oral dose of said factor Xa inhibitor. In some examples, the systemic delivery comprises a single oral dose, a daily oral dose, or a smallest oral dose of the direct factor Xa inhibitor. In some examples, the (AUC (0-24) or AUC (0-∞)) is measured using one of plasma blood, serum blood, or whole blood. In other examples, the median (AUC (0-24) or AUC (0-∞)) is 724 ng·h/ml, or 1437 ng·h/ml, or 2000 ng·h/ml, or 4000 ng·h/ml.

In some examples, the therapeutic composition is formulated to release a dose of the anti-proliferative agent sufficient to generate a blood concentration of the anti-proliferative agent which is smaller than a median maximum serum concentration (Cmax) of the anti-proliferative agent generated by systemic delivery of the anti-proliferative agent to achieve the same tissue concentration at the injury site. In some examples, the systemic delivery comprises a single oral dose, a daily oral dose, or a smallest oral dose of the anti-proliferative agent. In some examples, the blood concentration is larger than a median minimum serum concentration (Cmin) of the anti-proliferative agent generated by systemic delivery. In some examples, the blood concentration is smaller than a median minimum serum concentration (Cmin) of the anti-proliferative agent generated by systemic delivery. In some examples, the therapeutic composition is formulated to release a dose of the anti-proliferative agent sufficient to generate a plasma drug level area under the curve (AUC (0-∞)) in ng·h/ml which is smaller than a median AUC (0-∞) in ng·h/ml of the anti-proliferative agent generated by systemic delivery of the anti-proliferative agent to achieve the same tissue concentration at the injury site.

In some examples, the therapeutic composition is formulated to release a dose of the direct factor IIa inhibitor sufficient to generate a blood concentration of the direct factor IIa inhibitor which is smaller than a median maximum serum concentration (Cmax) of the direct factor IIa inhibitor generated by systemic delivery of the direct factor IIa inhibitor to achieve the same tissue concentration at the injury site. In other examples, the therapeutic composition is formulated to release a dose of the direct factor IIa inhibitor sufficient to generate a blood concentration of the direct factor IIa inhibitor which is smaller than a median maximum serum concentration (Cmax) of the direct factor IIa inhibitor generated by systemic delivery of the direct factor IIa inhibitor when taking one or more oral dose of said factor IIa inhibitor. In some examples, the systemic delivery comprises a single oral dose, a daily oral dose, or a smallest oral dose of the direct factor IIa inhibitor. In some examples, the blood concentration is larger than a median minimum serum concentration (Cmin) of the direct factor IIa inhibitor generated by systemic delivery. In some examples, the blood concentration is smaller than a median minimum serum concentration (Cmin) of the direct factor IIa inhibitor generated by systemic delivery. In some examples, the Cmax is measured using one of plasma blood, serum blood, or whole blood. In other examples, the median Cmax is 80 ng/ml, or 123 ng/ml, or 171 ng/ml, or 321 ng/ml, or 480 ng/ml of blood.

In some examples, the therapeutic composition is formulated to release a dose of the direct factor IIa inhibitor sufficient to generate a plasma drug level area under the curve (AUC (0-24) or AUC (0-∞)) in ng·h/ml which is smaller than a median (AUC (0-24) or AUC (0-∞)) in ng·h/ml of the direct factor IIa inhibitor generated by systemic delivery of the direct factor IIa inhibitor to achieve the same tissue concentration at the injury site. In other examples, the therapeutic composition is formulated to release a dose of the direct factor IIa inhibitor sufficient to generate a plasma drug level area under the curve (AUC (0-24) or AUC (0-∞)) in ng·h/ml which is smaller than a median (AUC (0-24) or AUC (0-∞)) in ng·h/ml of the direct factor IIa inhibitor generated by systemic delivery of the direct factor IIa inhibitor when taking one or more oral dose of said factor IIa inhibitor. In some examples, the systemic delivery comprises a single oral dose, a daily oral dose, or a smallest oral dose of the direct factor IIa inhibitor. In some examples, the (AUC (0-24) or AUC (0-∞)) is measured using one of plasma blood, serum blood, or whole blood. In other examples, the median (AUC (0-24) or AUC (0-∞)) is 724 ng·h/ml, or 1437 ng·h/ml, or 2000 ng·h/ml, or 4000 ng·h/ml.

In some examples, local delivery of one or more of the active substances may reduce the time a patient needs to spend on oral medications and/or obviate the need for such medications entirely.

In some examples, the dose of each of the one or more active substances for optional systemic administration on a one-time basis or over a certain time period described herein (e.g., 6 hr, 12 hr, 1 day, 2 days, 3 days, 1 week, 2 weeks, 3 weeks, 1 month, etc.) independently is at least about 1, 5, 10, 20, 50, 100 or 500 mg, or at least about 1, 5 or 10 g. In further examples, the amount of each of the one or more active substances loaded in and/or on a temporary or non-temporary device, or the amount of each such agent released from the device, independently is at least about 1, 10, 50, 100 or 500 μg, or at least about 1, 5, 10 or 20 mg. In certain examples, the amount of each of the fibrin/thrombus formation-inhibiting or fibrin/thrombus dissolution-promoting agent(s) loaded in and/or on the device, or the amount of each such agent released from the device, independently is about 1 μg to about 20 mg, or about 10 μg to about 10 mg, or about 50 μg to about 5 mg, or about 100 μg to about 1 mg, or about 100 μg to about 500 μg, or about 500 μg to about 1 mg.

In further examples, the concentration of each of the one or more active substances released from a temporary or non-temporary device, and optionally administered systemically in addition to locally, in blood or tissue at the site of injury or at an area adjacent thereto, and/or in blood or tissue adjacent to the device, independently is at least about 0.001, 0.01, 0.1, 1, 10, 50, 100 or 500 nM, or at least about 1, 10, 50, 100, 500 or 1000 μM. In certain examples, the concentration of each of the fibrin/thrombus formation-inhibiting or fibrin/thrombus dissolution-promoting agent(s) released from a temporary or non-temporary device, and optionally administered systemically in addition to locally, in blood or tissue at the site of injury or at an area adjacent thereto, and/or in blood or tissue adjacent to the device, independently is about 0.01 or 0.1 nM to about 1000 μM, or about 0.1 or 1 nM to about 500 μM, or about 1 or 10 nM to about 100 μM, or about 50 nM to about 50 μM, or about 10 or 100 nM to about 10 μM, or about 100 nM to about 1 μM, or about 1 μM to about 10 μM.

In still further examples, the concentration of each of the one or more active substances released from a temporary or non-temporary device, and optionally administered systemically in addition to locally, in tissue at the site of injury or at an area adjacent thereto, and/or in tissue adjacent to the device, independently is at least about 0.01, 0.1, 1, 10, 50, 100 or 500 ng/gm tissue, or at least about 1, 10, 50, 100, 500 or 1000 μg/gm tissue. In certain examples, the concentration of each of the fibrin/thrombus formation-inhibiting or fibrin/thrombus dissolution-promoting agent(s) released from a temporary or non-temporary device, and optionally administered systemically in addition to locally, in tissue at the site of injury or at an area adjacent thereto, and/or in tissue adjacent to the device, independently is about 0.01 or 0.1 ng/gm tissue to about 1000 μg/gm tissue, or about 0.1 or 1 ng/gm tissue to about 500 μg/gm tissue, or about 1 or 10 ng/gm tissue to about 100 μg/gm tissue, or about 50 ng/gm tissue to about 50 μg/gm tissue, or about 10 or 100 ng/gm tissue to about 10 μg/gm tissue, or about 100 ng/gm tissue to about 1 μg/gm tissue, or about 1 μg/gm tissue to about 10 μg/gm tissue.

In additional examples, the concentration of each of the one or more active substances released from a temporary or non-temporary device (that may or may not cause an injury to a tissue, surface, vessel/lumen wall or other body part), and optionally administered systemically in addition to locally, in blood or tissue at the site of injury or at an area adjacent thereto, and/or in blood or tissue adjacent to the device, independently is at least about 0.001, 0.01, 0.1, 1, 10, 50, 100 or 500 nM, or at least about 1, 10, 50 or 100 μM, within about 1 day, 12 hr, 6 hr, 3 hr, 2 hr, 1 hr, 30 min., 15 min., 5 min, or 1 min. before, during and/or within about 1 day, 12 hr, 6 hr, 3 hr. 2 hr, 1 hr, 30 min., 15 min., 5 min, or 1 min. after delivery or deployment of the device and/or the injury. In further examples, the concentration of each of the one or more active substances released from a temporary or non-temporary device (that may or may not cause an injury to a tissue, surface, vessel/lumen wall or other body part), and optionally administered systemically in addition to locally, in tissue at the site of injury or at an area adjacent thereto, and/or in tissue adjacent to the device, independently is at least about 0.01, 0.1, 1, 10, 50, 100 or 500 ng/gm tissue, or at least about 1, 10, 50 or 100 μg/gm tissue, within about 1 day, 12 hr, 6 hr. 3 hr, 2 hr, 1 hr, 30 min., 15 min., 5 min, or 1 min. before, during and/or within about 1 day, 12 hr, 6 hr, 3 hr, 2 hr, 1 hr, 30 min., 15 min., 5 min, or 1 min. after delivery or deployment of the device and/or the injury.

In some examples, the dose of each of the one or more optional other kinds of bioactive agents (e.g., anti-proliferative agents, anti-inflammatory agents, etc.) for optional systemic administration on a one-time basis or over a certain time period described herein (e.g., 6 hr, 12 hr, 1 day, 2 days, 3 days, 1 week, 2 weeks, 3 weeks, 1 month, etc.) independently is at least about 1, 5, 10, 20, 50, 100 or 500 mg, or at least about 1, 5 or 10 g. In additional examples, the amount of each of the one or more optional other kinds of bioactive agents (e.g., anti-proliferative agents, anti-inflammatory agents, etc.) loaded in and/or on a temporary or non-temporary device, or the amount of each such agent released from the device, independently is at least about 1, 10, 50, 100 or 500 μg, or at least about 1, 5, 10 or 20 mg. In certain examples, the amount of each of the optional other kind(s) of bioactive agent(s) loaded in and/or on the device, or the amount of each such agent released from the device, independently is about 1 μg to about 20 mg, or about 10 μg to about 10 mg, or about 50 μg to about 5 mg, or about 100 μg to about 1 mg, or about 100 μg to about 500 μg, or about 500 μg to about 1 mg, or about 50 μg to about 200 μg.

In further examples, the concentration of each of the one or more optional other kinds of bioactive agents (e.g., anti-proliferative agents, anti-inflammatory agents, etc.) released from a temporary or non-temporary device, and optionally administered systemically in addition to locally, in tissue at the site of injury or at an area adjacent thereto, and/or in tissue adjacent to the device, independently is at least about 0.01, 0.1, 1, 10, 50, 100 or 500 ng/gm tissue, or at least about 1, 10, 50, 100, 500 or 1000 μg/gm tissue. In certain examples, the concentration of each of the optional other kind(s) of bioactive agent(s) released from a temporary or non-temporary device, and optionally administered systemically in addition to locally, in tissue at the site of injury or at an area adjacent thereto, and/or in tissue adjacent to the device, independently is about 0.01 or 0.1 ng/gm tissue to about 1000 μg/gm tissue, or about 0.1 or 1 ng/gm tissue to about 500 μg/gm tissue, or about 1 or 10 ng/gm tissue to about 100 μg/gm tissue, or about 50 ng/gm tissue to about 50 μg/gm tissue, or about 10 or 100 ng/gm tissue to about 10 μg/gm tissue, or about 100 ng/gm tissue to about 1 μg/gm tissue, or about 1 μg/gm tissue to about 10 μg/gm tissue.

In some examples, the device contains the bioactive agent(s) in the body and/or on at least one surface of the device. In certain examples, the bioactive agent(s) are contained in one or more layers in the body and/or at the surface of the device.

In further examples, the bioactive agent(s) are contained in one or more coatings disposed over the body of the device. The coating(s) can be disposed over any desired portion(s) and any desired surface(s) of the body of the device. As a non-limiting example, for a tubular vascular device such as a stent, the coating(s) can be disposed over the luminal (lumen-facing) surface, the abluminal (tissue-facing) surface or the side surface(s) of the stent, or a combination thereof (e.g., all surfaces of the stent).

In additional examples, the device comprises the bioactive agent(s) in the body of the device and in one or more coatings disposed over the body of the device.

A temporary or non-temporary device can comprise openings in and/or on the body (including at the surface) of the device, and/or in one or more coatings disposed over the body structure of the device. Examples of openings include without limitation pores (including partial pores and through pores), holes (including partial holes and through holes), voids, recesses, pits, cavities, trenches, reservoirs, and channels. In some examples, a temporary or non-temporary device contains one or more anti-coagulant, and optionally one or more other kinds of bioactive agents (e.g., anti-proliferative agents, anti-inflammatory agents, etc.) in openings in and/or on the body (including at the surface) of the device, and/or in one or more coatings disposed over the body of the device.

The device may comprise one or more coatings disposed over an exterior surface of a structure of the device, as described herein. In some embodiments, the coating(s) may comprise a homopolymer, a copolymer, a mixture of homopolymers, a mixture of copolymers, or a mixture of a homopolymer and a copolymer. In some examples, the coating(s) comprise a soft or hydrophilic, or a softer or more hydrophilic, polymeric material. In further examples, the coating(s) comprise a polymeric material and an additive (e.g., a monomer of the polymeric material) that softens the polymeric material.

In some examples, the device has a first coating that comprises a biodegradable or non-degradable polymeric material, or one or more bioactive agents, or both a biodegradable or non-degradable polymeric material and one or more bioactive agents. In further examples, the device has a second coating that comprises a biodegradable or non-degradable polymeric material, or one or more bioactive agents, or both a biodegradable or non-degradable polymeric material and one or more bioactive agents, wherein the second coating optionally is disposed over the first coating. In additional examples, the device has a third coating that comprises a biodegradable or non-degradable polymeric material, wherein the third coating is disposed over the first coating and/or the second coating. In some examples, the third coating serves as a top layer or coat or diffusion barrier that controls release of one or more bioactive agents from inner coating(s) and/or the body of the device.

In some examples, a bioactive agent that is intended to have an earlier or shorter time of action can be contained in an outer coating, on a surface uncovered by a coating, and/or in the body of the device closer to the surface, and a bioactive agent that is intended to have a later or longer time of action can be contained in an inner coating, in a coating covered by a barrier coating, on a surface covered by a coating, and/or in the body of the device farther from the surface. In further examples, a bioactive agent that is intended to have an earlier or shorter time of action is contained on a surface of the device, or contained in a coating on the device or in a layer of the body of the device which comprises a faster-degrading polymeric material, and a bioactive agent that is intended to have a later or longer time of action is contained within the device, or contained in a coating on the device or in a layer of the body of the device which comprises a slower-degrading or non-degrading polymeric material. In additional examples, a bioactive agent that is intended to have an earlier or shorter time of action is more soluble, and a bioactive agent that is intended to have a later or longer time of action is less soluble.

In certain examples, the concentration of a bioactive agent [e.g., anti-coagulant, anti-proliferative, etc.] in a coating comprising a polymeric material is at least about 10%, 20%, 30%, 40%, 50% or 60% by weight relative to the weight of the bioactive agent and the polymeric material.

In further examples, the thickness (e.g., average thickness) of each of the coating(s) independently is no more than about 20, 15, 10, 5, 3 or 1 micron.

In some examples, the coating(s) may comprise carrier material. Non-limiting examples of carrier materials include biodegradable polymeric materials, non-degradable polymeric materials, and other matrix materials.

In some examples, the carrier material may be porous. In certain examples, the porosity of each of the coating(s) of the carrier material may be within a range of about 10 nm to about 10 μm.

In some examples, the carrier material may be biodegradable. In certain examples, the carrier material may have a depredation rate within a range of about 1 month to about 36 months.

In some examples, the weight compositional ratio of the carrier material to the therapeutic composition of one or more bioactive agents may be within a range of about 1:5 to 3:2.

Non-limiting examples of polymeric materials that can compose the carrier material include polyesters, polylactide, polyglycolide, poly(ε-caprolactone), polydioxanone, poly(hydroxyalkanoates), poly(L-lactide-co-D-lactide), poly(L-lactide-co-D,L-lactide), poly(D-lactide-co-D,L-lactide), poly(lactide-co-glycolide) (including 70:30 to 99:1 PLA-co-PGA, such as 85:15 PLA-co-PGA), poly(lactide-co-r-caprolactone) (including 70:30 to 99:1 PLA-co-PCL, such as 90:10 PLA-co-PCL), poly(glycolide-co-ε-caprolactone), poly(lactide-co-dioxanone), poly(glycolide-co-dioxanone), poly(lactide-co-trimethylene carbonate), poly(glycolide-co-trimethylene carbonate), poly(lactide-co-ethylene carbonate), and copolymers and combinations thereof, wherein lactide includes L-lactide, D-lactide and D,L-lactide. The polymeric material may comprise a material selected from a group of non-degradable polymeric materials consisting of polyacrylates, polymethacrylates, poly(n-butyl methacrylate), poly(hydroxyethylmethacrylate), polyamides, nylons, nylon 12, Dacron, Polyethylene terephthalate, poly(ethylene glycol), polyethylene oxide (PEO), polydimethylsiloxane, polyvinylpyrrolidone, ethylene-vinyl acetate, phosphorylcholine-containing polymers, poly(2-methacryloyloxyethylphosphorylcholine), poly(2-methacryloyloxyethylphosphorylcholine-co-butyl methacrylate), and copolymers and combinations thereof.

Non-limiting examples of biodegradable polymeric materials that can compose the body of the device, a layer of the body, or a coating include polyesters, poly(α-hydroxyacids), polylactide, polyglycolide, poly(s-caprolactone), polydioxanone, poly(hydroxyalkanoates), poly(hydroxypropionates), poly(3-hydroxypropionate), poly(hydroxybutyrates), poly(3-hydroxybutyrate), poly(4-hydroxybutyrate), poly(hydroxypentanoates), poly(3-hydroxypentanoate), poly(hydroxyvalerates), poly(3-hydroxyvalerate), poly(4-hydroxyvalerate), poly(hydroxyoctanoates), poly(3-hydroxyoctanoate), polysalicylate, polysalicylic acid, polycarbonates, poly(trimethylene carbonate), poly(ethylene carbonate), poly(propylene carbonate), tyrosine-derived polycarbonates, L-tyrosine-derived polycarbonates, polyiminocarbonates, poly(DTH iminocarbonate), poly(bisphenol A iminocarbonate), poly(amino acids), poly(ethyl glutamate), poly(propylene fumarate), polyanhydrides, polyorthoesters, poly(DETOSU-1,6HD), poly(DETOSU-t-CDM), polyurethanes, polyphosphazenes, polyamides, nylons, nylon 12, polyoxyethylated castor oil, poly(ethylene glycol), polyethylene oxide (PEO), polyvinylpyrrolidone, poly(L-lactide-co-D-lactide), ethylene-vinyl acetate, poly(L-lactide-co-D,L-lactide), poly(D-lactide-co-D,L-lactide), poly(lactide-co-glycolide) (including 70:30 to 99:1 PLA-co-PGA, such as 85:15 PLA-co-PGA), poly(lactide-co-ε-caprolactone) (including 70:30 to 99:1 PLA-co-PCL, such as 90:10 PLA-co-PCL), poly(glycolide-co-ε-caprolactone), poly(lactide-co-dioxanone), poly(glycolide-co-dioxanone), poly(lactide-co-trimethylene carbonate), poly(glycolide-co-trimethylene carbonate), poly(lactide-co-ethylene carbonate), poly(glycolide-co-ethylene carbonate), poly(lactide-co-propylene carbonate), poly(glycolide-co-propylene carbonate), poly(lactide-co-2-methyl-2-carboxyl-propylene carbonate), poly(glycolide-co-2-methyl-2-carboxyl-propylene carbonate), poly(lactide-co-hydroxybutyrate), poly(lactide-co-3-hydroxybutyrate), poly(lactide-co-4-hydroxybutyrate), poly(glycolide-co-hydroxybutyrate), poly(glycolide-co-3-hydroxybutyrate), poly(glycolide-co-4-hydroxybutyrate), poly(lactide-co-hydroxyvalerate), poly(lactide-co-3-hydroxyvalerate), poly(lactide-co-4-hydroxyvalerate), poly(glycolide-co-hydroxyvalerate), poly(glycolide-co-3-hydroxyvalerate), poly(glycolide-co-4-hydroxyvalerate), poly(3-hydroxybutyrate-co-4-hydroxybutyrate), poly(hydroxybutyrate-co-hydroxyvalerate), poly(3-hydroxybutyrate-co-3-hydroxyvalerate), poly(3-hydroxybutyrate-co-4-hydroxyvalerate), poly(4-hydroxybutyrate-co-3-hydroxyvalerate), poly(4-hydroxybutyrate-co-4-hydroxyvalerate), poly(a-caprolactone-co-fumarate), poly(ε-caprolactone-co-propylene fumarate), poly(ester-co-ether), poly(lactide-co-ethylene glycol), poly(glycolide-co-ethylene glycol), poly(ε-caprolactone-co-ethylene glycol), poly(ester-co-amide), poly(DETOSU-1,61HD-co-DETOSU-t-CDM), poly(lactide-co-cellulose ester), poly(lactide-co-cellulose acetate), poly(lactide-co-cellulose butyrate), poly(lactide-co-cellulose acetate butyrate), poly(lactide-co-cellulose propionate), poly(glycolide-co-cellulose ester), poly(glycolide-co-cellulose acetate), poly(glycolide-co-cellulose butyrate), poly(glycolide-co-cellulose acetate butyrate), poly(glycolide-co-cellulose propionate), poly(lactide-co-glycolide-co-ε-caprolactone), poly(lactide-co-glycolide-co-trimethylene carbonate), poly(lactide-co-ε-caprolactone-co-trimethylene carbonate), poly(glycolide-co-ε-caprolactone-co-trimethylene carbonate), poly(3-hydroxybutyrate-co-3-hydroxyvalerate-co-4-hydroxybutyrate), poly(3-hydroxybutyrate-co-4-hydroxyvalerate-co-4-hydroxybutyrate), collagen, casein, polysaccharides, cellulose, cellulose esters, cellulose acetate, cellulose butyrate, cellulose acetate butyrate, cellulose propionate, chitin, chitosan, dextran, starch, modified starch, and copolymers and combinations thereof, wherein lactide includes L-lactide, D-lactide and D,L-lactide.

Examples of non-degradable polymeric materials that can compose the body of the device, a layer of the body, or a coating include without limitation polyacrylates, polymethacrylates, poly(n-butyl methacrylate), poly(hydroxyethylmethacrylate), poly(styrene-b-isobutylene-b-styrene), phosphorylcholine polymer, poly(ethylene-co-vinyl acetate), poly(n-butyl methacrylate), blend of thermoplastic Silicone-Polycarbonate-urethane with poly n-butyl methacrylate, poly(vinylidene-co-hexafluoropropylene), Blend of polyvinylpyrrolidone, poly(hexylmethacrylate)-co-polyvinylpyrrolidone-co-poly vinyl acetate, and poly(n-butyl methacrylate)-co-poly(vinyl acetate), Poly(styrene-butylene styrene), poly(tyrosine-derived polycarbonate), polyamides, nylons, nylon 12, poly(ethylene glycol), polyethylene oxide (PEO), polydimethylsiloxane, polyvinylpyrrolidone, ethylene-vinyl acetate, phosphorylcholine-containing polymers, poly(2-methacryloyloxyethylphosphorylcholine), poly(2-methacryloyloxyethylphosphorylcholine-co-butyl methacrylate), polyvinylpyridine block with poly methyl methacrylate (PMMA), poly N-(2-Hydroxypropyl) methacrylamide, Polyethylenimine (PEI), dextran, dextrin, chitosans, poly(L-lysine), and poly(aspartamides), polyamides, Polyethylene glycol (PEG), Silicones, poly(anhydride), poly ortho esters, polystyrene-b-polyvinylpyridine, poly(styrene)-poly(butadiene)-poly(vinyl pyridine), poly(styrene-poly(methacrylic acid), poly(styrene)-poly(ethylene oxide), poly(vinyl pyridine)-poly(butadiene)-poly(vinyl pyridine), and poly(styrene)-poly(vinyl pyridine)-poly(ethylene oxide) and copolymers and/or combinations thereof.

Non-limiting examples of corrodible metals and metal alloys that can compose the body of the device, a layer of the body, or a coating include cast ductile irons (e.g., 80-55-06 grade cast ductile iron), corrodible steels (e.g., AISI 1010 steel, AISI 1015 steel, AISI 1430 steel, AISI 5140 steel and AISI 8620 steel), melt-fusible metal alloys, bismuth-tin alloys (e.g., 40% bismuth-60% tin and 58% bismuth-42% tin), bismuth-tin-indium alloys, magnesium, magnesium alloys, tungsten alloys, zinc alloys, shape-memory metal alloys, and superelastic metal alloys. Examples of non-corrodible metals and metal alloys that can compose the body of the device, a layer of the body, or a coating include without limitation stainless steels (e.g., 316L stainless steel), cobalt-chromium alloys (e.g., L-605 and MP35N cobalt-chromium alloys), gold, molybdenum-rhenium alloys, nickel-titanium alloys, palladium, platinum, platinum-iridium alloys, tantalum, and alloys thereof.

In some examples, the device is coated. The coating layer may comprise a therapeutic agent and an additive. In some examples, the coating layer overlying an exterior surface of the exterior surface of the medical device consists essentially of the therapeutic agent and the additive.

In some examples, the additive is selected from PEG (polyethylene glycol), polyalkylene oxide, e.g., polyethylene oxide, polypropylene oxide, or a copolymer thereof (e.g., a polyethylene oxide-polypropylene oxide-polyethylene oxide copolymers), polyphenylene oxide, copolymers of PEG and polyalkylene oxide, poly (methoxyethyl methacrylate benzoate), poly (a methacryloyloxy one phosphatidylcholine), perfluorinated polyether, dextran or poly vinylpyrrolidone, poly (ethylene-vinyl acetate), polypeptides, water soluble surfactants, water soluble vitamins, and proteins, PEG fatty esters and alcohols, glycerol fatty esters, sorbitan fatty esters, PEGylation (PEG-drug conjugation), PEG glyceryl fatty esters, PEG sorbitan fatty esters, sugar fatty esters, PEG sugar esters, vitamins and derivatives, amino acids, multi amino acids and derivatives, peptides, polypeptides, oligomers, copolymers, block polymers, proteins, albumin, quaternary ammonium salts such as but not limited to benzalkonium chloride, benzethonium chloride, docecyl trimethyl ammonium bromide, sodium dodecylsulfates, dialkyl methylbenzyl ammonium chloride, dialkylesters of sodium sulfosuccinic acid, organic acids, salts and anhydrides and combinations thereof.

In another example, a direct factor Xa inhibitor, preferably Rivaroxaban, more preferably Apixaban released locally in combination with Argatroban from an implant to inhibit fibrin or clot formation.

In another example, a direct factor Xa inhibitor, preferably Rivaroxaban, more preferably Apixaban released locally in combination with Argatroban from an implant to inhibit fibrin, clot formation, and/or smooth muscle cell proliferation.

In another example, a direct factor Xa inhibitor, preferably Rivaroxaban, more preferably Apixaban released locally by an implant to inhibit clot formation.

In one example, a device delivery one or more drugs locally, wherein locally comprises delivering said one or more drugs to one or more of site-specific location, to a vessel wall, adjacent to a vessel wall, in a body lumen, to a body organ, within a body organ, to the device surface in a body lumen, to a tissue, or to an injured tissue.

In another example, a direct factor Xa inhibitor, preferably Rivaroxaban, more preferably Apixaban is released locally in combination with an m-TOR inhibitor to inhibit fibrin formation or clot formation, or to inhibit fibrin formation or clot formation through 7 days, or to inhibit fibrin formation or clot formation through 28 days.

In another example, a third antiproliferative drug is configured to be released from the device in combination with Argatroban and Rivaroxaban or Apixaban, at similar dose and release rate or different dose and release rate. In a specific example, the anti-proliferative drug is sirolimus or its analogs (including deuterated analog), metabolites, or salts.

In one example, a device delivery one or more drugs locally, wherein locally comprises delivery of said one or more drugs to one or more of site-specific location, adjacent to a vessel wall, to a vessel wall, in a body lumen, to the device surface in a body lumen, to a tissue, to an injured tissue, wherein the local concentration of the one or more dugs maybe higher than in the systemic concentration of the one or more drugs.

In a preferred example, a device releasing factor Xa inhibitor in a body lumen wherein said device inhibits fibrin formation thereby inhibiting clot formation.

It will be understood by one of ordinary skill in the art that the devices and methods described herein may be used in combination with one or more additional bioactive agents. Such agents optionally include anti-mitotic agents, cytostatic agents, anti-migratory agents, immunomodulators, immunosuppressants, anti-inflammatory agents, anti-ischemia agents, anti-hypertensive agents, vasodilators, anti-hyperlipidemia agents, anti-diabetic agents, anti-cancer agents, anti-tumor agents, anti-angiogenic agents, angiogenic agents, anti-chemokine agents, healing-promoting agents, anti-bacterial agents, anti-fungal agents, and combinations thereof. It is understood that a bioactive agent may exert more than one biological effect.

In a preferred example, a device releasing one or more calcium chelating agent, factor Xa inhibitors, and/or one or more factor IIa inhibitors, and/or one or more antiproliferative agents, wherein said one or more agents inhibit thrombin formation and/or fibrin formation thereby inhibiting clot formation and smooth muscle cell proliferation.

In a preferred example, a device releasing one or calcium chelating agent of EDTA ammonium slat complex, factor Xa inhibitors, and/or one or more factor IIa inhibitors, and/or one or more antiproliferative agents, wherein said one or more agents inhibit thrombin formation and/or fibrin formation thereby inhibiting clot formation and smooth muscle cell proliferation.

In a preferred example, a device releasing one or more cationic anti-coagulation enhancer, factor Xa inhibitors, and/or one or more factor IIa inhibitors, and/or one or more antiproliferative agents, wherein said one or more agents inhibit thrombin formation and/or fibrin formation thereby inhibiting clot formation and smooth muscle cell proliferation.

In a preferred example, a device releasing one or more calcium chelating agent, one or more a calcium chelating agent of EDTA ammonium slat complex, factor Xa inhibitors, and/or one or more factor IIa inhibitors, and/or one or more antiproliferative agents, wherein said one or more agents inhibit thrombin formation and/or fibrin formation thereby inhibiting clot formation and smooth muscle cell proliferation.

In a preferred example, a device releasing one or more a calcium chelating agent, a direct factor Xa inhibitors, and/or one or more factor IIa inhibitors, and/or one or more antiproliferative agents, wherein said one or more agents inhibit thrombin formation and/or fibrin formation thereby inhibiting clot formation and smooth muscle cell proliferation.

In some examples, the injury to a tissue, surface, vessel/lumen wall, or other body part is the first substantial injury resulting from a surgery or intervention. In certain examples, the surgery or intervention is selected from the group consisting of vascular surgeries and interventions, cardiovascular surgeries and interventions, peripheral vascular surgeries and interventions, vascular grafting, vascular replacement, vascular angioplasty, thrombectomy, vascular stent placement, vascular laser therapy, coronary by-pass surgery, coronary angiography, coronary stent placement, carotid artery procedures, peripheral stent placement, organ transplants, artificial heart transplant, and plastic and cosmetic surgeries and interventions. In additional examples, the injury is the first substantial injury caused by the device delivering the one or more active substances, and optionally one or more other kinds of bioactive agents (e.g., anti-proliferative agents, anti-inflammatory agents, etc.). In some examples, a substantial injury to a tissue, surface, vessel/lumen wall or other body part results from contact of a device with the tissue, surface, vessel/lumen wall or other body part in a surgery or intervention (e.g., contact of the device causing damage to the endothelium lining a blood vessel, a surgical cutting instrument cutting a tissue, a deployed stent embedding into the wall of a blood vessel, etc.). In further examples, a substantial injury to a tissue, surface, vessel/lumen wall or other body part has a potential to elicit fibrin/thrombus formation, cell migration, cell proliferation or inflammation, or a combination thereof, at the site of injury or at an area adjacent thereto.

In some examples, the therapeutic composition is formulated to release the one or more active substances at a rate of 1 μg/second/mm device to about 50 μg/day/mm device, preferably at a rate of 1 μg/min/mm device to about 30 μg/day/mm device, more preferably at a rate of 1 μg/hour/mm device to about 30 μg/day/mm device.

In some examples, each of the one or more active substances is released from a temporary or non-temporary device at a rate within a range of about 1 μg/hour/mm device length to about 30 μg/day/mm device length, for example about 1 μg/hour/mm device length to about 20 μg/day-mm device length. In some examples, the therapeutic composition is formulated to release the one or more active substances at a rate of 1 μg/hour/mm device to about 20 μg/day/mm device. In some examples, the therapeutic composition may be formulated to release the one or more active substances at a rate within a range of about 1 μg/hour/mm device length to about 14 μg/hour/mm device length. In some examples, the therapeutic composition may be formulated to release the one or more active substances at a rate within a range bounded by any two of the following values: about 1 μg/hour/mm device length, about 2 μg/hour/mm device length, about 3 μg/hour/mm device length, about 4 μg/hour/mm device length, about 5 μg/hour/mm device length, about 6 μg/hour/mm device length, about 7 μg/hour/mm device length, about 8 μg/hour/mm device length, about 9 μg/hour/mm device length, about 10 μg/hour/mm device length, about 11 μg/hour/mm device length, about 12 μg/hour/nm device length, about 13 μg/hour/mm device length, about 14 μg/hour/mm device length, about 15 μg/hour/mm device length, about 16 μg/hour/mm device length, about 17 μg/hour/mm device length, about 18 μg/hour/mm device length, about 19 μg/hour/mm device length, about 20 μg/hour/mm device length, about 21 μg/hour/mm device length, about 22 μg/hour/mm device length, about 23 μg/hour/mm device length, about 24 μg/hour/mm device length, about 25 μg/hour/mm device length, about 26 μg/hour/mm device length, about 27 μg/hour/nm device length, about 28 μg/hour/mm device length, about 29 μg/hour/mm device length, or about 30 μg/hour/mm device length.

In some examples, the therapeutic composition is formulated to begin releasing the one or more active substances within about 1 minute, 5, 10, 15, 20, 25, or 30 minutes after the external surface of the structure is positioned adjacent the injury site.

In some examples, substantially all of each of the one or more active substances is released from a temporary or non-temporary device within about 1 day to about 180 days or more, for example within about 1 day to about 90 days. In some examples, the therapeutic composition is formulated to release substantially all of the one or more active substances within about 7 days or about 28 days. In some examples, the therapeutic composition may be formulated to release substantially all of the one or more active substances within a range bounded by any two of the following values: 1 day, 3 days, 7 days, 14 days, 21 days, 28 days, 45 days, 90 days, 180 days, or more.

In some examples, the therapeutic composition is formulated to release substantially all of the one or more active substances within about 3 hours, about 6 hours, about 12 hours, about 1 day, or about 3 days. In some examples, the therapeutic composition is formulated to release at least 50%, at least 60%, or at least 70% of the one or more active substances within about 3 hours, about 6 hours, about 12 hours, about 1 day, about 3 days, about 7 days, or about 28 days.

In another example, the device releases the one or more active substances from 1 microgram per mm of device length to 25 micrograms per mm of device length, and preferably releases said agent from 5 micrograms per mm of device length to 20 micrograms per mm of device length.

In some examples, the therapeutic composition is formulated to release the one or more active substances at a rate within a range of about 2 μg/mm device to about 100 μg/mm device, about 5 μg/mm device to about 100 μg/mm device, about 7 μg/mm device to about 100 μg/mm device, or about 10 μg/mm device to about 100 μg/mm device within about 3 hours, 12 hours, 1 day, 3 days, 7 days, 28 days, 90 days, or 180 days. In some examples, the therapeutic composition is formulated to release the one or more active substances at a rate within a range of about 5 μg/mm device to about 100 μg/mm device within about 3 hours. In some examples, the therapeutic composition is formulated to release the one or more active substances at a rate within a range of about 5 μg/mm device to about 100 μg/mm device within about 12 hours. In some examples, the therapeutic composition is formulated to release the one or more active substances at a rate within a range of about 5 μg/mm device to about 100 μg/mm device within about 7 days. In some examples, the therapeutic composition is formulated to release the one or more active substances at a rate within a range of about 5 μg/mm device to about 100 μg/mm device within about 28 days.

In some examples, the therapeutic composition is formulated to release the one or more active substances at a dose within a range of about 0.5 μg/mm2 device to about 15 μg/mm2 device, or of about 1 μg/mm2 device to about 12 μg/mm2 device, or of about 2 μg/mm2 device to about 12 μg/mm2 device, or of about 5 μg/mm2 device to about 12 μg/mm2 device, or of about 7 μg/mm2 device to about 12 μg/mm2 device, within about 3 hours or about 12 hours or about 1 day or about 3 days or about 7 days. In some examples, the therapeutic composition is formulated to release the one or more active substances at a dose within a range of about 1 μg/mm2 device to about 12 μg/mm2 device within about 3 hours. In some examples, the therapeutic composition is formulated to release the one or more active substances at a dose within a range of about 1 μg/mm2 device to about 12 μg/mm2 device within about 12 hours. In some examples, the therapeutic composition is formulated to release the one or more active substances at a dose within a range of about 1 μg/mm2 device to about 12 μg/mm2 device within about 7 days. In some examples, the therapeutic composition is formulated to release the one or more active substances at a dose within a range of about 1 μg/mm2 device to about 12 μg/mm2 device within about 28 days, about 90 days, or about 180 days.

In some examples, each of the one or more agents is released from a temporary or non-temporary device at the same rate. In other examples, one or more of the one or more agents that inhibit fibrin/thrombus formation or promote fibrin/thrombus dissolution and/or other bioactive agents is released from a temporary or non-temporary device at a different rate.

In some examples, the therapeutic composition is formulated to release the calcium chelating agent, direct factor Xa inhibitor and/or the direct factor IIa inhibitor faster than the anti-proliferative agent.

In some examples, the therapeutic composition is formulated to release a larger dose of a calcium chelating agent, the direct factor Xa inhibitor than the anti-proliferative agent. In some examples, the dose of a calcium chelating agent, the direct factor Xa inhibitor is about 1.25 to about 5 times larger, about 1.5 to about 3 times larger, or about 1.5 to about 2.5 times larger than a dose of the anti-proliferative agent.

In some examples, the therapeutic composition is formulated to release a dose of colchicine and/or tirofiban sufficient to generate a blood concentration of the colchicine and/or tirofiban which is smaller than a median maximum serum concentration (Cmax) of the colchicine and/or tirofiban inhibitor generated by systemic delivery of the colchicine and/or tirofiban to achieve the same tissue concentration from 5 to 20 picograms per milliliter (pg/ml) at the injury site. In other examples, the therapeutic composition is formulated to release a dose of the colchicine and/or tirofiban sufficient to generate a blood concentration of the colchicine and/or tirofiban which is smaller than a median maximum serum concentration (Cmax) of the colchicine and/or tirofiban generated by systemic delivery of the colchicine and/or tirofiban when taking one or more oral dose of up to 2 mg of colchicine and/or tirofiban. In some examples, the systemic delivery comprises a single oral dose, a daily oral dose, or a smallest oral dose of the colchicine and/or tirofiban. In some examples, the blood concentration is larger than a median minimum serum concentration (Cmin) of the colchicine and/or tirofiban generated by systemic delivery. In some examples, the blood concentration is smaller than a median minimum serum concentration (Cmin) of the colchicine and/or tirofiban generated by systemic delivery. In some examples, the Cmax is measured using one of plasma blood, serum blood, or whole blood. In other examples, the median Cmax is 1 ng/ml, or 2 ng/ml, or 4 ng/ml, or 6 ng/ml, or 8 ng/ml of blood.

In some examples, the therapeutic composition is formulated to release a dose of the tirofiban sufficient to generate a blood concentration of the tirofiban which is smaller than a median maximum serum concentration (Cmax) of the tirofiban inhibitor generated by systemic delivery of the tirofiban to achieve the same tissue concentration at the injury site. In other examples, the therapeutic composition is formulated to release a dose of the tirofiban sufficient to generate a blood concentration of the tirofiban which is smaller than a median maximum serum concentration (Cmax) of the tirofiban generated by systemic delivery of the tirofiban when taking one or more oral dose of said tirofiban. In some examples, the systemic delivery comprises a single oral dose, a daily oral dose, or a smallest oral dose of the tirofiban. In some examples, the blood concentration is larger than a median minimum serum concentration (Cmin) of tirofiban generated by systemic delivery. In some examples, the blood concentration is smaller than a median minimum serum concentration (Cmin) of the tirofiban generated by systemic delivery. In some examples, the Cmax is measured using one of plasma blood, serum blood, or whole blood. In other examples, the median Cmax is 10 ng/ml, or 100 ng/ml, or 500 ng/ml, or 1 μg/ml, or 25 μg/mi of blood. In some examples, the therapeutic composition is formulated to release a dose of the direct factor Xa inhibitor sufficient to generate a blood concentration of the direct factor Xa inhibitor which is smaller than a median maximum serum concentration (Cmax) of the direct factor Xa inhibitor generated by systemic delivery of the direct factor Xa inhibitor to achieve the same tissue concentration at the injury site. In other examples, the therapeutic composition is formulated to release a dose of the direct factor Xa inhibitor sufficient to generate a blood concentration of the direct factor Xa inhibitor which is smaller than a median maximum serum concentration (Cmax) of the direct factor Xa inhibitor generated by systemic delivery of the direct factor Xa inhibitor when taking one or more oral dose of said factor Xa inhibitor. In some examples, the systemic delivery comprises a single oral dose, a daily oral dose, or a smallest oral dose of the direct factor Xa inhibitor. In some examples, the blood concentration is larger than a median minimum serum concentration (Cmin) of the direct factor Xa inhibitor generated by systemic delivery. In some examples, the blood concentration is smaller than a median minimum serum concentration (Cmin) of the direct factor Xa inhibitor generated by systemic delivery. In some examples, the Cmax is measured using one of plasma blood, serum blood, or whole blood. In other examples, the median Cmax is 80 ng/ml, or 123 ng/ml, or 171 ng/ml, or 321 ng/ml, or 480 ng/ml of blood.

In some examples, the therapeutic composition is formulated to release a dose of the direct factor Xa inhibitor sufficient to generate a plasma drug level area under the curve (AUC (0-24) or AUC (0-∞)) in ng·h/ml which is smaller than a median (AUC (0-24) or AUC (0-∞)) in ng·h/ml of the direct factor Xa inhibitor generated by systemic delivery of the direct factor Xa inhibitor to achieve the same tissue concentration at the injury site. In other examples, the therapeutic composition is formulated to release a dose of the direct factor Xa inhibitor sufficient to generate a plasma drug level area under the curve (AUC (0-24) or AUC (0-∞)) in ng·h/ml which is smaller than a median (AUC (0-24) or AUC (0-∞)) in ng·h/ml of the direct factor Xa inhibitor generated by systemic delivery of the direct factor Xa inhibitor when taking one or more oral dose of said factor Xa inhibitor. In some examples, the systemic delivery comprises a single oral dose, a daily oral dose, or a smallest oral dose of the direct factor Xa inhibitor. In some examples, the (AUC (0-24) or AUC (0-∞)) is measured using one of plasma blood, serum blood, or whole blood. In other examples, the median (AUC (0-24) or AUC (0-∞)) is 724 ng·h/ml, or 1437 ng·h/ml, or 2000 ng·h/ml, or 4000 ng·h/ml.

In some examples, the therapeutic composition is formulated to release a dose of the anti-proliferative agent sufficient to generate a blood concentration of the anti-proliferative agent which is smaller than a median maximum serum concentration (Cmax) of the anti-proliferative agent generated by systemic delivery of the anti-proliferative agent to achieve the same tissue concentration at the injury site. In some examples, the systemic delivery comprises a single oral dose, a daily oral dose, or a smallest oral dose of the anti-proliferative agent. In some examples, the blood concentration is larger than a median minimum serum concentration (Cmin) of the anti-proliferative agent generated by systemic delivery. In some examples, the blood concentration is smaller than a median minimum serum concentration (Cmin) of the anti-proliferative agent generated by systemic delivery. In some examples, the therapeutic composition is formulated to release a dose of the anti-proliferative agent sufficient to generate a plasma drug level area under the curve (AUC (0-∞)) in ng·h/ml which is smaller than a median AUC (0-∞) in ng·h/ml of the anti-proliferative agent generated by systemic delivery of the anti-proliferative agent to achieve the same tissue concentration at the injury site.

In some examples, the therapeutic composition is formulated to release a dose of the direct factor IIa inhibitor sufficient to generate a blood concentration of the direct factor IIa inhibitor which is smaller than a median maximum serum concentration (Cmax) of the direct factor IIa inhibitor generated by systemic delivery of the direct factor IIa inhibitor to achieve the same tissue concentration at the injury site. In other examples, the therapeutic composition is formulated to release a dose of the direct factor IIa inhibitor sufficient to generate a blood concentration of the direct factor IIa inhibitor which is smaller than a median maximum serum concentration (Cmax) of the direct factor IIa inhibitor generated by systemic delivery of the direct factor IIa inhibitor when taking one or more oral dose of said factor IIa inhibitor. In some examples, the systemic delivery comprises a single oral dose, a daily oral dose, or a smallest oral dose of the direct factor IIa inhibitor. In some examples, the blood concentration is larger than a median minimum serum concentration (Cmin) of the direct factor IIa inhibitor generated by systemic delivery. In some examples, the blood concentration is smaller than a median minimum serum concentration (Cmin) of the direct factor IIa inhibitor generated by systemic delivery. In some examples, the Cmax is measured using one of plasma blood, serum blood, or whole blood. In other examples, the median Cmax is 80 ng/ml, or 123 ng/ml, or 171 ng/ml, or 321 ng/ml, or 480 ng/ml of blood.

In some examples, the therapeutic composition is formulated to release a dose of the direct factor IIa inhibitor sufficient to generate a plasma drug level area under the curve (AUC (0-24) or AUC (0-∞)) in ng·h/ml which is smaller than a median (AUC (0-24) or AUC (0-∞)) in ng·h/ml of the direct factor IIa inhibitor generated by systemic delivery of the direct factor IIa inhibitor to achieve the same tissue concentration at the injury site. In other examples, the therapeutic composition is formulated to release a dose of the direct factor IIa inhibitor sufficient to generate a plasma drug level area under the curve (AUC (0-24) or AUC (0-∞)) in ng·h/ml which is smaller than a median (AUC (0-24) or AUC (0-∞)) in ng·h/ml of the direct factor IIa inhibitor generated by systemic delivery of the direct factor IIa inhibitor when taking one or more oral dose of said factor IIa inhibitor. In some examples, the systemic delivery comprises a single oral dose, a daily oral dose, or a smallest oral dose of the direct factor IIa inhibitor. In some examples, the (AUC (0-24) or AUC (0-∞)) is measured using one of plasma blood, serum blood, or whole blood. In other examples, the median (AUC (0-24) or AUC (0-m)) is 724 ng·h/ml, or 1437 ng·h/ml, or 2000 ng·h/ml, or 4000 ng·h/ml.

In some examples, local delivery of one or more of the active substances may reduce the time a patient needs to spend on oral medications and/or obviate the need for such medications entirely.

In some examples, the dose of each of the one or more active substances for optional systemic administration on a one-time basis or over a certain time period described herein (e.g., 6 hr, 12 hr, 1 day, 2 days, 3 days, 1 week, 2 weeks, 3 weeks, 1 month, etc.) independently is at least about 1, 5, 10, 20, 50, 100 or 500 mg, or at least about 1, 5 or 10 g. In further examples, the amount of each of the one or more active substances loaded in and/or on a temporary or non-temporary device, or the amount of each such agent released from the device, independently is at least about 1, 10, 50, 100 or 500 μg, or at least about 1, 5, 10 or 20 mg. In certain examples, the amount of each of the fibrin/thrombus formation-inhibiting or fibrin/thrombus dissolution-promoting agent(s) loaded in and/or on the device, or the amount of each such agent released from the device, independently is about 1 μg to about 20 mg, or about 10 μg to about 10 mg, or about 50 μg to about 5 mg, or about 100 μg to about 1 mg, or about 100 μg to about 500 μg, or about 500 μg to about 1 mg.

In further examples, the concentration of each of the one or more active substances released from a temporary or non-temporary device, and optionally administered systemically in addition to locally, in blood or tissue at the site of injury or at an area adjacent thereto, and/or in blood or tissue adjacent to the device, independently is at least about 0.001, 0.01, 0.1, 1, 10, 50, 100 or 500 nM, or at least about 1, 10, 50, 100, 500 or 1000 μM. In certain examples, the concentration of each of the fibrin/thrombus formation-inhibiting or fibrin/thrombus dissolution-promoting agent(s) released from a temporary or non-temporary device, and optionally administered systemically in addition to locally, in blood or tissue at the site of injury or at an area adjacent thereto, and/or in blood or tissue adjacent to the device, independently is about 0.01 or 0.1 nM to about 1000 μM, or about 0.1 or 1 nM to about 500 μM, or about 1 or 10 nM to about 100 μM, or about 50 nM to about 50 μM, or about 10 or 100 nM to about 10 μM, or about 100 nM to about 1 μM, or about 1 μM to about 10 μM.

In still further examples, the concentration of each of the one or more active substances released from a temporary or non-temporary device, and optionally administered systemically in addition to locally, in tissue at the site of injury or at an area adjacent thereto, and/or in tissue adjacent to the device, independently is at least about 0.01, 0.1, 1, 10, 50, 100 or 500 ng/gm tissue, or at least about 1, 10, 50, 100, 500 or 1000 μg/gm tissue. In certain examples, the concentration of each of the fibrin/thrombus formation-inhibiting or fibrin/thrombus dissolution-promoting agent(s) released from a temporary or non-temporary device, and optionally administered systemically in addition to locally, in tissue at the site of injury or at an area adjacent thereto, and/or in tissue adjacent to the device, independently is about 0.01 or 0.1 ng/gm tissue to about 1000 μg/gm tissue, or about 0.1 or 1 ng/gm tissue to about 500 μg/gm tissue, or about 1 or 10 ng/gm tissue to about 100 μg/gm tissue, or about 50 ng/gm tissue to about 50 μg/gm tissue, or about 10 or 100 ng/gm tissue to about 10 μg/gm tissue, or about 100 ng/gm tissue to about 1 μg/gm tissue, or about 1 μg/gm tissue to about 10 μg/gm tissue.

In additional examples, the concentration of each of the one or more active substances released from a temporary or non-temporary device (that may or may not cause an injury to a tissue, surface, vessel/lumen wall or other body part), and optionally administered systemically in addition to locally, in blood or tissue at the site of injury or at an area adjacent thereto, and/or in blood or tissue adjacent to the device, independently is at least about 0.001, 0.01, 0.1, 1, 10, 50, 100 or 500 nM, or at least about 1, 10, 50 or 100 μM, within about 1 day, 12 hr, 6 hr, 3 hr, 2 hr, 1 hr, 30 min., 15 min., 5 min, or 1 min. before, during and/or within about 1 day, 12 hr, 6 hr. 3 hr, 2 hr, 1 hr, 30 min., 15 min., 5 min, or 1 min. after delivery or deployment of the device and/or the injury. In further examples, the concentration of each of the one or more active substances released from a temporary or non-temporary device (that may or may not cause an injury to a tissue, surface, vessel/lumen wall or other body part), and optionally administered systemically in addition to locally, in tissue at the site of injury or at an area adjacent thereto, and/or in tissue adjacent to the device, independently is at least about 0.01, 0.1, 1, 10, 50, 100 or 500 ng/gm tissue, or at least about 1, 10, 50 or 100 μg/gm tissue, within about 1 day, 12 hr. 6 hr, 3 hr, 2 hr, 1 hr, 30 min., 15 min., 5 min, or 1 min. before, during and/or within about 1 day, 12 hr, 6 hr, 3 hr, 2 hr, 1 hr, 30 min., 15 min., 5 min, or 1 min. after delivery or deployment of the device and/or the injury.

In some examples, the dose of each of the one or more optional other kinds of bioactive agents (e.g., anti-proliferative agents, anti-inflammatory agents, etc.) for optional systemic administration on a one-time basis or over a certain time period described herein (e.g., 6 hr, 12 hr, 1 day, 2 days, 3 days, 1 week, 2 weeks, 3 weeks, 1 month, etc.) independently is at least about 1, 5, 10, 20, 50, 100 or 500 mg, or at least about 1, 5 or 10 g. In additional examples, the amount of each of the one or more optional other kinds of bioactive agents (e.g., anti-proliferative agents, anti-inflammatory agents, etc.) loaded in and/or on a temporary or non-temporary device, or the amount of each such agent released from the device, independently is at least about 1, 10, 50, 100 or 500 μg, or at least about 1, 5, 10 or 20 mg. In certain examples, the amount of each of the optional other kind(s) of bioactive agent(s) loaded in and/or on the device, or the amount of each such agent released from the device, independently is about 1 μg to about 20 mg, or about 10 μg to about 10 mg, or about 50 μg to about 5 mg, or about 100 μg to about 1 mg, or about 100 μg to about 500 μg, or about 500 μg to about 1 mg, or about 50 μg to about 200 μg.

In further examples, the concentration of each of the one or more optional other kinds of bioactive agents (e.g., anti-proliferative agents, anti-inflammatory agents, etc.) released from a temporary or non-temporary device, and optionally administered systemically in addition to locally, in tissue at the site of injury or at an area adjacent thereto, and/or in tissue adjacent to the device, independently is at least about 0.01, 0.1, 1, 10, 50, 100 or 500 ng/gm tissue, or at least about 1, 10, 50, 100, 500 or 1000 μg/gm tissue. In certain examples, the concentration of each of the optional other kind(s) of bioactive agent(s) released from a temporary or non-temporary device, and optionally administered systemically in addition to locally, in tissue at the site of injury or at an area adjacent thereto, and/or in tissue adjacent to the device, independently is about 0.01 or 0.1 ng/gm tissue to about 1000 μg/gm tissue, or about 0.1 or 1 ng/gm tissue to about 500 μg/gm tissue, or about 1 or 10 ng/gm tissue to about 100 μg/gm tissue, or about 50 ng/gm tissue to about 50 μg/gm tissue, or about 10 or 100 ng/gm tissue to about 10 μg/gm tissue, or about 100 ng % gm tissue to about 1 μg/gm tissue, or about 1 μg/gm tissue to about 10 μg/gm tissue.

In some examples, the device contains the bioactive agent(s) in the body and/or on at least one surface of the device. In certain examples, the bioactive agent(s) are contained in one or more layers in the body and/or at the surface of the device.

In further examples, the bioactive agent(s) are contained in one or more coatings disposed over the body of the device. The coating(s) can be disposed over any desired portion(s) and any desired surface(s) of the body of the device. As a non-limiting example, for a tubular vascular device such as a stent, the coating(s) can be disposed over the luminal (lumen-facing) surface, the abluminal (tissue-facing) surface or the side surface(s) of the stent, or a combination thereof (e.g., all surfaces of the stent).

In additional examples, the device comprises the bioactive agent(s) in the body of the device and in one or more coatings disposed over the body of the device.

A temporary or non-temporary device can comprise openings in and/or on the body (including at the surface) of the device, and/or in one or more coatings disposed over the body structure of the device. Examples of openings include without limitation pores (including partial pores and through pores), holes (including partial holes and through holes), voids, recesses, pits, cavities, trenches, reservoirs, and channels. In some examples, a temporary or non-temporary device contains one or more anti-coagulant, and optionally one or more other kinds of bioactive agents (e.g., anti-proliferative agents, anti-inflammatory agents, etc.) in openings in and/or on the body (including at the surface) of the device, and/or in one or more coatings disposed over the body of the device.

The device may comprise one or more coatings disposed over an exterior surface of a structure of the device, as described herein. In some embodiments, the coating(s) may comprise a homopolymer, a copolymer, a mixture of homopolymers, a mixture of copolymers, or a mixture of a homopolymer and a copolymer. In some examples, the coating(s) comprise a soft or hydrophilic, or a softer or more hydrophilic, polymeric material. In further examples, the coating(s) comprise a polymeric material and an additive (e.g., a monomer of the polymeric material) that softens the polymeric material.

In some examples, the device has a first coating that comprises a biodegradable or non-degradable polymeric material, or one or more bioactive agents, or both a biodegradable or non-degradable polymeric material and one or more bioactive agents. In further examples, the device has a second coating that comprises a biodegradable or non-degradable polymeric material, or one or more bioactive agents, or both a biodegradable or non-degradable polymeric material and one or more bioactive agents, wherein the second coating optionally is disposed over the first coating. In additional examples, the device has a third coating that comprises a biodegradable or non-degradable polymeric material, wherein the third coating is disposed over the first coating and/or the second coating. In some examples, the third coating serves as a top layer or coat or diffusion barrier that controls release of one or more bioactive agents from inner coating(s) and % or the body of the device.

In some examples, a bioactive agent that is intended to have an earlier or shorter time of action can be contained in an outer coating, on a surface uncovered by a coating, and/or in the body of the device closer to the surface, and a bioactive agent that is intended to have a later or longer time of action can be contained in an inner coating, in a coating covered by a barrier coating, on a surface covered by a coating, and/or in the body of the device farther from the surface. In further examples, a bioactive agent that is intended to have an earlier or shorter time of action is contained on a surface of the device, or contained in a coating on the device or in a layer of the body of the device which comprises a faster-degrading polymeric material, and a bioactive agent that is intended to have a later or longer time of action is contained within the device, or contained in a coating on the device or in a layer of the body of the device which comprises a slower-degrading or non-degrading polymeric material. In additional examples, a bioactive agent that is intended to have an earlier or shorter time of action is more soluble, and a bioactive agent that is intended to have a later or longer time of action is less soluble.

In certain examples, the concentration of a bioactive agent [e.g., anti-coagulant, anti-proliferative, etc.] in a coating comprising a polymeric material is at least about 10%, 20%, 30%, 40%, 50% or 60% by weight relative to the weight of the bioactive agent and the polymeric material.

In further examples, the thickness (e.g., average thickness) of each of the coating(s) independently is no more than about 20, 15, 10, 5, 3 or 1 micron.

In some examples, the coating(s) may comprise carrier material. Non-limiting examples of carrier materials include biodegradable polymeric materials, non-degradable polymeric materials, and other matrix materials.

In some examples, the carrier material may be porous. In certain examples, the porosity of each of the coating(s) of the carrier material may be within a range of about 10 nm to about 10 μm.

In some examples, the carrier material may be biodegradable. In certain examples, the carrier material may have a depredation rate within a range of about 1 month to about 36 months.

In some examples, the weight compositional ratio of the carrier material to the therapeutic composition of one or more bioactive agents may be within a range of about 1:5 to 3:2.

Non-limiting examples of polymeric materials that can compose the carrier material include polyesters, polylactide, polyglycolide, poly(ε-caprolactone), polydioxanone, poly(hydroxyalkanoates), poly(L-lactide-co-D-lactide), poly(L-lactide-co-D,L-lactide), poly(D-lactide-co-D,L-lactide), poly(lactide-co-glycolide) (including 70:30 to 99:1 PLA-co-PGA, such as 85:15 PLA-co-PGA), poly(lactide-co-ε-caprolactone) (including 70:30 to 99:1 PLA-co-PCL, such as 90:10 PLA-co-PCL), poly(glycolide-co-ε-caprolactone), poly(lactide-co-dioxanone), poly(glycolide-co-dioxanone), poly(lactide-co-trimethylene carbonate), poly(glycolide-co-trimethylene carbonate), poly(lactide-co-ethylene carbonate), and copolymers and combinations thereof, wherein lactide includes L-lactide, D-lactide and D,L-lactide. The polymeric material may comprise a material selected from a group of non-degradable polymeric materials consisting of polyacrylates, polymethacrylates, poly(n-butyl methacrylate), poly(hydroxyethylmethacrylate), polyamides, nylons, nylon 12, Dacron, Polyethylene terephthalate, poly(ethylene glycol), polyethylene oxide (PEO), polydimethylsiloxane, polyvinylpyrrolidone, ethylene-vinyl acetate, phosphorylcholine-containing polymers, poly(2-methacryloyloxyethylphosphorylcholine), poly(2-methacryloyloxyethylphosphorylcholine-co-butyl methacrylate), and copolymers and combinations thereof.

Non-limiting examples of biodegradable polymeric materials that can compose the body of the device, a layer of the body, or a coating include polyesters, poly(α-hydroxyacids), polylactide, polyglycolide, poly(ε-caprolactone), polydioxanone, poly(hydroxyalkanoates), poly(hydroxypropionates), poly(3-hydroxypropionate), poly(hydroxybutyrates), poly(3-hydroxybutyrate), poly(4-hydroxybutyrate), poly(hydroxypentanoates), poly(3-hydroxypentanoate), poly(hydroxyvalerates), poly(3-hydroxyvalerate), poly(4-hydroxyvalerate), poly(hydroxyoctanoatcs), poly(3-hydroxyoctanoate), polysalicylate/polysalicylic acid, polycarbonates, poly(trimethylene carbonate), poly(ethylene carbonate), poly(propylene carbonate), tyrosine-derived polycarbonates, L-tyrosine-derived polycarbonates, polyiminocarbonates, poly(DTH iminocarbonate), poly(bisphenol A iminocarbonate), poly(amino acids), poly(ethyl glutamate), poly(propylene fumarate), polyanhydrides, polyorthoesters, poly(DETOSU-1,6HD), poly(DETOSU-t-CDM), polyurethanes, polyphosphazenes, polyamides, nylons, nylon 12, polyoxyethylated castor oil, poly(ethylene glycol), polyethylene oxide (PEO), polyvinylpyrrolidone, poly(L-lactide-co-D-lactide), ethylene-vinyl acetate, poly(L-lactide-co-D,L-lactide), poly(D-lactide-co-D,L-lactide), poly(lactide-co-glycolide) (including 70:30 to 99:1 PLA-co-PGA, such as 85:15 PLA-co-PGA), poly(lactide-co-ε-caprolactone) (including 70:30 to 99:1 PLA-co-PCL, such as 90:10 PLA-co-PCL), poly(glycolide-co-ε-caprolactone), poly(lactide-co-dioxanone), poly(glycolide-co-dioxanone), poly(lactide-co-trimethylene carbonate), poly(glycolide-co-trimethylene carbonate), poly(lactide-co-ethylene carbonate), poly(glycolide-co-ethylene carbonate), poly(lactide-co-propylene carbonate), poly(glycolide-co-propylene carbonate), poly(lactide-co-2-methyl-2-carboxyl-propylene carbonate), poly(glycolide-co-2-methyl-2-carboxyl-propylene carbonate), poly(lactide-co-hydroxybutyrate), poly(lactide-co-3-hydroxybutyrate), poly(lactide-co-4-hydroxybutyrate), poly(glycolide-co-hydroxybutyrate), poly(glycolide-co-3-hydroxybutyrate), poly(glycolide-co-4-hydroxybutyrate), poly(lactide-co-hydroxyvalerate), poly(lactide-co-3-hydroxyvalerate), poly(lactide-co-4-hydroxyvalerate), poly(glycolide-co-hydroxyvalerate), poly(glycolide-co-3-hydroxyvalerate), poly(glycolide-co-4-hydroxyvalerate), poly(3-hydroxybutyrate-co-4-hydroxybutyrate), poly(hydroxybutyrate-co-hydroxyvalerate), poly(3-hydroxybutyrate-co-3-hydroxyvalerate), poly(3-hydroxybutyrate-co-4-hydroxyvalerate), poly(4-hydroxybutyrate-co-3-hydroxyvalerate), poly(4-hydroxybutyrate-co-4-hydroxyvalerate), poly(ε-caprolactone-co-fumarate), poly(s-caprolactone-co-propylene fumarate), poly(ester-co-ether), poly(lactide-co-ethylene glycol), poly(glycolide-co-ethylene glycol), poly(ε-caprolactone-co-ethylene glycol), poly(ester-co-amide), poly(DETOSU-1,61HD-co-DETOSU-t-CDM), poly(lactide-co-cellulose ester), poly(lactide-co-cellulose acetate), poly(lactide-co-cellulose butyrate), poly(lactide-co-cellulose acetate butyrate), poly(lactide-co-cellulose propionate), poly(glycolide-co-cellulose ester), poly(glycolide-co-cellulose acetate), poly(glycolide-co-cellulose butyrate), poly(glycolide-co-cellulose acetate butyrate), poly(glycolide-co-cellulose propionate), poly(lactide-co-glycolide-co-ε-caprolactone), poly(lactide-co-glycolide-co-trimethylene carbonate), poly(lactide-co-ε-caprolactone-co-trimethylene carbonate), poly(glycolide-co-ε-caprolactone-co-trimethylene carbonate), poly(3-hydroxybutyrate-co-3-hydroxyvalerate-co-4-hydroxybutyrate), poly(3-hydroxybutyrate-co-4-hydroxyvalerate-co-4-hydroxybutyrate), collagen, casein, polysaccharides, cellulose, cellulose esters, cellulose acetate, cellulose butyrate, cellulose acetate butyrate, cellulose propionate, chitin, chitosan, dextran, starch, modified starch, and copolymers and combinations thereof, wherein lactide includes L-lactide, D-lactide and D,L-lactide.

Examples of non-degradable polymeric materials that can compose the body of the device, a layer of the body, or a coating include without limitation polyacrylates, polymethacrylates, poly(n-butyl methacrylate), poly(hydroxyethylmethacrylate), poly(styrene-b-isobutylene-b-styrene), phosphorylcholine polymer, poly(ethylene-co-vinyl acetate), poly(n-butyl methacrylate), blend of thermoplastic Silicone-Polycarbonate-urethane with poly n-butyl methacrylate, poly(vinylidene-co-hexafluoropropylene), Blend of polyvinylpyrrolidone, poly(hexylmethacrylate)-co-polyvinylpyrrolidone-co-poly vinyl acetate, and poly(n-butyl methacrylate)-co-poly(vinyl acetate), Poly(styrene-butylene styrene), poly(tyrosine-derived polycarbonate), polyamides, nylons, nylon 12, poly(ethylene glycol), polyethylene oxide (PEO), polydimethylsiloxane, polyvinylpyrrolidone, ethylene-vinyl acetate, phosphorylcholine-containing polymers, poly(2-methacryloyloxyethylphosphorylcholine), poly(2-methacryloyloxyethylphosphorylcholine-co-butyl methacrylate), polyvinylpyridine block with poly methyl methacrylate (PMMA), poly N-(2-Hydroxypropyl) methacrylamide, Polyethylenimine (PEI), dextran, dextrin, chitosans, poly(L-lysine), and poly(aspartamides), polyamides, Polyethylene glycol (PEG), Silicones, poly(anhydride), poly ortho esters, polystyrene-b-polyvinylpyridine, poly(styrene)-poly(butadiene)-poly(vinyl pyridine), poly(styrene-poly(methacrylic acid), poly(styrene)-poly(ethylene oxide), poly(vinyl pyridine)-poly(butadiene)-poly(vinyl pyridine), and poly(styrene)-poly(vinyl pyridine)-poly(ethylene oxide) and copolymers and/or combinations thereof.

Non-limiting examples of corrodible metals and metal alloys that can compose the body of the device, a layer of the body, or a coating include cast ductile irons (e.g., 80-55-06 grade cast ductile iron), corrodible steels (e.g., AISI 1010 steel, AISI 1015 steel, AISI 1430 steel, AISI 5140 steel and AISI 8620 steel), melt-fusible metal alloys, bismuth-tin alloys (e.g., 40% bismuth-60% tin and 58% bismuth-42% tin), bismuth-tin-indium alloys, magnesium, magnesium alloys, tungsten alloys, zinc alloys, shape-memory metal alloys, and superelastic metal alloys. Examples of non-corrodible metals and metal alloys that can compose the body of the device, a layer of the body, or a coating include without limitation stainless steels (e.g., 316L stainless steel), cobalt-chromium alloys (e.g., L-605 and MP35N cobalt-chromium alloys), gold, molybdenum-rhenium alloys, nickel-titanium alloys, palladium, platinum, platinum-iridium alloys, tantalum, and alloys thereof.

In some examples, the device is coated. The coating layer may comprise a therapeutic agent and an additive. In some examples, the coating layer overlying an exterior surface of the exterior surface of the medical device consists essentially of the therapeutic agent and the additive.

In some examples, the additive is selected from PEG (polyethylene glycol), polyalkylene oxide, e.g., polyethylene oxide, polypropylene oxide, or a copolymer thereof (e.g., a polyethylene oxide-polypropylene oxide-polyethylene oxide copolymers), polyphenylene oxide, copolymers of PEG and polyalkylene oxide, poly (methoxyethyl methacrylate benzoate), poly (a methacryloyloxy one phosphatidylcholine), perfluorinated polyether, dextran or poly vinylpyrrolidone, poly (ethylene-vinyl acetate), polypeptides, water soluble surfactants, water soluble vitamins, and proteins, PEG fatty esters and alcohols, glycerol fatty esters, sorbitan fatty esters, PEGylation (PEG-drug conjugation), PEG glyceryl fatty esters, PEG sorbitan fatty esters, sugar fatty esters, PEG sugar esters, vitamins and derivatives, amino acids, multi amino acids and derivatives, peptides, polypeptides, oligomers, copolymers, block polymers, proteins, albumin, quaternary ammonium salts such as but not limited to benzalkonium chloride, benzethonium chloride, docecyl trimethyl ammonium bromide, sodium docecylsulfates, dialkyl methylbenzyl ammonium chloride, dialkylesters of sodium sulfonsuccinic acid, organic acids, salts and anhydrides and combinations thereof.

In another example, a direct factor Xa inhibitor, preferably Rivaroxaban, more preferably Apixaban released locally in combination with Argatroban from an implant to inhibit fibrin or clot formation.

In another example, a direct factor Xa inhibitor, preferably Rivaroxaban, more preferably Apixaban released locally in combination with Argatroban from an implant to inhibit fibrin, clot formation, and/or smooth muscle cell proliferation.

In another example, a direct factor Xa inhibitor, preferably Rivaroxaban, more preferably Apixaban released locally by an implant to inhibit clot formation.

In one example, a device delivery one or more drugs locally, wherein locally comprises delivering said one or more drugs to one or more of site-specific location, to a vessel wall, adjacent to a vessel wall, in a body lumen, to a body organ, within a body organ, to the device surface in a body lumen, to a tissue, or to an injured tissue.

In another example, a direct factor Xa inhibitor, preferably Rivaroxaban, more preferably Apixaban is released locally in combination with an m-TOR inhibitor to inhibit fibrin formation or clot formation, or to inhibit fibrin formation or clot formation through 7 days, or to inhibit fibrin formation or clot formation through 28 days.

In another example, a third antiproliferative drug is configured to be released from the device in combination with Argatroban and Rivaroxaban or Apixaban, at similar dose and release rate or different dose and release rate. In a specific example, the anti-proliferative drug is sirolimus or its analogs (including deuterated analog), metabolites, or salts.

In one example, a device delivery one or more drugs locally, wherein locally comprises delivery of said one or more drugs to one or more of site-specific location, adjacent to a vessel wall, to a vessel wall, in a body lumen, to the device surface in a body lumen, to a tissue, to an injured tissue, wherein the local concentration of the one or more dugs maybe higher than in the systemic concentration of the one or more drugs.

In a preferred example, a device releasing factor Xa inhibitor in a body lumen wherein said device inhibits fibrin formation thereby inhibiting clot formation.

It will be understood by one of the ordinary skill in the art that the devices and methods described herein may be used in combination with one or more additional bioactive agents. Such agents optionally include anti-mitotic agents, cytostatic agents, anti-migratory agents, immunomodulators, immunosuppressants, anti-inflammatory agents, anti-ischemia agents, anti-hypertensive agents, vasodilators, anti-hyperlipidemia agents, anti-diabetic agents, anti-cancer agents, anti-tumor agents, anti-angiogenic agents, angiogenic agents, anti-chemokine agents, healing-promoting agents, anti-bacterial agents, anti-fungal agents, and combinations thereof. It is understood that a bioactive agent may exert more than one biological effect.

### EXPERIMENTAL EXAMPLES

### Example 1: Anti-Proliferative Activity of Apixaban, Argatroban and Rapamycin Combination

Anti-proliferative activity of Apixaban, Argatroban, and Rapamycin was tested in Human Aortic SMC (HAoSMC, ATCC, PCS-100-012). Cell proliferation assay was done in 96-well format. Low passage cells were trypsinized and seeded in 96 well plates at a density of ~4000 cells/well. The cells are allowed to attach overnight in a CO₂ incubator. Next day, the medium was removed and replaced with fresh complete medium containing various concentrations of the test compounds. The final concentration of vehicle (DMSO) in the test medium was 0.1%. After adding test compounds, the cells were incubated for 72 hours. Following this period, the medium was removed and then added fresh medium (100 μl) containing CellTiter Aqueous (1× concentration final) to the wells and incubated for 2 hours in the CO2 incubator. At the end of incubation measured fluorescence with a plate-reader. Controlled incubations with untreated cells and blank incubations containing only medium were included and tested similarly. Based on the cell viability assay the percentage inhibition of the cell proliferation was determined at the different concentrations of the drug tested.

The cell proliferation assay was performed with different concentrations of Apixaban and Argatroban when combined with Rapamycin. Following the cell proliferation assay as indicated earlier, the percent cell proliferation inhibition was determined, and the assay results plotted to determine the IC50.

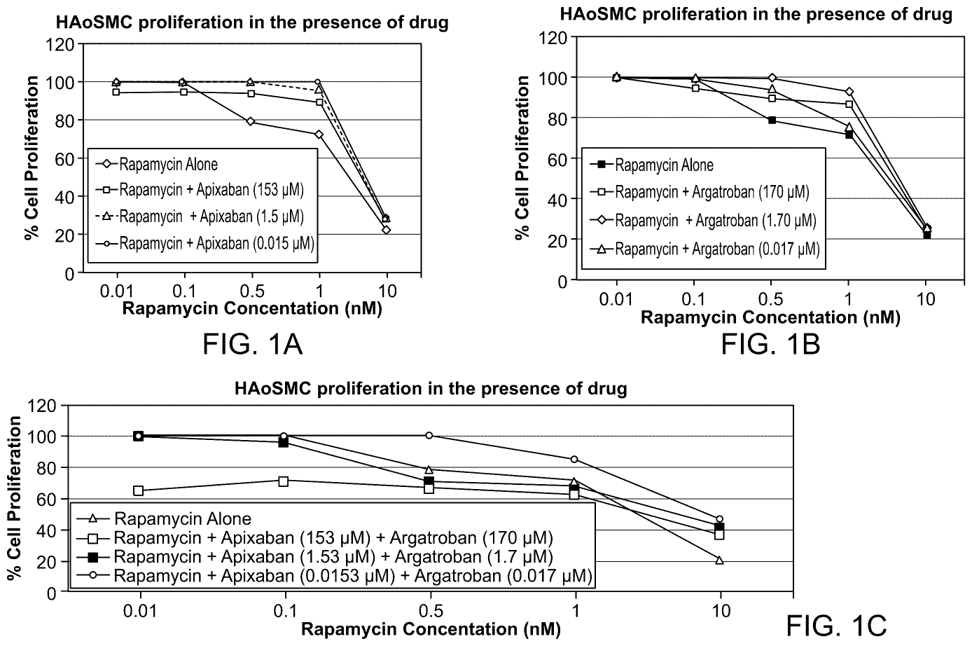

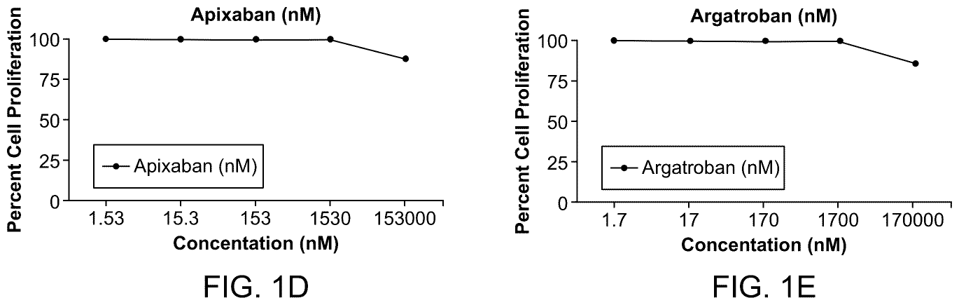

FIG. 1A, FIG. 1B, and FIG. 1C show HAoSMC proliferation inhibition in the presence of different drug combinations. The data shows the combination of Apixaban, Argatroban surprisingly and unexpectedly enhanced the anti-proliferative effects of rapamycin on smooth muscle cell proliferation as measured by cell proliferation test when Apixaban and Argatroban were combined with rapamycin, i.e., the combination of Apixaban, Argatroban, and rapamycin were more potent than rapamycin alone at inhibiting SMS proliferation.

FIG. 1D and FIG. 1E show HAoSMC proliferation in presence of different concentrations of Apixaban or Argatroban. In order to determine if Apixaban or Argatroban independently had inhibitory effect on the proliferation of HAoSMC, a proliferation assay in the presence of either of these two drugs at different concentrations were tested as described earlier. Various concentrations of Apixaban alone or Argatroban alone had small to no inhibition of HAoSMC proliferation was observed as shown in FIGS. 1D-1E.

### Example 2: Activated Clotting Time (ACT) Evaluation of Direct Factor Xa Inhibitor (Apixaban), Direct Factor IIa Inhibitor (Argatroban) or a Combination of Direct Factor Xa Inhibitor (Apixaban) and Direct Factor IIa Inhibitor (Argatroban)

The activated clotting time (ACT) evaluation of anticoagulants was performed in calcium citrate sheep blood and recorded employing the Hemochron® Response device.

The ACT measurements were made in citrated sheep blood. 1.9 ml of citrated sheep blood was added to a test tube containing an activator (Hemochron@Celite@ ACT tubes, Lot F8FTE026 from Accriva Diagnostics, Inc.). A target amount of drug solution was then added into the test tube. The test tube was gently swirled so that the blood and drug was well-mixed. 0.1 ml of 0.3M calcium chloride was then added. The tube was gently shaken before being inserted into the Hemochron Response detector. The ACT read out was recorded and reported. The ACT of the control blood in the absence of any drug as first determined to establish a baseline. Then ACT was determined in the presence of different concentrations of the drug as a single component. Selected drug combinations were then tested to evaluate for potential synergy in action between the two drugs.

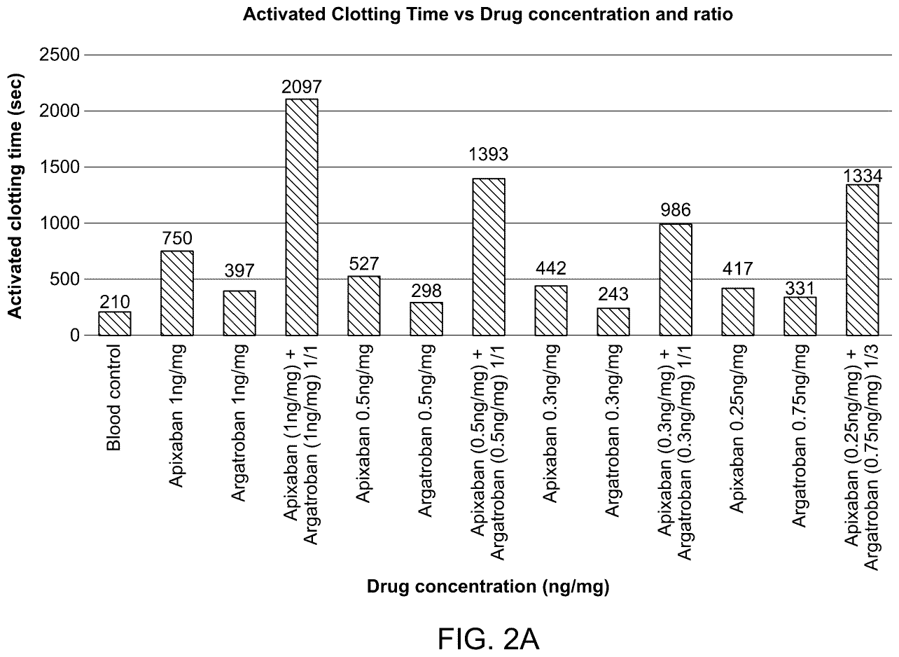

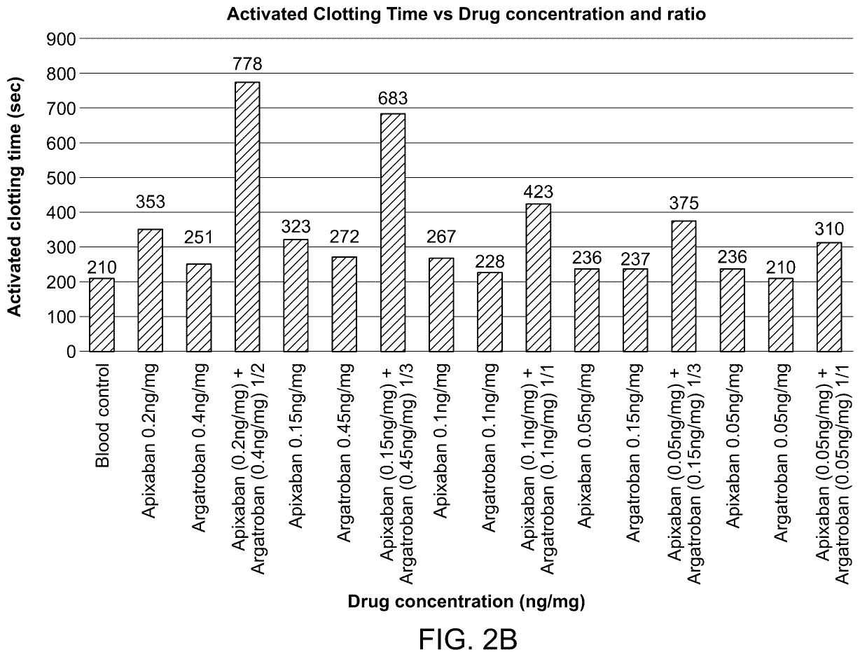

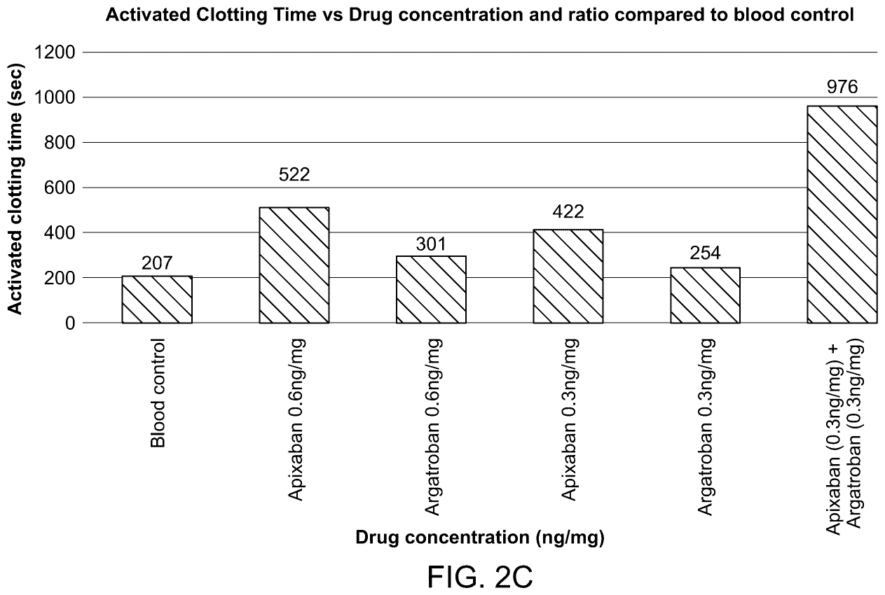

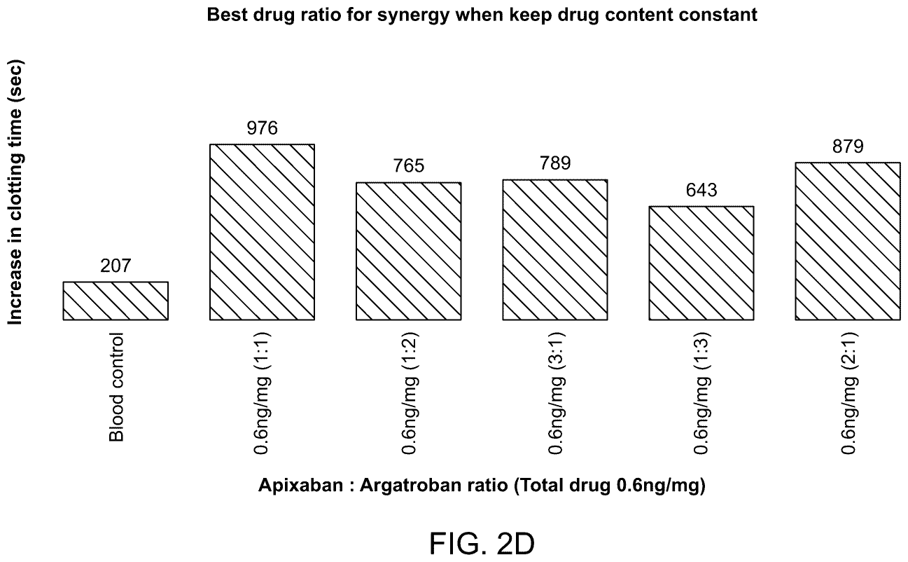

As shown in FIGS. 2A-2D, the clotting time was observed to be significantly extended or increased at a higher drug combination concentration. The Apixaban/Argatroban combination achieved ACT levels that were higher than the sum of the individual ACT values, indicating a synergistic effect between these drug combinations. This may be particularly important when delivering these drugs locally (adjacent to injured tissue) to inhibit clot formation. The figures are presented in ng/mg wherein the density of blood and tissue are approximately the same.

It was found, unexpectedly, that the combination drug concentrations of 0.05 ng/mg for each Apixaban and Argatroban drug extended the ACT by a larger time (as shown in FIG. 2B).

It was found, unexpectedly, that the combination drug concentrations of 0.3 ng/mg for each Apixaban and Argatroban drug (0.6 ng/mg total) extended the ACT by a larger time (i.e., was more effective) than the ACT for each individual drug at 0.6 ng/mg concentration (ACT of 976 for the combination versus 522 for Apixaban versus 301 for Argatroban as shown in FIG. 2C). FIG. 2C further shows, unexpectedly, that the combination drug concentrations of 0.3 ng/mg for each drug (0.6 ng/mg total) extended the ACT by a larger time (i.e., was more effective) than the ACT for the sum of each individual drug ACT at 0.3 ng/mg or at 0.6 ng/mg concentration. (ACT of 976 for the combination versus 676 (for the sum of individual drugs having 0.3 ng/mg concentrations).

It is important to note that drug tissue concentrations for factor Xa inhibitors of Rivaroxaban, Apixaban, Edoxaban, Betrixaban alone or in combination with factor IIa inhibitor Argatroban to have sufficient tissue concentrations in the implanted tissue segment and in the adjacent tissue segment to have therapeutic levels for each drug to be larger than 0.02 ng/mg, larger than 0.1 ng/mg, preferably larger than 0.2 ng/mg of tissue, preferably 0.3 ng/mg of tissue, more preferably larger than 1 ng/mg of tissue at or within 3 hours after implantation, or at or within 1 day after implantation, to inhibit clot formation.

### Example 3: Activated Clotting Time (ACT) Evaluation of Direct Factor Xa Inhibitor (Rivaroxaban) Alone, Direct Factor IIa Inhibitor (Argatroban) Alone or a Combination of Direct Factor Xa Inhibitor (Rivaroxaban) and Direct Factor IIa Inhibitor (Argatroban)

The activated clotting time (ACT) evaluation of anticoagulants was performed in calcium citrate sheep blood and recorded employing the Hemochron® Response device. The ACT measurements were made in citrated sheep blood. 1.9 ml of citrated sheep blood was added to a test tube containing an activator (Hemochron@Celite@ ACT tubes, Lot F8FTE026 from Accriva Diagnostics, Inc.). A target amount of drug solution was then added into the test tube. The test tube was gently swirled so that the blood and drug were well-mixed. 0.1 ml of 0.3M calcium chloride was then added. The tube was gently shaken before being inserted into the Hemochron Response detector. The ACT read out was recorded and reported. The ACT of the control blood in the absence of any drug as first determined to establish a baseline. Here the blood control measured is 165 seconds. Then ACT was determined in the presence of different concentrations of the drug as a single component. In the following study, the net ACT (measured ACT minus blood control ACT) was utilized to visualize the synergistic effect of direct factor Xa inhibitor (Rivaroxaban) and direct factor IIa inhibitor (Argatroban) drug combinations. The figures are presented in ng/mg wherein the density of blood and tissue are approximately the same.

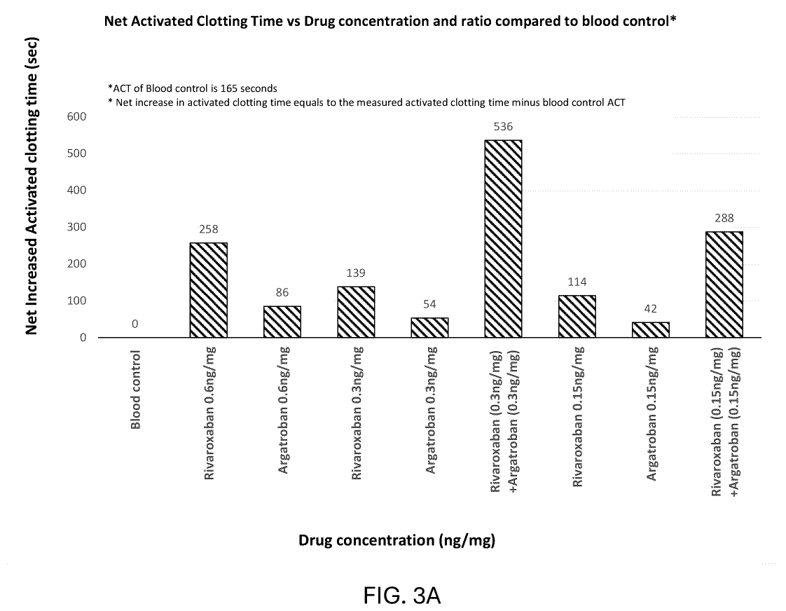

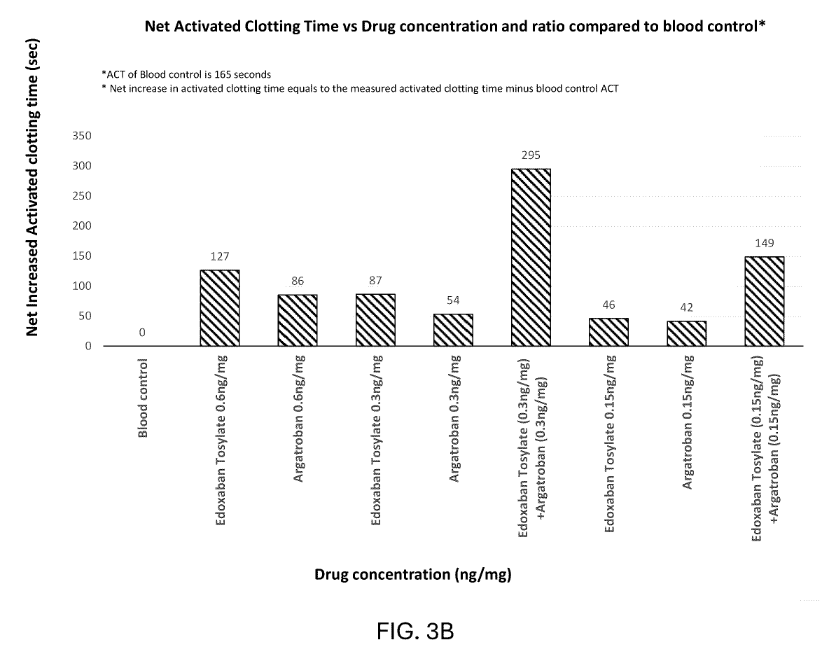

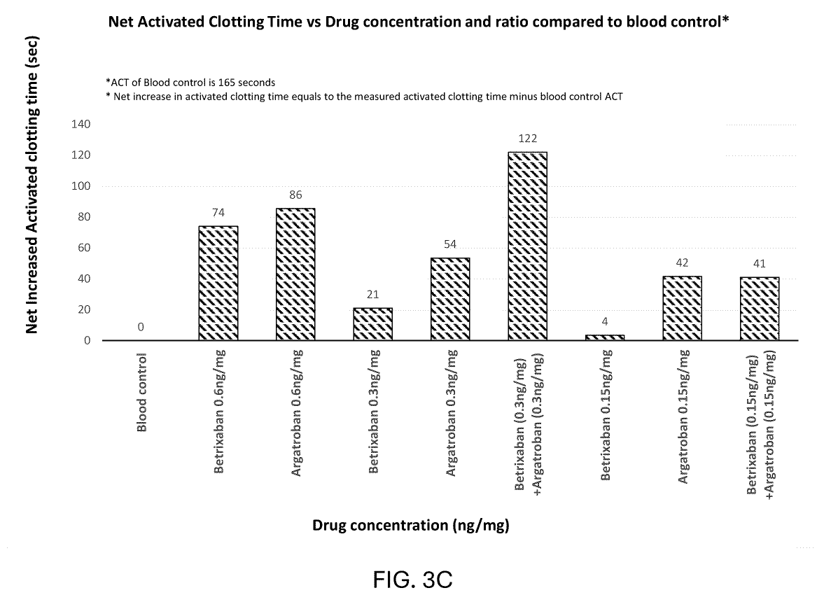

FIG. 3A illustrated the net ACT of the combination of Rivaroxaban and Argatroban. It was found, Unexpectedly, that the combination of at least of 0.3 ng/mg for each of the drugs Argatroban and Rivaroxaban (0.6 ng/mg total) extended the net ACT by a larger time (more effective) than the net ACT for each drug at 0.3 ng/mg concentration (with net ACT of 536 for the combination versus 193 for the sum of individual drugs with 139 for Rivaroxaban and 54 for Argatroban as shown in FIG. 3A), which is an increase of approximately 2.8 times, suggesting a synergistic effect between direct factor Xa inhibitor Rivaroxaban and direct Factor IIa inhibitor Argatroban.

Furthermore, as shown in FIG. 3A, the net ACT was extended by a greater amount when the combination drug concentrations of at least of 0.15 ng/mg for each of the drug Argatroban and Rivaroxaban (0.3 ng/mg total) were compared to the net ACT for each individual drug at 0.15 ng/mg concentration (the net ACT of 288 for the combination versus 156 for the sum of individual drug with 114 for Rivaroxaban versus 42 for Argatroban). This represents an increase of approximately 1.8 times, suggesting a synergistic effect between direct factor Xa inhibitor Rivaroxaban and direct Factor IIa inhibitor Argatroban.

FIG. 3A demonstrated an unexpected synergistic effect between direct factor Xa inhibitor Rivaroxaban and direct Factor IIa inhibitor Argatroban.

### Example 4: Activated Clotting Time (ACT) Evaluation of Direct Factor Xa Inhibitor (Edoxaban) Alone, Direct Factor IIa Inhibitor (Argatroban) Alone or a Combination of Direct Factor Xa Inhibitor (Edoxaban) and Direct Factor IIa Inhibitor (Argatroban)

Example 4 is the continuation of example 3, which employed the same method as in example 3. The only difference is that the direct factor Xa inhibitor Edoxaban is used in place of direct factor Xa inhibitor Rivaroxaban. The data is shown in FIG. 3B.

With the combination of Argatroban and Edoxaban, the net ACT increased (FIG. 3B). Surprisingly, it was discovered that the combination drug concentrations of 0.3 ng/mg for both Argatroban and Edoxaban (0.6 ng/mg total) extended the net ACT by a large time (more effective) than the net ACT for each drug alone at 0.6 ng/mg concentration (the net ACT of 295 for the combination versus 141 for the sum of individual drug with 87 for Edoxaban and 54 for Argatroban as shown in FIG. 3B), which is approximately 2.1 times, suggesting a synergistic effect between direct factor Xa inhibitor Edoxaban and direct Factor IIa inhibitor Argatroban. At drug concentrations of 0.15 ng/mg for each of the two drugs (0.3 ng/mg total), the combination of Argatroban and Edoxaban extended the net ACT by a large time than the net ACT for each drug alone at 0.15 ng/mg concentration (the net ACT of 149 for the combination versus 88 for the sum of individual drug with 46 for Argatroban and 42 for Edoxaban as shown in FIG. 3B), or by approximately 1.7 times, suggesting a synergistic effect between direct factor Xa inhibitor Edoxaban and direct Factor IIa inhibitor Argatroban.

FIG. 3B demonstrates an unexpected synergistic effect between direct factor Xa inhibitor Edoxaban and direct Factor IIa inhibitor Argatroban.

### Example 5: Activated Clotting Time (ACT) Evaluation of Direct Factor Xa Inhibitor (Betrixaban) Alone, Direct Factor IIa Inhibitor (Argatroban) Alone or a Combination of Direct Factor Xa Inhibitor (Betrixaban) and Direct Factor IIa Inhibitor (Argatroban)

Example 5 is the continuation of example 3, which employed the same method as in example 3. The only difference is that the direct factor Xa inhibitor Betrixaban is used in place of direct factor Xa inhibitor Rivaroxaban. The data is shown in FIG. 3C.

When betrixaban and argatroban were combined, the net ACT increased (FIG. 3C). The unexpected finding was that the net ACT was longer for the combination drug concentrations of 0.3 ng/mg for each of the drugs Argatroban and Betrixaban (0.6 ng/mg total) than for the net ACT for each drug at 0.3 ng/mg concentration (the net ACT of 122 for the combination versus 75 for the sum of individual drug with 21 for Betrixaban and 54 for Argatroban as shown in FIG. 3C), which is approximately 1.6 times, suggesting a synergistic effect between direct factor Xa inhibitor Betrixaban and direct Factor IIa inhibitor Argatroban at 0.3 ng/mg.

### Example 6: Activated Clotting Time (AC) Evaluation Initiated by Platelet Activation Factor (PAF) and Calcium Chloride (CaCl2) of Direct Factor Xa Inhibitor Rivaroxaban, Direct Factor IIa Inhibitor Argatroban, Glycoprotein IIb/IIIa Inhibitor Tirofiban, P2Y12 Inhibitor Ticagrelor and Anti-Inflammatory Colchicine Alone or a Combination

In order to examine the potential synergistic effects between anti-inflammatory agents, direct factor Xa inhibitors, direct factor IIa inhibitors, antiplatelets (P2Y12 inhibitor and glycoprotein IIb/IIIa inhibitor, platelet activation factor and CaCl2 were utilized by measuring ACT. Heparinized sheep blood was used to preserve platelet function.

The activated clotting time (ACT) evaluation initiated by platelet activation factor (PAF) and calcium chloride (CaCl2) was performed in heparinized sheep blood and recorded employing the Hemochron® Response device. 1.8 ml of Heparinized sheep blood (Hemostat Lab, Lot 806597-1, Heparin 3 IU/ml, gently swirled it before use) was added to a test tube containing an activator (Hemochron@Celite@ ACT tubes, Lot C3FTE011 from Accriva Diagnostics, Inc.). A target amount of drug solution was then added into the test tube. The test tube was gently swirled so that the blood and drug were well-mixed. 0.1 ml of 0.3M calcium chloride (Sigma-Aldrich, Lot SLBP9500C) was then added and 0.1 ml of 5 mg/ml platelet activation factor (Sigma-Aldrich Lot 4094200-5 mg, stored at −20° C.) in sterilized water was then added. The tube was gently shaken before being inserted into the Hemochron Response detector. The ACT read out was recorded and reported. The ACT of the control heparinized sheep blood in the absence of any drug as first measured to establish a baseline. The blood control is 235 seconds in this study. Then ACT was measured in the presence of different concentrations of the drug measured. Selected drug combinations were then tested to evaluate for potential synergy in action between the drug combination. The net ACT (ACT minus blood control ACT) was utilized to visualize the synergistic effect. The synergistic effect may be particularly important when delivering these drugs locally (to tissue) to inhibit clot formation. The figures are presented in ng/mg wherein the density of blood and tissue are approximately the same.

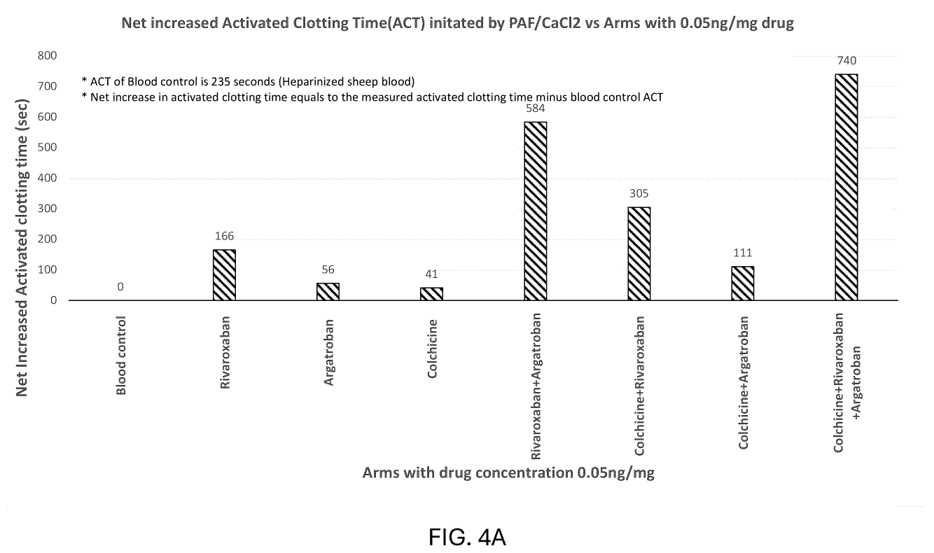

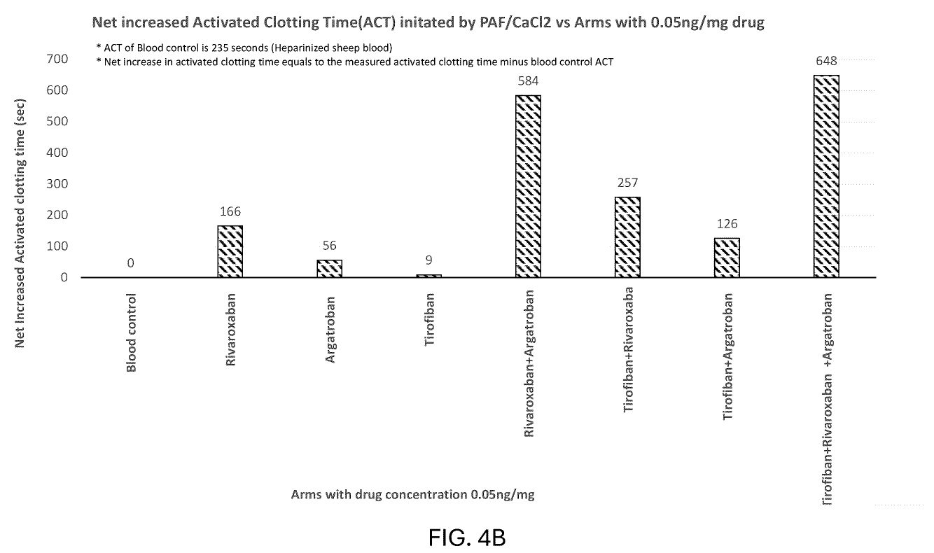

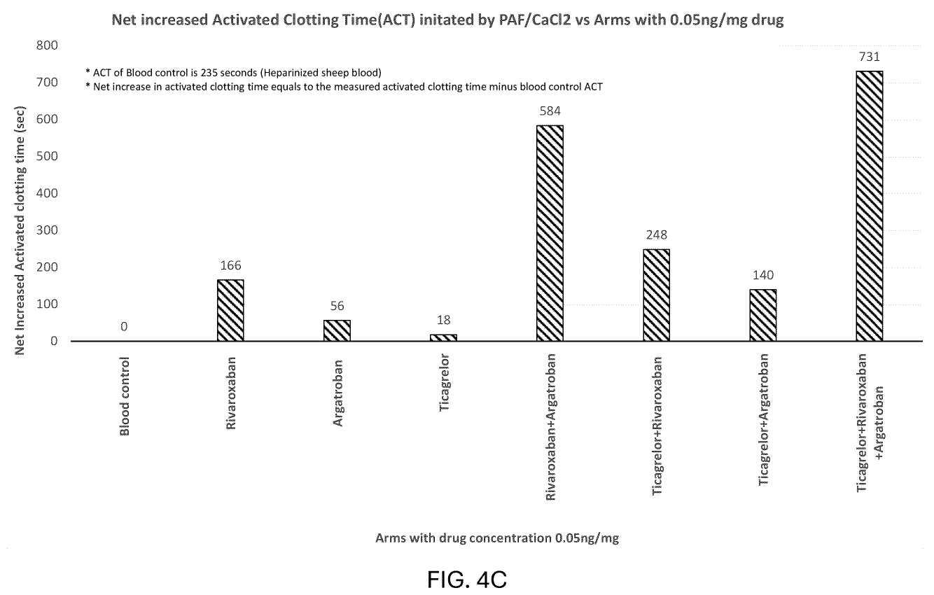

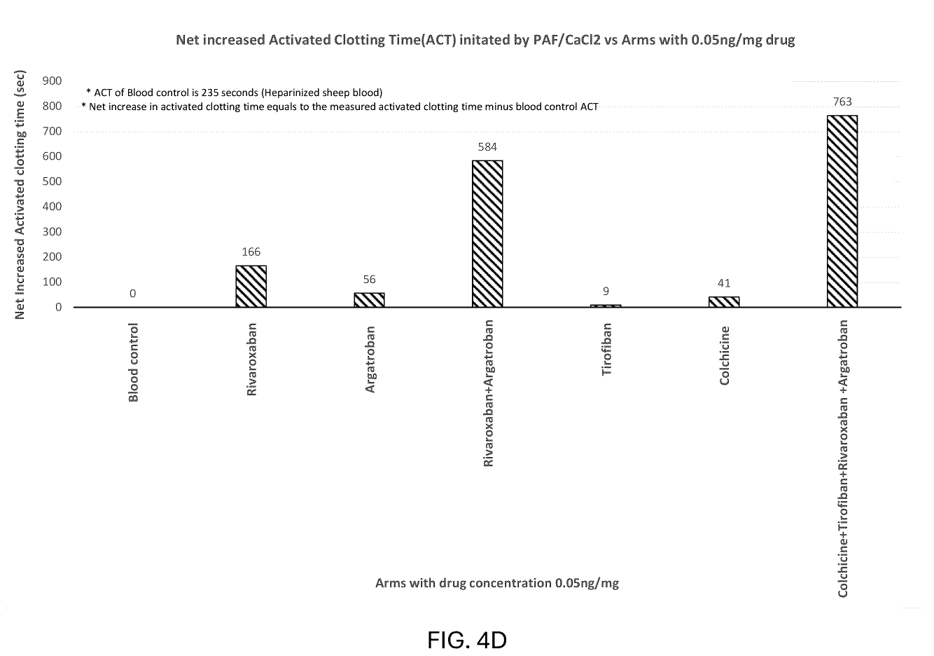

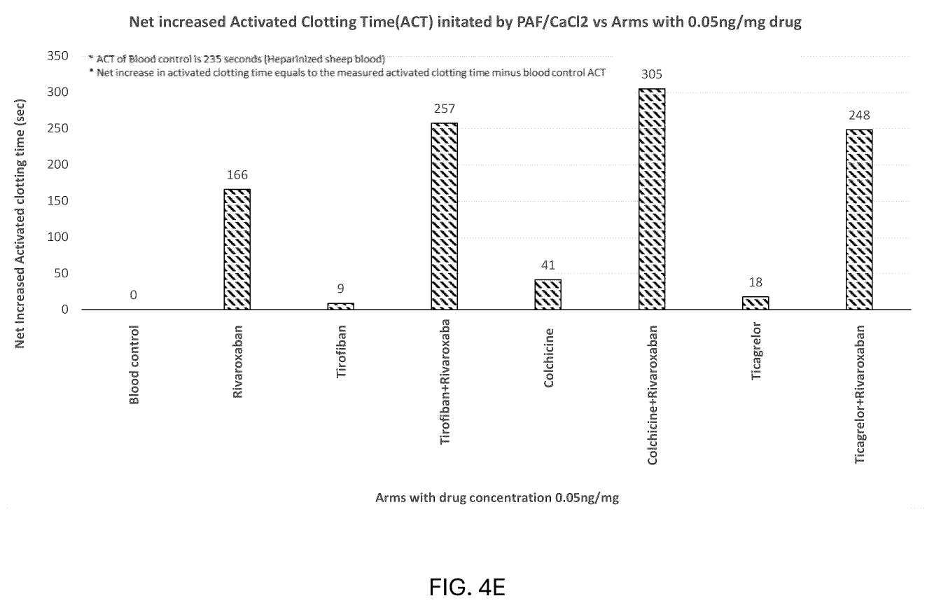

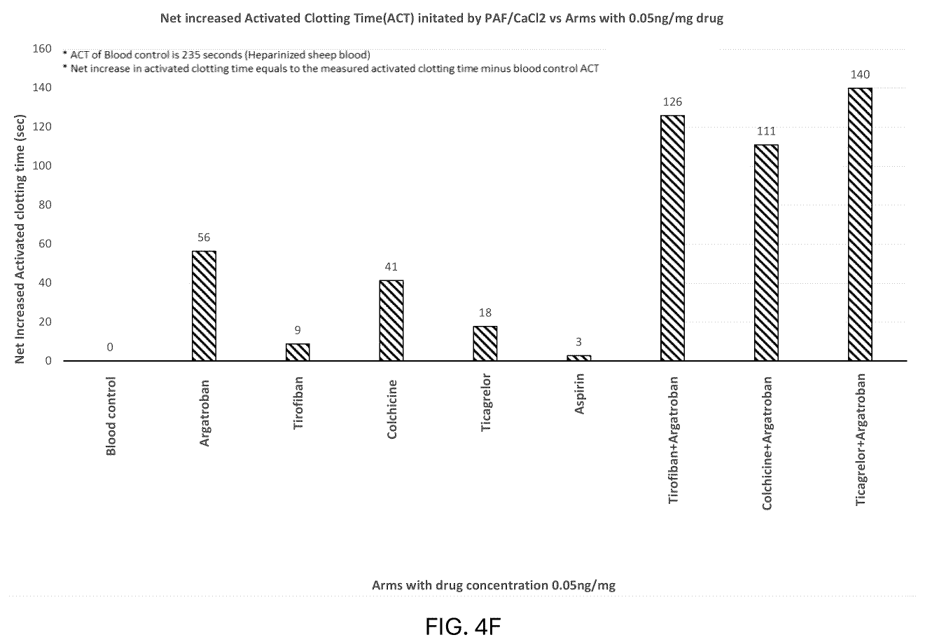

FIG. 4A illustrated the net ACT when Colchicine was combined with direct factor Xa inhibitor (Rivaroxaban) and direct factor IIa inhibitor (Argatroban). It was found, unexpectedly, that the net ACT was extended at a larger time when Colchicine was combined with Rivaroxaban at 0.05 ng/mg of each drug than when each drug was used alone at concentration of 0.05 ng/mg. The combination of rivaroxaban and colchicine resulted in a net increase in ACT of 305 seconds, which was greater than the total of the individual net ACT values of 205 seconds (166 seconds for rivaroxaban and 41 seconds for colchicine), indicating a synergistic effect between Colchicine and Rivaroxaban combination; however, when Colchicine and Argatroban were combined at 0.05 ng/mg of each drug, it showed lesser synergetic effect. The net increase in ACT that was about the same (111 seconds for the combination of Argatroban and Colchicine than the total of the individual net ACT values (97 seconds for the total of the individual drugs with 56 seconds for Argatroban and 41 seconds for Colchicine).

The combination of Colchicine with Argatroban and Rivaroxaban at least 0.05 ng/mg increased the net ACT by approximately 2.8 times (FIG. 4A) compared to the net ACT for each drug alone at 0.05 ng/mg concentration (740 seconds for the combination of Colchicine and Rivaroxaban and Argatroban vs 263 seconds for the total of the individual drugs with 166 seconds for Rivaroxaban, 56 seconds for Argatroban and 41 seconds for Colchicine), by approximately 2.8 times, suggesting a synergistic effect between Colchicine with Argatroban and Rivaroxaban combinations.

The study above demonstrates an unexpected synergistic effect of anti-inflammatory Colchicine with direct factor Xa inhibitor Rivaroxaban, but to a lesser degree with direct factor IIa inhibitor Argatroban; however, there is a larger synergistic effect with Colchicine in combination with Argatroban and Rivaroxaban. This finding is particularly important when delivering these drugs site specifically, locally (to tissue) to inhibit inflammation and/or clot formation.

### Example 7: Activated Clotting Time (ACT) Evaluation Initiated by Platelet Activation Factor (PAF) and Calcium Chloride (CaCl2)) of Direct Factor Xa Inhibitor Rivaroxaban, Direct Factor IIa Inhibitor Argatroban, Glycoprotein IIb/IIIa Inhibitor Tirofiban, P2Y12 Inhibitor Ticagrelor and Anti-Inflammatory Colchicine Alone or a Combination

Example 7 is the continuation of example 6. The data is shown in FIG. 4B.

FIG. 4B evaluated the synergy effect between glycoprotein IIb/IIIa inhibitor Tirofiban with Rivaroxaban and/or Argatroban at 0.05 ng/mg each drug extended the net ACT initiated by PAF& CaCl2.

It was found, unexpectedly, that the combination of Tirofiban with Rivaroxaban or Argatroban at 0.05 ng/mg each drug extended the ACT by a larger time (as shown in FIG. 4B) than the ACT for each individual drug at 0.05 ng/mg concentration. The net increase was higher (257 seconds for the combination of Rivaroxaban and Tirofiban) than the sum of the individual net ACT values (178 seconds for the sum of individual drugs with 166 seconds for Rivaroxaban and 9 seconds for Tirofiban), or by approximately 1.4 times, indicating a synergistic effect between Tirofiban and Rivaroxaban combinations.

The same trend was observed with the combination of Tirofiban with Argatroban at 0.05 ng/mg each drug. The net ACT was higher (126 seconds for the combination of Argatroban and Tirofiban) than the sum of the individual net ACT values (65 seconds for the sum of individual drugs with 56 seconds for Argatroban and 9 seconds for Tirofiban), or by approximately 1.9 times, indicating a synergistic effect between Tirofiban and Argatroban drug combinations.

When Tirotiban combined with Argatroban and Rivaroxaban at concentration of 0.05 ng/mg, the net ACT increased by around 2.8 times (648 seconds for the combination vs 231 seconds for the total of the individual drugs with 166 seconds for Rivaroxaban, 56 seconds for Argatroban, and 9 seconds for Tirotiban), or by approximately 2.8 times, indicating a synergistic effect between all three drug combinations.

The study above demonstrated a synergistic effect between anti-platelet (glycoprotein IIb/IIIa inhibitor) and anti-coagulation (direct factor Xa inhibitor and/or direct factor IIa inhibitor). This finding is particularly important when delivering these drugs locally (adjacent to injured tissue) to clot formation.

### Example 8: Activated Clotting Time (ACT) Evaluation Initiated by Platelet Activation Factor (PAF) and Calcium Chloride (CaCl2)) of Direct Factor Xa Inhibitor Rivaroxaban, Direct Factor IIa Inhibitor Argatroban, Glycoprotein IIb/IIIa Inhibitor Tirofiban, P2Y12 Inhibitor Ticagrelor and Anti-Inflammatory Colchicine Alone or a Combination

Example 8 is the continuation of example 6. The data is shown in FIG. 4C.

FIG. 4C investigated the synergy effect between anti-platelet (P2Y12 inhibitor) and anti-coagulation (direct factor Xa inhibitor and/or direct factor IIa inhibitor).

It was found, unexpectedly, that the combination of Ticagrelor with Rivaroxaban or Argatroban at 0.05 ng/mg each drug extended the ACT by a larger time (as shown in FIG. 4C) than the ACT for each individual drug at 0.05 ng/mg concentration. The net increase ACT was higher (248 seconds for the combination of Rivaroxaban and Ticagrelor) than the sum of the individual net ACT values (184 seconds for the sum of individual drugs with 166 seconds for Rivaroxaban and 18 seconds for Ticagrelor), or by approximately 1.3 times, indicating a synergistic effect between Rivaroxaban and Ticagrelor drug combinations.

The same trend was observed of the combination of Tirofiban with Argatroban at 0.05 ng/mg each drug. The net increase ACT was higher (140 seconds for the combination of Argatroban and Ticagrelor) than the sum of the individual net ACT values (74 seconds for the sum of individual drugs with 56 seconds for Argatroban and 18 seconds for Ticagrelor), or by approximately 1.9 times, indicating a synergistic effect between Tirofiban with Argatroban drug combinations.

When Ticagrelor combined with Rivaroxaban and Argatroban at 0.05 ng/mg each drug extended the net ACT by about 3.0 times (731 seconds for the combination of Rivaroxaban and Argatroban and Ticagrelor) than the net ACT for each individual drug at 0.05 ng/mg concentration (240 seconds for the sum of individual drugs with 166 seconds for Rivaroxaban and 56 seconds for Argatroban and 18 seconds for Ticagrelor), or by approximately 3.0 times, indicating a synergistic effect between Rivaroxaban and Argatroban and Ticagrelor drug combinations.

The study above demonstrated an unexpected synergistic effect of glycoprotein IIb/IIIa anti-platelet Tirofiban with direct factor Xa inhibitor Rivaroxaban and direct factor IIa inhibitor Argatroban. This finding is particularly important when delivering these drugs site specifically, locally (to tissue) to inhibit clot formation.

### Example 9: Activated Clotting Time (ACT) Evaluation Initiated by Platelet Activation Factor (PAF) and Calcium Chloride (CaCl2) of Direct Factor Xa Inhibitor Rivaroxaban, Direct Factor IIa Inhibitor Argatroban, Glycoprotein IIb/IIIa Inhibitor Tirofiban, P2Y12 Inhibitor Ticagrelor and Anti-Inflammatory Colchicine Alone or a Combination

Example 9 is the continuation of example 6. The data is shown in FIG. 4D.

FIG. 4D examined the synergistic effects of anti-coagulation (direct factor Xa inhibitor and/or direct factor IIa inhibitor), anti-platelet P2Y12 inhibitor, anti-inflammatory, and anti-platelet glycoprotein IIb/IIIa inhibitor. The active clotting time was shown to be considerably prolonged when drugs were combined, as illustrated in FIG. 4D. The Rivaroxaban and Argatroban combination achieved ACT levels higher (The net ACT is 584 seconds) than the sum of the individual net ACT values (222 seconds with Rivaroxaban 166 seconds and Argatroban 56 seconds, by approximately 2.6 times, indicating a synergistic effect between these two drug combinations.

It was found, unexpectedly, that the combination drug concentrations of Rivaroxaban and Argatroban and Tirofiban and Colchicine at 0.05 ng/mg each drug extended the ACT by a larger time (as shown in FIG. 4D) than the ACT for each individual drug at 0.05 ng/mg concentration. The net increase ACT was higher (763 seconds for the combination of Rivaroxaban and Argatroban and Colchicine) than the sum of the individual net ACT values (269 seconds for the sum of individual drugs with 166 seconds for Rivaroxaban and 56 seconds for Argatroban and 9 seconds for Tirofiban and 41 seconds for Colchicine), or by approximately 2.8 times, indicating a synergistic effect between Rivaroxaban and Argatroban and Tirofiban and Colchicine combinations.

The study above demonstrated an unexpected synergistic effect of anti-inflammatory Colchicine, direct factor Xa inhibitor Rivaroxaban, direct factor Fla inhibitor Argatroban and glycoprotein IIb/IIa anti-platelet Tirofiban. This finding is particularly important when delivering these drugs site specifically, locally (to tissue) to inhibit inflammation and/or clot formation.

### Example 10: Activated Clotting Time (ACT) Evaluation Initiated by Platelet Activation Factor (PAF) and Calcium Chloride (CaCl2)) of Direct Factor Xa Inhibitor Rivaroxaban, Direct Factor IIa Inhibitor Argatroban, Glycoprotein IIb/IIIa Inhibitor Tirofiban, P2Y12 Inhibitor Ticagrelor and Anti-Inflammatory Colchicine Alone or a Combination

Example 10 is the continuation of example 6. The data is shown in FIG. 4E.

FIG. 4E investigated the synergy effect between direct factor Xa inhibitor and anti-inflammatory/anti-platelet glycoprotein IIb/IIIa inhibitor/anti-platelet P2Y12 inhibitor. As shown in FIG. 4E, the activated clotting time was observed to be significantly extended or increased when direct factor Xa inhibitor combined with another drug. The same trend was observed with the combination of Rivaroxaban with Tirofiban or Colchicine or Ticagrelor. The Rivaroxaban/Tirofiban combination achieved ACT levels that were higher (257 seconds with the combination) than the sum of the individual net ACT values (178 seconds total with Rivaroxaban 166 seconds and Tirofiban 9 seconds), or by approximately 1.7 times, indicating a synergistic effect between Rivaroxaban and Tirofiban combinations. The Rivaroxaban/Colchicine combination achieved ACT levels that were higher (The net ACT is 305 seconds for the combination) than the sum of the individual net ACT values (207 seconds total with Rivaroxaban 166 seconds and Colchicine 41 seconds), or by approximately 1.5 times, indicating a synergistic effect between these Rivaroxaban and Colchicine combinations. The Rivaroxaban/Ticagrelor combination achieved ACT levels that were higher (248 seconds with the combination) than the sum of the individual net ACT values (184 seconds total with Rivaroxaban 166 seconds and Ticagrelor 18 seconds), or by approximately 1.3 times, indicating a synergistic effect between Rivaroxaban and Ticagrelor combinations.

The study above demonstrated an unexpected synergistic effect of direct factor Xa inhibitor Rivaroxaban with Glycoprotein IIb/IIIa inhibitor Tirofiban, with anti-inflammatory Colchicine P2Y12 inhibitor Ticagrelor. This finding is particularly important when delivering these drugs site specifically, locally (to tissue) to inhibit inflammation and/or clot formation.

### Example 11: Activated Clotting Time (ACT) Evaluation Initiated by Platelet Activation Factor (PAF) and Calcium Chloride (CaCl2)) of Direct Factor Xa Inhibitor Rivaroxaban, Direct Factor IIa Inhibitor Argatroban, Glycoprotein IIb/IIIa Inhibitor Tirofiban, P2Y12 Inhibitor Ticagrelor and Anti-Inflammatory Colchicine Alone or a Combination

Example 11 is the continuation of example 6. The data is shown in FIG. 4F. FIG. 4F investigated the synergistic effect between direct factor IIa inhibitor and anti-inflammatory, and anti-platelet glycoprotein IIb/IIIa inhibitor, and anti-platelet P2Y12 inhibitor. As shown in FIG. 4F, the activated clotting time was observed to be significantly extended or increased when direct factor Xa inhibitor combined with another drug. The Argatroban and Tirofiban combination achieved ACT levels higher (126 seconds for the combination) than the sum of the individual net ACT values (67 seconds total with Argatroban 56 seconds and Tirotiban 9 seconds), or by approximately 1.9 times, indicating a synergistic effect between Argatroban and Tirofiban combinations. The Argatroban and Ticagrelor combination achieved ACT levels higher (The net ACT is 140 seconds for the combination) than the sum of the individual net ACT values (74 seconds total with Argatroban 56 seconds and Ticagrelor 18 seconds), or by approximately 1.9 times, indicating a synergistic effect between Argatroban and Ticagrelor combinations. However, the Argatroban/Colchicine combination achieved ACT levels is about the same (The net ACT is 111 seconds of the combination) as the sum of the individual net ACT values (97 seconds with Argatroban 56 seconds and Colchicine 41 seconds), indicating lesser degree of synergistic effect between Argatroban and Colchicine combinations.

The study above demonstrated an unexpected synergistic effect of direct factor IIa inhibitor with anti-inflammatory Colchicine, with anti-platelet glycoprotein IIb/IIIa inhibitor Tirotiban, with anti-platelet P2Y12 inhibitor Ticagrelor. This finding is particularly important when delivering these drugs site specifically locally (to tissue) to inhibit inflammation and/or clot formation.

### Example 12: Activated Clouting Time (ACT) Evaluation of EDTA or Analogues in Fresh Pig Blood

The activated clotting time (ACT) evaluation of EDTA or analogues was performed in fresh pig blood and recorded employing the Hemochron® Response device.

The ACT measurements were made in fresh pig blood. 1.0 ml of fresh pig blood was added to a test tube containing an activator (Hemochron@Celite@ ACT tubes, Lot F8FTE026 from Accriva Diagnostics, Inc.). A target amount of drug solution was then added into the test tube. The test tube was gently swirled so that the blood and drug was well-mixed. 0.1 ml of 0.3M calcium chloride was then added. The tube was gently shaken before being inserted into the Hemochron Response detector. The ACT read out was recorded and reported. The ACT of the control blood in the absence of any drug as first determined to establish a baseline. Then ACT was determined in the presence of different concentrations of EDTA or analogues as a single component.

As shown in Table 1B, the clotting time was observed to be significantly extended or increased at a higher drug combination concentration. The EDTA or analogues achieved ACT levels that were higher than the control ACT values, indicating anti-coagulant effect. This may be particularly important when delivering EDTA or analogues locally (adjacent to injured tissue) to inhibit clot formation. The figures are presented in ng/mg wherein the density of blood and tissue are approximately the same.

TABLE 1B
Activated clotting time (ACT) versus EDTA or analogues
concentration in fresh pig blood

| | | | Increase in |
|---|---|---|---|
| | | | clotting |
| | Final Drug | | time |
| | Con- | Clotting | compared |
| | centration | time | to control |
| Sample | (ng/mg) | (sec) | (sec) |
| Blood control | 0 | 154 | 0 |
| Dimercaptosuccinic acid | 200 | 151 | −3 |
| (DMSA) 200 μg/ml | | | |
| EDTA tetra acetoxymethyl | 500 | 158 | 4 |
| ester 500 μg/ml | | | |
| EDTA 75 μg/ml | 75 | 179 | 25 |
| EDTA 100 μg/ml | 100 | 247 | 93 |
| EDTA 150 μg/ml | 150 | 275 | 121 |
| EDTA 200 μg/ml | 200 | >500 | >346 |
| EDTA•4Na•xH2O 100 μg/ml | 100 | 204 | 50 |
| EDTA•4Na•xH2O 200 μg/ml | 200 | 275 | 121 |
| EDTA•4Na•xH2O 250 μg/ml | 250 | 329 | 175 |
| EDTA•4Na•xH2O 300 μg/ml | 300 | >500 | >346 |
| EDTA•2Na•2H2O 100 μg/ml | 100 | 191 | 37 |
| EDTA•2Na•2H2O 200 μg/ml | 200 | 264 | 110 |
| EDTA•2Na•2H2O 300 μg/ml | 300 | 374 | 220 |

As shown in Table 1B, EDTA at 200 ng/mg in fresh pig blood, had significant delayed blood coagulation compared to the blood control. The same trend was observed for EDTA tetra sodium salt and EDTA disodium salt at 300 ng/mg. It is important to note that drug tissue concentrations for EDTA or analogue to have sufficient tissue concentrations in the stented tissue segment and in the adjacent tissue segment to have therapeutic levels to be larger than 300 ng/mg of tissue at or within 3 hours after implantation, or at or within 1 day after implantation, to inhibit clot formation.

Based on the pH of EDTA or analogues in water and in 0.1M phosphate buffered saline as show in Table 2, EDTA disodium salt or dipotassium salt has a pH close to neutral pH in human body (6.42-6.92), whereas EDTA tetrasodium or trisodium has a very basic pH (>8.0) in human body. EDTA disodium or dipotassium is preferred in the examples.

TABLE 2
pH of 1 mg/ml EDTA or analogue in Water
or 0.1 M pH 7.4 phosphate buffer.

| | | |
|---|---|---|
| Sample | pH | Temperature,° C. |

1 mg/ml in d-water

| | | |
|---|---|---|
| EDTA disodium | 6.42 | 21.8 |
| EDTA tetra sodium | 9.61 | 21.9 |
| EDTA tri sodium | 9.21 | 21.8 |
| EDTA dipotassium | 6.25 | 22.0 |

1 mg/ml in 0.01 M pH 7.4 phosphate buffer

| | | |
|---|---|---|
| EDTA disodium | 6.92 | 22.3 |
| EDTA tetra sodium | 8.27 | 21.9 |
| EDTA tri sodium | 7.77 | 21.8 |
| EDTA dipotassium | 6.93 | 22.3 |

### Example 13: Activated Clotting Time (ACT) Evaluation of EDTA or Other Anti-Coagulant Compounds in Citrated Sheep Blood

The activated clotting time (ACT) evaluation of anticoagulants was performed in Calcium-reconstituted sheep blood and recorded employing the Hemochron® Response device.

The ACT measurements were made in citrated sheep blood. 1.9 ml of citrated sheep blood was added to a test tube containing an activator (Hemochron@Celite@ ACT tubes, Lot F8FTE026 from Accriva Diagnostics, Inc.). A target amount of EDTA or other anti-coagulant compounds solution was then added into the test tube. The test tube was gently swirled so that the blood and EDTA or other anti-coagulant compounds was well-mixed. 0.1 ml of 0.3M calcium chloride was then added. The tube was gently shaken before being inserted into the Hemochron Response detector. The ACT read out was recorded and reported. The ACT of the control blood in the absence of any EDTA or other anti-coagulant compounds as first determined to establish a baseline. Then ACT was determined in the presence of different concentrations of EDTA or analogues as a single component.

As shown in Table 3, the clotting time was observed to be significantly extended or increased at a higher EDTA or other anti-coagulant compounds concentration. The EDTA or other anti-coagulant compounds achieved ACT levels that were higher than the control ACT values, indicating anti-coagulant effect. This may be particularly important when delivering EDTA or other anti-coagulant compounds locally (adjacent to injured tissue) to inhibit clot formation. The figures are presented in ng/mg wherein the density of blood and tissue are approximately the same.

TABLE 3
Activated clotting time (ACT) versus EDTA or other anti-
coagulant compounds concentration in citrated sheep blood

| | | | | Increase |
|---|---|---|---|---|
| | | | | in clotting |
| | Final | | | time |
| | Concen- | Clotting | Blood | compared |
| | tration | time | control | to control |
| Sample | (ng/mg) | (sec) | (sec) | (sec) |
| EDTA.2Na.28,0 with | 200 | >500 | 236 | >264 |
| Benzyldimethyltetradecyl- | | | | |
| ammonium chloride | | | | |
| complex 2(0) μg/ml | | | | |
| Benzyldimethyltetradecyl- | 67 | 316 | | 114 |
| ammonium chloride | | | | |
| 66.7 μg/ml | | | | |
| Benzyldimethyltetradecyl- | 100 | 421 | | 219 |
| ammonium chloride | | | | |
| 100 μg/ml | | | | |
| Benzyldimethyltetradecyl- | 133 | 983 | | 781 |
| ammonium chloride | | | | |
| 133.3 μg/ml | | | | |
| Benzyldimethyltetradecyl- | 167 | 1370 | 202 | 1168 |
| ammonium chloride | | | | |
| 166.7 μg/ml | | | | |
| Polyethylenimine, Linear, | 3 | 203 | | −14 |
| MW(250 K), 3.3 μg/ml | | | | |
| Polyethylenimine, Linear, | 8 | 188 | | −29 |
| MW(250 K), 8.3 μg/ml | | | | |
| Polyethylenimine, Linear, | 17 | >500 | | >283 |
| MW(250 K), 16.7 μg/ml | | | | |
| Polyethylenimine, Linear, | 33 | >500 | 217 | >283 |
| MW(250 K), 33.3 μg/ml | | | | |
| Polyethylenimine, Linear, | 3 | 198 | | −19 |
| MW(100 K), 3.3 μg/ml | | | | |
| Polyethylenimine, Linear, | 8 | 188 | 217 | −29 |
| MW(100 K), 8.3 μg/ml | | | | |
| Polyethylenimine, Linear, | 1.7 | >500 | | >283 |
| MW(100 K), 16.7 μg/ml | | | | |
| Polyethylenimine, Linear, | 17 | 303 | | >283 |
| MW(2.5 K), 16.7 μg/ml | | | | |
| Polyethylenimine, Linear, | 25 | 331 | | 129 |
| MW(2.S K), 25.0 μg/m | | | | |
| Polyethylenimine, Linear, | 33 | 668 | | 466 |
| MW(2.5 K), 33.3 μg/ml | | | | |
| Polyethylenimine, Linear, | 67 | 814 | | 612 |
| MW(2.5 K), 66.7 μg/ml | | | | |
| Polyethylenimine, Linear, | 100 | >793 | 202 | >591 |
| MW(2.5 K), 100 μg/ml | | | | |

It is important to note that drug tissue concentrations for EDTA or analogue to have sufficient tissue concentrations in the stented tissue segment and in the adjacent tissue segment to have therapeutic levels to be larger than 200 ng/mg of tissue with EDTA complex, or to be larger than 100 ng/mg of tissue with Benzyldimethyltetradecylammonium chloride, or to be larger than 33 ng/mg of tissue with linear Polyethylenimine or within 3 hours after implantation, or within 1 day after implantation, to inhibit clot formation.

### Example 14: Preparation of Anticoagulant EDTA Eluting Stents

Poly(L-lactide acid) polymer was dissolved into tetrahydrofuran (THF) at room temperature or heated 50° C. when needed, vortexed until the polymer had uniformly dissolved/dispersed. EDTA·4Na·xH₂O was dissolved into HPLC water at room temperature, stirred overnight until the drug was uniformly dissolved/dispersed.

Each polymer solution and each EDTA solution were combined together (EDTA to Poly(L-lactide acid) polymer by weight ratio was 7:3), according to the target EDTA dose. Top coated Poly(L-lactide) as needed per target release profile.

A microprocessor-controlled ultrasonic sprayer was used to coat each of the stents' 14 mm length uniformly with each of the drug/polymer matrix solutions. After coating, the stents were placed in a vacuum chamber to remove the solvent. The stents were then mounted on balloon catheters and crimped. The catheters were then inserted in coils and packaged. The pouches were sterilized.

### Example 15: In Vitro Testing with Fast Release of Stent with EDTA in 0.01M Phosphate Buffered Saline Buffer

In-Vitro Elution evaluation of the EDTA coated stent above prepared was performed in 0.01M phosphate buffered saline at 37° C. water shaking bath then tested on High Performance Liquid Chromatography (HPLC). EDTA was separated from interfering peaks by a Carbon18 reverse phase column and quantified with an external EDTA calibration standard solution prepared at similar concentrations. The percentage of EDTA eluted was calculated during each time point.

TABLE 4
Cumulative percent release of EDTA percentage of
release profile in 0.01 M phosphate buffered saline

| Time | 5 min | 10 min |
|---|---|---|
| 800 μg EDTA•4Na•xH₂O in Poly(L-Lactide | 99.8 | 100.0 |
| acid) matrix with 200 μg Poly(L-Lactide acid) | | |
| topcoat | | |
| 800 μg EDTA•4Na•xH₂O in Poly(L-Lactide | 99.8 | 100.0 |
| acid) matrix EDTA.4Na.xH2O with 300 μg | | |
| Poly(L-Lactide acid) topcoat | | |

The data in Table 4 shows that about 99.8% EDTA tetra sodium eluted out about 5 minutes with top coated 200 μg Poly(L-lactide acid-co-glycolic acid) or with top coated 300 μg Poly(L-lactide acid-co-glycolic acid).

### Example 16: Preparation of Anticoagulant Tetra Acetoxymethyl Ester EDTA Eluting Stens

Poly(L-lactide acid-co-glycolic acid) or Poly(L-lactide acid) polymer was dissolved into dichloromethane (DCM) at room temperature, vortexed until the polymer had uniformly dissolved dispersed. Tetra acetoxymethyl ester EDTA was dissolved into dichloromethane (DCM) at room temperature, stirred overnight until uniformly dissolved/dispersed.

Arm A was coated with 800 μg Tetra acetoxymethyl ester EDTA only; Arm B was coated with 800 μg Tetra acetoxymethyl ester EDTA solution then top coated 100 μg Nolvolimus in poly(L-lactide acid-co-glycolic acid) matrix (drug to polymer ratio of 2:3); Arm C was coated with 300 μg Tetra acetoxymethyl ester EDTA in 600 μg poly(L-lactide acid) matrix.

A microprocessor-controlled ultrasonic sprayer was used to coat each of the stents' 14 mm length uniformly with each of the drug/polymer matrix solutions. After coating, the stents were placed in a vacuum chamber to remove the solvent. The stents were then mounted on balloon catheters and crimped. The catheters were then inserted in coils and packaged. The pouches were sterilized.

### Example 17: In Vino Testing of Controlled Release of Stent with Tetra Acetoxymethyl Ester EDTA in 0.01M Phosphate Buffered Saline

In-Vitro Elution evaluation of Tetra acetoxymethyl ester EDTA coated stent was performed in 0.01M phosphate buffered saline at 37° C. water shaking bath then tested on High Performance Liquid Chromatography (HPLC). Tetra acetoxymethyl ester EDTA was separated from interfering peaks by a Carbon18 reverse phase column and quantified with an external tetra acetoxymethyl ester EDTA calibration standard solution prepared at similar concentrations. The percentage of tetra acetoxymethyl ester EDTA eluted was calculated during each time point.

TABLE 5
Cumulative percent release of tetra acetoxymethyl ester EDTA
percentage of release profile in 0.01 M phosphate buffered saline

| | 5 | 10 | 15 | Left after |
|---|---|---|---|---|
| Time | min | min | min | 15 min |
| Arm A: 800 μg Tetra acetoxymethyl | 71.4 | 76.0 | 77.0 | 23.0 |
| ester EDTA only | | | | |
| Arm B: 800 μg Tetra acetoxymethyl | 46.0 | 68.3 | 77.4 | 22.6 |
| ester EDTA with top coated with | | | | |
| 100 μg Novolimus in 175 μg Poly(L- | | | | |
| lactide acid-co-glycolic acid) matrix | | | | |
| Arm C: 300 μg Tetra acetoxymethyl | 6.8 | 14.0 | 14.0 | 86.0 |
| ester EDTA in 600 μg Poly(L-lactide | | | | |
| acid) matrix | | | | |

The data in Table 5 shows that with top coated 175 μg of Poly(L-lactide acid-co-glycolic acid) and 100 μg of Novolimus, the release rate of Tetra acetoxymethyl ester EDTA was extended to about 10 minutes to 15 minutes (Arm B). With tetra acetoxymethyl ester EDTA and poly(L-lactide acid) matrix (Arm C), the release rate was further slowdown beyond 15 minutes. Arm B released around 200 μg of EDTA between 5 minutes to 10 minutes in the studied dose, having an effective therapeutic level to inhibit clot formation.

### Example 18: Preparation of Anticoagulant EDTA Complex Eluting Stents

EDTA complex was made as below: 3 moles of EDTA·2Na·2H2O with 1 mole of Benzyldimethyltetradecylammonium chloride was reacted in room temperature overnight. The complex was purified by removing sodium chloride and excessive EDTA·2Na·2H2O then vacuum dried.

Poly(L-lactide acid-co-glycolic acid) polymer was dissolved into dichloromethane (DCM) at room temperature and vortexed until the polymer had uniformly dissolved/dispersed. EDTA·2Na·2H2O with benzyldimethyltetradecylammonium complex was dissolved into ethanol at room temperature and stirred until uniformly dissolved/dispersed.

Each polymer solution and each EDTA complex were combined together (EDTA to Poly(L-lactide acid-co-glycolic acid) polymer by weight ratio was 3:2), according to the target EDTA dose as shown in Arm D, Arm E and Arm F. Top coated Poly(L-lactide acid-co-glycolic acid) as needed per release profile.

A microprocessor-controlled ultrasonic sprayer was used to coat each of the stents' 14 mm length uniformly with each of the excipient/polymer matrix solutions. After coating, the stents were placed in a vacuum chamber to remove the solvent. The stents were then mounted on balloon catheters and crimped. The catheters were then inserted in coils and packaged. The pouches were sterilized.

### Example 19: In Vitro Testing of Controlled Release of Stent with EDTA Complex in 0.01M Phosphate Buffered Saline

In-Vitro Elution evaluation of the EDTA complex coated stent was performed in 0.01M phosphate buffered saline at 37° C. water shaking bath then tested on High Performance Liquid Chromatography (HPLC). EDTA disodium was separated from interfering peaks by a Carbon18 reverse phase column and quantified with an external EDTA disodium calibration standard solution prepared at similar concentrations. The percentage of EDTA disodium eluted was calculated during each time point as listed below.

TABLE 6
Cumulative percent release of EDTA disodium percentage of release profile in 0.01 M
phosphate buffered saline

| | | | | | | | | | Total |
|---|---|---|---|---|---|---|---|---|---|
| | | | | | | | | | eluted |
| | | | | | | | | | +24 hrs |
| Time | 2 min | 10 min | 15 min | 20 min | 25 min | 30 min | 3 hrs | 24 hrs | left |
| Arm D: 1200 μg EDTA.2Na.2H₂O with | 72.0 | 75.2 | 76.9 | 79.1 | 80.4 | 83.0 | 87.2 | 92.0 | 99.9 |
| benzyldimethyltetradecylammonium complex is | | | | | | | | | |
| 800 μg Poly(L-lactide acid-co-glycolic acid) | | | | | | | | | |
| matrix | | | | | | | | | |
| Arm E: 1200 μg EDTA.2Na.2H₂O with | 10.2 | 16.7 | 23.8 | 33.1 | 37.3 | 43.0 | 88.6 | 97.9 | 99.9 |
| benzyldime thy letradecylammonium complex in | | | | | | | | | |
| 800 μg Poly(L-lactide acid-co-glycolie acid) | | | | | | | | | |
| matrix with 200 μg Poly(L-lactide acid-co- | | | | | | | | | |
| glycolic acid) topcoat | | | | | | | | | |
| Arm F: 1200 μg EDTA.2Na.2H₂O with | 8.6 | 12.4 | 35.4 | N/A | N/A | 21.2 | 45.7 | 62.3 | 100 |
| benzyldimethyltetradecy lammonium complex in | | | | | | | | | |
| 800 μg Poly(L-lactide acid-co-glycolic acid) | | | | | | | | | |
| matrix with 400 μg Poly(L-lactide acid-co- | | | | | | | | | |
| glycolic acid) topcoat | | | | | | | | | |

The data in Table 6 shows that about 72% EDTA disodium eluted out from EDTA complex at 2 minutes without topcoat (Arm D); extended release of EDTA disodium is achieved with 200 μg topcoat Poly(L-lactide acid-co-glycolic acid) (Arm E) to 3 hours (about 88.6% eluted out up to 3 hours), with increased topcoat of 400 μg of Poly(L-lactide acid-co-glycolic acid) (Arm E), the release extended beyond 24 hours (about 62.1% eluted out up to 24 hours). Arm E and Arm F released around 300 μg to 550 μg of EDTA between 30 minutes to 3 hours in the studied dose, indicating an effective therapeutic level to inhibit local clot formation.

### Example 20: Preparation of Metal Stents with Cationic Anti-Coagulation Enhancer Benzyldimethyltetradecyl Ammonium Chloride

Poly(L-lactide acid-co-glycolic acid) polymer was dissolved into dichloromethane (DCM) at room temperature and vortexed until the polymer had uniformly dissolved/dispersed. Cationic anti-coagulation enhancers Benzyl dimethyltetradecyl ammonium chloride was dissolved into ethanol at room temperature and stirred overnight until uniformly dissolved/dispersed.

Each polymer solution and each cationic anti-coagulation enhancers solution were combined together (cationic anti-coagulation enhancers to Poly(L-lactide acid-co-glycolic acid) polymer by weight ratio 7:10), according to the target cationic anti-coagulation enhancers released dose. Top coated Poly(L-lactide acid-co-glycolic acid) as needed per target release profile.

A microprocessor-controlled ultrasonic sprayer was used to coat each of the stents' 14 mm length uniformly with each of the cationic anti-coagulation enhancer/polymer matrix solutions. After coating, the stents were placed in a vacuum chamber to remove the solvent. The stents were then mounted on balloon catheters and crimped. The catheters were then inserted in coils and packaged. The pouches were sterilized.

### Example 21: In Vitro Testing of Extended Release of Stent Coated with Cationic Anti-Coagulation Enhancer Benzyl Dimethyltetradecyl Ammonium Chloride with Poly(L-Lactide Acid-Co-Glycolic Acid) Polymer

In-Vitro Elution evaluation of the stent coated with cationic anti-coagulation enhancer Benzyl dimethyl tetradecyl ammonium chloride and Poly(L-lactide acid-co-glycolic acid) was performed in 0.01M phosphate buffered saline at 37° C. water shaking bath then tested on High Performance Liquid Chromatography (HPLC). Benzyl dimethyl tetradecyl ammonium chloride was separated from interfering peaks by a Carbon18 reverse phase column and quantified with an external Benzyl dimethyl tetradecyl ammonium chloride calibration standard solution prepared at similar concentrations. The percentage of cationic anti-coagulation enhancer eluted was calculated during each time point as listed below.

TABLE 7
Cumulative percent release of cationic anti-coagulation enhancer Benzyl dimethyl
tetradecyl ammonium chloride percentage of release profile in 0.01 M phosphate buffered saline.

| | | | | | | | | | Total eluted |
|---|---|---|---|---|---|---|---|---|---|
| Time | 2 min | 10 min | 15 min | 20 min | 25 min | 30 min | 3 hrs | 24 hrs | +24 hrs left |
| Arm G: 1500 μg benzyl | 20.3 | 30 | 34.6 | 40.4 | 44.9 | 49.1 | 92.0 | 97.8 | 98.3 |
| dimethyl tetradecyl | | | | | | | | | |
| ammonium complex in | | | | | | | | | |
| 643 μg Poly(L-lactide acid- | | | | | | | | | |
| co-glycolic acid) matrix with | | | | | | | | | |
| 200 μg Poly(L-lactide acid- | | | | | | | | | |
| co-glycolic acid) topcoat | | | | | | | | | |
| Arm H: 1500 μg benzyl | 21.5 | 28.7 | 31.9 | 35.3 | 38.1 | 41.2 | 83.9 | 97.1 | 100 |
| dimethyl tetradecyl | | | | | | | | | |
| ammonium complex in | | | | | | | | | |
| 643 μg Poly(L-lactide acid- | | | | | | | | | |
| co-glycolic acid) matrix with | | | | | | | | | |
| 400 μg Poly(L-lactide acid- | | | | | | | | | |
| co-glycolic acid) topcoat | | | | | | | | | |

The data in Table 7 shows that extended release of Benzyl dimethyl tetradecyl ammonium chloride is achieved with 200 μg top coated poly(L-lactide acid-co-glycolic acid) (Arm G) to 3 hours (about 92.0% eluted out up to 3 hours), with increased topcoat (400 μg of Poly(L-lactide acid-co-glycolic acid) Arm H), the release extended to 24 hours (about 97.1% eluted out up to 24 hours). Arm G and Arm H released about 600 μg to 650 μg of EDTA between 30 minutes to 3 hours in the studied dose, indicating an effective therapeutic level to inhibit local clot formation.

### Example 22: Preparation of Novolimus and Cationic Anti-Coagulation Enhancer Linear Polyethylenimine Coated Stent

Basecoat solution was prepared as below: Poly(L-lactide acid-co-glycolic acid) polymer was dissolved into dichloromethane (DCM) at room temperature, vortexed until the polymer had uniformly dissolved/dispersed. Novolimus was dissolved into dichloromethane (DCM) at room temperature and stir overnight until uniformly dissolved/dispersed. The polymer solution and Novolimus solution were combined together (Poly(L-lactide acid-co-glycolic acid) to Novolimus by weight ratio was 3:2), according to the target Novolimus dose of 100 μg for a 14 mm stent.

Topcoat solution was prepared as below: Poly(L-lactide acid-co-glycolic acid) polymer was dissolved into dichloromethane (DCM) at room temperature and vortexed until the polymer had uniformly dissolved/dispersed. Cationic anti-coagulation enhancers Linear Polyethylenimine (MW=2,500) was dissolved into ethanol at room temperature and stir overnight until uniformly dissolved/dispersed. Each polymer solution and each cationic anti-coagulation enhancers solution were combined together according to the target cationic anti-coagulation enhancers and polymer weight.

A microprocessor-controlled ultrasonic sprayer was used to coat each of the stents' 14 mm length uniformly with each of the base coat and topcoat matrix solutions. After coating, the stents were placed in a vacuum chamber to remove the solvent. The stents were then mounted on balloon catheters and crimped. The catheters were then inserted in coils and packaged. The pouches were sterilized.

### Example 23: In Vitro Testing of Extended Release of Stent with Novolimus and Cationic Anti-Coagulation Enhancer Linear Polyethylenimine

In-Vitro Elution evaluation of the stent with Novolimus and cationic anti-coagulation enhancer linear polyethylenimine was performed in 0.01M phosphate buffered saline at 37° C. water shaking bath then tested on High Performance Liquid Chromatography (H1PLC). Polyethylenimine was separated from interfering peaks by a Carbon18 reverse phase column and quantified with an external Benzyl dimethyl tetradecyl ammonium chloride calibration standard solution prepared at similar concentrations. The percentage of cationic anti-coagulation enhancer eluted was calculated during each time point as listed below.

TABLE 8
Cumulative percent release of cationic anti-coagulation enhancer polyethylenimine
percentage of release profile in 0.01 M phosphate buffered saline

| Time | 2 min | 5 min | 30 min | 2 hrs | 8 hrs | 24 hrs |
|---|---|---|---|---|---|---|
| Arm I: 560 μg linear | 13.8 | 73.5 | 81.5 | 90.2 | 97.6 | 100.0 |
| polyethylenimine with 100 μg | | | | | | |
| Novolimus in 400 μg Poly(L-lactide | | | | | | |
| acid-co-glycolic acid) matrix | | | | | | |
| Arm J: Base coated 100 μg | 6.6 | 81.6 | 87.5 | 93.3 | 98.5 | 100.0 |
| Novolimus in Poly(L-lactide acid- | | | | | | |
| co-glycolic acid) matrix and top | | | | | | |
| coated 800 μg linear | | | | | | |
| polyethylenimine with 200 μg | | | | | | |
| Poly(L-lactide acid-co-glycolic acid) | | | | | | |
| Arm K: Base coated 100 μg | 6.9 | 82.4 | 87.6 | 93.6 | 98.6 | 100.0 |

Novolimus in Poly(L-lactide acid-
co-glycolic acid) matrix and top
coated 800 μg linear
polyethylenimine with 400 μg
Poly(L-lactide acid-co-glycolic acid)

The data in Table 8 shows that the release of polyethylenimine was similar with polyethylenimine and poly(L-lactide acid-co-glycolic acid) matrix either with one layer or only in the topcoat. Arm J and Arm K released around 600 μg of polyethylenimine between 2 minutes to 5 minutes in the studied dose, which is more than an effective dose of 33 μg/mi of Linear Polyethylenimine, indicating an effective therapeutic level to inhibit local clot formation.

### Example 24: Preparation of Anticoagulant (Rivaroxaban, Argatroban, and Dalteparin) Eluting Stents

Poly(n-butyl methacrylate) polymer was dissolved into dichloromethane (tetrahydrofuran (THF) was used for Dalteparin) at room temperature and vortexed until the polymer had uniformly dissolved/dispersed. Rivaroxaban was dissolved into dichloromethane at room temperature and vortexed until the drug was uniformly dissolved/dispersed. Argatroban (and Argatroban in combination with Rivaroxaban) was dissolved in Methanol and dichloromethane and vortexed at room temperature until the drug was uniformly dispersed/dissolved. Dalteparin was dissolved in water and THF until fully dissolved.

Each polymer solution and each drug solution were combined together (rivaroxaban to poly(n-buty) methacrylate) by weight ratio was 6:1), (Argatroban to poly(n-butyl methacrylate) by weight ratio was 3:4), (Dalteparin to poly(n-butyl methacrylate) by weight ratio was 2:3), and (Rivaroxaban in combination with Argatroban to Poly (n-butyl methacrylate) weight ratio was 3:2:2) according to the target drug dose of 150 μg for each drug (and 100 μg each for the rivaroxaban and Argatroban combination).

A microprocessor-controlled ultrasonic sprayer was used to coat each of the stents' 14 mm length uniformly with each of the drug/polymer matrix solutions. After coating, the stents were placed in a vacuum chamber to remove the solvent. The stents were then mounted on balloon catheters and crimped. The catheters were then inserted in coils and packaged. The pouches were sterilized. The bare metal control stents were the same as the other stents without a drug or polymer coating.

### Example 25: In Vivo Testing of Drug Eluting Stent with Different Drugs

The drug eluting stent systems containing different anticoagulants prepared as described in Example 11 were evaluated at 3 hours, 6 hours, 1 day, 3 days, 6 days, 7 days, or 28 days following implantation in a porcine coronary artery model.

The porcine model was chosen as this model has been used extensively for stent and angioplasty studies resulting in a large volume of data on the vascular response properties and its correlation to human vascular response (Schwartz et al, Circulation. 2002; 106:1867 1873). The animals were housed and cared for in accordance with the Guide for the Care and Use of Laboratory Animals as established by the National Research Council.

After induction of anesthesia, the left or right femoral artery was accessed using standard techniques and an arterial sheath was introduced and advanced into the artery. Vessel angiography was performed under fluoroscopic guidance, a 7 Fr. guide catheter was inserted through the sheath and advanced to the appropriate location where intracoronary nitroglycerin was administered. An appropriate implantation segment of coronary artery was randomly selected and a 0.014″ guidewire inserted. Quantitative Coronary Angiography (QCA) was performed to document the results. The appropriately size stent was advanced to the deployment site. The balloon was inflated at a steady rate to a pressure sufficient to achieve a balloon to artery ratio of approximately 1.1 to 1.0.

Follow up angiography was performed at the designated timepoint for each of the animals. Late lumen loss (LLL) can be expressed as:

LLL=Post-stent minimum lumen diameter−Final minimum lumen diameter

The LLL is an indicator of the amount smooth muscle cell (SMC) proliferation or inhibition. It is used to measure efficacy between drugs for SMC proliferation inhibition. The smaller the LLL, the better the efficacy of the drug.

Stented portions of coronary arteries were embedded in methyl methacrylate (MMA), then divided into a target of at least three blocks of approximately similar lengths for histology evaluation. Quantitative histopathological evaluation of stented artery sections was then performed and scored as indicated. For Fibrin formation, scores ranged from 0 to 3, with a score of 0 indicating absent or rare minimal spotting around struts of the stent, a score of 1 indicating the presence of fibrin in small amounts localized only around the struts, a score of 2 indicating the moderately abundant or denser presence of fibrin around and extending beyond the struts, and a score of 3 indicating the presence of abundant and dense fibrin and/or bridging of the fibrin between the struts. The mean score was calculated and reported. The mean of each section was then averaged to provide a mean fibrin score per stent. The smaller the fibrin score, the better efficacy.

The percentage and/or amount of each anticoagulant drug for or by each time point indicated were analyzed for each stent from the different devices in the example and the average drug tissue concentration reported.

Tissue concentrations and the amount of drug released from the stents were measured using stents implanted in porcine arteries for the drugs as indicated. The arteries at the designated time point were excised and a length of stented artery spanning from 5 mm proximal to the stented segment to 5 mm distal to the stented segment was cut. The stented artery was cut longitudinally with surgical scissors. The stents were separated from the tissue. The tissue content of each drug was analyzed using liquid chromatography mass spectroscopy (LCMS) and reported as a mean for each of the timepoint indicated. For drug remaining on each stent, each drug was extracted from the stent, measured using HPLC, and reported as a mean for each of the timepoint indicated as drug released or drug remaining on a stent (where drug remaining is equal to 100% minus the percentage of drug released).

TABLE 9
Fibrin score, PK, and late lumen loss of Rivaroxaban, Argatroban, and Dalteparin (low
molecular weight heparin) released from 14 mm stents at day 7.

| | Fibrin | Cumulative Percent | Tissue | |
|---|---|---|---|---|
| Stent coating information | score | Release of drug by | concentration at | LLL at |
| (n = 3 for each arm) | at day 7 | day 7 | day 7 (ng/mg) | day 7 |
| 150 μg Rivaroxaban & Poly(n-buty) | 0.72 ± 0.12 | 99.6% | 3.9 ± 0.6 | 0.31 ± 0.20 |
| methacrylate) matrix coated stent | | | | |
| 150 μg Argatroban & Poly(n-butyl | 1.25 ± 0.87 | 47.7% | 5.7 ± 1.8 | 0.34 ± 0.20 |
| methacrylate) matrix coated stent | | | | |
| 100 μg Argatroban and 100ug | 0.80 ± 0.39 | 70.9% for | 48.1 ± 43.6 for | 0.04 ± 0.06 |
| Rivaroxaban in Poly(n-butyl | | Rivaroxaban | rivaroxaban | |
| methacrylate) matrix coated stent | | 96.5% for | 8.9 ± 6.3 for | |
| | | Argatroban | Argatroban | |
| 150 μg Dalteparin(low molecular | 1.50 ± 0.82 | 98.3% | Not tested | 0.23 ± 0.20 |
| weight heparin) in Poly(n-butyl | | | | |
| methacrylate) matrix coated stent | | | | |
| Bare metal control stent (BMS) | 1.02 ± 0.35 | N/A | N/A | 0.17 ± 0.22 |

As shown in Table 9, Rivaroxaban released from stents was effective at inhibiting fibrin formation compared to bare metal control stents, while Argatroban released from stents or Dalteparin released from stents were not effective at inhibiting fibrin formation compared to bare metal control stents.

As shown in Table 9, Rivaroxaban, Argatroban, or Dalteparin released from stents as single agents had larger LLLs compared to control and thus were not effective at inhibiting smooth muscle cell proliferation compared to bare metal control stents.

As shown in Table 9, the combination of Rivaroxaban and Argatroban released from stents had a smaller LLL compared to control and thus was effective at inhibiting smooth muscle cell proliferation compared to bare metal control stents. Furthermore, the combination of Rivaroxaban and Argatroban released from stents was effective at inhibiting fibrin formation compared to bare metal control stents.

As shown in Table 9, Rivaroxaban released from stents at a dose of at least 150 μg within 7 days from implant (or from vessel injury) was effective at inhibiting fibrin formation.

As shown in Table 9, Rivaroxaban released from stents at a dose of at least 1.8 μg/mm2 within 7 days from implant (or from vessel injury) was effective at inhibiting fibrin formation.

As shown in Table 9, Rivaroxaban released from stents at a dose of at least 10.7 μg/mm of stent length within 7 days from implant (or from vessel injury) was effective at inhibiting fibrin formation.

As shown in Table 9, Rivaroxaban dose of at least 150 μg, or of at least 1.8 μg/mm², or at least 10.7 μg/mm of device length, released from a stents device at a release rate of about 99.6% within 7 days from implant (or from time of injury) was effective at inhibiting fibrin formation.

As shown in Table 9, Rivaroxaban released from stents at a release rate of at least 70.9% when combined with Argatroban at a release rate of at least 96.9% within 7 days from implant (or from time of injury) was effective at inhibiting fibrin formation.

Table 9 shows Rivaroxaban released from a stent at a dose of at least 100 μg, or at a dose of at least 1.2 μg/mm2, or at a dose of at least 7.14 μg/mm of stent length, at a release rate of at least 70.9% within 7 days when combined with Argatroban released from a stent at a dose of at least 100 μg, or at a dose of at least 1.2 μg/mm2, or at a dose of at least 7.14 μg/mm of stent length, at a release rate of at least 96.9% within 7 days from implant (or from time of injury) was effective at inhibiting fibrin formation.

### Example 26: Preparation of Rivaroxaban and m-TOR Inhibitor Releasing Stent

Base coat of Novolimus (m-TOR inhibitor) and Poly (n-butyl methacrylate) matrix: Poly(n-butyl methacrylate) polymer was dissolved into dichloromethane at room temperature and vortex until the polymer had uniformly dissolved/dispersed. Novolimus was placed in another vial and dissolved in dichloromethane at room temperature until uniformly dissolved or dispersed, The polymer solution and drug solutions were mixed together and coated as a matrix (the drug to polymer weight ratio was 2:3 by weight).

Topcoat of Rivaroxaban and poly (n-butyl methacrylate) matrix: Poly(n-butyl methacrylate) polymer was dissolved in dichloromethane at room temperature and vortex until the polymer had uniformly dissolved-dispersed. Rivaroxaban was dissolved into dichloromethane at room temperature and vortex until the drug was uniformly dissolved/dispersed. Each polymer solution and each drug solutions were mixed together as a matrix (rivaroxaban to poly(n-butyl methacrylate) by weight ratio was 6:1 for the rivaroxaban fast formulation without m-TOR). Rivaroxaban to poly (n-butyl methacrylate) ratio was 4:1 for the fast release formulation with m-TOR base coat matrix, and 2:1 for the slow-release formulation with m-TOR base coat matrix according to the target drug dose of 100 μg Rivaroxaban and 25 μg poly(n-butyl methacrylate) for fast release formulation, and 100 μg Rivaroxaban and 50 μg poly(n-butyl methacrylate) for the slow-release formulation.

A microprocessor-controlled ultrasonic sprayer was used to coat each of the stents' 14 mm length uniformly with each of the drug/polymer matrix solution with the base coat matrix first, placing the stents in vacuum chamber to remove the solvent, followed by the topcoat matrix. The stents were placed in a vacuum chamber again to remove the solvents. The stents were then mounted on balloon catheters and crimped. The catheters were then inserted in coils and packaged. The pouches were sterilized. The Novolimus (m-TOR inhibitor) stents controls (DES) consisted of only the base coat drug/polymer matrix, without the topcoat drug/polymer matrix, otherwise being the same as the other stents. The bare metal control stents (BMS) were the same as the other stents without a drug or polymer coating.

TABLE 10
Fibrin score and PK of Rivaroxaban releasing 14 mm stents at day 7 and at day 28.

| | 7 days n = 5 | | | 28 days n = 5 | | |
|---|---|---|---|---|---|---|
| Stent coating | | Cumulative | | | Cumulative | |
| information n = 5 | | Percent | Tissue | | Percent | Tissue |
| for each arm for | Fibrin | Release of | concentration | Fibrin | Release | concentration |
| each time point | score | drug | (ng/mg) | score | of drug | (ng/mg) |
| 25 μg Poly(n-butyl | 0.79 ± | 99.7% | 3.95 ± 1.57 | 0.07 ± | 100% | 3.93 ± 2.43 |
| methacrylate) & 150 μg | 0.14 | | | 0.05 | | |
| Rivaroxaban matrix | | | | | | |
| 66 μg Novolimus (m-TOR | 1.16 ± | 88.9% | 6.18 ± 5.37 | 1.06 ± | 92.5% | 14.61 ± 17.68 |
| inhibitor) and 100 μg Poly | 0.60 | | | 0.46 | | |
| (n-butyl methacrylate) matrix | | | | | | |
| as a base coat then 100 μg | | | | | | |
| Rivaroxaban and 25 μg | | | | | | |
| Poly(n-butyl methacrylate) | | | | | | |
| matrix as a topcoat | | | | | | |
| 66 μg Novolimus (m-TOR | 1.18 ± | 68.1% | 8.56 ± 3.56 | 1.49 ± | 72.7% | 15.23 ± 8.12 |
| inhibitor) and 100 μg Poly | 0.53 | | | 0.37 | | |
| (n-butyl methacrylate) matrix | | | | | | |
| as a base coat then 100 μg | | | | | | |
| Rivaroxaban and 50 μg | | | | | | |
| Poly(n-butyl methacrylate) | | | | | | |
| matrix as a topcoat | | | | | | |
| 66 μg Novolimus (m-TOR | 1.51 ± | — | — | 1.37 ± | — | — |
| inhibitor) and 100 μg Poly | 0.71 | | | 0.44 | | |
| (n-butyl methacrylate) matrix | | | | | | |
| (DES) control | | | | | | |
| Bare metal control (BMS) | 1.35 ± | — | — | — | — | — |
| | 0.41 | | | | | |

As shown in Table 10, Rivaroxaban released from a stent was effective at inhibiting fibrin formation compared to control at 7 days and at 28 days when it was released at a faster rate and/or at a larger dose whether it was released alone or in combination with an m-TOR inhibitor, while it (Rivaroxaban) was not effective at inhibiting fibrin formation compared to control at 28 days when it was released at a slower rate and/or at a smaller dose.

As shown in Table 10, Rivamomaban released from a stent was more effective at inhibiting fibrin formation compared to control at 7 days and at 28 days when it was released at a rate of at least 99.7% within 7 days and/or when a dose of at least 150 μg, or a dose of at least 2.0 μg/mm², or a dose of at least 10.7 μg/mm of stent length, was released within 7 days from vessel injury (or from implantation).

As shown in Table 10, Rivaroxaban released from a stent was more effective at inhibiting fibrin formation compared to control at 7 days and at 28 days when it was released at a rate of at least 100% within 28 days and/or when a dose of at least 150 μg, or a dose of at least 2.0 μg/mm², or a dose of at least 610.7 μg/mm of stent length, was released within 28 days from vessel injury (or from implantation).

As shown in Table 7, Rivaroxaban released from a stent was more effective at inhibiting fibrin formation compared to control at 7 days and at 28 days when it was released at a rate of at least 88.9% within 7 days and/or when a dose of at least 88.9 μg, or a dose of at least 1.2 μg/mm², or a dose of at least 7.14 μg/mm of stent length, was released within 7 days from vessel injury (or from implantation), and at a rate of at least 92.5% within 28 days and/or when a dose of at least 92.5 μg, or a dose of at least 1.1 μg/mm², or a dose of at least 6.6 μg/mm of stent length, was released within 28 days from vessel injury (or from implantation).

As shown in Table 10, Rivaroxaban released from a stent was not effective at inhibiting fibrin formation compared to control at 28 days when it was released at a slower rate and/or at a smaller dose.

As shown in Table 10, Rivaroxaban released in combination with an m-TOR inhibitor from a stent was effective at inhibiting fibrin formation compared to control at 7 days and at 28 days only when it was released faster and/or at a larger dose.

As shown in Table 10, Rivaroxaban released in combination with an m-TOR inhibitor from a stent was effective at inhibiting fibrin formation compared to control at 7 days and at 28 days when Rivaroxaban was released at a rate of at least 88.9 μg, or a dose of at least 1.2 μg/mm², or a dose of at least 7.14 μg/mm of stent length, within 7 days, and/or released at a rate of at least 92.5 μg, or a dose of at least 1.1 μg/mm², or a dose of at least 6.6 μg/mm of stent length, within 28 days.

As shown in Table 10, Rivaroxaban released in combination with an m-TOR inhibitor from a stent was effective at inhibiting fibrin formation compared to control at 7 days when Rivaroxaban was released at a rate of at least 68.1 μg, or at rate of 0.84 μg/mm², or at a rate of at least 4.86 μg/mm of stent length, within 7 days.

As shown in Table 10, Rivaroxaban tissue concentration ranges from at least 3.96 ng/mg of tissue adjacent to the stented segment to at least 15 ng/mg of tissue adjacent to the stented segment, within or at 7 days, or within or 28 days from implant (or tissue injury)

It was reported that Rivaroxaban IC₅₀ for factor Xa inhibition to be about 21 nM or 0.0092 ng/mg. As shown in Table 10, the tissue concentration for Rivaroxaban was at least 426 times Rivaroxaban IC 50 for factor Xa inhibition.

TABLE 11
Tissue concentration of Rivaroxaban and Argatroban at 7 days show folds higher (or
times higher) than IC50 for Anti-factor Xa/IIa and antiplatelet for the respective drugs.

| | Rivaroxaban* | | Argatroban** | |
|---|---|---|---|---|
| | Tissue | Tissue | Tissue | Tissue |
| Stent coated | concentration at | concentration at | concentration at | concentration at |
| with combination | day 7 in folds | day 7 in folds | day 7 in folds | day 7 in folds |
| of Rivaroxaban | higher than IC50 | higher than IC50 | higher than IC50 | higher than IC50 |
| andArgatroban | of anti-Factor Xa | of anti-platelet | of anti-Factor IIa | of anti-platelet |
| 100 μg Argatroban and | 5253 | 354 | 835 | 1223 |
| 100 μg Rivaroxaban | | | | |
| in Poly(n-butyl | | | | |
| methacrylate) matrix | | | | |

*Rivaroxaban ICSO for Anti-Factor Xa is 21 nM or 0.00916 ng/mg
*Rivaroxaban IC50 for Tissue factor generated antiplatelet is 312 nM or 0.136 ng/mg
**Argatroban IC50 for Anti-Factor Ila is 21 nM or 0.0107 ng/mg
**Argatroban IC50 for Tissue factor generated antiplatelet is 79 nM or 0.04 ng/mg

Table 11 shows the tissue PK data for Rivaroxaban and Argatroban at or by or within 7 days from implants of stented vessels. It shows Rivaroxaban and Argatroban has therapeutic tissue concentrations in the tissue segment up to 7 days. Table 8 is Rivaroxaban and Argatroban concentration (ng/mg) in the tissue of treated area of the implanted device fold higher than IC₅₀ for anti-Factor Xa or Anti-Factor IIa and anti-platelet. It shows that Rivaroxaban and Argatroban in tissue concentrations have several order of magnitudes, has from 2 to 4 orders of magnitude) of tissue concentration for each of the drugs compared to their IC₅₀, in the treated tissue segments up to 7 days, therefore inhibiting fibrin formation or clot formation on the device surfaces, the stented segment tissue, and the tissue adjacent to the stented segment.

### Example 27: Preparation of Anticoagulant1/Anticoagulant2/mTOR Eluting Stents

Poly(L-lactide acid-co-glycolic acid) polymer was dissolved into dichloromethane at room temperature and vortex until the polymer had uniformly dissolved/dispersed. Sirolimus and anticoagulants (Apixaban or Rivaroxaban and Argatroban) were placed in a vial and dissolved in dichloromethane or dichloromethane/Methanol at room temperature and vortex until all the drug was uniformly dissolved/dispersed.

Each polymer solution and each drug (or combined drugs) solutions were combined together (SS7 arm anticoagulant (Apixaban to Argatroban was 1:1) to poly(L-lactide acid-co-glycolic acid) matrix by weight ratio was 3:1 as a base coat and Siroliums to poly(L-lactide acid-co-glycolic acid) matrix by weight ratio was 2:3 and coated as a top coat), (SS9 arm Siroliums and anticoagulant Apixaban and Argatroban was (1:1:1) to poly(L-lactide acid-co-glycolic acid) by weight ratio was 5:2 on matrix), (SS15 arm Sirolimus and Apixaban and Argatroban were combined in a ratio of (1:1:1) with poly(L-lactide acid-co-glycolic acid) by weight ratio which was (1:2) (by weight of 23 μg Sirolimus, 23 μg Apixaban and 23 μg Argatroban combined with 138 μg poly(L-lactide acid-co-glycolic acid)) and mixed together, and coated as a base coat (drug/polymer matrix as a base coat). In addition, Sirolimus and Apixaban and Argatroban were combined in the ratio of (3:4:4) with poly(L-lactide acid-co-glycolic acid) by weight ratio which was (5:3) and coated as a top layer or coat (drug/polymer matrix top layer or coat), (by weight of 71 μg Sirolimus, 94 μg Apixaban and 94 μg Argatroban and combined with 155 μg poly(L-lactide acid-co-glycolic acid) and coated as a top layer or coat, for cumulative total target drug dose of 117 μg for each anticoagulant and 94 μg for Sirolimus for a 14 mm stent length, (Slider II Arm1 (SS16) Sirolimus and Rivaroxaban and Argatroban were combined together in the ratio of (1:1:1) and were combined with poly(L-lactide acid-co-glycolic acid) by weight ratio which was (1:2) and coated as a base coat (drug/polymer matrix as base coat). In addition, Sirolimus and Rivaroxaban and Argatroban were combined in the ratio of (3:4:4) and combined with poly(L-lactide acid-co-glycolic acid) by weight ratio was (5:3) and coated as a top layer or coat (drug/polymer matrix as top layer or coat), (by weight of 23 μg Sirolimus, 23 μg Rivaroxaban and 23 μg Argatroban and 138 μg poly(L-lactide acid-co-glycolic acid) mixed together and coated as base coat; and by weight of 71 μg Sirolimus, 94 μg Rivaroxaban and 94 μg Argatroban and 155 μg poly(L-lactide acid-co-glycolic acid) mix together in a matrix and coated as top layer or coat, for a total target drug dose of 117 μg for each anticoagulant and 94 μg for Sirolimus for a 14 mm stent length, (Slider II Arm2 (SS17) Sirolimus and Rivaroxaban and Argatroban were combined in a ratio of (4:1:1) and combined with poly(L-lactide acid-co-glycolic acid) by weight ratio which was (1:1) and coated as a base coat (drug/polymer matrix as base coat). In addition, Rivaroxaban and Argatroban were combined in a ratio of (1:1) and combined with poly(L-lactide acid-co-glycolic acid) by weight ratio which was (5:3) and coated as a top layer or coat on the stent (drug/polymer matrix as a top layer or coat), (by weight of 94 μg Sirolimus, 23 μg Rivaroxaban and 23 μg Argatroban and 140 μg poly(L-lactide acid-co-glycolic acid) mixed together and coated as base coat; and by weight of 94 μg Rivaroxaban and 94 μg Argatroban and 113 μg poly(L-lactide acid-co-glycolic acid) were mixed together and coated as top layer or coat, for a total target drug dose of 117 μg for each anticoagulant and 94 μg for Sirolimus for a 14 mm stent length. The preceding doses for SS7, SS9, SS15, SS16, and SS17 were for 14 mm stent lengths. Drug and polymer doses are adjusted accordingly for each stent length. Control was 14 mm stent length eluting 65 μg Novolimus (m-TOR inhibitor). A microprocessor controlled ultrasonic sprayer was used to coat each of the stents' 14 mm length uniformly with each of the drug/polymer matrix solution. After coating, the stents were placed in a 70° C. oven for about 2 hours to remove the solvent. The stents were then mounted on balloon catheters and crimped. The catheters were then inserted in coils and packaged. The pouches were sterilized.

The following Tables 12A-12D describe results from in vivo testing for the following arms of SS7, SS9, SS15, SS16, and SS17.

TABLE 12A
In-vivo cumulative percent drug release profile of Rapamycin, Apixaban/
Rivaroxaban and Argatroban in stented segments.

| | Sample | | | | | | | | |
|---|---|---|---|---|---|---|---|---|---|
| Sample Matrix | Size | Time period | 1 H | 3 H | 24 H | 6 D | 7 D | 28 D | 90 D |
| SS7 Argatroban/ | n = 1 | Apixaban, % | N/A | 30 | 91 | 98 | 99 | 99 | N/A |
| Apixaban/Sirolimus | | Argatroban, % | | 91 | 99 | 98 | 99 | 99 | N/A |
| | | Sirolimus, % | N/A | 50 | 70 | 69 | 73 | 77 | N/A |
| SS9 Argatroban/ | n = 1 | Apixaban, % | N/A | 68 | 97 | 98 | 98 | 99 | N/A |
| Apixaban/Sirolimus | | Argatroban, % | N/A | 79 | 98 | 98 | 98 | 99 | N/A |
| | | Sirolimus, % | N/A | 60 | 91 | 93 | 94 | 97 | N/A |
| SS15 Argatroban/ | n = 5 | Apixaban, % | 49 | 61 | 77 | N/A | 80 | 84(n = 3) | 87(n = 3) |
| Apixaban/Sirolimus | | Argatroban, % | 51 | 63 | 77 | N/A | 80 | 84(n = 3) | 86(n = 3) |
| | | Sirolimus, % | 44 | 55 | 71 | N/A | 81 | 90(n = 3) | 97(n = 3) |
| SS16 Argatroban | n = 5 | Rivaroxaban, % | 36 | 47 | 83 | N/A | 86 | 89 | N/A |
| Rivaroxaban/Sirolimus | | Argatroban, % | 35 | 49 | 82 | N/A | 85 | 87 | N/A |
| | | Sirolimus, % | 29 | 44 | 73 | N/A | 87 | 94 | N/A |
| SS17 Argatroban/ | n = 5 | Rivaroxaban, % | 65 | 71 | 86 | N/A | 92 | 94 | N/A |
| Rivaroxaban/Sirolimus | | Argatroban, % | 78 | 80 | 86 | N/A | 91 | 93 | N/A |
| | | Sirolimus, % | 8 | 18 | 63 | N/A | 77 | 87 | N/A |

N/A: Not available

Table 12A: SS7 and SS9 provide a therapeutic composition where about 90% of the direct factor Xa and factor IIa inhibitors are released from the stent within 24 hours. It also shows that these agents are released substantially completely within about 28 days.

Table 12A: SS15, SS16, and SS17 provide therapeutic compositions where each composition providing a bolus drug release from time of injury and/or implant, and an extended drug release from time of injury and/or implant for each of Apixaban, Rivaroxaban, and Argatroban.

Table 12A: SS15 provides a therapeutic composition providing a bolus drug release phase (or formulation) from time of injury and/or implant and an extended drug release phase (or formulation) from time of injury and/or implant for the combination of Apixaban and Argatroban, wherein the bolus drug release occurs within an hour, within 3 hours, or within 24 hours, from time of injury and/or implant; and the extended drug release extends beyond 7 day, extends beyond 28 days, or extends beyond 90 days from time of injury and/or implant.

Table 12A: SS15 provides a therapeutic composition a bolus drug release phase (or formulation) from time of injury and/or implant and an extended drug release phase (or formulation) from time of injury and/or implant for the combination of Apixaban, Argatroban, and Sirolimus, wherein the bolus drug release occurs within an hour to within 24 hours from time of injury and/or implant and the extended drug release extends beyond 7 day, extends beyond 28 days, or extends beyond 90 days from time of injury and/or implant.

Table 12A: SS15 provides a therapeutic composition providing a bolus drug release phase (or formulation) from time of injury and/or implant and an extended drug release phase (or formulation) from time of injury and/or implant for the combination of Apixaban and Argatroban, wherein the bolus drug release occurs within an hour from time of injury and/or implant and wherein Apixaban bolus release is about 49% within an hour and wherein Argatroban bolus release is about 51% within an hour and the extended drug release of each of the drugs is about 80% within 7 days, about 84% within 28 days, and about 86% within 90 days from time of injury and/or implant. In this arm, the drugs are released or commence release substantially about the same time.

Table 12A: SS16 and SS17 provide therapeutic composition providing a bolus drug release phase (or formulation) from time of injury and/or implant and an extended drug release phase (or formulation) from time of injury and/or implant for the combination of Rivaroxaban and Argatroban, wherein the bolus drug release occurs within an hour to within 24 hours from time of injury and/or implant and the extended drug release extends beyond 7 day, or extends beyond 28 days from time of injury and/or implant.

Table 12A: SS16 provides a therapeutic composition providing a bolus drug release phase (or formulation) from time of injury and/or implant and an extended drug release phase (formulation) from time of injury and/or implant for the combination of Rivaroxaban, Argatroban, and Sirolimus, wherein the bolus drug release occurs within an hour to within 24 hours from time of injury and/or implant and the extended drug release extends beyond 7 day, or extends beyond 28 days from time of injury and/or implant. In this arm, the drugs are released or commence release substantially about the same time.

Table 12A: SS16 and SS17 provide therapeutic compositions providing a bolus drug release phase (or formulation) from time of injury and/or implant and an extended drug release phase (or formulation) from time of injury and/or implant for the combination of Rivaroxaban and Argatroban, wherein the bolus drug release occurs within an hour to within 24 hours from time of injury and/or implant and wherein Rivaroxaban bolus release ranges from 36% to 68% within an hour and wherein Argatroban bolus release ranges from 35% to 78% within an hour and the extended drug release of each of the drugs ranges from 85% to 92% for Rivaroxaban within 7 days, 86%-91% for Argatroban within 7 days, ranges from 89%-94% within 28 days for Rivaroxaban and 87%-93% for Argatroban from time of injury and/or implant to within 28 days.

Table 12A: SS16 and SS17 formulations each has one formulation providing a bolus drug release and another formulation providing an extended drug release for the combination of Rivaroxaban and Argatroban, wherein the bolus drug release occurs within an hour to within 24 hours of injury or implantation and the extended drug release extends beyond 7 day or extends beyond 28 days.

Table 12A: SS16 and SS17 shows multiple formulations providing a bolus drug release formulation and an extended drug release formulation for the combination of each of Rivaroxaban and Argatroban, wherein the extended release extends beyond 7 day, or extends beyond 28 days.

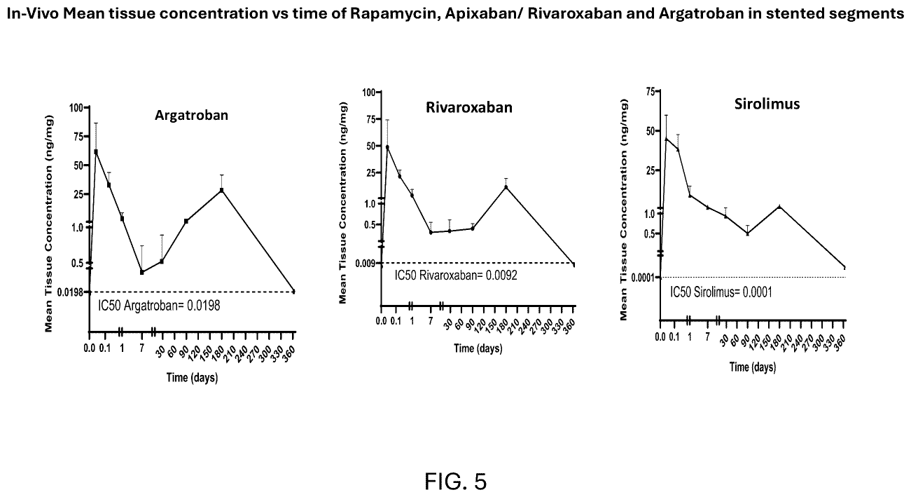

Table 12A: SS15, SS16, and SS17 Provides therapeutic compositions comprising two drugs/polymer formulations each, wherein each formulation contains at least two drugs: a direct factor Xa inhibitor and a direct factor IIA inhibitor. A third drug being an M-tor inhibitor is present in each of the formulations except in SS17 where it is present in only one formulation (base formulation) configured to delay the release of M-tor in SS17 providing a smaller bolus within the first hour for M-tor. All formulations provide an extended release of the drugs beyond 7 days, or beyond 28 days. In another study, preclinical porcine model shows sustained therapeutic tissue drug concentrations (above IC50) in treated vessel segments for about 365 days as shown in FIG. 5.

Arm SS17 factor IIa inhibitor and factor Xa inhibitor commence release prior to the anti-proliferative which was intended/configured to delay commence of its release compared to the other two drugs.

TABLE 12B
Tissue drug concentration (ng/mg) of Apixaban, Rivaroxaban, Argatroban and
Rapamycin in the stented segment tissue at the indicated time points following implantation.

| Sample | Sample | | | | | | | | |
|---|---|---|---|---|---|---|---|---|---|
| Matrix | Size | Time period | 1 H | 3 H | 24 H | 6 D | 7 D | 28 D | 90 D |
| SS7 | n = 1 | Apixaban | N/A | 102.9 | 16.5 | 0.07 | 0.03 | 0.12 | N/A |
| Argatroban/ | | Argatroban | N/A | 54 | 0.75 | 0.09 | 0.05 | 0.15 | N/A |
| Apixaban/ | | Sirolimus | N/A | 7.34 | 4.46 | 1.38 | 0.73 | 1.79 | N/A |
| Sirolimus | | | | | | | | | |
| SS9 | n = 1 | Apixaban | N/A | 91.8 | 4.17 | 0.28 | 2.65 | 1.69 | N/A |
| Argatroban/ | | Argatroban | N/A | 123.1 | 0.18 | 0.33 | 2.54 | 1.76 | N/A |
| Apixaban/ | | Sirolimus | N/A | 40.57 | 0.93 | 1.61 | 3.24 | 2.48 | N/A |
| Sirolimus | | | | | | | | | |
| SS15 | n = 5 | Apixaban | 66.94 ± | 25.31 ± | 13.25 ± | NA | 1.15 ± | 1.28 ± 0.47 | 3.05 ± 1.77 |
| Argatroban/ | | | 27.33 | 11.21 | 10.17 | | 0.52 | (n = 3) | (n = 3) |
| Apixaban/ | | Argatroban | 71.37 ± | 27.65 ± | 15.64 ± | NA | 1.41 ± | 1.69 ± 0.64 | 3.74 ± 1.89 |
| Sirolimus | | | 31.32 | 15.00 | 12.08 | | 0.69 | (n = 3) | (n = 3) |
| | | Sirolimus | 43.22 ± | 23.37 ± | 29.17 ± | NA | 1.54 ± | 1.67 ± 0.22 | 1.28 ± 0.07 |
| | | | 14.73 | 6.88 | 18.65 | | 0.35 | (n = 3) | (n = 3) |
| SS16 | n = 5 | Rivaroxaban | 48.75 ± | 21.48 ± | 3.674 ± | N/A | 0.31 ± | 0.34 ± 0.27 | N/A |
| Argatroban/ | | | 25.52 | 5.80 | 5.59 | | 0.24 | | |
| Rivaroxaba/ | | Argatroban | 61.87 ± | 32.81 ± | 3.80 ± | N/A | 0.42 ± | 0.52 ± 0.37 | N/A |
| Sirolimus | | | 24.60 | 10.96 | 4.87 | | 0.32 | | |
| | | Sirolimus | 45.10 ± | 38.30 ± | 9.18 ± | N/A | 1.46 ± | 0.94 ± 0.19 | N/A |
| | | | 14.77 | 9.29 | 5.69 | | 0.38 | | |
| SS17 | n = 5 | Rivaroxaban | 38.31 ± | 26.23 ± | 1.31 ± | N/A | 1.07 ± | 0.52 ± 0.63 | N/A |
| Argatroban/ | | | 16.08 | 23.50 | 0.28 | | 1.88 | | |
| Rivaroxaban/ | | Argatroban | 11.80 ± | 8.06 ± | 1.19 ± | N/A | 1.35 ± | 0.67 ± 0.87 | N/A |
| Sirolimus | | | 2.69 | 3.48 | 0.46 | | 2.41 | | |
| | | Sirolimus | 21.34 ± | 27.32 ± | 10.85 ± | N/A | 3.80 ± | 1.73 ± 1.52 | N/A |
| | | | 7.5 | 6.86 | 3.55 | | 4.86 | | |

Table 12B shows drug concentration in tissue adjacent to the stented segment for each of the drugs: Apixaban of about 67 ng/mg within one hour, of about 25 ng/mg tissue within 3 hours, of about 1.15 ng/mg tissue within 7 days, 1.28 ng/mg tissue within 28 days, and of about 3 ng/mg tissue within 90 days from time of injury and/or implant; Rivaroxaban of about 38 ng/mg, or of about 49 ng/mg within one hour, of about 21 ng/mg, or of about 26 ng/mg tissue within 3 hours, of about 0.3 ng/mg, or of about 1.1 ng/mg tissue within 7 days, of about 0.34 ng/mg, or of about 0.52 ng/mg tissue within 28 days, from time of injury and/or implant; Argatroban of about 12 ng/mg, of about 62 ng/mg tissue, or of about 71 ng/mg tissue within 1 hour, of about 8 ng/mg tissue, of about 33 ng/mg tissue, or of about 27 ng/mg tissue within 3 hours, of about 0.42 ng/ng tissue, of about 1.35 ng/mg tissue, or of about 1.41 ng/mg tissue within 7 days, of about 0.52 ng/mg tissue, of about 0.67 ng/mg tissue, or of about 1.69 ng/mg tissue within 28 days, and of about 3.74 ng/mg tissue within 90 days from time of injury and/or implant; and Sirolimus of about 21 ng/mg tissue, of about 45 ng/mg tissue, or of about 43 ng/mg tissue within one hour, of about 27 ng/mg tissue, or about 38 ng/mg tissue, or of about 24 ng/mg tissue within 3 hours, of about 1.46 ng/mg tissue, of about 3.8 ng/mg tissue, or of about 1.54 ng/mg tissue within 7 days, of about 0.94 ng/mg tissue, of about 1.73 ng/mg tissue, or of about 1.67 ng/mg tissue within 28 days, and of about 1.28 ng/mg tissue within 90 days, from time of tissue injury and/or implant.

TABLE 12C
In Vivo drug remaining on stent (μg) and average cumulative percentage releases (%)
of Apixaban, Rivaroxaban, Argatroban and Sirolimus in the stent at the indicated time points
following implantation.

| Sample | | | | | | | | | |
|---|---|---|---|---|---|---|---|---|---|
| matrix* | | | | | | | | | |
| (n = 5) | | | | | | | | | |
| Drug | | | | | | | | | |
| remminiog | | | | | | | | | |
| percentage | SS15 Argatroban/Apixaban/ | | | SS16 Argatroban/Rivaroxaban/ | | | SS17 Argatroban/Rivaroxaban/ | | |
| released | Sirolimus in base coat and in | | | Sirolimus in base coat and in | | | Sirolimus in base coat and in | | |
| Time | topcoat | | | topcoat | | | topcoat | | |
| (hrs) | Apixaban | Argatroban | Sirolimus | Rivaroxaban | Argatroban | Sirolimus | Rivaroxaban | Argatroban | Sirolimus |
| 0 | 119 | 123 | 96 | 120 | 119 | 90 | 121 | 121 | 96 |
| 1 H | 61 ± 4.8 | 60 ± 5.5 | 54 ± 2.8 | 76 ± 7.1 | 77 ± 59.2 | 63 ± 4.7 | 43 ± 3.5 | 26 ± 0.8 | 88 ± 1.5 |
| | (49%) | (51%) | (44%) | (36%) | (35%) | (29%) | (65%) | (78%) | (8%) |
| 3 H | 46 ± 5.2 | 46 ± 5.1 | 43 ± 3.9 | 64 ± 9.5 | 61 ± 13.9 | 50 ± 6.1 | 34 ± 5.1 | 24 ± 1.9 | 79 ± 3.5 |
| | (61%) | (63%) | (55%) | (47%) | (49%) | (44%) | (71%) | (80%) | (18%) |
| 24 H | 27 ± 3.6 | 28 ± 1.4 | 28 ± 0.8 | 21 ± 4.8 | 213 ± 1.6 | 24 ± 7.1 | 17 ± 5.9 | 17 ± 3.8 | 36 ± 7.3 |
| | (77%) | (77%) | (71%) | (83%) | (82%) | (73%) | (86%) | (86%) | (63%) |
| 7 D | 24 ± 1.4 | 25 ± 1.0 | 18 ± 0.3 | 16 ± 2.5 | 18 ± 1.7 | 12 ± 1.2 | 9 ± 0.3 | 11 ± 0.4 | 22 ± 0.5 |
| | (80%) | (80%) | (81%) | (86%) | (85%) | (87%) | (92%) | (91%) | (77%) |
| 28 D | 19 ± 0.9 | 20 ± 6.1 | 9 ± 0.3 | 14 ± 1.0 | 15 ± 0.8 | 5 ± 0.4 | 7 ± 0.4 | 93 ± 0.7 | 13 ± 0.7 |
| | (84%) | (84%) | (90%) | (89%) | (87%) | (94%) | (94%) | (93%) | (87%) |
| 90 D | 16 ± 1.6 | 17 ± 0.6 | 3 ± 0.1 | N/A | N/A | N/A | N/A | N/A | N/A |
| | (87%) | (86%) | (97%) | | | | | | |

N/A: Not available
*SS15 28 D and 90 D (n = 3)

TABLE 12D
In Vivo drug concentration (ng/mg) in the tissue within 5 mm proximal and 5 mm
distal (tissue adjacent to the stented segment) to the stented segment.

| Sample | | | | | | | | | | | | | | | |
|---|---|---|---|---|---|---|---|---|---|---|---|---|---|---|---|
| Mauix | | 1 Hour | | 3 Hour | | 1 Day | | 6 Day | | 7 Day | | 28 Day | | 90 Day | |
| (Sample | | Proxi- | | Proxi- | | Proxi- | | Proxi- | | | | Proxi- | | Proxi- | |
| Size) | Drug | mal | Distal | mal | Distal | mal | Distal | mal | Distale | Proximal | Distale | mal | Distal | mal | Distal |
| SS15 | Apixaban, | 7.86 ± | 3.53 ± | 5.15 ± | 6.77 ± | 0.34 ± | 0.36 ± | N/A | N/A | 0.01 ± 0.01 | 0.01 ± | 0.04 | 0.02 | 0.0003 ± | 0.0002 ± |
| (n = 5 | ng/mg | 3.03 | 1.95 | 1.59 | 2.22 | 0.08 | 0.29 | | | | 0.01 | (n = 1) | (n = 1) | 0.0002 | 0.0001 |
| except | | | | | | | | | | | | | | (n = 2) | (n = 3) |
| 28 D & | Argatroban | 8.57 ± | 3.84 ± | 5.43 ± | 6.46 ± | 0005 ± | 0.37 ± | N/A | N/A | | | BQL | BQL | BQL | |
| 90 D | ng/mg | 3.66 | 2.42 | 2.67 | 3.14 | 0.03 | 0.09 | | | | | (n = 2) | (n = 2) | (n = 1) | |
| n = 3) | Sirolimus | 2.82 ± | 1.66 ± | 3.52 ± | 3.81 ± | 0.32 ± | 3.38 ± | N/A | N/A | 0.08 ± 0.01 | 0.01 ± | 0.04 | 0.02 | 0.008 | 0.0006 ± |
| | ng/mg | 1.35 | 0.78 | 0.51 | 0.6% | 0.11 | 3.01 | | | | 0.003 | (n = 1) | (n = 1) | (n = 1) | 0.0004 |
| | | | | | | | | | | | | | | BQL | (n = 2) |
| | | | | | | | | | | | | | | (n = 2) | |
| SS16 | Rivaroxaban | 2.63 ± | 6.51 ± | 3.72 ± | 2.16 ± | 0.09 ± | 0.09 ± | N/A | N/A | BQL | BOL | 0.02 | 0.02 | N/A | N/A |
| (Slider II | ng/mg | 1.14 | 2.48 | 0.53 | 0.83 | 0.03 | 0.02 | | | | | (n = 1) | (n = 1) | | |
| Arm 1 | | | | | | | | | | | | BQL | BQL | | |
| (n = 5)) | | | | | | | | | | | | (n = 5) | (n = 5) | | |
| except | Argsinohan, | 3.74 ± | 7.87 ± | 2.48 ± | 2.63 ± | 0.07 ± | 0.09 ± | N/A | N/A | BQL | BQL | BQL | BQL | N/A | N/A |
| 3 H n = 4, | ng/mg | 1.93 | 3.04 | 0.36 | 0.87 | 0.03 | 0.03 | | | | | | | | |
| 28 d n = 6) | Sirolimus, | 2.30 ± | 3.25 ± | 2.62 ± | 3.38 ± | 0.18 ± | 1.10 ± | N/A | N/A | 0.04 ± 0.03 | 0.55 ± | 0.05 ± | 0.11 ± | N/A | N/A |
| | ng/mg | 1.22 | 3.62 | 0.82 | 0.88 | 0.06 | 0.44 | | | | 0.22 | 0.05 | 0.05 | | |
| | | | | | | | | | | | | (n = 4) | | | |
| | | | | | | | | | | | | BQL | | | |
| | | | | | | | | | | | | (n = 2) | | | |
| SS17 | Rivaroxaban | 1.52 ± | 2.61 ± | 3.00 ± | 2.72 ± | 0.09 ± | 0.07 ± | N/A | N/A | 0.01 (n = 1); | 0.01 | 0.02 ± | BQL | N/A | N/A |
| (Slider II | ng/mg | 0.70 | 1.38 | 1.50 | 8.66 | 0.05 | 0.02 | | | BQL (n = 4) | (n = 1); | 0.001 | | | |
| Arm 1 | | | | | | | | | | | BQL | (n = 2) | | | |
| (n = 5 | | | | | | | | | | | (n = 4) | BQL | | | |
| except | | | | | | | | | | | | (n = 4) | | | |
| 3 H n = 4)) | Argsinohan | 0.65 ± | 0.91 ± | 0.68 ± | 0.87 ± | 0.04 ± | 0.03 ± | N/A | N/A | BQL | BQL | BQL | BQL | N/A | NA |
| 28 d n = 6) | ng/mg | 0.23 | 0.51 | 0.28 | 0.52 | 0.0l | 0.01 | | | | | | | | |
| | Sirolimus, | 1.39 ± | 1.81 ± | 2.49 ± | 2.95 ± | 0.26 ± | 0.86 ± | N/A | N/A | 0.01 (n = 3) | 0.34 ± | 0.03 ± | 0.13 ± | N/A | N/A |
| | ng/mg | 0.83 | 0.83 | 1.02 | 3.09 | 0.27 | 0.48 | | | BQL (n = 2) | 0.21 | 0.02 | 0.06 | | |
| | | | | | | | | | | | | (n = 4) | | | |
| | | | | | | | | | | | | BQL | | | |
| | | | | | | | | | | | | (n = 2) | | | |

BQL: Below Quantification Limit
N/A: Not Available

### Example 28: In Vivo Animal Study of Anticoagulant1/Anticoagulant2/mTOR Eluting Stents

The test drug eluting stent systems containing anticoagulants were prepared as described in Example 4 and were evaluated at 28 days and 90 days following implantation in a porcine coronary artery. The control device was the Novolimus (m-TOR) eluting DESyne X2 stent.

The porcine artery was chosen as this model has been used extensively for stent and angioplasty studies resulting in a large volume of data on the pulmonary response properties and its correlation to human pulmonary response (Schwartz et al, Circulation. 2002; 106:1867 1873). The animals were housed and cared for in accordance with the Guide for the Care and Use of Laboratory Animals as established by the National Research Council.

All animals were pretreated with aspirin (325 mg) and Clopidogrel (75 mg) per oral dose beginning at least 3 days prior to the intervention and continuing for the duration of the study. After induction of anesthesia, the left or right femoral artery was accessed using standard techniques and an arterial sheath was introduced and advanced into the artery. Vessel angiography was performed under fluoroscopic guidance, a 7 Fr. guide catheter was inserted through the sheath and advanced to the appropriate location where intracoronary nitroglycerin was administered. An appropriate implantation segment of coronary artery was randomly selected and a 0.014″ guidewire inserted. Quantitative Coronary Angiography (QCA) was performed to document the results. The appropriately sized stent (3.0×14 mm or 3.5×14 mm) was advanced to the deployment site. The balloon was inflated at a steady rate to a pressure sufficient to achieve a balloon to artery ratio of approximately 1.1 to 1.0 but less than 1:2:1. Pressure was maintained for approximately 10 seconds before the balloon was deflated. Each pig was implanted with 3 test devices and one control device in the coronary arteries. Each time point a whole blood was drawn from animals for blood drug concentration test.

Follow up angiography imaging was performed at the designated endpoint for each of the animals. Quantitative coronary angiographic analysis was performed and the average percent diameter stenosis values and late lumen loss for the test arms and control DESyne X2 for the 28 days and 3-month time points are shown in Table 13.

Upon completion of follow-up angiography imaging, the animals were euthanized. The hearts were harvested from each animal. Any myocardial lesions or unusual observations were reported. The coronary arteries were perfused with 10% buffered formalin at 100 to 120 mm Hg with the animal's car tag until processed for histology.

Stented portions of coronary arteries were embedded in methyl methacrylate (MMA), then divided into a target of three blocks of approximately similar length (about 4 mm), identified as proximal, mid, and distal segments. From three blocks, 3 to 5 cuts were made for histology evaluation.

Quantitative histopathological evaluation of stented artery sections was then performed and scored as indicated. The mean of each section was recorded and then averaged to provide a mean score per stent for the different parameters (Table 14). The smaller the score, the better the efficacy.

### Fibrin (Strut-by-Strut)

0=absent, or rare minimal spotting around struts
1=fibrin in small amounts, localized only around struts
2=fibrin moderately abundant or denser, extending beyond struts
3=abundant, dense fibrin, bridging between struts
   Each strut in the section was scored; the mean fibrin score for each section was calculated and reported. The mean of the section means was calculated and reported, providing a mean fibrin score per stent.
   Injury Based on Schwartz et al. J Am Coll Cardiol 1992; 19:267-274, (Strut-by-Strut):
0=IEL intact
1=IEL lacerated
2=media completely lacerated
3=EEL lacerated
   Each strut in the section as scored and the mean injury score for each section was calculated and reported. The mean of the section means was calculated and reported, providing a mean injury score per stent.
   Inflammation (strut-by-strut)
0=no or very few (≤3) inflammatory cells around strut
1=few (~4-10) inflammatory cells around strut
2=many (>10) inflammatory cells around strut, can extend into but does not efface surrounding tissue
3=many (>10) inflammatory cells, effacing surrounding tissue
   Each strut in the section was scored and the mean inflammation score for each section was calculated and reported. The mean of the section means was calculated and reported, providing a mean inflammation score per device.

TABLE 13
Histopathology Scores and Quantitative Coronary Angiography data
Apixaban/Rivaroxaban, Argatroban and Sirolimus releasing 14 mm
stents at 7 days, 28 days and 3 months

| | | | | | Diameter | |
|---|---|---|---|---|---|---|
| Time Point | Device | Injury | Inflammation | Fibrin | Stenosis % | LLL, mm |
| 7 Day | SS7(n = 1) | 0.15 | 1.52 | 0.62 | N/A | N/A |
| | SS9(n = 1) | 0.61 | 1.66 | 0.44 | N/A | N/A |
| 28 Day | SS7(n = 1) | 0.21 | 0.49 | 0.94 | 16.7 | 0.71 |
| | SS9(n = 1) | 0.36 | 0.32 | 0.64 | 21.3 | 0.76 |
| | SS15 (n = 3) | 0.38 ± 0.34 | 0.62 ± 0.12 | 1.67 ± 0.38 | 19.8 ± 4.1 | 0.45 ± 0.15 |
| | DESyne | 1.34 | 1.83 | 1.87 | 51.6 | 1.14 |
| | X2(n = 1) | | | | | |
| | SS16(Slider II | 0.91 ± 0.95 | 1.52 ± 1.03 | 1.90 ± 0.26 | 19.4 ± 13.8 | 0.72 ± 0.32 |
| | Arm1, n = 6) | | | | | |
| | DESyne | 1.17 ± 1.60 | 1.97 ± 1.46 | 1.32 ± 1.11 | 36.6 ± 27.3 | 0.99 ± 0.48 |
| | X2(n = 2) | | | | | |
| | SS17(Slider II | 1.01 ± 0.47 | 1.46 ± 0.43 | 1.88 ± 0.48 | 33.1 ± 12.8 | 0.86 ± 0.34 |
| | Arm2, n = 6) | | | | | |
| | DESyne X2 | 0.87 ± 0.74 | 1.59 ± 0.95 | 1.93 ± 0.31 | 21.9 ± 11.6 | 0.71 ± 0.62 |
| | (n = 2) | | | | | |
| 3 Month | SS15 (n = 3) | 0.15 ± 0.18 | 0.52 ± 0.19 | 0.08 ± 0.06 | 18.2 ± 9.3 | 0.25 ± 0.29 |
| | DESyne | 0.43 | 0.59 | 0.76 | 22.3 | 0.52 |
| | X2(n = 1) | | | | | |

LLL is an indicator of the amount cell proliferation or inhibition potency. It is used to measure efficacy between drugs for proliferation inhibition in mammalian arteries. The smaller the LLL, the better the efficacy of the drug.

As shown in Table 13, SS15 composition providing the combination of Sirolimus, Apixaban and Argatroban released from stents had a smaller LLL compared to control which only had m-TOR inhibitor (Novolimus) and thus was unexpectedly more effective at inhibiting smooth muscle cell proliferation compared to Novolimus releasing stents at 28 days, and at 90 days. This was an unexpected finding for the test SS15 stents in comparison to the control DESyne X2 stents at the 28-day time point and/or at 90 days.

In an unexpected finding, SS15 stents composition eluting Apixaban, Argatroban, and the M-Tor inhibitor rapamycin exhibited more efficacy at inhibiting one or more of the following at 28 days and/or 90-day time points: cell proliferation, inflammation, injury, fibrin formation inhibition, and fibrin dissolution acceleration.

The LLL is an indicator of the amount cell proliferation or inhibition potency. It is used to measure efficacy between drugs for proliferation inhibition in mammalian arteries. The smaller the LLL, the better the efficacy of the drug.

As shown in Table 14, SS16 shows the combination of Sirolimus, Rivaroxaban and Argatroban released from stents had a smaller LLL compared to control which only had m-TOR inhibitor (Novolimus) and thus was unexpectedly more effective at inhibiting smooth muscle cell proliferation compared to Novolimus releasing stents at 28 days.

As shown in Table 13. SS17 composition configured to delay the release and tissue concentration of rapamycin within the first 1 hour and/or within the first 3 hours by incorporating rapamycin in the base coating shows the combination of Sirolimus, and/or lower tissue concentration of Rivaroxaban and Argatroban within at least the first hour showed less inhibition of SMC proliferation at 28 days.

### Example 29: Ex Vivo Testing of Drug Eluting Stent Compared with 2 Anticoagulants and mTOR Eluting Stents

The thrombogenicity of a drug eluting stent system with two anticoagulant Apixaban and Argatroban in combination with rapamycin at two different loading drug doses was evaluated at 1 hour in an arteriovenous ex vivo shunt in a porcine model wherein the devices were deployed in a blood compatible polymeric tubing.

The control stents were 16-o-demethyl rapamycin m-TOR inhibitor (Novolimus) drug eluting coronary stent (DESyne, Elixir) and m-TOR inhibitor Zotarolimus eluting coronary stent (Resolute, Medtronic, USA).

The test arm for this experiment were SS9, SS9*, and SS10* and were manufactured as follows: Each polymer solution and each drug solutions were combined together ((Sirolimus and anticoagulant Apixaban and Argatroban was 1:1:1) to poly(L-lactide acid-co-glycolic acid) by weight ratio was 5:2 matrix) according to the target drug dose of 235 μg for each anticoagulant and 94 μg for Sirolimus for SS9, SS9* test arm was about ⅓ of each of the drugs dose as follows: Sirolimus and anticoagulant Apixaban and Argatroban was 1:1:1) to poly(L-lactide acid-co-glycolic acid) by weight ratio was 5:2 on matrix) according to the target drug dose of 78.3 μg for each anticoagulant and 31.3 μg for Sirolimus, and SS10* arm was Sirolimus and anticoagulant Apixaban and Argatroban was 1:1:3) to poly(L-lactide acid-co-glycolic acid) by weight ratio was 5:2 matrix) according to the target drug dose of 39.2 μg for Apixaban and 117.5 μg for Argatroban and 31.3 μg for Sirolimus.

A microprocessor controlled ultrasonic sprayer was used to coat each of the stents 14 mm length uniformly with each of the drug/polymer matrix solution. After coating, the stents were placed in a 70° C. oven for about 2 hours to remove the solvent. The stents were then mounted on balloon catheters and crimped. The catheters were then inserted in coils and packaged. The pouches were sterilized.

The ex-vivo shunt model to evaluate thrombogenicity has been extensively employed to evaluate the biocompatibility of different drug eluting stents (Waksman et al. Circ Cardiovasc Interv. 2017; 10:e004762, Otsuka et al. J Am Coll Cardiol Intv 2015; 8:1248-60, Lipinski et al. EuroInterv 2018; Jaa-369 2018) The animals were housed and cared for in accordance with the Guide for the Care and Use of Laboratory Animals as established by the National Research Council.

The two pigs that were employed in this study did not receive any aspirin or clopidogel pretreatment. Further all procedures were performed in the absence of any anticoagulant including heparin. After induction of anesthesia, an arterial bypass shunt from the femoral artery to the femoral vein was created. Blood flow was established through the shunt. Flow rates through the shunt was continuously monitored during the procedure with a flow probe that was placed on the shunt tubing proximal to the arterial flow.

In the first pig three control devices were deployed in the first shunt and the blood flow through the shunt was performed for a period of 1 hour. Following perfusion, the shunt tubing containing the stents was rinsed with saline and then fixed in situ with 10% buffered formalin in order to capture the thrombus, if any, that are deposited on the stent surface.

Similar procedure with 3 shunts with only one stent SS9* or SS9 in each shunt was tested with a perfusion time of 1 hour for each of the shunts.

In the second pig three control devices were deployed in the first shunt and the blood flow through the shunt was performed for a period of 1 hour. Following perfusion, the shunt tubing containing the stents was rinsed with saline and then fixed in situ with 10% buffered formalin in order to capture the thrombus, if any, that are deposited on the stent surface.

Similar procedure with 2 shunts with only one stent SS10* in each shunt was tested with a perfusion time of 1 hour for each of the shunts.

Promptly following perfusion in each of the shunts, the tubing containing the stents was gently rinsed with saline under gravity flow and then fixed in situ with 10% buffered formalin in order to anchor the thrombus, if any, that are deposited on the stent surface.

The stents were then removed from the tubing and bisected longitudinally. Low magnification photographs of the luminal side of two halves of the control and test stents were recorded.

The two halves of the stents were then processed for scanning electron microscopy (SEM) so as to examine the thrombus on the luminal side of the stent. Low (15×) and high (200×) magnification images of the stent surface were captured to evaluate the extent of thrombus deposition on the luminal surface of the stent.

Significant number of thrombus was observed on the luminal surface (inner surface) of the control DES stents as seen on the low and high magnification SEM images whereas there was little or no thrombus deposits on the test stents with combinations of Apixaban and Argatroban and m-TOR. The number of thrombus deposits on the control and test stents as evaluated from SEM images are shown in Table 14. The data shows that the combinations of Apixaban and Argatroban and m-TOR inhibited thrombus formation in the shunt model better than control.

Table 14 shows several therapeutic compositions of factor Xa inhibitor, factor IIa, and M-tor inhibitor releasing stents had less thrombus (clot formation) compared to M-Tor inhibitor alone releasing stents.

The composition comprising a combination of factor Xa inhibitor, a direct factor II inhibitor and an anti-proliferative were surprisingly more effective than the anti-proliferative alone.

TABLE 14
Thrombus deposits on the control and
test stents as evaluated from SEM images

| | Control/ | | Number of |
|---|---|---|---|
| Animal # | Test DES | Device | Thrombus deposits |
| 1 | Control | DESyne-1 | 18 |
| | | DESyne-2 | 40 |
| | | Resolute | 14 |
| | Test-Apixaban: | SS9* | 4 |
| | Argatroban(1:1) | SS9* | 0 |
| | | SS9 | 3 |
| 2 | Control | DESyne-1 | 17 |
| | | DESyne-2 | 17 |
| | | Resolute | 15 |
| | Test-Apixaban: | SS10* | 2 |
| | Argatroban(1:3) | SS10* | 2 |

### Example 15: Preparation of Amino Acid and Anticoagulant of Apixaban or Rivaroxaban and Argatroban and a Spacer Eluting Stents

A surface-binding moiety, and one or more cell adhesion amino acid via one or more linkers could make the amino acid more hydrophobic, which can extend anticoagulant Apixaban or Rivaroxaban and Argatroban release in a biocompatible environmental and inhibit inflammation effectively. For example, dihydroxyphenylalanine and L-lysine is immobilized onto 316L stainless steel with polyethylene glycol molecule as spacer arm by using cold plasma-induced grafting technique. To immobilize peptide more effectively, polyethylene glycol is first coated onto 316LSS. After L-lysine immobilized on the stent surface, anticoagulant Apixaban or Rivaroxaban and Argatroban can be coated on the stent. After coating, the stents are placed in a vacuum oven for about 12 hours to remove the solvent. The stents are then mounted on balloon catheters and crimped. The catheters are then inserted in coils and packaged. The pouches are sterilized.

### Example 31: Preparation of Crosslinked Peptide and Anticoagulant Apixaban or Rivaroxaban and Argatroban Eluting Stents

Efficient stent implantation depends in part on avoiding the aggregation of platelets in the blood vessels and appropriate proliferation of endothelial cells and controlled proliferation of smooth muscle cells, which reduces the development of pathology, such as neointimal hyperplasia, thrombosis, and restenosis. Peptide immobilized on the stent surface can make stent more biocompatible. A crosslinked peptide will have a folded structure wrapped the anticoagulant Apixaban or Rivaroxaban and Argatroban inside; once it is inside body wet environmental, it will unfold and release the Anticoagulant Apixaban or Rivaroxaban and Argatroban.

The crosslinking of peptide is achieved with oxidizing agents, such as periodate ion and Fe(III) on the stent surface, resulting in oxidative crosslinking (covalent crosslinking) and coordinative one (non-covalent crosslinking), respectively with dopamine, producing adhesive peptide on the stent surface.

After coating, the stents are placed in a vacuum oven for about 12 hours to remove the solvent. The anticoagulant can then be coated on the stent. After coating, the stents are placed in a vacuum oven for about 12 hours to remove the solvent. The stents are then mounted on balloon catheters and crimped. The catheters are then inserted in coils and packaged. The pouches are sterilized.

### Example 32: Preparation of Polypeptide Anticoagulant Apixaban or Rivaroxaban and Argatroban Eluting Stents

Nature provides us with unusual adhesives in the form of L-DOPA-containing proteins, existing within the mussel feet and commonly called “mussel foot proteins.” A unique property of these bioadhesives is their ability to adhere to many different surfaces, even in a wet environment. The modification of stents and other implantable device surfaces with such adhesive polypeptides can provide for rapid endothelialization after implantation. The surface-binding cell adhesion polypeptides are deposited on the surface, forming an amino-containing hydrophobic coating to enhance the corrosion resistance by a surface binding moiety, such as 3,4-dihydroxyphenylalanine (DOPA). Catechol moieties (3,4-dihydroxyphenyl) in DOPA are attached to cell adhesion polypeptides. Attachment may be directly to a cell adhesion polypeptide or indirect, for example via a linker. Alternatively, this modified polypeptide can be directly coated on the stent surface with anticoagulant Apixaban or Rivaroxaban and Argatroban. After coating, the stents are placed in a vacuum oven for about 12 hours to remove the solvent. The stents are then mounted on balloon catheters and crimped. The catheters are then inserted in coils and packaged. The pouches are sterilized.

### Example 33: Preparation of Polysaturated Fatty Acid Poly(Glycerol Sebacic Acid) and Anticoagulant Apixaban or Rivaroxaban and Argatroban Eluting Stents

Poly(glycerol sebacic acid) is a simple glycerol-ester polysaturated fatty acid created from the basic mammalian metabolites of glycerol and sebacic acid. Poly(glycerol sebacic acid) coating in the stent has enhanced mechanical properties, improved biocompatibility, and antimicrobial properties. Poly(glycerol sebacic acid) is easily reducible in a wide range of solvents (e.g., ethyl acetate, THF, acetone, 1,3-dioxolane, and various alcohols) resulting in a solution that can be used in dip and spray coating applications.

Poly(glycerol sebacic acid) is dissolved into tetrahydrofuran and dichloromethane (THF:DCM) at room temperature or heated when needed, vortexed until the polymer had uniformly dissolved/dispersed. Anticoagulant (Apixaban/or Rivaroxaban & Argatroban) are placed in a vial and dissolved in dichloromethane/Methanol at room temperature and vortex until all the drug is uniformly dissolved/dispersed. Each polymer solution and each drug solutions are combined (anticoagulant (Apixaban/Rivaroxaban & Argatroban with weight ratio 1 to 1) to Poly(glycerol sebacic acid) by weight ratio is 3:1) according to the target drug dose.

The stent can optionally undergo surface treatment if the surface is not porous (i.e., plasma treatment or other surface friction treatment).

A microprocessor-controlled ultrasonic sprayer is used to coat each of the stents' 14 mm length uniformly with each of the drug/polymer matrix solutions. After coating, the stents are placed in a vacuum chamber to remove the solvent. The stents are then mounted on balloon catheters and crimped. The catheters are then inserted in coils and packaged. The pouches are sterilized.

### Example 34: Preparation of Crosslinked Polyunsaturated Fatty Acid and Anticoagulant Apixaban or Rivaroxaban, Argatroban Eluting Stents

Polyunsaturated fatty acids (PUFAs) such as Omega-3 or Omega-6 can modify platelet responsiveness to dual antiplatelet therapy in stable coronary artery disease patients undergoing percutaneous coronary intervention. Higher platelet inhibition might be achieved with increased doses of polyunsaturated fatty acids coated on the stent surface.

Polyunsaturated fatty acid (PUFA) is precure under Geotrichum sp. Lipase at 40° C. for 8-10 hours or stir at 90° C. and oxygen without enzyme for 24 hours. This partially crosslinked PUFA is washed with water and used for stent coating. This jelly PUFA product and anticoagulant Apixaban or Rivaroxaban, Argatroban are dissolved in a mixture of dichloromethane and methanol. Each partially crosslinked PUFA jelly solution and each drug solutions are combined (anticoagulant (Apixaban & Argatroban with weight ratio 1 to 1) to solid PUFA by weight ratio is 3:1) according to the target drug dose.

The stent can optionally undergo surface treatment if the surface is not porous (i.e., plasma treatment or other surface friction treatment).

A microprocessor-controlled ultrasonic sprayer is used to coat each of the stents' 14 mm length uniformly with each of the drug/polymer matrix solutions. After coating, the stents are cured at 90° C. for 24 hours in a convection oven. The stents are then mounted on balloon catheters and crimped. The catheters are then inserted in coils and packaged. The pouches are sterilized.

### Example 35: Preparation of Anticoagulant1/Anticoagulant2/mTOR/Cationic Anti-Coagulation Enhancer Polyethylenimine Eluting Stents

Poly(L-lactide acid-co-glycolic acid) polymer was dissolved into dichloromethane at room temperature and vortex until the polymer had uniformly dissolved/dispersed. Linear polyethylenimine polymer was dissolved into methanol at room temperature and vortex until the polymer had uniformly dissolved/dispersed and diluted with dichloromethane. Sirolimus and anticoagulants (Apixaban or Rivaroxaban and Argatroban) were placed in a vial and dissolved in dichloromethane or dichloromethane/Methanol at room temperature and vortex until all the drug was uniformly dissolved/dispersed.

Each polymer solution and each drug (or combined drugs) solutions were combined together (Sirolimus and Rivaroxaban and Argatroban were combined together in the ratio of (1:1:1) and were combined with poly(L-lactide acid-co-glycolic acid) by weight ratio which was (1:2) and coated as a base coat (drug/polymer matrix as base coat). In addition, Sirolimus and Rivaroxaban and Argatroban were combined in the ratio of (3:4:4) and combined with poly(L-lactide acid-co-glycolic acid) by weight ratio which was (5:3) and coated as a middle layer or coat (drug/polymer matrix as middle layer or coat), (by weight of 23 μg Sirolimus, 23 μg Rivaroxaban and 23 μg Argatroban and 138 μg poly(L-lactide acid-co-glycolic acid) mixed together and coated as base coat; and by weight of 71 μg Sirolimus, 94 μg Rivaroxaban and 94 μg Argatroban and 155 μg poly(L-lactide acid-co-glycolic acid) mix together in a matrix and coated as middle layer or coat, for a total target drug dose of 117 μg for each anticoagulant and 94 μg for Sirolimus for a 14 mm stent length. Lastly, linear polyethylenimine polymer combined with poly(L-lactide acid-co-glycolic acid) by weight ratio which was (1/1 to 21 based on the coating integrity and drug elution profile) and coated as a top layer or coat.

A microprocessor controlled ultrasonic sprayer was used to coat each of the stents' 14 mm length uniformly with each of the drug/polymer matrix solutions. If needed, after coating, the stents were placed in a 70° C. oven for about half an hour to remove the solvent after base coat and middle coat. After top coating, the stents were placed in a 70° C. oven for about 2 hours to remove the solvent. The stents were then mounted on balloon catheters and crimped. The catheters were then inserted in coils and packaged. The pouches were sterilized.

### Example 36: Colchicine/Anticoagulant1/Anticoagulant2/mTOR Eluting Stents with Colchicine Only on the Base Coating

Poly(L-lactide acid-co-glycolic acid) polymer was dissolved into dichloromethane at room temperature and vortex until the polymer had uniformly dissolved/dispersed. Colchicine and Sirolimus and anticoagulants (Apixaban or Rivaroxaban and Argatroban) were placed in a vial and dissolved in dichloromethane or dichloromethane/Methanol at room temperature and vortex until all the drug was uniformly dissolved/dispersed.

Each polymer solution and each drug (or combined drugs) solutions were combined together (Colchicine and Sirolimus and Rivaroxaban and Argatroban were combined together in the ratio of (10:3:3:3) and were combined with poly(L-lactide acid-co-glycolic acid) by weight ratio which was (1:2) and coated as a base coat (drug/polymer matrix as base coat). In addition, Sirolimus and Rivaroxaban and Argatroban were combined in the ratio of (3:4:4) and combined with poly(L-lactide acid-co-glycolic acid) by weight ratio was (5:3) and coated as a top layer or coat (drug/polymer matrix as top layer or coat), (by weight of 100 microgram (μg) Colchicine, 30 μg Sirolimus. 30 μg Rivaroxaban and 30 μg Argatroban and 380 μg poly(L-lactide acid-co-glycolic acid) mixed together and coated as base coat; and by weight of 90 μg Sirolimus, 120 μg Rivaroxaban and 120 μg Argatroban and 198 μg poly(L-lactide acid-co-glycolic acid) mix together in a matrix and coated as top layer or coat, for a total target drug dose of 100 μg for Colchicine, 150 μg for each anticoagulant and 120 μg for Sirolimus for a 18 mm stent length. Drug and polymer doses are adjusted accordingly for each stent length. A microprocessor controlled ultrasonic sprayer was used to coat each of the stents' 18 mm length uniformly with each of the drug/polymer matrix solution. After coating, the stents with basecoat were placed in a 70° C. oven for about half hours to remove the solvent; the stents with topcoat were placed in a 70° C. oven for about 2 hours to remove the solvent. The stents were then mounted on balloon catheters and crimped.

### Example 37: Preparation of Colchicine/Anticoagulant1/Anticoagulant2/mTOR Eluting Stents with Colchicine on Both Base and Top Coating

Poly(L-lactide acid-co-glycolic acid) polymer was dissolved into dichloromethane at room temperature and vortex until the polymer had uniformly dissolved/dispersed. Colchicine and Sirolimus and anticoagulants (Apixaban or Rivaroxaban and Argatroban) were placed in a vial and dissolved in dichloromethane or dichloromethane/Methanol at room temperature and vortex until all the drug was uniformly dissolved/dispersed.

Each polymer solution and each drug (or combined drugs) solutions were combined together (Colchicine and Sirolimus and Rivaroxaban and Argatroban were combined together in the ratio of (7:3:3:3) and were combined with poly(L-lactide acid-co-glycolic acid) by weight ratio which was (1:2) and coated as a base coat (drug/polymer matrix as base coat). In addition, Colchicine and Sirolimus and Rivaroxaban and Argatroban were combined in the ratio of (1:3:4:4) and combined with poly(L-lactide acid-co-glycolic acid) by weight ratio was (5:3) and coated as a top layer or coat (drug/polymer matrix as top layer or coat), (by weight of 70 μg Colchicine, 30 μg Sirolimus, 30 μg Rivaroxaban and 30 μg Argatroban and 240 μg poly(L-lactide acid-co-glycolic acid) mixed together and coated as base coat; and by weight of 30 μg Colchicine, 90 μg Sirolimus, 120 μg Rivaroxaban and 120 μg Argatroban and 216 μg poly(L-lactide acid-co-glycolic acid) mix together in a matrix and coated as top layer or coat, for a total target drug dose of 100 μg for Colchicine, 150 μg for each anticoagulant and 120 μg for Sirolimus for a 18 mm stent length. Drug and polymer doses are adjusted accordingly for each stent length.

A microprocessor controlled ultrasonic sprayer was used to coat each of the stents' 18 mm length uniformly with each of the drug/polymer matrix solution. After coating, the stents with basecoat were placed in a 70° C. oven for about half hours to remove the solvent; the stents with topcoat were placed in a 70° C. oven for about 2 hours to remove the solvent. The stents were then mounted on balloon catheters and crimped.

### Example 38: Preparation of Colchicine/mTOR Eluting Stents

Poly(L-lactide acid-co-glycolic acid) polymer was dissolved into dichloromethane at room temperature and vortex until the polymer has uniformly dissolved/dispersed. Colchicine and Sirolimus are placed in a vial and dissolved in dichloromethane at room temperature and vortex until all the drug is uniformly dissolved/dispersed.

Each polymer solution and each drug (or combined drugs) solutions are combined together (Colchicine and Sirolimus are combined together in the ratio of (1:2) and were combined with poly(L-lactide acid-co-glycolic acid) by weight ratio which was (10:3) and coated as a base coat (drug/polymer matrix as base coat). In addition, Sirolimus is combined with poly(L-lactide acid-co-glycolic acid) by weight ratio was (5:3) and coated as a top layer or coat (drug/polymer matrix as top layer or coat), (by weight of 100 μg Colchicine, 30 μg Sirolimus, and 260 μg poly(L-lactide acid-co-glycolic acid) mixed together and coated as base coat; and by weight of 90 μg Sirolimus and 44 μg poly(L-lactide acid-co-glycolic acid) mix together in a matrix and coated as top layer or coat, for a total target drug dose of 100 μg for Colchicine and 120 μg for Sirolimus for a 18 mm stent length. Drug and polymer doses are adjusted accordingly for each stent length.

A microprocessor controlled ultrasonic sprayer is used to coat each of the stents' 18 mm length uniformly with each of the drug/polymer matrix solution. After coating, the stents with basecoat are placed in a 70° C. oven for about half hours to remove the solvent; the stents with topcoat were placed in a 70° C. oven for about 2 hours to remove the solvent. The stents are then mounted on balloon catheters and crimped.

### Example 39: Preparation of Tirofiban/Anticoagulant1/Anticoagulant2/mTOR Eluting Stents with Tirofiban Only on the Top Coating

Poly(L-lactide acid-co-glycolic acid) polymer is dissolved into dichloromethane at room temperature and vortex until the polymer has uniformly dissolved/dispersed. Tirofiban and Sirolimus and anticoagulants (Apixaban or Rivaroxaban and Argatroban) are placed in a vial and dissolved in dichloromethane or dichloromethane/Methanol at room temperature and vortex until all the drug is uniformly dissolved/dispersed.

Each polymer solution and each drug (or combined drugs) solutions are combined together (Sirolimus and Rivaroxaban and Argatroban are combined together in the ratio of (1:1:1) and are combined with poly(L-lactide acid-co-glycolic acid) by weight ratio which is (1:2) and coated as a base coat (drug/polymer matrix as base coat). In addition, Tirofiban and Sirolimus and Rivaroxaban and Argatroban are combined in the ratio of (5:3:4:4) and combined with poly(L-lactide acid-co-glycolic acid) by weight ratio is (5:3) and coated as a top layer or coat (drug/polymer matrix as top layer or coat), (by weight of 30 μg Sirolimus, 30 μg Rivaroxaban and 30 μg Argatroban and 180 μg poly(L-lactide acid-co-glycolic acid) mixed together and coated as base coat; and by weight of 150 μg Tirofiban hydrochloride, 90 μg Sirolimus, 120 μg Rivaroxaban and 120 μg Argatroban and 288 μg poly(L-lactide acid-co-glycolic acid) mix together in a matrix and coated as top layer or coat, for a total target drug dose of 150 μg for Tirofiban hydrochloride, 150 μg for each anticoagulant and 120 μg for Sirolimus for a 18 mm stent length. Drug and polymer doses are adjusted accordingly for each stent length.

A microprocessor controlled ultrasonic sprayer is used to coat each of the stents' 18 mm length uniformly with each of the drug/polymer matrix solution. After coating, the stents with basecoat are placed in a 70° C. oven for about half hours to remove the solvent; the stents with topcoat are placed in a 70° C. oven for about 2 hours to remove the solvent. The stents are then mounted on balloon catheters and crimped.

### Example 40: Preparation of Tirofiban Hydrochloride/Anticoagulant1/Eluting Stents with Tirofiban Hydrochloride on Both Base and to Coating

Poly(L-lactide acid-co-glycolic acid) polymer is dissolved into dichloromethane at room temperature and vortex until the polymer has uniformly dissolved/dispersed. Tirofiban hydrochloride and anticoagulants (Apixaban or Rivaroxaban or Argatroban) are placed in a vial and dissolved in dichloromethane or dichloromethane/Methanol at room temperature and vortex until all the drug is uniformly dissolved/dispersed.

Each polymer solution and each drug (or combined drugs) solutions are combined together. Tirofiban hydrochloride and Rivaroxaban or Argatroban are combined together in the ratio of (1:1) and are combined with poly(L-lactide acid-co-glycolic acid) by weight ratio which was (1:2) and coated as a base coat (drug/polymer matrix as base coat). In addition, Tirofiban hydrochloride and Rivaroxaban or Argatroban are combined in the ratio of (1:1) and combined with poly(L-lactide acid-co-glycolic acid) by weight ratio is (5:3) and coated as a top layer or coat (drug polymer matrix as top layer or coat), (by weight of 30 μg Tirofiban hydrochloride, 30 μg Rivaroxaban and 30 μg Argatroban and 180 μg poly(L-lactide acid-co-glycolic acid) mixed together and coated as base coat; and by weight of 120 μg Tirofiban hydrochloride, 90 μg Sirolimus, 120 μg Rivaroxaban or 120 μg Argatroban and 198 μg poly(L-lactide acid-co-glycolic acid) mix together in a matrix and coated as top layer or coat, for a total target drug dose of 150 μg for Tirofiban hydrochloride. 150 μg for each anticoagulant for a 18 mm stent length. Drug and polymer doses are adjusted accordingly for each stent length.

A microprocessor controlled ultrasonic sprayer is used to coat each of the stents' 18 mm length uniformly with each of the drug/polymer matrix solution. After coating, the stents with basecoat are placed in a 70° C. oven for about half hours to remove the solvent; the stents with topcoat are placed in a 70° C. oven for about 2 hours to remove the solvent. The stents are then mounted on balloon catheters and crimped.

### Example 41: Preparation of Anti-Platelet/Direct Factor Xa Inhibitor/mTOR Eluting Stents

Poly(L-lactide acid-co-glycolic acid) polymer is dissolved into dichloromethane at room temperature and vortex until the polymer is uniformly dissolved/dispersed. Tirofiban and Sirolimus and anticoagulants (Apixaban or Rivaroxaban and Argatroban) are placed in a vial and dissolved in dichloromethane or dichloromethane/Methanol at room temperature and vortex until all the drug is uniformly dissolved/dispersed.

Each polymer solution and each drug (or combined drugs) solutions are combined together (Sirolimus and Rivaroxaban or Apixaban are combined together in the ratio of (4:5) and are combined with poly(L-lactide acid-co-glycolic acid) by weight ratio which is (1:2) and coated as a base coat (drug/polymer matrix as base coat). In addition, Tirofiban and Sirolimus and Rivaroxaban or Apixaban are combined in the ratio of (5:3:4) and combined with poly(L-lactide acid-co-glycolic acid) by weight ratio is (1:2) and coated as a top layer or coat (drug/polymer matrix as top layer or coat), (by weight of 30 μg Sirolimus, 30 Ig Rivaroxaban or Apixaban and 120 μg poly(L-lactide acid-co-glycolic acid) mixed together and coated as base coat; and by weight of 150 μg Tirofiban hydrochloride, 90 μg Sirolimus, 120 μg Rivaroxaban or Apixaban and 720 μg poly(L-lactide acid-co-glycolic acid) mix together in a matrix and coated as top layer or coat, for a total target drug dose of 150 μg for Tirofiban hydrochloride, 150 μg for direct factor Xa inhibitor and 120 μg for Sirolimus for a 18 mm stent length. Drug and polymer doses are adjusted accordingly for each stent length.

A microprocessor controlled ultrasonic sprayer is used to coat each of the stents' 18 mm length uniformly with each of the drug/polymer matrix solution. After coating, the stents with basecoat are placed in a 70° C. oven for about half hours to remove the solvent; the stents with topcoat are placed in a 70° C. oven for about 2 hours to remove the solvent. The stents are then mounted on a drug coated balloon catheters and crimped, where the drug is the antiplatelet aspirin at dose 1 mg to 5 mg coated on the balloon.

### Example 42: Preparation of Anti-Platelet/Direct Factor Xa Inhibitor/mTOR Eluting Stents with Aspirin Coated as Topcoat on Stent

Poly(L-lactide acid-co-glycolic acid) polymer is dissolved into dichloromethane at room temperature and vortex until the polymer is uniformly dissolved/dispersed. Tirofiban and Sirolimus and anticoagulants (Apixaban or Rivaroxaban) are placed in a vial and dissolved in dichloromethane or dichloromethane/Methanol at room temperature and vortex until all the drug is uniformly dissolved.

Each polymer solution and each drug (or combined drugs) solutions are combined together (Sirolimus and Rivaroxaban or Apixaban are combined together in the ratio of (4:5) and are combined with poly(L-lactide acid-co-glycolic acid) by weight ratio which is (1:2) and coated as a base coat (drug/polymer matrix as base coat).

In addition. Poly(L-lactide acid-co-glycolic acid) polymer is dissolved into dichloromethane at room temperature and vortex until the polymer has uniformly dissolved/dispersed. Aspirin is placed in a vial and dissolved in ethanol at room temperature and vortex until all the drug is uniformly dissolved/dispersed.

Each polymer solution and each drug solutions are combined together with poly(L-lactide acid-co-glycolic acid) by weight ratio which is (1:2) and coated as a topcoat (drug/polymer matrix as topcoat).

The total target drug dose is 150 μg for Apixaban or Rivaroxaban and 120 μg for Sirolimus and 150 μg for Aspirin for an 18 mm stent length. Drug and polymer doses are adjusted accordingly for each stent length.

A microprocessor controlled ultrasonic sprayer is used to coat each of the stents' 18 mm length uniformly with each of the drug/polymer matrix solution. After coating, the stents with basecoat are placed in a 70° C. oven for about half hours to remove the solvent; the stents with topcoat are placed in a 70° C. oven for about 2 hours to remove the solvent. The stents are then mounted on balloon catheters and crimped.

### Example 43: Preparation of Tirofiban/Colchicine/Anticoagulant1/Anticoagulant2/mTOR Eluting Stents with all Drugs on Both Base and Top Coating

Poly(L-lactide acid-co-glycolic acid) polymer dissolved into dichloromethane at room temperature and vortex until the polymer uniformly dissolved/dispersed. Tirofiban hydrochloride, Colchicine, anticoagulant (Apixaban or Rivaroxaban and Argatroban) and Sirolimus were placed in a vial and dissolved in dichloromethane or dichloromethane/Methanol/ethanol at room temperature and vortex until all the drug was uniformly dissolved/dispersed.

Each polymer solution and each drug (or combined drugs) solutions were combined together (Tirofiban hydrochloride and Colchicine and Rivaroxaban or Apixaban and Argatroban and Sirolimus were combined together in the ratio of (5:5:5:5:6) and are combined with poly(L-lactide acid-co-glycolic acid) by weight ratio which was (1:2) and coated as a base coat with drug dose in base coat. In addition, Tirofiban hydrochloride and Colchicine and Rivaroxaban or Apixaban and Argatroban and Sirolimus were combined together in the ratio of (5:5:5:5:6) and were combined with poly(L-lactide acid-co-glycolic acid) by weight ratio which was (1:2) and coated as a top layer with half drug dose in top layer. In addition, each layer was coated with 120 μg to 60 μg poly(L-lactide acid-co-glycolic acid) as a protection layer for underneath drug depending on the target release rate. Optionally, Sirolimus was coated on the topmost layer with poly(L-lactide acid-co-glycolic acid) by weight ratio which was (2:3).

The total target drug dose is 100 μg for Tirofiban, 100 μg for Colchicine, 100 μg for Apixaban or Rivaroxaban, 100 μg for Argatroban and 120 μg for Sirolimus for an 18 mm stent length. Drug and polymer doses are adjusted accordingly for each stent length.

A microprocessor controlled ultrasonic sprayer was used to coat each of the stents' 18 mm length uniformly with each of the drug/polymer matrix solution. After coating, the stents with basecoat were placed in a 70° C. oven for about half hours to remove the solvent; the stents with topcoat were placed in a 70° C. oven for about 2 hours to remove the solvent. The stents were then mounted on balloon catheters and crimped.

### Example 44: Preparation of Tirofiban/Colchicine/Anticoagulant1/Anticoagulant2/mTOR Eluting with mTOR on the Top Coating

Poly(L-lactide acid-co-glycolic acid) polymer dissolved into dichloromethane at room temperature and vortex until the polymer uniformly dissolved/dispersed. Tirofiban hydrochloride, Colchicine and anticoagulant (Apixaban or Rivaroxaban and Argatroban) were placed in a vial and dissolved in dichloromethane or dichloromethane/Methanol/ethanol at room temperature and vortex until all the drug was uniformly dissolved/dispersed.

Each polymer solution and each drug (or combined drugs) solutions were combined together (Tirofiban hydrochloride and Colchicine and Rivaroxaban or Apixaban and Argatroban were combined together in the ratio of (1:1:1:1) and are combined with poly(L-lactide acid-co-glycolic acid) by weight ratio which was (1:2) and coated as a base coat (drug/polymer matrix as base coat). In addition, Sirolimus was dissolved in dichloromethane and was combined with poly(L-lactide acid-co-glycolic acid) by weight ratio which was (2:3) and coated as a top layer (drug/polymer matrix as top layer). Optionally, the middle layer coated with 120 μg to 60 μg poly(L-lactide acid-co-glycolic acid) as a protection layer for underneath drug depending on the target release rate.

The total target drug dose is 100 μg for Tirotiban, 100 μg for Colchicine, 100 μg for Apixaban or Rivaroxaban, 100 μg for Argatroban and 120 μg for Sirolimus for an 18 mm stent length. Drug and polymer doses are adjusted accordingly for each stent length.

A microprocessor controlled ultrasonic sprayer was used to coat each of the stents' 18 mm length uniformly with each of the drug/polymer matrix solution. After coating, the stents with basecoat were placed in a 70° C. oven for about half an hour to remove the solvent; the stents with topcoat were placed in a 70° C. oven for about 2 hours to remove the solvent. The stents were then mounted on balloon catheters and crimped.

### Example 45: Preparation of Colchicine/Anti-Platelet/Anticoagulant1/Anticoagulant2/mTOR Eluting Stents with Four Layers Coating

Poly(L-lactide acid-co-glycolic acid) polymer was dissolved into dichloromethane at room temperature and vortex until the polymer had uniformly dissolved/dispersed. Colchicine, Tirofiban and Sirolimus and anticoagulants (Apixaban or Rivaroxaban and Argatroban) were placed in a vial and dissolved in dichloromethane or dichloromethane/Methanol/Ethanol at room temperature and vortex until all the drug was uniformly dissolved/dispersed.

Each polymer solution and each drug (or combined drugs) solutions were combined together (Colchicine and Tirofiban and Rivaroxaban and Argatroban were combined together in the ratio of (1:1:1:1) and were combined with poly(L-lactide acid-co-glycolic acid) by weight ratio which was (1:2) and coated as a base drug coat (drug/polymer matrix as base coat). In addition. Colchicine and Tirofiban and Rivaroxaban and Argatroban and Sirolimus were combined together in the ratio of (5:5:5:5:12) and were combined with poly(L-lactide acid-co-glycolic acid) by weight ratio which was (1:2) and coated as a top drug coat, by weight of 50 μg Colchicine and 50 μg Tirofiban and 50 μg Rivaroxaban and 50 μg Argatroban, and 400 μg poly(L-lactide acid-co-glycolic acid) mixed together and coated as base drug coat; and by weight of 50 μg Colchicine and 50 μg Tirofiban and 50 μg Rivaroxaban and 50 μg Argatroban and 120 μg Sirolimus, and 640 μg poly(L-lactide acid-co-glycolic acid) mixed together and coated as top drug layer coat, for a total target drug dose of 100 μg for Colchicine, 100 μg for Tirofiban, 100 μg for each anticoagulant and 120 μg for Sirolimus for a 18 mm stent length. Drug and polymer doses are adjusted accordingly for each stent length. Optionally, each layer was coated with 60 μg or 120 μg of poly(L-lactide acid-co-glycolic acid) before the additional drug layer depending on the target drug release profiles. In addition, optionally, all the drug except Sirolimus could be coated in base layer with Sirolimus on the top layer.

A microprocessor controlled ultrasonic sprayer was used to coat each of the stents' 18 mm length uniformly with each of the drug/polymer matrix solutions. After coating, the stents with basecoat were placed in a 70° C. oven for about half an hour to remove the solvent; the stents with topcoat were placed in a 70° C. oven for about 2 hours to remove the solvent. The stents were then mounted on balloon catheters and crimped.

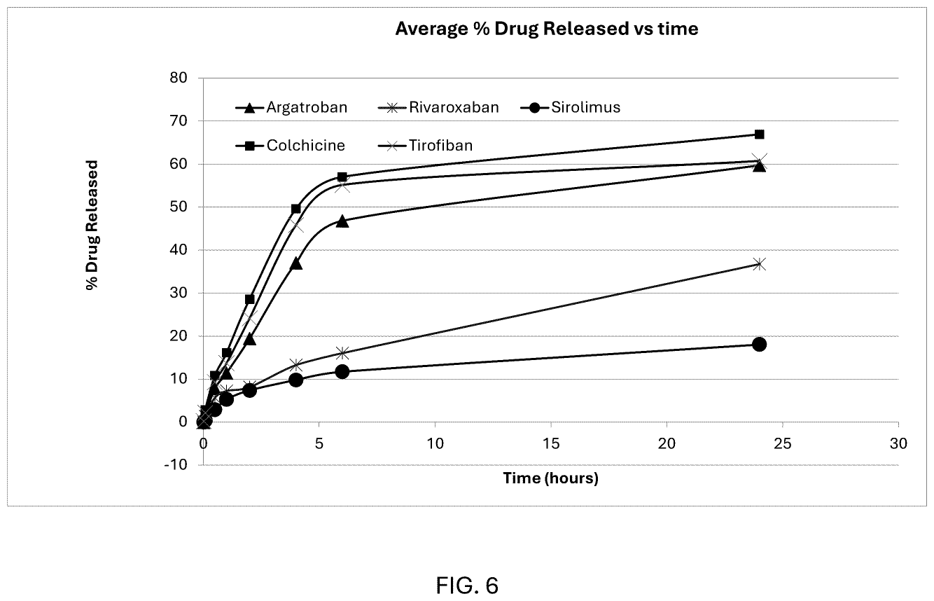

In-Vitro Elution evaluation of the stent coated above with Colchicine/anti-platelet/Anticoagulant 1/Anticoagulant2mTOR was performed in 0.01M phosphate buffered saline at 37° C. water shaking bath then tested on High Performance Liquid Chromatography (HPLC). The percentage of drug eluted was calculated during each time point as shown in FIG. 6 with 60 μg of poly(L-lactide acid-co-glycolic acid) as topcoat.

FIG. 6 demonstrated that despite the multi drug combinations, a controlled drug release of drug combination was achieved by having two or more drug matrices separated by a polymer layer and optionally as in this example a top polymer layer.

### Example 46: Preparation of Colchicine/Anti-Platelet/Anticoagulant1/Anticoagulant2/mTOR Eluting Stents with Two Layers Coating

Poly(L-lactide acid-co-glycolic acid) polymer was dissolved into dichloromethane at room temperature and vortex until the polymer had uniformly dissolved/dispersed. Colchicine, Tirofiban and Sirolimus and anticoagulants (Apixaban or Rivaroxaban and Argatroban) were placed in a vial and dissolved in dichloromethane or dichloromethane/Methanol/Ethanol at room temperature and vortex until all the drug was uniformly dissolved/dispersed.

Each polymer solution and each drug (or combined drugs) solutions were combined together (Colchicine and Tirofiban and Rivaroxaban and Argatroban and Sirolimus were combined together in the ratio of (5:5:5:5:6) and were combined with poly(L-lactide acid-co-glycolic acid) by weight ratio which was (2:3) and coated as a base drug coat (drug/polymer matrix as base coat); by weight of 100 μg Colchicine and 100 μg Tirofiban and 100 μg Rivaroxaban and 100 μg Argatroban and 120 μg Sirolimus, and 780 μg poly(L-lactide acid-co-glycolic acid) mixed together and coated as base drug coat, for a total target drug dose of 100 μg for Colchicine, 100 μg for Tirofiban, 100 μg for each anticoagulant and 120 μg for Sirolimus for a 18 mm stent length. Drug and polymer doses are adjusted accordingly for each stent length. In addition, poly(L-lactide acid-co-glycolic acid) was coated as a topcoat, by weight of 660 μg to 240 ug of poly(L-lactide acid-co-glycolic acid).

A microprocessor controlled ultrasonic sprayer was used to coat each of the stents' 18 mm length uniformly with each of the drug/polymer matrix solutions. After coating, the stents with basecoat were placed in a 70° C. oven for about half an hour to remove the solvent; the stents with topcoat were placed in a 70° C. oven for about 2 hours to remove the solvent. The stents were then mounted on balloon catheters and crimped.

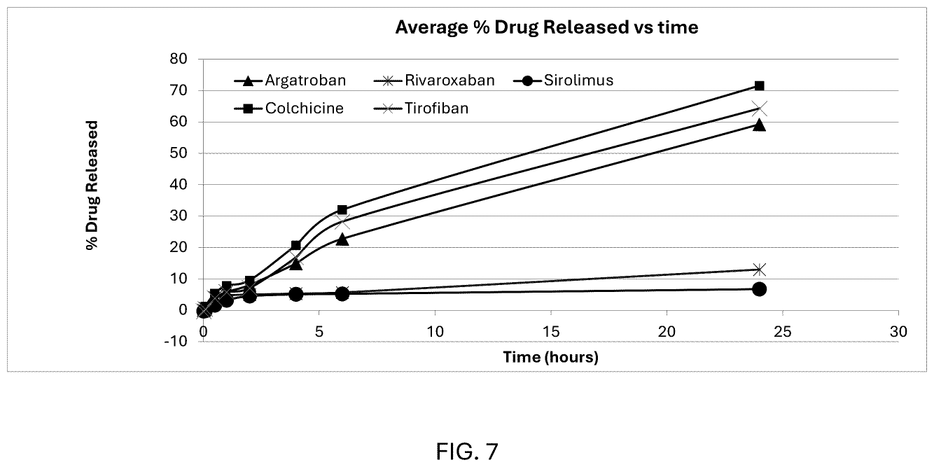

In-Vitro Elution evaluation of the stent coated above with Colchicine/anti-platelet/Anticoagulant1/Anticoagulant2/mTOR was performed in 0.01M phosphate buffered saline at 370 C water shaking bath then tested on High Performance Liquid Chromatography (HPLC). The percentage of drug eluted was calculated during each time point as shown in FIG. 7 with 60 μg of poly(L-lactide acid-co-glycolic acid) as topcoat.

FIG. 7 demonstrated, despite the multi drug combinations, a controlled drug release of drug combination was achieved by having one drug matrix with optionally as in this example a top polymeric materials layer.

### Example 47: Preparation of Colchicine/Anti-Platelet/Anticoagulant1/Anticoagulant2/mTOR Eluting Stents with Four Layers Coating with mTOR on the Topcoat

Poly(L-lactide acid-co-glycolic acid) polymer was dissolved into dichloromethane at room temperature and vortex until the polymer had uniformly dissolved/dispersed. Colchicine, Tirofiban and Sirolimus and anticoagulants (Apixaban or Rivaroxaban and Argatroban) were placed in a vial and dissolved in dichloromethane or dichloromethane/Methanol/Ethanol at room temperature and vortex until all the drug was uniformly dissolved/dispersed.

Each polymer solution and each drug (or combined drugs) solutions were combined together (Colchicine and Tirofiban and Rivaroxaban and Argatroban were combined together in the ratio of (1:1:1:1) and were combined with poly(L-lactide acid-co-glycolic acid), by weight of 50 μg Colchicine and 50 μg Tirofiban and 50 μg Rivaroxaban and 50 μg Argatroban, and 400 μg poly(L-lactide acid-co-glycolic acid) mixed together and coated as base drug coat; then 120 μg of poly(L-lactide acid-co-glycolic acid) coated as 2^{nd} layer. The 3^{rd} layer coated with by weight of 50 μg Colchicine and 50 μg Tirofiban and 50 μg Rivaroxaban and 50 μg Argatroban, and 400 μg poly(L-lactide acid-co-glycolic acid) mixed. The top layer was coated with 120 μg Sirolimus and 180 μg of poly(L-lactide acid-co-glycolic acid) mixed. The target drug dose comprised of 100 μg for Colchicine, 100 μg for Tirofiban, 100 μg for each anticoagulant and 120 μg for Sirolimus for an 18 mm stent length. Drug and polymer doses are adjusted accordingly for each stent length.

A microprocessor controlled ultrasonic sprayer was used to coat each of the stents' 18 mm length uniformly with each of the drug/polymer matrix solutions. After coating, the stents with basecoat were placed in a 70° C. oven for about half an hour to remove the solvent; the stents with topcoat were placed in a 70° C. oven for about 2 hours to remove the solvent. The stents were then mounted on balloon catheters and crimped.

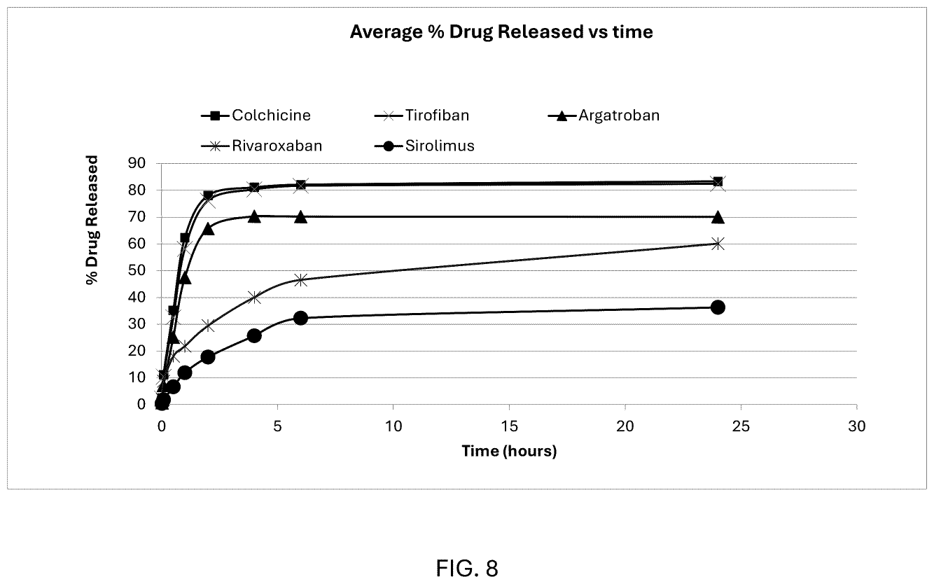

In-Vitro Elution evaluation of the stent coated above with Colchicine/anti-platelet/Anticoagulant1/Anticoagulant2/mTOR was performed in 0.01M phosphate buffered saline at 37° C. water shaking bath then tested on High Performance Liquid Chromatography (HPLC). The percentage of drug eluted was calculated during each time point as shown in FIG. 8.

FIG. 8 demonstrated despite the multi drug combinations, a controlled drug release of drug combination was achieved by having two or more drug matrices separated by a polymer layer and with anti-proliferative agent being on the top polymer layer as a matrix as in this example.

### Example 48: Preparation of Colchicine/Anti-Platelet/Anticoagulant1/Anticoagulant2/mTOR Eluting Stents with Three Layers Coating with mTOR on the Top Layer

Poly(L-lactide acid-co-glycolic acid) polymer was dissolved into dichloromethane at room temperature and vortex until the polymer had uniformly dissolved/dispersed. Colchicine, Tirofiban and Sirolimus and anticoagulants (Apixaban or Rivaroxaban and Argatroban) were placed in a vial and dissolved in dichloromethane or dichloromethane/Methanol/Ethanol at room temperature and vortex until all the drug was uniformly dissolved/dispersed.

Each polymer solution and each drug (or combined drugs) solutions were combined together (Colchicine and Tirofiban and Rivaroxaban and Argatroban were combined together in the ratio of (1:1:1:1) and were combined with poly(L-lactide acid-co-glycolic acid), by weight of 100 μg Colchicine and 100 μg Tirofiban and 100 μg Rivaroxaban and 100 μg Argatroban, and 800 μg poly(L-lactide acid-co-glycolic acid) mixed together and coated as base drug coat; then 120 μg of poly(L-lactide acid-co-glycolic acid) coated as 2^{nd} layer. The 3^{rd} layer was coated with 120 μg Sirolimus and 180 μg of poly(L-lactide acid-co-glycolic acid) mixed. The target drug dose comprised of 100 μg for Colchicine, 100 μg for Tirofiban, 100 μg for each anticoagulant and 120 μg for Sirolimus for an 18 mm stent length. Drug and polymer doses are adjusted accordingly for each stent length.

A microprocessor controlled ultrasonic sprayer was used to coat each of the stents' 18 mm length uniformly with each of the drug/polymer matrix solutions. After coating, the stents with basecoat were placed in a 70° C. oven for about half an hour to remove the solvent; the stents with topcoat were placed in a 70° C. oven for about 2 hours to remove the solvent. The stents were then mounted on balloon catheters and crimped.

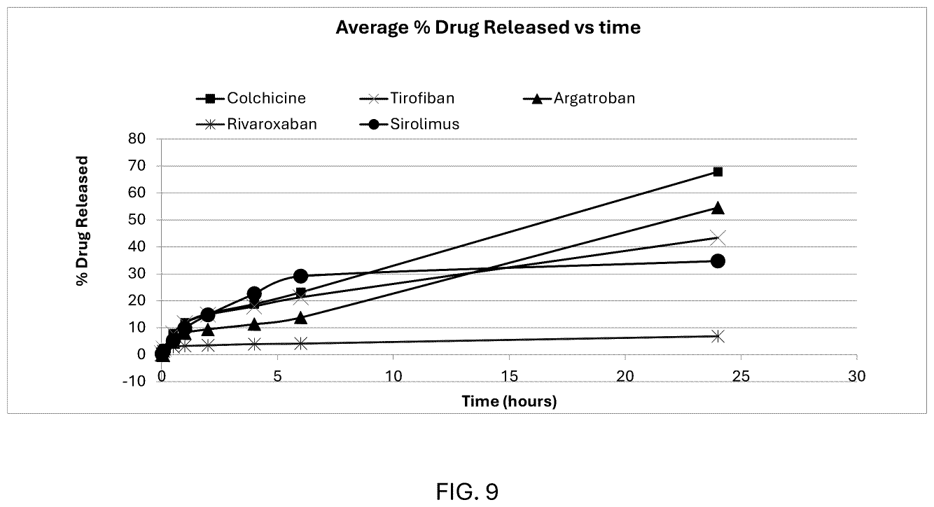

In-Vitro Elution evaluation of the stent coated above with Colchicine/anti-platelet/Anticoagulant1/Anticoagulant2/mTOR was performed in 0.01M phosphate buffered saline at 37° C. water shaking bath then tested on High Performance Liquid Chromatography (HPLC). The percentage of drug eluted was calculated during each time point as shown in FIG. 9.

FIG. 9 demonstrated that despite the multi drug combinations, a controlled drug release of drug combination was achieved by having one drug matrix except an anti-proliferative agent on the top of a polymeric material layer. The data shows that an extended release of drugs with 24 hours in the studied dose, indicating an effective level to inhibition in local clot formation.

### Example 49: Preparation of Colchicine/Tirofiban/Anticoagulant1/Anticoagulant2/Ticagrelor/Aspirin/mTOR Eluting Stents with mTOR on the Top Coating

Poly(L-lactide acid-co-glycolic acid) polymer was dissolved into dichloromethane at room temperature and vortex until the polymer had uniformly dissolved/dispersed. Colchicine and Tirofiban and Ticagelor and Sirolimus and anticoagulants (Apixaban or Rivaroxaban and Argatroban) were placed in a vial and dissolved in dichloromethane or dichloromethane/Methanol at room temperature; Aspirin was dissolve in water then diluted with ethanol, finally mixed with above drug solution and vortex until all the drug was uniformly dissolved/dispersed. Sirolimus was dissolve in dichloromethane at room temperature and vortex until the drug was uniformly dissolved/dispersed.

Each polymer solution and each drug (or combined drugs) solutions were combined together (Colchicine and Tirofiban and Rivaroxaban and Argatroban and Ticagrelor and Aspirin were combined together in the ratio of (1:1:1:1:1) and were combined with poly(L-lactide acid-co-glycolic acid) by weight ratio which was (1:2) and coated as a base drug coat (drug/polymer matrix as base drug coat. In addition, poly(L-lactide acid-co-glycolic acid) was coated as a 2w drug layer, and Sirolimus was combined together with poly(L-lactide acid-co-glycolic acid) by weight ratio which was (2:3) and coated as a top drug layer, by weight of 75 μg Colchicine and 75 μg Tirofiban and 75 μg Rivaroxaban and 75 μg Argatroban and 75 μg Ticagrelor and 75 μg Aspirin and 900 μg poly(L-lactide acid-co-glycolic acid) mixed together and coated as base drug coat; and by weight of 120 μg Sirolimus and 180 μg poly(L-lactide acid-co-glycolic acid) mixed together and coated as a top layer coat, for a total target drug dose of 75 μg for Colchicine, 75 μg for Tirofiban, 75 μg for each anticoagulant and 75 μg Ticagrelor and 75 μg Aspirin and 120 μg for Sirolimus for a 18 mm stent length. Drug and polymer doses are adjusted accordingly for each stent length. Optionally, each layer was top coated with 60 μg or 120 μg of poly(L-lactide acid-co-glycolic acid) before the additional drug layer depending on the target drug release profiles.

A microprocessor controlled ultrasonic sprayer was used to coat each of the stents' 18 mm length uniformly with each of the drug/polymer matrix solutions. After coating, the stents with basecoat were placed in a 70° C. oven for about half an hour to remove the solvent; the stents with topcoat were placed in a 70° C. oven for about 2 hours to remove the solvent. The stents were then mounted on balloon catheters and crimped.

### Example 50 Preparation of Direct Factor Xa Inhibitor Rivaroxaban, Direct Factor IIa Inhibitor Argatroban, Glycoprotein IIb/IIIa Inhibitor Tirofiban, P2Y12 Inhibitor Ticagrelor and Anti Inflammatory Colchicine and/or mTOR Coated Balloon with Excipient

The balloon can optionally adopt carrier excipient to coat to facilitate drug transfer to the vessel wall and control release rate. A variety of carrier excipients and techniques can be used. The selected excipient could be contrast agent (i.e., iopromide), urea, dextrane, shellac, shelloic acid, keratosis (a naturally derived protein), Plasticizer (i.e. butyryl-tri-hexyl citrate, acetyl tributyl citrate, citrate ester, glycerol, other organic ester), hydrophilic space, Polyvinylpyrrolidone (PVP) and its hydrogels, Surfactants, Non-ionic surfactant Polysorbate/sorbitol (i.e. Tween20, Tween60 or Tween80), nordihydroguaiaretic acid (NDGA), excipient such as phospholipid, amphiphilic polymer such as Poly(ethylene glycol) (i.e., PEG 8000), poly(ethylene oxide) (PEO) (molecular weight range from 100,000 to 10,000,000), Polyethylenimine (PEI) or polyaziridine linear or branched, amphiphilic block co-polymers composed of poly(ethylene oxide) (PEO) as the hydrophilic block and poly(ether)s, poly(amino acid)s), hydrophobic polymer space, biodegradable polymers such as Poly DL lactide-co-glycolide, Poly L Lactide-co-caprolactone, durable polymers, individually or combinations thereof.

A balloon made of a biodegradable or non-degradable polymeric material (e.g., a nylon) and having openings (e.g., pores, holes, etc.) in the body and/or at the surface of the balloon was used. The drug was selected from a mixture containing a mTOR inhibitors, direct factor Xa inhibitor Rivaroxaban, direct factor IIa inhibitor Argatroban, glycoprotein IIb/IIIa inhibitor Tirofiban, P2Y12 inhibitor Ticagrelor and anti-inflammatory Colchicine. The mTOR inhibitors was selected from Sirolimus, Novolimus, temsirolimus, zotarolimus and everolimus etc. The anticoagulants were selected from Apixaban, Argatraban, rivaroxaban or a derivative thereof, and/or a fibrinolytic or thrombolytic agent, such as a plasminogen activator) individually or combinations thereof. For example, Siroliums and direct factor Xa inhibitor Rivaroxaban, direct factor IIa inhibitor Argatroban, glycoprotein IIb/IIIa inhibitor Tirofiban, P2Y12 inhibitor Ticagrelor and anti-inflammatory Colchicine were placed in a vial and dissolved in dichloromethane or dichloromethane/Methanol/Ethanol combination at 2 to 10 mg/ml. The carrier excipient was dissolved in a proper solvent. The solution and drug solutions were combined at a target ratio of 3 to 1, 1 to 1, 2 to 1, or 1 to 3 ratios according to the target drug loading. Further dilution with dichloromethane was conducted if needed. Optionally anti-solvent was used to control the coating morphology of particles and drug release rate.

Optionally the balloon can undergo physical surface treatment before coated such that the surface has microspores, micro-holes, or chemical surface treatment before coated such that the balloon materials have photo-link or other chemical function group that can be reacted under UV or other techniques to easy coating. After surface treatment, the coating can be spray coat or dip coat or use other coating techniques (i.e., 3D printer).

The coating can optionally cover any surfaces (e.g., the exterior surface, any other surfaces, or all surfaces) of the balloon. The coating solution can be homogeneous or non-homogeneous such as suspension or emulsions.

The coated balloon can optionally combine multi-strategies (e.g., electrospinning, plasma treatment, Layer-by-Layer Self-Assembly or a combination thereof) to form finely controlling structural, mechanical, and surface properties. With tuned coating techniques and solvent removal techniques, the produces powder particles can be optionally homogenous, porous, and uniform in size and shape. The morphology of particles could be micro-crystalline, nanoparticles, Nano-encapsulated to provide release rate control.

When spray coating, a microprocessor controlled ultrasonic sprayer was used to apply the drug containing drug solution to cover any surface of a balloon. A mandrel was placed through catheter tips and underneath an ultrasonic spray nozzle (Micromist System with Ultrasonic Atomizing Nozzle Sprayer, Sono-Tek, N.Y.), which was rotating at 80 rpm and moved longitudinally at a rate of 0.050 inches/minute. The coating parameter can optionally adjust to ideal coating texture and the morphology and the profile of the interface between drug and balloon surface.

After coating, the balloon was placed in a vacuum chamber to remove the residue solvent. Optionally the coated balloon can be tri-folded to protect coated drug with a folded and/or wrapped balloon thereon to a pre-annealing step to induce a fold/wrap memory in the resulting pre-annealed balloon and/or coated balloon has a protector which need to peel off before use.

The balloon catheter was then inserted in a coil and packaged. The pouch was sterilized by Ethylene oxide or E-beam. The pouch was further packaged in a foil pouch with oxygen scavengers and nitrogen purge and vacuum sealed.

The balloon (e.g., the balloon of a balloon-catheter or a balloon-delivery system) was advanced to a site to be treated (e.g., an occluded or weakened section of a blood vessel to be opened up or supported by a balloon) in the body of a subject and was inflated to a desired inflation diameter. Before, during and/or after inflation of the balloon, the balloon releases the fibrin/thrombus formation-inhibiting or fibrin/thrombus dissolution-promoting agent and the optional other kind of bioactive agent (e.g., an anti-proliferative agent or an anti-inflammatory agent) to, into or at the treatment site, or to, into or at an area adjacent thereto, by any suitable mechanism (e.g., concentration gradient, diffusion, pressure or mechanical force, or a combination thereof).

### Example 51 Preparation and Use of Direct Factor Xa Inhibitor Rivaroxaban, Direct Factor IIa Inhibitor Argatroban, Glycoprotein IIb/IIIa Inhibitor Tirofiban, P2Y12 Inhibitor Ticagrelor and Anti-Inflammatory Colchicine and/or mTOR Coated Balloon-Catheter

A first mixture containing a fibrin/thrombus formation-inhibiting or fibrin/thrombus dissolution-promoting agent (e.g., an anticoagulant, such as direct factor Xa inhibitor Rivaroxaban, direct factor IIa inhibitor Argatroban, or a derivative thereof, an antiplatelet such as glycoprotein IIb/IIIa inhibitor Tirofiban, P2Y12 inhibitor Ticagrelor or a fibrinolytic or thrombolytic agent, such as a plasminogen activator) and a solvent, and optionally a biodegradable or non-degradable polymeric material and/or another kind of bioactive agent (e.g., an anti-proliferative agent, such as rapamycin or a derivative thereof, or an anti-inflammatory agent colchicine), is applied (e.g., by spraying or dipping) to the balloon portion and/or the catheter portion of a balloon-catheter made of a biodegradable or non-degradable polymeric material (e.g., a nylon) to form a first coating on the balloon portion and/or the catheter portion. The first coating can cover any surfaces (e.g., the exterior surface, any other surfaces, or all surfaces) of the balloon and/or the catheter.

Optionally, a second mixture containing another type of bioactive agent (e.g., an anti-proliferative agent, such as rapamycin or a derivative thereof, or an anti-inflammatory agent) and a solvent, and optionally a biodegradable or non-degradable polymeric material and/or a fibrin/thrombus formation-inhibiting or fibrin/thrombus dissolution-promoting agent (e.g., an anticoagulant, such as rivaroxaban or a derivative thereof, or a fibrinolytic or thrombolytic agent, such as a plasminogen activator), is applied to the balloon portion and/or the catheter portion (e.g., by spraying or dipping) to form a second coating on the balloon portion and/or the catheter portion. The optional second coating can cover any surfaces (e.g., the exterior surface, any other surfaces, or all surfaces) of the balloon and/or the catheter. The first mixture and the optional second mixture can be applied to the balloon portion and/or the catheter portion in any order.

Optionally, a third mixture containing a biodegradable or non-degradable polymeric material and a solvent is applied to the balloon portion and/or the catheter portion (e.g., by spraying or dipping) to form a third coating over the first coating and the optional second coating. The optional third coating can be, e.g., a top layer or coat or a diffusion barrier that controls release of the fibrin/thrombus formation-inhibiting or fibrin/thrombus dissolution-promoting agent and the optional other kind of bioactive agent from the first coating and the optional second coating. The optional third coating can cover the exterior surface or any other surfaces, or all surfaces, of the balloon and/or the catheter.

The coated balloon-catheter is optionally heated to stabilize the coating(s) and is placed in a container (e.g., a pouch) and sterilized (e.g., by exposure to e-beam radiation).

The balloon-catheter (e.g., the balloon-catheter of a stent-delivery system) is advanced to a site to be treated (e.g., an occluded or weakened section of a blood vessel to be opened up or supported by a stent) in the body of a subject, and the balloon is inflated to a desired inflation diameter. Before, during and/or after inflation of the balloon, the balloon portion and/or the catheter portion of the balloon-catheter releases the fibrin/thrombus formation-inhibiting or fibrin/thrombus dissolution-promoting agent and the optional other kind of bioactive agent (e.g., an anti-proliferative agent or an anti-inflammatory agent) to, into or at the treatment site, or to, into or at an area adjacent thereto, by any suitable mechanism (e.g., concentration gradient, diffusion, pressure or mechanical force, or a combination thereof).

### Example 52: Preparation and Use of Direct Factor Xa Inhibitor Rivaroxaban, Direct Factor IIa Inhibitor Argatroban, Glycoprotein IIb/IIIa Inhibitor Tirofiban, P2Y12 Inhibitor Ticagrelor and Anti-Inflammatory Colchicine and/or mTOR Impregnated Balloon

A balloon made of a biodegradable or non-degradable polymeric material (e.g., a nylon) and having openings (e.g., pores, holes, etc.) in the body and/or at the surface of the balloon is immersed in a mixture containing a fibrin/thrombus formation-inhibiting or fibrin/thrombus dissolution-promoting agent (e.g., direct factor Xa inhibitor Rivaroxaban, direct factor IIa inhibitor Argatroban, or a derivative thereof, or a fibrinolytic or thrombolytic agent, such as a plasminogen activator) and a solvent, and optionally another kind of bioactive agent (e.g., an anti-proliferative agent, such as rapamycin or a derivative thereof, or an anti-inflammatory agent colchicine). The balloon can optionally have one or more coatings comprising a fibrin/thrombus formation-inhibiting or fibrin/thrombus dissolution-promoting agent and/or another kind of bioactive agent and can optionally have a coating comprising no bioactive agent.

The balloon (e.g., the balloon of a balloon-catheter of a stent-delivery system) is advanced to a site to be treated (e.g., an occluded or weakened section of a blood vessel to be opened up or supported by a stent) in the body of a subject and is inflated to a desired inflation diameter. Before, during and/or after inflation of the balloon, the balloon releases the fibrin/thrombus formation-inhibiting or fibrin/thrombus dissolution-promoting agent and the optional other kind of bioactive agent (e.g., an anti-proliferative agent or an anti-inflammatory agent colchicine) to, into or at the treatment site, or to, into or at an area adjacent thereto, by any suitable mechanism (e.g., concentration gradient, diffusion, pressure or mechanical force, or a combination thereof).

### Example 53: Preparation and Use of Direct Factor Xa Inhibitor Rivaroxaban, Direct Factor IIa Inhibitor Argatroban, Glycoprotein IIb/IIIa Inhibitor Tirofiban, P2Y12 Inhibitor Ticagrelor and Anti-Inflammation Colchicine and/or mTOR Coated Surgical Instrument

A surgical instrument (e.g., a cutting instrument, such as a knife) made of a biodegradable or non-degradable metal or metal alloy (e.g., stainless steel) optionally undergoes surface treatment (e.g., microblasting) to improve adhesion of a coating (e.g., a polymeric coating) to a metal surface.

A first mixture containing a fibrin/thrombus formation-inhibiting or fibrin/thrombus dissolution-promoting agent (e.g., direct factor Xa inhibitor Rivaroxaban, direct factor IIa inhibitor Argatroban, glycoprotein IIb/IIIa inhibitor Tirofiban, P2Y12 inhibitor Ticagrelor or a derivative thereof, or a fibrinolytic or thrombolytic agent, such as a plasminogen activator) and a solvent, and optionally a biodegradable or non-degradable polymeric material and/or another kind of bioactive agent (e.g., an anti-proliferative agent, such as rapamycin or a derivative thereof, or an anti-inflammatory agent colchicine), is applied to the surgical instrument (e.g., by dipping or spraying) to form a first coating on the surgical instrument. The first coating can cover the exterior surface, any other surfaces, or all surfaces of the surgical instrument.

Optionally, a second mixture containing another kind of bioactive agent (e.g., an anti-proliferative agent, such as rapamycin or a derivative thereof, or an anti-inflammatory agent colchicine) and a solvent, and optionally a biodegradable or non-degradable polymeric material and/or a fibrin/thrombus formation-inhibiting or fibrin/thrombus dissolution-promoting agent (e.g., direct factor Xa inhibitor Rivaroxaban, direct factor IIa inhibitor Argatroban, glycoprotein IIb/IIIa inhibitor Tirofiban, P2Y12 inhibitor Ticagrelor or a derivative thereof, or a fibrinolytic or thrombolytic agent, such as a plasminogen activator), is applied to the surgical instrument (e.g., by dipping or spraying) to form a second coating on the surgical instrument. The optional second coating can cover the exterior surface, any other surfaces, or all surfaces of the surgical instrument. The first mixture and the optional second mixture can be applied to the surgical instrument in any order.

Optionally, a third mixture containing a biodegradable or non-degradable polymeric material and a solvent is applied to the surgical instrument (e.g., by dipping or spraying) to form a third coating over the first coating and the optional second coating. The optional third coating can be, e.g., a top layer or coat or a diffusion barrier that controls release of the fibrin/thrombus formation-inhibiting or fibrin/thrombus dissolution-promoting agent and the optional other kind of bioactive agent from the first coating and the optional second coating. The optional third coating can cover the exterior surface, any other surfaces, or all surfaces of the surgical instrument.

The coated surgical instrument is optionally heated to stabilize the coating(s) and is placed in a container (e.g., a pouch) and sterilized (e.g., by exposure to e-beam radiation).

The surgical instrument (e.g., a cutting instrument, such as a knife) is advanced to a site in the body of a subject undergoing a surgery or intervention (e.g., a tissue to be cut or treated). Before, while and/or after the surgical instrument is positioned at the treatment site or at an area adjacent thereto, or before, while and/or after the surgical instrument contacts the tissue to be treated, the surgical instrument releases the fibrin/thrombus formation-inhibiting or fibrin/thrombus dissolution-promoting agent and the optional other kind of bioactive agent (e.g., an anti-proliferative agent or an anti-inflammatory agent such as colchicine) to, into or at the treatment site, or to, into or at an area adjacent thereto, by any suitable mechanism (e.g., concentration gradient, diffusion, pressure or mechanical force, or a combination thereof).

### Example 54: Preparation and Use of Direct Factor Xa Inhibitor Rivaroxaban, Direct Factor IIa Inhibitor Argatroban, Glycoprotein IIb/IIIa Inhibitor Tirofiban, P2Y12 Inhibitor Ticagrelor and Anti-Inflammatory Colchicine and/or mTOR Impregnated Surgical Instrument

A surgical instrument made of a biodegradable or non-degradable metal or metal alloy (e.g., stainless steel) and having openings (e.g., pores, holes, etc.) in the body and/or at the surface of the surgical instrument is immersed in a mixture containing a fibrin/thrombus formation-inhibiting or fibrin/thrombus dissolution-promoting agent (e.g., direct factor Xa inhibitor Rivaroxaban, direct factor IIa inhibitor Argatroban, glycoprotein IIb/IIIa inhibitor Tirofiban, P2Y12 inhibitor Ticagrelor or a derivative thereof, or a fibrinolytic or thrombolytic agent, such as a plasminogen activator) and a solvent, and optionally another kind of bioactive agent (e.g., an anti-proliferative agent, such as rapamycin or a derivative thereof, or an anti-inflammatory agent such as colchicine). The surgical instrument can optionally undergo surface treatment, can optionally have one or more coatings comprising a fibrin/thrombus formation-inhibiting or fibrin/thrombus dissolution-promoting agent and/or another kind of bioactive agent, and can optionally have a coating comprising no bioactive agent.

The surgical instrument (e.g., a cutting instrument, such as a knife) is advanced to a site in the body of a subject undergoing a surgery or intervention (e.g., a tissue to be cut or treated). Before, while and/or after the surgical instrument is positioned at the treatment site or at an area adjacent thereto, or before, while and/or after the surgical instrument contacts the tissue to be treated, the surgical instrument releases the fibrin/thrombus formation-inhibiting or fibrin/thrombus dissolution-promoting agent and the optional other kind of bioactive agent (e.g., an anti-proliferative agent or an anti-inflammatory agent such as colchicine) to, into or at the treatment site, or to, into or at an area adjacent thereto, by any suitable mechanism (e.g., concentration gradient, diffusion, pressure or mechanical force, or a combination thereof).

### Example 55: Use of Direct Factor Xa Inhibitor Rivaroxaban, Direct Factor IIa Inhibitor Argatroban, Glycoprotein IIb/IIIa Inhibitor Tirofiban, P2Y12 Inhibitor Ticagrelor and Anti-Inflammatory Colchicine and/or mTOR Delivering Infusion Surgical Instrument

An infusion surgical instrument [e.g., a cutting instrument (e.g., a knife) or an injection device (e.g., a needle)] contains one or more lumens for delivering one or more drugs. The surgical instrument is advanced to a site in or on the body of a subject undergoing a surgery or intervention (e.g., a tissue to be cut or treated). Before, while and/or after the surgical instrument is positioned at the treatment site or at an area adjacent thereto, or before, while and/or after the surgical instrument contacts the tissue to be treated, a first mixture containing a fibrin/thrombus formation-inhibiting or fibrin/thrombus dissolution-promoting agent (e.g., direct factor Xa inhibitor Rivaroxaban, direct factor IIa inhibitor Argatroban, glycoprotein IIb/IIIa inhibitor Tirofiban, P2Y12 inhibitor Ticagrelor or a derivative thereof, or a fibrinolytic or thrombolytic agent, such as a plasminogen activator) and a solvent (e.g., saline), and optionally another kind of bioactive agent (e.g., an anti-proliferative agent, such as rapamycin or a derivative thereof, or an anti-inflammatory agent such as colchicine), is injected through one or more drug-delivering lumens of the surgical instrument. Before, while and/or after the surgical instrument is positioned at the treatment site or at an area adjacent thereto, or before, while and/or after the surgical instrument contacts the tissue to be treated, a second mixture containing another kind of bioactive agent (e.g., an anti-proliferative agent or an anti-inflammatory agent such as colchicine) and a solvent (e.g., saline), and optionally a fibrin/thrombus formation-inhibiting or fibrin/thrombus dissolution-promoting agent, optionally is injected through one or more drug-delivering lumens of the surgical instrument. The surgical instrument delivers the fibrin/thrombus formation-inhibiting or fibrin/thrombus dissolution-promoting agent and the optional other kind of bioactive agent (e.g., an anti-proliferative agent or an anti-inflammatory agent such as colchicine) to, into or at the treatment site, or to, into or at an area adjacent thereto.

### Example 56: Preparation of Direct Factor Xa Inhibitor Rivaroxaban, Direct Factor IIa Inhibitor Argatroban, Glycoprotein IIb/IIIa Inhibitor Tirofiban, P2Y12 Inhibitor Ticagrelor and Anti-Inflammatory Colchicine and/or mTOR Coated Implanted Delivery System

An implanted delivery system can have a polymer coating containing direct factor Xa inhibitor Rivaroxaban, direct factor IIa inhibitor Argatroban, glycoprotein IIb/IIIa inhibitor Tirofiban, P2Y12 inhibitor Ticagrelor and anti-inflammatory Colchicine and/or a combination with mTOR on top, part of, and/or adjacent to the implant made from polymer selected from non-degradable polymers.

The implant can be infused with a polymer coating in a solvent solution with direct factor Xa inhibitor Rivaroxaban, direct factor IIa inhibitor Argatroban, glycoprotein IIb/IIIa inhibitor Tirofiban, P2Y12 inhibitor Ticagrelor and anti-inflammatory Colchicine and/or a combination with mTOR such as rapamycin, everolimus, biolimus, temsirolimus, ridaforolimus, zotarolimus, myolimus and novolimus drug solution into the said implant and said solvent can evaporate leaving either polymer coating or drug in the pores of the implant.

Poly (L-lactide acid-co-glycolic acid) polymer is dissolved into dichloromethane at room temperature and vortex until the polymer is uniformly dissolved/dispersed. Anticoagulant (Apixaban or Rivaroxaban & Argatroban) is placed in a vial and dissolved in dichloromethane/Methanol at room temperature and vortex until all the drug is uniformly dissolved/dispersed.

Each polymer solution and each drug solutions is combined direct factor Xa inhibitor Rivaroxaban, direct factor IIa inhibitor Argatroban, glycoprotein IIb/IIIa inhibitor Tirofiban, P2Y12 inhibitor Ticagrelor and anti-inflammatory Colchicine and/or mTOR with weight ratio 1 to 1 or other ratio) to poly (L-lactide acid-co-glycolic acid) by weight ratio was 3:1 or other ratio according to the target drug dose.

The implant can optionally undergo surface treatment if the surface is not porous (i.e., plasma treatment or other friction surface treatment). After surface treatment, the coating could be spray coated or dip coated. The coating can be inside the implant or dipped onto the implant or coated on the implant.

When spray coated, a microprocessor controlled ultrasonic sprayer is used to coat the implant containing drug/excipient solution to the entire surface of the implant. After coating, the implant is placed in a 70° C. oven for about 2 hours to remove the residue solvent. The implant is then mounted on the delivery catheter and then is inserted in a coil and packaged. The pouches were sterilized.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1.-43. (canceled)

44. A composition for coupling to a surface of an implantable article, the composition comprising:
an anti-coagulant comprising a direct factor Xa inhibitor and at least one of (a) an anti-platelet drug or (b) an anti-inflammatory drug comprising colchicine.

45. The composition of claim 44, comprising both (a) the anti-platelet drug, and (b) the anti-inflammatory drug comprising colchicine.

46. The composition of claim 44, wherein the direct factor Xa inhibitor comprises at least one drug selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), eribaxaban (PD 0348292), 2-(5-carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052), and a salt, isomer, solvate, derivative, metabolite, or prodrug of any preceding member of the group.

47. The composition of claim 46, wherein the direct factor Xa inhibitor comprises rivaroxaban, apixaban, or a salt, isomer, solvate, derivative, metabolite, or prodrug thereof.

48. The composition of claim 44, wherein the anti-platelet drug is selected from the group consisting of:
a Cox 1 inhibitor or a salt, isomer, solvate, derivative, metabolite, or prodrug thereof;
a Cox 2 inhibitor or a salt, isomer, solvate, derivative, metabolite, or prodrug thereof;
a glycoprotein IIb/IIIa inhibitor or a salt, isomer, solvate, derivative, metabolite, or prodrug thereof; and
a P2Y12 inhibitor or a salt, isomer, solvate, derivative, metabolite, or prodrug thereof.

49. The composition of claim 48, wherein the anti-platelet drug comprises: tirofiban, or a salt, isomer, solvate, derivative, metabolite, or prodrug thereof; or ticagrelor, or a salt, isomer, solvate, derivative, metabolite, or prodrug thereof.

50. The composition of claim 48, wherein the anti-platelet drug comprises the Cox 1 inhibitor or a salt, isomer, solvate, derivative, metabolite, or prodrug thereof or the Cox 2 inhibitor or a salt, isomer, solvate, derivative, metabolite, or prodrug thereof.

51. The composition of claim 48, wherein the anti-platelet drug comprises clopidogrel, or a salt, isomer, solvate, derivative, metabolite, or prodrug thereof.

52. The composition of claim 44, further comprising at least one direct factor IIa inhibitor selected from the group consisting of argatroban, dabigatran, ximelagatran, melagatran, efegatran, inogatran, atecegatran metoxil (AZD-0837), hirudin, hirudin analogs, bivalirudin, desirudin, and lepirudin.

53. The composition of claim 52, wherein the at least one direct factor IIa inhibitor comprises argatroban, or a salt, isomer, solvate, derivative, metabolite, or prodrug thereof.

54. A composition for coupling to a surface of an implantable article, the composition comprising:
an anti-coagulant comprising a direct factor IIa inhibitor and at least one of (a) an anti-platelet drug, or (b) an anti-inflammatory drug comprising colchicine.

55. The composition of claim 54, comprising both (a) the anti-platelet drug, and (b) the anti-inflammatory drug comprising colchicine.

56. The composition of claim 54, wherein the direct factor IIa inhibitor is selected from the group consisting of: argatroban, dabigatran, ximelagatran, melagatran, efegatran, inogatran, atecegatran metoxil (AZD-0837), hirudin, hirudin analogs, bivalirudin, desirudin, lepirudin, and a salt, isomer, solvate, derivative, metabolite, or prodrug of any preceding member of the group.

57. The composition of claim 56, wherein the direct factor IIa inhibitor comprises argatroban, or a salt, isomer, solvate, derivative, metabolite, or prodrug thereof.

58. The composition of claim 54, wherein the anti-platelet drug is selected from the group consisting of:
a Cox 1 inhibitor or a salt, isomer, solvate, derivative, metabolite, or prodrug thereof;
a Cox 2 inhibitor or a salt, isomer, solvate, derivative, metabolite, or prodrug thereof;
a glycoprotein IIb/IIIa inhibitor or a salt, isomer, solvate, derivative, metabolite, or prodrug thereof; and
a P2Y12 inhibitor or a salt, isomer, solvate, derivative, metabolite, or prodrug thereof.

59. The composition of claim 58, wherein the anti-platelet drug comprises tirofiban, or a salt, isomer, solvate, derivative, metabolite, or prodrug thereof; or ticagrelor, or a salt, isomer, solvate, derivative, metabolite, or prodrug thereof.

60. The composition of claim 58, wherein the anti-platelet drug comprises the Cox 1 inhibitor or a salt, isomer, solvate, derivative, metabolite, or prodrug thereof or the Cox 2 inhibitor or a salt, isomer, solvate, derivative, metabolite, or prodrug thereof.

61. The composition of claim 58, wherein the anti-platelet drug comprises:
clopidogrel or a salt, isomer, solvate, derivative, metabolite, or prodrug thereof; or
an active metabolite of clopidogrel or a salt, isomer, solvate, derivative, metabolite, or prodrug thereof.

62. The composition of claim 54, further comprising at least one direct factor Xa inhibitor selected from the group consisting of: apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), eribaxaban (PD 0348292), 2-(5-carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052), and a salt, isomer, solvate, derivative, metabolite, or prodrug of any preceding member of the group.

63. The composition of claim 62, wherein the direct factor Xa inhibitor comprises:
apixaban, or a salt or an analog thereof; or
rivaroxaban, or a salt, isomer, solvate, derivative, metabolite, or prodrug thereof.
